(19) United States
(12) Patent Application Publication
(10) Pub. No.: US 2025/0032646 A1
(43) Pub. Date: Jan. 30, 2025

(54) ENHANCERS FOR DIRECTED EXPRESSION OF GENES IN NEURONAL CELL POPULATIONS, COMPOSITIONS AND METHODS THEREOF

(71) Applicants: The Broad Institute, Inc., Cambridge, MA (US); President and Fellows of Harvard College, Cambridge, MA (US)

(72) Inventors: Jordane Dimidschstein, Cambridge, MA (US); Gordon Fishell, Cambridge, MA (US)

(21) Appl. No.: 18/912,335

(22) Filed: Oct. 10, 2024

Related U.S. Application Data
(63) Continuation of application No. PCT/US2023/018011, filed on Apr. 10, 2023.
(60) Provisional application No. 63/329,698, filed on Apr. 11, 2022.

(51) Int. Cl. A61K 48/00; C12N 15/86

(52) U.S. Cl. CPC A61K 48/0066; A61K 48/0058; C12N 15/86; C12N 2750/14143; C12N 2830/008

(57) ABSTRACT

Provided are isolated enhancer element sequences that regulate and restrict expression of a transgene, such as a therapeutic gene, to certain neuronal cell types and/or populations in the brain and CNS. Therapeutic virus vectors containing the cloned enhancer element sequences, particularly, recombinant adeno-associated virus (rAAV) vectors, and a transgene are described. The rAAV vectors, compositions and methods are useful for treating subjects afflicted with neuropsychiatric and neuropathological diseases, disorders and conditions and symptoms thereof. The vectors can be used to restore normal cellular function, e.g., by restoring expression of certain genes to the appropriate interneuron or neuron target cell populations, to address the root cause of the disease, e.g., by restoring the excitation-inhibition balance in the neuronal cell or cell population.

### CROSS REFERENCE TO RELATED APPLICATIONS

This application is a continuation of International Application No. PCT/US2023/018011 filed Apr. 10, 2023, which claims priority to and benefit of U.S. Provisional Application No. 63/329,698, filed on Apr. 11, 2022, the entire contents of which are incorporated by reference herein.

### STATEMENT REGARDING FEDERALLY SPONSORED RESEARCH

The invention was made with government support under grant numbers R01-MH111529 and UG3MH120096 awarded by the National Institutes of Health. The government has certain rights in the invention.

### SEQUENCE LISTING

This application contains a Sequence Listing which has been submitted electronically in XML format and is hereby incorporated by reference in its entirety. The Sequence Listing XML file, created on Oct. 18, 2023, is named “167741-024302US_SequenceListing.xml” and is 137 kilobytes in size.

### BACKGROUND

The balance between excitation and inhibition of neuronal cell activity must be carefully maintained for proper functioning of brain circuits and the activities of the neuronal cells that function within these circuits. An alteration, defect, or disruption in the activity of specific subtypes of neurons in the brain circuitry can result in a number of neurological diseases and disorders.

Abnormal or aberrant neuronal function and activity may be a consequence of a deviation from the course of neuronal development (e.g., aberrant fate specification during embryonic development due to genetic mutation) or acute insult (e.g., stroke, concussion). Aberrant neurotransmission and alterations in cortical and subcortical circuits may cause and induce the clinical features and symptoms that afflict patients having a number of serious neurological diseases and disorders. Therapeutic compositions and methods that are capable of modulating the activity of neuronal cells with specificity and sensitivity are needed and are beneficial to combat and treat the severe symptoms of devastating neurological diseases, disorders and conditions.

Approaches for understanding and treating neurological diseases and disorders, e.g., neuropsychiatric diseases and disorders, that are associated with the expression and the timing of expression of genes within certain neuronal cell types, require targeting and manipulating the neuronal cell types. Thus, gaining access to these cell populations both in human and non-human species is needed for successfully achieving treatment and therapeutics that target the affected neurons and neuronal cell populations associated with a neurological disease or disorder. Provided herein are products, compositions, methods and approaches to address and meet these needs.

### SUMMARY

Featured herein are isolated enhancer element sequences that function to restrict and regulate the expression of genes (polynucleotides encoding gene products) in certain neuronal cells or cell populations, such as neuronal, interneuronal, or cholinergic cell types and/or populations of the brain and/or central nervous system (CNS). In embodiments, the enhancer element sequences are cloned (e.g., molecularly cloned) and are components of viral vectors, e.g., recombinant adeno-associated virus (rAAV) vectors, virus particles, and compositions and methods or use thereof. The rAAV vectors contain (are molecularly engineered to contain) at least one transgene (e.g., a therapeutic gene, e.g., a gene encoding a regulatory protein or a protein, polypeptide, or peptide having a therapeutic, corrective, or treatment activity or function in a cell, a reporter gene, or an effector gene (e.g., a polynucleotide encoding the hM3Dq modified muscarinic receptor (Gq-DREADD), a polynucleotide encoding the pharmacologically selective actuator molecule (PSAM), a polynucleotide encoding a clustered regularly interspaced short palindromic repeats-Cas9 (CRISPR-Cas9) protein or variant thereof, a polynucleotide encoding a Zinc Finger Protein, a polynucleotide encoding a Transcription activator-like effector nuclease (TALEN), or engineered forms thereof, for gene editing, base editing, or gene expression modulation) for functional activity in a targeted cell (neuronal cell type), or a functional portion thereof; and a specific, isolated enhancer element sequence as described herein that directs, regulates, targets, or restricts expression of the transgene to a particular neuronal cell type or population, i.e., an intended target cell or cell population. In an embodiment, a transgene may encode an effector nuclease, e.g., Cas9 and the like, to disrupt a harmful or mutated gene, or to repair a mutated gene or polynucleotide, e.g., a gene or polynucleotide encoding a receptor protein, a channel protein, such as, for example, an ion channel or a ligand-gated ion channel, a transcription factor protein, or a disease-causing protein, and the like. It will be understood that the terms “enhancer,” “enhancer element,” “enhancer sequence,” enhancer element sequence (or polynucleotide),” and the like, are used interchangeably herein.

Provided in an aspect is an isolated, cloned enhancer element comprising a polynucleotide sequence having at least 75% identity to a polynucleotide sequence set forth in any one of SEQ ID NOs: 3, 4, 19, 20, 41, 42, 45, 46, 47, 48, 49, 50, 51, 52, 53, 54, 55, 56, 61, or 62, wherein the enhancer element regulates or restricts expression of a gene in a neuronal cell type or population is provided. In an embodiment, the enhancer element comprises a polynucleotide sequence having at least 85% identity to a polynucleotide sequence set forth in any one of SEQ ID NOs: NOs: 3, 4, 19, 20, 41, 42, 45, 46, 47, 48, 49, 50, 51, 52, 53, 54, 55, 56, 61, or 62. In an embodiment, the enhancer element comprises a polynucleotide sequence having at least 90% identity to a polynucleotide sequence set forth in any one of SEQ ID NOs: NOs: 3, 4, 19, 20, 41, 42, 45, 46, 47, 48, 49, 50, 51, 52, 53, 54, 55, 56, 61, or 62. In an embodiment, the enhancer element comprises a polynucleotide sequence having at least 95% identity to a polynucleotide sequence set forth in any one of SEQ ID NOs: NOs: 3, 4, 19, 20, 41, 42, 45, 46, 47, 48, 49, 50, 51, 52, 53, 54, 55, 56, 61, or 62. In an embodiment, the enhancer element of claim 1, comprising a polynucleotide sequence having at least 98% identity to a polynucleotide sequence set forth in any one of SEQ ID NOs: NOs: 3, 4, 19, 20, 41, 42, 45, 46, 47, 48, 49, 50, 51, 52, 53, 54, 55, 56, 61, or 62. In an embodiment, the enhancer element of claim 1, comprising or consisting of a polynucleotide sequence set forth in any one of SEQ ID NOs: NOs: 3, 4, 19, 20, 41, 42, 45, 46, 47, 48, 49, 50, 51, 52, 53, 54, 55, 56, 61, or 62. In an embodiment, the neuronal cell type or population is selected from inhibitory GABA-ergic neurons or basal forebrain neurons. In an embodiment, the inhibitory GABA-ergic neurons are selected from parvalbumin (PV)-expressing Chandelier interneurons, arkypallidal (ArkyP) neurons, or Somatostatin (SST)-expressing interneurons. In an embodiment, the basal forebrain neurons are selected from Dopamine-Receptor 1 (Drd1)-expressing neurons; Dopamine-Receptor 2 (Drd2)-expressing neurons; or cholinergic (Ch AT) neurons.

In an embodiment of the above-delineated aspects and/or embodiments thereof, the enhancer element comprises any one of S9E10 (SEQ ID NO: 19) or huS9E10 (SEQ ID NO: 20) having specificity for somatostatin (SST) neurons; S9E36 (SEQ ID NO: 49) or huS9E36 (SEQ ID NO: 50) having specificity for Arkypalladial (ArkyP) neuronal cell populations in the globus pallidus; S9E24 (SEQ ID NO: 55) or huS9E24 (SEQ ID NO: 56) having specificity for Arkypalladial (ArkyP) neuronal cell populations in the globus pallidus; S9E2 (SEQ ID NO: 3) or huS9E2 (SEQ ID NO: 4) having specificity for PV-expressing Chandelier interneurons. In an embodiment, the enhancer element comprises S9E10 (SST) (SEQ ID NO: 19) or huS9E10 (SEQ ID NO: 20) having specificity for somatostatin (SST) neurons. In an embodiment, the enhancer element comprises S9E36 (SEQ ID NO: 49) or huS9E36 (SEQ ID NO: 50); or S9E24 (SEQ ID NO: 55) or huS9E24 (SEQ ID NO: 56) having specificity for Arkypalladial (ArkyP) neuronal cell populations in the globus pallidus. In an embodiment, the enhancer element comprises S9E2 (SEQ ID NO: 3) or huS9E2 (SEQ ID NO: 4) having specificity for PV-expressing Chandelier interneurons. In an embodiment, the enhancer element comprises any one of S9E21 (SEQ ID NO: 41) or huS9E21 (SEQ ID NO: 42); S9E33 (SEQ ID NO: 45) or huS9E33 (SEQ ID NO: 46); or S9E34 (SEQ ID NO: 47) or huS9E34 (SEQ ID NO: 48) having specificity for Dopamine-Receptor 1 (Drd1)-expressing neurons; or S9E22 (SEQ ID NO: 51) or huS9E22 (SEQ ID NO: 52); S9E23 (SEQ ID NO: 53) or huS9E23 (SEQ ID NO: 54) having specificity for Dopamine-Receptor 2 (Drd2)-expressing neurons; or S9E27 (SEQ ID NO: 61) or huS9E27 (SEQ ID NO: 62) having specificity for cholinergic (ChAT) neurons. In an embodiment, the enhancer element comprises S9E21 (SEQ ID NO: 41) or huS9E21 (SEQ ID NO: 42); S9E33 (SEQ ID NO: 45) or huS9E33 (SEQ ID NO: 46); or S9E34 (SEQ ID NO: 47) or huS9E34 (SEQ ID NO: 48) having specificity for Dopamine-Receptor 1 (Drd1)-expressing neurons. In an embodiment, the enhancer element comprises S9E22 (SEQ ID NO: 51) or huS9E22 (SEQ ID NO: 52); S9E23 (SEQ ID NO: 53) or huS9E23 (SEQ ID NO: 54) having specificity for Dopamine-Receptor 2 (Drd2)-expressing neurons. In an embodiment, the enhancer element comprises S9E27 (SEQ ID NO: 61) or huS9E27 (SEQ ID NO: 62) having specificity for cholinergic (Ch AT) neurons. In an embodiment of the foregoing aspects and/or embodiments thereof, the regulated or restricted gene is a transgene or effector gene or polynucleotide.

Provided in another aspect is an isolated, cloned enhancer element comprising a polynucleotide sequence of S9E27 (SEQ ID NO: 61) or huS9E27 (SEQ ID NO: 62), which targets ChAT cholinergic neurons in the brain.

Provided in another aspect is an isolated, cloned enhancer element comprising a polynucleotide sequence of S9E10 (SEQ ID NO: 19) or huS9E10 (SEQ ID NO: 20) which targets SST interneurons in the brain.

Provided in another aspect is an isolated, cloned enhancer element comprising a polynucleotide sequence of S9E36 (SEQ ID NO: 49) or huS9E36 (SEQ ID NO: 50); or S9E24 (SEQ ID NO: 55) or huS9E24 (SEQ ID NO: 56), which targets Arkypalladial (ArkyP) neuronal cell populations in the globus pallidus.

Provided in another aspect is an isolated, cloned enhancer element comprising a polynucleotide sequence of S9E21 (SEQ ID NO: 41) or huS9E21 (SEQ ID NO: 42); S9E33 (SEQ ID NO: 45) or huS9E33 (SEQ ID NO: 46); or S9E34 (SEQ ID NO: 47) or huS9E34 (SEQ ID NO: 48), which targets Dopamine-Receptor 1 (Drd1)-expressing neurons.

Provided in another aspect is an isolated, cloned enhancer element comprising a polynucleotide sequence of S9E22 (SEQ ID NO: 51) or huS9E22 (SEQ ID NO: 52); or S9E23 (SEQ ID NO: 53) or huS9E23 (SEQ ID NO: 54) which targets Dopamine-Receptor 2 (Drd2)-expressing neurons.

Provided in another aspect is an isolated, cloned enhancer element comprising a polynucleotide sequence of S9E2 (SEQ ID NO: 3) or huS9E2 (SEQ ID NO: 4) which targets PV-expressing Chandelier interneurons in the brain cortex.

In another aspect, a viral vector comprising the isolated enhancer element of any one of the above-delineated aspects and/or embodiments thereof is provided. In an embodiment, the viral vector further comprises a transgene polynucleotide sequence.

In another aspect, a viral vector comprising an enhancer element comprising the sequence of any one of SEQ ID NOS: 3, 4, 19, 20, 41, 42, 45, 46, 47, 48, 49, 50, 51, 52, 53, 54, 55, 56, 61, or 62 is provided. In an embodiment, the viral vector further comprises a transgene polynucleotide sequence.

In an embodiment of any of the above-delineated aspects of the viral vector and/or embodiments thereof, the enhancer element comprises S9E10 (SEQ ID NO: 19) or huS9E10 (SEQ ID NO: 20) having at least 85% specificity for SST interneurons in the brain cortex.

In an embodiment of any of the above-delineated aspects of the viral vector and/or embodiments thereof, the enhancer element comprises S9E27 (SEQ ID NO: 61) or huS9E27 (SEQ ID NO: 62) having at least 90% specificity for CHAT cholinergic interneurons in the striatum of the brain and cholinergic projection neurons in the basal nuclei of the brain.

In an embodiment of any of the above-delineated aspects of the viral vector and/or embodiments thereof, the enhancer element comprises a polynucleotide sequence of S9E36 (SEQ ID NO: 49) or huS9E36 (SEQ ID NO: 50); or S9E24 (SEQ ID NO: 55) or huS9E24 (SEQ ID NO: 56), which targets Arkypalladial (ArkyP) neuronal cell populations in the globus pallidus of the brain.

In an embodiment of any of the above-delineated aspects of the viral vector and/or embodiments thereof, the enhancer element comprises S9E2 (SEQ ID NO: 3) or huS9E2 (SEQ ID NO: 4) having at least 70% specificity for parvalbumin (PV)-expressing Chandelier interneurons in the brain.

In an embodiment of any of the above-delineated aspects of the viral vector and/or embodiments thereof, the transgene is a reporter gene, a therapeutic gene encoding a therapeutically or enzymatically active polypeptide, or an effector gene or polynucleotide. In embodiments, the transgene is a polynucleotide encoding a CRISPR-Cas9 protein, a polynucleotide encoding a Zinc Finger Protein, a polynucleotide encoding a Transcription activator-like effector nuclease (TALEN), or an engineered form thereof.

In an embodiment of any of the above-delineated aspects of the viral vector and/or embodiments thereof, the viral vector is a lentivirus vector, an adeno-associated virus (AAV) vector, or a recombinant adeno-associated virus (rAAV) vector. In an embodiment, the viral vector is a recombinant adeno-associated virus (rAAV) vector. In an embodiment, the capacity of the vector to package polynucleotide sequences of greater than about 4.7 kb comprises reassembly of multiple rAAV vectors by homologous recombination or by splicing mediated by acceptor sites.

In an embodiment of any of the above-delineated aspects of the viral vector and/or embodiments thereof, the vector delivers the transgene to GABA-ergic interneurons or basal forebrain neurons in the brain, and wherein the transgene is functionally expressed in the neurons following administration of the vector to a subject. In an embodiment, the subject is a human patient.

In another aspect a viral particle or virus-like particle comprising the viral vector of any one of the above-delineated aspects and/or embodiments thereof is provided.

In another aspect, a cell comprising the viral vector of any one of the above-delineated aspects and/or embodiments thereof is provided.

In another aspect, a cell comprising the viral particle or virus-like particle of the above-delineated aspect and/or embodiments thereof is provided.

In another aspect a pharmaceutical composition comprising the viral vector of any one of the above-delineated aspects and/or embodiments thereof, and a pharmaceutically acceptable vehicle, carrier, or diluent is provided. In an embodiment, the pharmaceutical composition is in liquid dosage form.

In another aspect, a pharmaceutical composition comprising the viral particle or virus-like particle of the above-delineated aspect and/or embodiments thereof, and a pharmaceutically acceptable vehicle, carrier, or diluent is provided. In an embodiment, the pharmaceutical composition is in liquid dosage form.

In an aspect, a method of restoring normal levels of target gene expression in GABA-ergic neuronal cells in which expression levels of the gene are deficient or defective is provided, in which the method involves contacting the cells with an effective amount of the viral vector of any one of the above-delineated aspects and/or an embodiment thereof, a viral particle, a virus-like particle, or a pharmaceutical composition thereof, to restore normal levels of expression of the target gene in the GABAergic neuronal cells.

In an aspect, a method of restoring normal levels of target gene expression in CHAT Cholinergic (ChAT) neurons, Cholinergic interneurons in the striatum, and Cholinergic projection neurons in which expression levels of the gene are deficient or defective is provided, in which the method involves contacting the cells with an effective amount of the viral vector of any one of the above-delineated aspects and/or an embodiment thereof, a viral particle, a virus-like particle, or a pharmaceutical composition thereof, to restore normal levels of expression of the target gene in the Cholinergic neurons (Chat neurons), Cholinergic interneurons in the striatum and Cholinergic projection neurons.

In an embodiment of the above methods, the viral vector, viral particle, or virus-like particle comprises a recombinant adeno-associated virus (rAAV).

In another aspect, a method of delivering a transgene for restricted expression in an inhibitory GABA-ergic neuronal cell is provided, in which the method involves contacting a neuronal cell with a recombinant adeno-associated virus (rAAV) vector, a viral particle, a virus-like particle, or a pharmaceutical composition thereof, comprising the transgene polynucleotide sequence, or a functional portion thereof, and an enhancer element polynucleotide sequence selected from S9E10 (SEQ ID NO: 19) or huS9E10 (SEQ ID NO: 20); S9E36 (SEQ ID NO: 49) or huS9E36 (SEQ ID NO: 50); S9E24 (SEQ ID NO: 55) or huS9E24 (SEQ ID NO: 56); or S9E2 (SEQ ID NO: 3) or huS9E2 (SEQ ID NO: 4) that restricts expression of the transgene in target GABA-ergic neuron cells of the cerebral cortex of the subject, thereby delivering the transgene to the GABA-ergic neuronal cell in the subject.

In an embodiment of the above-delineated methods and/or an embodiment thereof, the GABA-ergic neuronal cell is selected from a parvalbumin (PV)-expressing Chandelier interneuron, a Somatostatin (SST)-expressing interneuron, or an Arkypalladial (ArkyP) neuron.

In an embodiment, the enhancer element is selected from at least one of S9E2 (SEQ ID NO: 3) or huS9E2 (SEQ ID NO: 4); S9E10 (SEQ ID NO: 19) or huS9E10 (SEQ ID NO: 20); S9E36 (SEQ ID NO: 49) or huS9E36 (SEQ ID NO: 50); or S9E24 (SEQ ID NO: 55) or huS9E24 (SEQ ID NO: 56).

In another aspect, a method of delivering a transgene for restricted expression in a basal forebrain neuron is provided, in which the method involves contacting the neuron with a recombinant adeno-associated virus (rAAV) vector, a viral particle, a virus-like particle, or a pharmaceutical composition thereof, comprising the transgene polynucleotide sequence, or a functional portion thereof, and an enhancer element polynucleotide sequence that restricts expression of the transgene in basal forebrain neurons of the subject, thereby delivering the transgene to basal forebrain neurons in the subject. In an embodiment, the basal forebrain neuron is selected from a Dopamine-Receptor 1 (D1)-expressing medium-spiny neuron (Drd1), a Dopamine-Receptor 2 (D2)-expressing medium-spiny neuron (Drd2), or a Cholinergic (ChAT) neuron (CHAT).

In an embodiment of the above-delineated methods and/or embodiments thereof, the enhancer element is selected from at least one of S9E27 (SEQ ID NO: 61) or huS9E27 (SEQ ID NO: 62); S9E21 (SEQ ID NO: 41) or huS9E21 (SEQ ID NO: 42); S9E33 (SEQ ID NO: 45) or huS9E33 (SEQ ID NO: 46); or S9E34 (SEQ ID NO: 47) or huS9E34 (SEQ ID NO: 48); or S9E22 (SEQ ID NO: 51) or huS9E22 (SEQ ID NO: 52); or S9E23 (SEQ ID NO: 53) or huS9E23 (SEQ ID NO: 54). In an embodiment of the methods, the viral vector, viral particle, virus-like particle, or a pharmaceutical composition thereof, is administered systemically, parenterally, intravenously, or intracerebrally. In an embodiment of the methods, the viral vector, viral particle, virus-like particle, or a pharmaceutical composition thereof, is administered as a prophylactic or a therapeutic.

Provided in another aspect is a viral vector comprising an enhancer polynucleotide sequence selected from S9E10 (SEQ ID NO: 19) or huS9E10 (SEQ ID NO: 20); S9E36 (SEQ ID NO: 49) or huS9E36 (SEQ ID NO: 50); S9E24 (SEQ ID NO: 55) or huS9E24 (SEQ ID NO: 56); or S9E2 (SEQ ID NO: 3) or huS9E2 (SEQ ID NO: 4), or a functional portion thereof, and a transgene for expression in GABA-ergic interneurons of the brain cortex, wherein the vector specifically targets the GABA-ergic interneurons of the brain cortex and delivers the transgene thereto. In an embodiment, the enhancer polynucleotide sequence is S9E10 (SEQ ID NO: 19) or huS9E10 (SEQ ID NO: 20) or a functional portion thereof.

Provided in another aspect is a viral vector comprising isolated enhancer polynucleotide sequence selected from S9E27 (SEQ ID NO: 61) or huS9E27 (SEQ ID NO: 62), or a functional portion thereof, and a transgene for expression in Cholinergic neurons, Cholinergic interneurons in the striatum, or Cholinergic projection neurons in the basal nuclei, wherein the vector specifically targets the Cholinergic neurons, Cholinergic interneurons in the striatum, or Cholinergic projection neurons in the basal nuclei and delivers the transgene thereto.

In an embodiment of the above-delineated viral vectors and/or embodiments thereof, the viral vector is an adeno-associated viral vector (AAV) or a recombinant adeno-associated viral vector (rAAV), or a virus particle or virus-like particle thereof.

In another aspect, a method of treating, abating, or ameliorating a neurological, neurodevelopmental, neurodegenerative, neuropsychiatric, neurogenetic, or neuromuscular, disease, disorder, or pathology, and/or the symptoms thereof, in a subject is provided, in which the method involves administering to a subject in need thereof an effective amount of the viral vector of the above-delineated aspects and embodiments thereof. In an embodiment of the method, the disease, disorder, or pathology is Alzheimer's disease, Parkinson's disease, Dystonia, amyotrophic lateral sclerosis (ALS), bipolar disorder, Down Syndrome, or seizures. In embodiments of the method, the symptoms of the disease, disorder, or pathology are reduced, abated, or alleviated in the subject. In an embodiment, the subject is a human or a human patient.

In an embodiment of any one of the above-delineated aspects describing an enhancer element and/or embodiments thereof, the transgene or effector gene or polynucleotide is a therapeutic gene, a reporter gene, a polynucleotide encoding a CRISPR-Cas9 protein, a polynucleotide encoding a Zinc Finger Protein, a polynucleotide encoding a Transcription activator-like effector nuclease (TALEN), or an engineered form thereof.

In an embodiment of any one of the above-delineated aspects describing viral vector and/or embodiments thereof, the transgene or effector gene or polynucleotide is a therapeutic gene, a reporter gene, a polynucleotide encoding a CRISPR-Cas9 protein, a polynucleotide encoding a Zinc Finger Protein, a polynucleotide encoding a Transcription activator-like effector nuclease (TALEN), or an engineered form thereof.

In an embodiment of any one of the above-delineated aspects describing a method and/or embodiments thereof, the transgene is selected from a therapeutic gene, a reporter gene, an effector gene, a polynucleotide encoding a CRISPR-Cas9 protein, a polynucleotide encoding a Zinc Finger Protein, a polynucleotide encoding a Transcription activator-like effector nuclease (TALEN), or an engineered form thereof.

In an embodiment of any of the above-delineated aspects and/or embodiments thereof describing the enhancer element, the viral vector, or the method, the transgene is a therapeutic gene that encodes a therapeutically or enzymatically active, functional, and/or beneficial polypeptide.

In an aspect, a method of treating, abating, or ameliorating a neurological, neurodevelopmental, neurodegenerative, neuropsychiatric, neurogenetic, or neuromuscular, disease, disorder, or pathology, and/or the symptoms thereof, in a subject is provided, in which the method involves administering to a subject in need thereof an effective amount of delivery vehicle, or a pharmaceutical composition thereof, comprising the isolated enhancer element of any one of the above-delineated aspects and/or embodiments thereof, and a transgene. In an embodiment of the method, the disease, disorder, or pathology is one or more of a neuropsychiatric disorder, cognition, or seizure.

In an aspect, a method of treating, abating, or ameliorating a neurological, neurodevelopmental, neurodegenerative, neuropsychiatric, neurogenetic, neuromuscular, or movement disease, disorder, or pathology, and/or the symptoms thereof, in a subject is provided, in which the method involves administering to a subject in need thereof an effective amount of a delivery vehicle, or a pharmaceutical composition thereof, comprising an enhancer element comprising any one of S9E10 (SEQ ID NO: 19) or huS9E10 (SEQ ID NO: 20) having specificity for somatostatin (SST) neurons; S9E36 (SEQ ID NO: 49) or huS9E36 (SEQ ID NO: 50) having specificity for Arkypalladial (ArkyP) neuronal cell populations in the globus pallidus; S9E24 (SEQ ID NO: 55) or huS9E24 (SEQ ID NO: 56) having specificity for Arkypalladial (ArkyP) neuronal cell populations in the globus pallidus; S9E2 (SEQ ID NO: 3) or huS9E2 (SEQ ID NO: 4) having specificity for PV-expressing Chandelier interneurons, and a transgene. In a particular embodiment, the enhancer element comprises S9E36 (SEQ ID NO: 49) or huS9E36 (SEQ ID NO: 50); or S9E24 (SEQ ID NO: 55) or huS9E24 (SEQ ID NO: 56) having specificity for Arkypalladial (ArkyP) neuronal cell populations in the globus pallidus. In an embodiment of the method, the disease, disorder, or pathology is selected from one or more of ataxia, dystonia, tremors, Essential Tremor, Lewy Body dementia, motor stereotypies and Parkinson's Disease, obsessive-compulsive disorder (OCD), epilepsy, and/or the symptoms thereof.

In an embodiment of the above-delineated method and/or embodiments thereof, the transgene comprises a therapeutic gene or effector gene or polynucleotide. In an embodiment of any one of the methods, the symptoms of the disease, disorder, or pathology are reduced, abated, or alleviated in the subject. In an embodiment, the subject is a human or a human patient.

Another aspect provides an isolated, cloned enhancer element comprising the polynucleotide sequence of SEQ ID NO: 19 or 20, or a viral vector comprising the enhancer element, or a functional portion thereof, wherein the enhancer element targets SST interneurons expressing Hpse.

Another aspect provides an isolated, cloned enhancer element comprising the polynucleotide sequence of SEQ ID NO: 41 or 42, or a viral vector comprising the enhancer element, or a functional portion thereof, wherein the enhancer element targets Dopamine-Receptor 1 (D1)-expressing medium-spiny neurons (Drd1 neurons) expressing Slc35d3.

Another aspect provides an isolated, cloned enhancer element comprising the polynucleotide sequence of any one of SEQ ID NOs: 45-48, or a viral vector comprising the enhancer element, or a functional portion thereof, wherein the enhancer element targets Dopamine-Receptor 1 (D1)-expressing medium-spiny neurons (Drd1 neurons) expressing Chrm4.

Another aspect provides an isolated, cloned enhancer element comprising the polynucleotide sequence of SEQ ID NO: 49 or 50, or a viral vector comprising the enhancer element, or a functional portion thereof, wherein the enhancer element targets Dopamine-Receptor 1 (D1)-expressing medium-spiny neurons (Drd1 neurons) expressing Tac1.

Another aspect provides an isolated, cloned enhancer element comprising the polynucleotide sequence of SEQ ID NO: 55 or 56, or a viral vector comprising the enhancer element, or a functional portion thereof, wherein the enhancer element targets Dopamine-Receptor 2 (D2)-expressing medium-spiny neurons (Drd2 neurons) expressing Adora2a.

Another aspect provides an isolated, cloned enhancer element comprising the polynucleotide sequence of SEQ ID NO: 61 or 62, or a viral vector comprising the enhancer element, or a functional portion thereof, wherein the enhancer element targets Cholinergic neurons expressing Chat.

Another aspect provides an isolated, cloned enhancer element comprising the polynucleotide sequence of any one of SEQ ID NOs: 51-54, or a viral vector comprising the enhancer element, or a functional portion thereof, wherein the enhancer element targets Dopamine-Receptor 2 (D2)-expressing medium-spiny neurons (Drd2 neurons) expressing Gpr6.

Another aspect provides an isolated, cloned enhancer element comprising the polynucleotide sequence of SEQ ID NO: 3 or 4, or a viral vector comprising the enhancer element, or a functional portion thereof, wherein the enhancer element targets PV interneurons expressing Lpl.

In another aspect, an isolated, cloned enhancer element comprising a polynucleotide sequence having at least 75% identity to a polynucleotide sequence set forth in any one of SEQ ID NOs: 1-80, wherein the enhancer element regulates or restricts expression of a gene in a neuronal cell type or population, is provided. In an embodiment, the enhancer element comprises a functional portion or fragment of a polynucleotide sequence having at least 75% identity to a polynucleotide sequence set forth in any one of SEQ ID NOs: 1-80. In embodiments, the enhancer element comprises a polynucleotide sequence, or a functional portion or fragment of the polynucleotide sequence, having at least 85%, at least 90%, at least 95%, or at least 98% or greater identity to a polynucleotide sequence set forth in any one of SEQ ID NOs: 1-80. In an embodiment, the enhancer element comprises or consists of a polynucleotide sequence set forth in any one of SEQ ID NOs: 1-80. In an embodiment, the neuronal cell type or population is selected from inhibitory GABA-ergic neurons, cortical interneurons, striatal interneurons, basal nuclei projection neurons, neurons of the globus pallidus, neurons of thalamic and/or subthalamic structures, or basal forebrain neurons. In embodiments, the inhibitory GABA-ergic neurons are selected from parvalbumin (PV)-expressing interneurons, PV-expressing Chandelier interneurons, projection neurons, arkypallidal (ArkyP) neurons; Somatostatin (SST)-expressing interneurons; Vaso-active Intestinal Peptide (VIP)-expressing interneurons; or non-VIP/CGE-derived (ID2) interneurons. In embodiments, the basal forebrain neurons are selected from Dopamine-Receptor 1 (Drd1)-expressing medium-spiny neurons; Dopamine-Receptor 2 (Drd2)-expressing medium-spiny neurons; Cholinergic neurons; Cholinergic interneurons of the striatum (Ch-IN); Cholinergic projection neurons of the basal ganglia or nuclei (Ch-PN), or vasoactive intestinal peptide (ChAT-VIP) neurons (ChAT). In embodiments, isolated enhancers as described herein (e.g., mouse and human S9E24 and S9E36) target arkypallidal (ArkyP) neuronal cells in the globus pallidus. In embodiments, isolated enhancer elements as described herein (e.g., mouse and human S9E21, S9E22, S9E23, S9E23, S9E33, S9E34 and S9E36) target specific regions in the ventral central nervous system (CNS), including the globus pallidus, and/or thalamic structures and subthalamic structures of the brain.

In embodiments of the above aspect, the enhancer element comprises any one of S9E1-S9E5 (SEQ ID NOs: 1, 3, 5, 7, 9) or huS9E1-huS9E5 (SEQ ID NOs: 2, 4, 6, 8, 10); S9E8 (SEQ ID NO: 15) or huS9E8 (SEQ ID NO: 16); S9E9 (SEQ ID NO: 15) or huS9E8 (SEQ ID NO: 16); S9E12 (SEQ ID NO: 23) or huS9E12 (SEQ ID NO: 24); S9E13 (SEQ ID NO: 25) or huS9E13 (SEQ ID NO: 26); S9E15 (SEQ ID NO: 29) or huS9E15 (SEQ ID NO: 30); or S9E18 (SEQ ID NO: 35) or huS9E18 (SEQ ID NO: 36) having at least 70% specificity for parvalbumin (PV)-expressing interneurons in the brain. In embodiments, the enhancer element comprises S9E1-S9E5 (SEQ ID NOs: 1, 3, 5, 7, 9) or huS9E1-huS9E5 (SEQ ID NOs: 2, 4, 6, 8, 10). In an embodiment, the enhancer element S9E1 (SEQ ID NO: 1) or huS9E1 (SEQ ID NO: 2) has at least 80% specificity for PV-expressing interneurons in the brain. In an embodiment, the enhancer element comprises S9E10 (SEQ ID NO: 19) or huS9E10 (SEQ ID NO: 20) having at least 75% specificity for SST interneurons in the brain. In an embodiment, the enhancer element comprises any one of S9E27 (SEQ ID NO: 61) or huS9E27 (SEQ ID NO: 62); S9E28 (SEQ ID NO: 63) or huS9E28 (SEQ ID NO: 65); or S9E39 (SEQ ID NO: 67) or huS9E39 (SEQ ID NO: 68) having specificity for ChAT cholinergic neurons in the brain. In an embodiment, the enhancer element comprises S9E27 (SEQ ID NO: 61) or huS9E27 (SEQ ID NO: 62) having at least 90% specificity for ChAT cholinergic neurons in the brain. In an embodiment, the regulated or restricted gene is a transgene.

Another aspect provides an isolated enhancer element comprising a polynucleotide sequence of any one of S9E1-S9E5 (SEQ ID NOs: 1, 3, 5, 7, or 9, respectively), or huS9E1-huS9E5 (SEQ ID NOs: 2, 4, 6, 8, or 10, respectively, which targets parvalbumin (PV)-expressing interneurons in the brain. In an embodiment, the isolated enhancer element is cloned into a delivery vector or vehicle, e.g., a viral vector, such as an AAV or recombinant AAV vector.

Another aspect provides an isolated enhancer element comprising a polynucleotide sequence of S9E10 (SEQ ID NO: 19) or huS9E10 (SEQ ID NO: 20) which targets SST interneurons in the brain. In an embodiment, the isolated enhancer element is cloned (e.g., molecularly cloned) into a delivery vector or vehicle, e.g., a viral vector, such as an AAV or recombinant AAV vector.

Another aspect provides an isolated enhancer element comprising a polynucleotide sequence of S9E27 (SEQ ID NO: 61) or huS9E27 (SEQ ID NO: 62) which targets ChAT cholinergic neurons in the brain. In an embodiment, the isolated enhancer element is cloned (e.g., molecularly cloned) into a delivery vector or vehicle, e.g., a viral vector, such as an AAV or recombinant AAV vector.

Another aspect provides a viral vector comprising the isolated enhancer element as delineated and described in any of the above aspects and/or embodiments thereof. In an embodiment, the viral vector further comprises a transgene polynucleotide sequence.

Another aspect provides a viral vector comprising an isolated enhancer element comprising the sequence as set forth in any one of SEQ ID NOS: 1-40. In an embodiment, the viral vector further comprises a transgene polynucleotide sequence. In an embodiment, the enhancer element comprises one or more of S9E1-S9E5 (SEQ ID NOs: 1, 3, 5, 7, 9) or huS9E1-huS9E5 (SEQ ID NOs: 2, 4, 6, 8, 10); S9E8 (SEQ ID NO: 15) or huS9E8 (SEQ ID NO: 16); S9E9 (SEQ ID NO: 15) or huS9E8 (SEQ ID NO: 16); S9E12 (SEQ ID NO: 23) or huS9E12 (SEQ ID NO: 24); S9E13 (SEQ ID NO: 25) or huS9E13 (SEQ ID NO: 26); S9E15 (SEQ ID NO: 29) or huS9E15 (SEQ ID NO: 30); or S9E18 (SEQ ID NO: 35) or huS9E18 (SEQ ID NO: 36) which targets parvalbumin (PV)-expressing interneurons in the brain. In an embodiment, the enhancer element comprises S9E1-S9E5 (SEQ ID NOs: 1, 3, 5, 7, 9) or huS9E1-huS9E5 (SEQ ID NOs: 2, 4, 6, 8, 10) having at least 70% specificity for parvalbumin (PV)-expressing interneurons in the brain. In an embodiment, the enhancer element comprises S9E1 (SEQ ID NO: 1) or huS9E1 (SEQ ID NO: 2) having at least 80% specificity for parvalbumin (PV)-expressing interneurons in the brain. In an embodiment, the enhancer element comprises S9E10 (SEQ ID NO: 19) or huS9E10 (SEQ ID NO: 20) having at least 75% specificity for SST interneurons in the brain. In an embodiment, the enhancer element comprises S9E27 (SEQ ID NO: 61) or huS9E27 (SEQ ID NO: 62)) having at least 90% specificity for ChAT cholinergic neurons in the brain. In an embodiment, the transgene is a reporter gene or a therapeutic gene encoding a therapeutically or enzymatically active polypeptide, such as an effector nuclease. In an embodiment, the isolated enhancer (enhancer element) is cloned (e.g., molecularly cloned) into a delivery vector or vehicle, e.g., a viral vector, such as an AAV or recombinant AAV vector. In an embodiment, a transgene may encode an effector nuclease, e.g., Cas9 and the like, to disrupt a harmful or mutated gene or polynucleotide, or to repair a mutated gene or polynucleotide, e.g., a gene or polynucleotide encoding a receptor protein, a channel protein, such as, for example, an ion channel or a ligand-gated ion channel, a transcription factor protein, or a disease-causing protein, and the like. In an embodiment, the viral vector is a lentivirus vector, an adeno-associated virus (AAV) vector, or a recombinant adeno-associated virus (rAAV) vector. In an embodiment, the capacity of the vector to package polynucleotide sequences of greater than about 4.7 kb comprises reassembly of multiple rAAV vectors by homologous recombination or by splicing mediated by acceptor sites. In an embodiment, the vector targets and/or delivers the transgene to GABA-ergic interneurons or basal forebrain neurons in the brain, and wherein the transgene is functionally expressed in the neurons following administration of the vector to a subject. In an embodiment, the subject is a human patient.

In another aspect, a viral particle or virus-like particle comprising the viral vector as delineated and described in any of the above aspects and/or embodiments thereof, is provided.

In another aspect, a cell comprising the viral vector as delineated and described in any of the above aspects and/or embodiments thereof, is provided.

In another aspect, a cell comprising the viral particle or virus-like particle the above-delineated and described aspect and embodiments thereof, is provided.

In another aspect, a pharmaceutical composition comprising the viral vector, the viral particle, or the virus-like particle as delineated and described in any of the above aspects and/or embodiments thereof, and a pharmaceutically acceptable vehicle, carrier, or diluent is provided. In an embodiment, the pharmaceutical composition is in liquid dosage form.

In another aspect, a method of restoring normal levels of target gene expression in GABA-ergic neuronal cells in which expression levels of the gene are deficient or defective is provided, in which the method involves contacting the cells with an effective amount of the viral vector of any one of 19-24, a viral particle, a virus-like particle, or a pharmaceutical composition thereof, to restore normal levels of expression of the target gene in the GABAergic neuronal cells.

In another aspect, a method of restoring normal levels of target gene expression in ChAT Cholinergic neurons (ChAT neurons), Cholinergic interneurons in the striatum, and Cholinergic projection neurons in which expression levels of the gene are deficient or defective is provided, in which the method involves contacting the cells with an effective amount of the viral vector, the viral particle, the virus-like particle, or pharmaceutical compositions thereof, as delineated and described in any of the above aspects and/or embodiments thereof, to restore normal levels of expression of the target gene in the Cholinergic neurons (ChAT neurons), Cholinergic interneurons in the striatum and Cholinergic projection neurons. In embodiments, the viral vector, viral particle, or virus-like particle comprises a recombinant adeno-associated virus (rAAV).

Another aspect provides a method of delivering a transgene for restricted expression in an inhibitory GABA-ergic neuronal cell, in which the method involves contacting a neuronal cell with a recombinant adeno-associated virus (rAAV) vector comprising the transgene polynucleotide sequence, or a functional portion thereof, and an enhancer element polynucleotide sequence selected from S9E1-S9E5 (SEQ ID NOs: 1, 3, 5, 7, 9) or huS9E1-huS9E5 (SEQ ID NOs: 2, 4, 6, 8, 10); or S9E6-S9E20 (SEQ ID NOs: 11, 13, 15, 17, 19, 21, 23, 25, 27, 29, 31, 33, 35, 37, 39) or huS9E6-huS9E20 (SEQ ID NOs: 12, 14, 16, 18, 20, 22, 24, 26, 28, 30, 32, 34, 36, 38, 40) that restricts expression of the transgene in target GABA-ergic neuron cells of the cerebral cortex of the subject, thereby delivering the transgene to the GABA-ergic neuronal cell in the subject. In an embodiment, the GABA-ergic neuronal cell is selected from a parvalbumin (PV)-expressing interneuron, a Somatostatin (SST)-expressing interneuron, a Vaso-active Intestinal Peptide (VIP)-expressing interneuron, or a non-VIP/CGE-derived interneuron (ID2). In an embodiment, the enhancer element is selected from at least one of SEQ ID NOs: 1-SEQ ID NO: 10, 19, or 20.

Another aspect provides a method of delivering a transgene for restricted expression in a basal forebrain neuron, in which the method involves contacting the neuron with a recombinant adeno-associated virus (rAAV) vector comprising the transgene polynucleotide sequence, or a functional portion thereof, and an enhancer element polynucleotide sequence that restricts expression of the transgene in basal forebrain neurons of the subject, thereby delivering the transgene to basal forebrain neurons in the subject. In an embodiment, the basal forebrain neuron is selected from a Dopamine-Receptor 1 (D1)-expressing medium-spiny neuron (Drd1), a Dopamine-Receptor 2 (D2)-expressing medium-spiny neuron (Drd2), a Cholinergic neuron (ChAT), a Cholinergic interneuron of the striatum (Ch-IN), or a Cholinergic projection neuron of the basal ganglia (Ch-PN). In an embodiment of the method, the enhancer element is selected from at least one of SEQ ID NOs: 41-80. In an embodiment of the method, the enhancer element comprises SEQ ID NO: 61 or 62.

In an embodiment of the methods of the above-delineated aspects and/or embodiments thereof, the viral vector, viral particle, virus-like particle, or a pharmaceutical composition thereof, is administered systemically, parenterally, intravenously, or intracerebrally. In another embodiment of the methods, the viral vector, viral particle, virus-like particle, or a pharmaceutical composition thereof, is administered as a prophylactic. In another embodiment of the methods, the viral vector, viral particle, virus-like particle, or a pharmaceutical composition thereof, is administered as a therapeutic.

In another aspect, a viral vector comprising an enhancer polynucleotide sequence selected from SEQ ID NOs: 1-40, or a functional portion thereof, and a transgene for expression in GABA-ergic interneurons of the brain cortex, wherein the vector specifically targets the GABA-ergic interneurons of the brain cortex and delivers the transgene thereto, is provided. In an embodiment, the enhancer polynucleotide sequence is selected from SEQ ID NOs: 1-10, 19, or 20, or a functional portion thereof.

In another aspect, a viral vector comprising an enhancer polynucleotide sequence selected from SEQ ID NOs: 41-80, or a functional portion thereof, and a transgene for expression in Cholinergic neurons, Cholinergic interneurons in the striatum, or Cholinergic projection neurons in the basal nuclei, wherein the vector specifically targets the Cholinergic neurons, Cholinergic interneurons in the striatum, or Cholinergic projection neurons in the basal nuclei and delivers the transgene thereto, is provided. In an embodiment, the enhancer polynucleotide sequence comprises SEQ ID NOs: 61 or 62, or a functional portion thereof.

In an embodiment, the viral vector of the above-delineated aspects and/or embodiments thereof, is an adeno-associated viral vector (AAV) or a recombinant adeno-associated viral vector (rAAV), or a virus particle or virus-like particle thereof.

In an aspect, a method of treating, abating, or ameliorating a neurological, neurodevelopmental, neurodegenerative, neuropsychiatric, neurogenetic, or neuromuscular, disease, disorder, or pathology, and/or the symptoms thereof, in a subject is provided, in which the method involves administering to a subject in need thereof an effective amount of the viral vector of any one of the above-delineated aspects and/or embodiments thereof. In an embodiment, the disease, disorder, or pathology is Alzheimer's disease, Parkinson's disease, Dystonia, amyotrophic lateral sclerosis (ALS), bipolar disorder, Down Syndrome, or seizures. In an embodiment, the symptoms of the disease, disorder, or pathology are reduced, abated, or alleviated in the subject. In an embodiment, the subject is a human or a human patient.

In an aspect, an isolated, cloned enhancer element comprising the polynucleotide sequence of SEQ ID NO: 1 or 2, or a viral vector comprising the enhancer element, or a functional portion thereof, wherein the enhancer element targets PV-expressing interneurons expressing Prss23, is provided.

In an aspect, an isolated, cloned enhancer element comprising the polynucleotide sequence of SEQ ID NO: 5 or 6, or a viral vector comprising the enhancer element, or a functional portion thereof, wherein the enhancer element targets PV interneurons expressing Cntnap5b, is provided.

In an aspect, an isolated, cloned enhancer element comprising the polynucleotide sequence of SEQ ID NO: 7 or 8, or a viral vector comprising the enhancer element, or a functional portion thereof, wherein the enhancer element targets PV interneurons expressing Plcxd3, is provided.

In an aspect, an isolated, cloned enhancer element comprising the polynucleotide sequence of SEQ ID NO: 9 or 10, or a viral vector comprising the enhancer element, or a functional portion thereof, wherein the enhancer element targets PV interneurons expressing Elf5, is provided.

In an aspect, an isolated, cloned enhancer element comprising the polynucleotide sequence of SEQ ID NO: 11 or 12, or a viral vector comprising the enhancer element, or a functional portion thereof, wherein the enhancer element targets Somatostatin (SST)-expressing interneurons expressing Satb1, is provided.

In an aspect, an isolated, cloned enhancer element comprising the polynucleotide sequence of SEQ ID NO: 13 or 14, or a viral vector comprising the enhancer element, or a functional portion thereof, wherein the enhancer element targets SST interneurons expressing Ccna1, is provided.

In an aspect, an isolated, cloned enhancer element comprising the polynucleotide sequence of SEQ ID NO: 15 or 16, or a viral vector comprising the enhancer element, or a functional portion thereof, wherein the enhancer element targets SST interneurons expressing Calb1, is provided.

In an aspect, an isolated, cloned enhancer element comprising the polynucleotide sequence of SEQ ID NO: 17 or 18, or a viral vector comprising the enhancer element, or a functional portion thereof, wherein the enhancer element targets SST interneurons expressing Smc2, is provided.

In an aspect, an isolated, cloned enhancer element comprising the polynucleotide sequence of any one of SEQ ID NOs: 21-24, or a viral vector comprising the enhancer element, or a functional portion thereof, wherein the enhancer element targets Vaso-active Intestinal Peptide (VIP) interneurons expressing Prox1, is provided.

In an aspect, an isolated, cloned enhancer element comprising the polynucleotide sequence of SEQ ID NO: 25 or 26, or a viral vector comprising the enhancer element, or a functional portion thereof, wherein the enhancer element targets VIP interneurons expressing Vip, is provided.

In an aspect, an isolated, cloned enhancer element comprising the polynucleotide sequence of SEQ ID NO: 27 or 28, or a viral vector comprising the enhancer element, or a functional portion thereof, wherein the enhancer element targets VIP interneurons expressing Npy5r, is provided.

In an aspect, an isolated, cloned enhancer element comprising the polynucleotide sequence of SEQ ID NO: 29 or 30, or a viral vector comprising the enhancer element, or a functional portion thereof, wherein the enhancer element targets VIP interneurons expressing Grpr, is provided.

In an aspect, an isolated, cloned enhancer element comprising the polynucleotide sequence of SEQ ID NO: 31 or 32, or a viral vector comprising the enhancer element, or a functional portion thereof, wherein the enhancer element targets non-VIP/CGE-derived interneurons (ID2 interneurons) expressing Sv2c, is provided.

In an aspect, an isolated, cloned enhancer element comprising the polynucleotide sequence of SEQ ID NO: 33 or 34, or a viral vector comprising the enhancer element, or a functional portion thereof, wherein the enhancer element targets non-VIP/CGE-derived interneurons (ID2 interneurons) expressing Pde11a, is provided.

In an aspect, an isolated, cloned enhancer element comprising the polynucleotide sequence of SEQ ID NO: 35 or 36, or a viral vector comprising the enhancer element, or a functional portion thereof, wherein the enhancer element targets non-VIP/CGE-derived interneurons (ID2 interneurons) expressing Wt1, is provided.

In an aspect, an isolated, cloned enhancer element comprising the polynucleotide sequence of any one of SEQ ID NOs: 37-40, or a viral vector comprising the enhancer element, or a functional portion thereof, wherein the enhancer element targets non-VIP/CGE-derived interneurons (ID2 interneurons) expressing Lamp5, is provided.

In an aspect, an isolated, cloned enhancer element comprising the polynucleotide sequence of SEQ ID NO: 43 or 44, or a viral vector comprising the enhancer element, or a functional portion thereof, wherein the enhancer element targets Dopamine-Receptor 1 (D1)-expressing medium-spiny neurons (Drd1 neurons) expressing Drd1, is provided.

In an aspect, an isolated, cloned enhancer element comprising the polynucleotide sequence of any one of SEQ ID NOs: 57-60, or a viral vector comprising the enhancer element, or a functional portion thereof, wherein the enhancer element targets Dopamine-Receptor 2 (D2)-expressing medium-spiny neurons (Drd2 neurons) expressing Sp9, is provided.

In an aspect, an isolated, cloned enhancer element comprising the polynucleotide sequence of any one of SEQ ID NOs: 61-64, or a viral vector comprising the enhancer element, or a functional portion thereof, wherein the enhancer element targets Cholinergic neurons expressing Chat, is provided.

In an aspect, an isolated, cloned enhancer element comprising the polynucleotide sequence of any one of SEQ ID NOs: 65-68, or a viral vector comprising the enhancer element, or a functional portion thereof, wherein the enhancer element targets Cholinergic neurons expressing Zic1, is provided.

In an aspect, an isolated, cloned enhancer element comprising the polynucleotide sequence of SEQ ID NO: 69 or 70, or a viral vector comprising the enhancer element, or a functional portion thereof, wherein the enhancer element targets Cholinergic interneurons of the striatum (Ch-IN interneurons) expressing Isl1, is provided.

In an aspect, an isolated, cloned enhancer element comprising the polynucleotide sequence of SEQ ID NO: 71 or 72, or a viral vector comprising the enhancer element, or a functional portion thereof, wherein the enhancer element targets Cholinergic interneurons of the striatum (Ch-IN interneurons) expressing Tshz2, is provided.

In an aspect, an isolated, cloned enhancer element comprising the polynucleotide sequence of SEQ ID NO: 73 or 74, or a viral vector comprising the enhancer element, or a functional portion thereof, wherein the enhancer element targets Cholinergic interneurons of the striatum (Ch-IN interneurons) expressing Zic1, is provided.

In an aspect, an isolated, cloned enhancer element comprising the polynucleotide sequence of SEQ ID NO: 75 or 76, or a viral vector comprising the enhancer element, or a functional portion thereof, wherein the enhancer element targets Cholinergic projection neurons of the basal ganglia (Ch-PN neurons) expressing Chat, is provided.

In an aspect, an isolated, cloned enhancer element comprising the polynucleotide sequence of SEQ ID NO: 77 or 78, or a viral vector comprising the enhancer element, or a functional portion thereof, wherein the enhancer element targets Cholinergic projection neurons of the basal ganglia (Ch-PN neurons) expressing Pcdh9, is provided.

In an aspect, an isolated, cloned enhancer element comprising the polynucleotide sequence of SEQ ID NO: 79 or 80, or a viral vector comprising the enhancer element, or a functional portion thereof, wherein the enhancer element targets Cholinergic projection neurons of the basal ganglia (Ch-PN neurons) expressing Zic1, is provided.

In an embodiment of the isolated, enhancer element of any one of the above-delineated aspects and/or embodiments thereof, the viral vector is an adenoviral vector, an adeno-associated virus (AAV) vector, or a recombinant adeno-associated virus (rAAV) vector, or a viral particle or virus-like particle thereof. In an embodiment of any one of the above-delineated aspects and/or embodiments thereof, the isolated enhancer element is molecularly cloned into the viral vector, such as an AAV or recombinant AAV vector, a viral particle, or virus-like particle.

In an aspect, a cell comprising the viral vector, viral particle, or virus-like particle thereof of any one of the above-delineated aspects and/or embodiments thereof is provided.

In an aspect, a pharmaceutical composition comprising the viral vector, or the viral particle or virus-like particle thereof, of any one of the above-delineated aspects and/or embodiments thereof, and a pharmaceutically acceptable vehicle, carrier, or diluent is provided.

In an aspect, isolated enhancer element S9E2 (SEQ ID NO: 3) or huS9E2 (SEQ ID NO: 4) having specificity for PV-expressing Chandelier interneurons in the brain cortex is provided.

In an aspect, isolated enhancer elements S9E24 (SEQ ID NO: 55), huS9E24 (SEQ ID NO: 56), S9E36 (SEQ ID NO: 49), or huS9E36 (SEQ ID NO: 50) having specificity for Arkypalladial (ArkyP) neuronal populations in the globus pallidus are provided.

In an aspect, isolated enhancer elements S9E21 (SEQ ID NO: 41), huS9E21 (SEQ ID NO: 42), S9E22 (SEQ ID NO: 51), huS9E22 (SEQ ID NO: 52), S9E23 (SEQ ID NO: 53), huS9E23 (SEQ ID NO: 54), S9E24 (SEQ ID NO: 55), huS9E24 (SEQ ID NO: 56) S9E33 (SEQ ID NO: 45), huS9E33 (SEQ ID NO: 46), S9E34 (SEQ ID NO: 47), huS9E34 (SEQ ID NO: 48), S9E36 (SEQ ID NO: 49), or huS9E36 (SEQ ID NO: 50) having specificity for globus pallidus, thalamic structures, and/or subthalamic structures of the ventral central nervous system (CNS) and the brain are provided. In an embodiment of any of the above-delineated aspects, the isolated enhancer element is molecularly cloned into a delivery vector or vehicle, such as a viral vector, e.g., an AAV or recombinant AAV vector.

In an aspect, a method of treating, abating, or ameliorating a neurological, neurodevelopmental, neurodegenerative, neuropsychiatric, neurogenetic, or neuromuscular, disease, disorder, or pathology, and/or the symptoms thereof, in a subject is provided, in which the method involves administering to a subject in need thereof an effective amount of a delivery vehicle or vector, such as a viral vector, viral particle, virus-like particle, or a pharmaceutical composition thereof, comprising isolated enhancer element S9E2 (SEQ ID NO: 3) or huS9E2 (SEQ ID NO: 4) and a transgene. In an embodiment, the transgene is a therapeutic gene. In an embodiment, the transgene is an effector nuclease. In embodiments, the disease, disorder, or pathology is one or more of a neuropsychiatric disorder, cognition, seizure, and/or symptoms thereof.

In an aspect, a method of treating, abating, or ameliorating a neurological, neurodevelopmental, neurodegenerative, neuropsychiatric, neurogenetic, neuromuscular, or movement disease, disorder, or pathology, and/or the symptoms thereof, in a subject, comprising administering to a subject in need thereof an effective amount of a delivery vehicle or vector, such as a viral vector, viral particle, virus-like particle, or a pharmaceutical composition thereof, comprising at least one isolated enhancer element selected from S9E21 (SEQ ID NO: 41), huS9E21 (SEQ ID NO: 42), S9E22 (SEQ ID NO: 51), huS9E22 (SEQ ID NO: 52), S9E23 (SEQ ID NO: 53), huS9E23 (SEQ ID NO: 54), S9E24 (SEQ ID NO: 55), huS9E24 (SEQ ID NO: 56), S9E33 (SEQ ID NO: 45), huS9E33 (SEQ ID NO: 46), S9E34 (SEQ ID NO: 47), huS9E34 (SEQ ID NO: 48), S9E36 (SEQ ID NO: 49), or huS9E36 (SEQ ID NO: 50) and a transgene. In an embodiment, the transgene is a therapeutic gene. In an embodiment, the transgene encodes an effector nuclease, e.g., a CRISPR/Cas protein, Zinc Finger Protein, a TALEN protein, or engineered form thereof. In embodiments, the disease, disorder, or pathology is selected from one or more of ataxia, dystonia, tremors, Essential Tremor, Lewy Body dementia, motor stereotypies and Parkinson's Disease, obsessive-compulsive disorder (OCD), epilepsy, and/or symptoms thereof.

In embodiments of the above-delineated methods, the symptoms of the disease, disorder, or pathology are reduced, abated, or alleviated in the subject. In an embodiment, the subject is a human or a human patient.

In embodiments of any one of the above-delineated aspects and/or embodiments thereof, a transgene, e.g., within a vector, viral vector, virus particle, virus-like particle, or pharmaceutical composition thereof, is selected from a therapeutic gene, a reporter gene, an effector gene, a gene or polynucleotide encoding, for example, a CRISPR-Cas9 protein, a Zinc Finger Protein, a Transcription activator-like effector nuclease (TALEN), or an engineered form thereof.

In embodiments of the viral vector of any one of the above-delineated aspects and/or embodiments thereof, the transgene is selected from a therapeutic gene, a reporter gene, an effector gene, a gene or polynucleotide encoding a CRISPR-Cas9 protein, a Zinc Finger Protein, a Transcription activator-like effector nuclease (TALEN), or an engineered form thereof.

In embodiments of the methods of any one of the above-delineated aspects and/or embodiments thereof, the transgene is selected from a therapeutic gene, a reporter gene, an effector gene, a gene or polynucleotide encoding a CRISPR-Cas9 protein, a Zinc Finger Protein, a Transcription activator-like effector nuclease (TALEN), or an engineered form thereof.

In embodiments, the enhancer element, the viral vector, or the method of any one of the above-delineated aspects and/or embodiments thereof, the transgene is a therapeutic gene that encodes a therapeutically or enzymatically active, functional, and/or beneficial polypeptide, or an effector nuclease. In an embodiment, the transgene provides, supplies, corrects, or restores function and/or functional activity to the target interneuronal or neuronal cell.

### Definitions

Unless defined otherwise, all technical and scientific terms used herein have the meaning commonly understood by a person skilled in the art to which the described aspects and embodiments belong. The following references provide one of skill with a general definition of many of the terms used in the described embodiments: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “administering” is meant giving, supplying, dispensing a composition, agent, therapeutic product, e.g., a virus vector (rAAV) harboring a transgene (e.g., an effector gene or polynucleotide, a therapeutic gene or polynucleotide, or a gene or polynucleotide encoding an effector nuclease), and the like to a subject, or applying or bringing the composition and the like into contact with the subject. Administering or administration may be accomplished by any of a number of routes, such as, for example, and without limitation, parenteral or systemic, intravenous (IV injection), subcutaneous (SC), intrathecal (IT), intracranial (IC), intramuscular (IM), dermal, intradermal (ID), inhalation, rectal, intravaginal, topical, oral, subcutaneous, intramuscular, or intraocular. In embodiments, administration is systemic, such as by inoculation, injection, or intravenous injection.

By “agent” is meant a peptide, polypeptide, nucleic acid molecule, or small molecule chemical compound, antibody, or a fragment thereof.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, or a 50% or greater change in expression levels. An alteration may reflect a change based on a reference or a control. In some embodiments, the reference or control can include a normal, standard, or customary expression level or activity of a gene or polypeptide. In some embodiments, the reference or control can be an abnormal, diseased, nonstandard, or noncustomary expression level or activity of a gene or polypeptide.

By “ameliorate” and “amelioration” is meant decrease, suppress, attenuate, diminish, abate, arrest, or stabilize the development or progression of a disease.

By “analog” or “derivative” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide.

Such biochemical modifications could increase the analog's protease resistance, membrane permeability, or half-life, without altering, for example, polynucleotide binding activity. In another example, a polynucleotide analog retains the biological activity of a corresponding naturally-occurring polynucleotide while having certain modifications that enhance the analog's function relative to a naturally occurring polynucleotide. Such modifications could increase the polynucleotide's affinity for DNA, half-life, and/or nuclease resistance, an analog may include an unnatural nucleotide or amino acid.

As used herein, the term “at risk” as it applies to a neurological, neurodevelopmental, neurodegenerative, neuropsychiatric, neurogenetic, or neuromuscular disease, disorder, or pathology, such as seizures or epilepsy, refers to patients or individuals who have a family history or genetic risk factor genes for a neurological, neurodevelopmental, neurodegenerative, neuropsychiatric, neurogenetic, or neuromuscular disease, disorder, or pathology, and/or symptoms thereof. Such patients or individuals may be considered to be susceptible to the aforementioned disease, disorder, pathology, and/or the symptoms thereof.

As used herein, the term “carrier” refers to a diluent, adjuvant, excipient, or vehicle with which a composition or pharmaceutical composition, e.g., comprising a polynucleotide, viral vector, or viral particle) can be administered. Pharmaceutical and pharmaceutically acceptable carriers include sterile liquids, such as water and oils, including those of petroleum, animal, vegetable or synthetic origin, such as peanut oil, soybean oil, mineral oil, sesame oil, and the like. Water or aqueous saline solutions and aqueous dextrose and glycerol solutions may be employed as carriers, particularly for injectable solutions. Carriers may also include solid dosage forms, including, but not limited to, one or more of a binder (for compressed pills), a glidant, an encapsulating agent, a flavorant, and a colorant. Suitable pharmaceutical carriers are described in “Remington's Pharmaceutical Sciences” by E. W. Martin.

The term “ChAT” as used herein refers to cholinergic neurons that express choline acetyltransferase (ChAT) and utilize acetylcholine as a neurotransmitter and includes ChAT-IN and ChAT-PN neurons, which are differentiated by their locations (IN is located in striatum and PN is located in the basal ganglia).

As used herein, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited are not changed by the presence of more than that which is recited, but excludes prior art embodiments.

“DREADD” is an acronym for “designer receptor exclusively activated by a designer drug,” which is a modified G protein coupled receptor (GPCR) that may be administered or specifically introduced into a subject, or cells thereof, e.g., PV-expressing interneurons, by use of a viral vector (which contains a polynucleotide sequence encoding the DREADD) or through genetic breeding. DREADDs, which are known as chemical genetic or “chemogenetic” molecules, allow for a precise level of temporal control over the excitation and inhibition of neurons. Following expression of the DREADD, it may be activated by a specific ligand (or agonist), which may be administered by intravenous injection or orally. The DREADD and its ligand are designed to be orthogonal, i.e., they bind specifically to each other and do not cross-react. By way of nonlimiting example, five different classes of DREADDs are available for use: hM3Dq raises calcium levels in a cell, causing burst firing; hM4Di lowers cAMP and the activation of a particular potassium channel, causing neuronal silencing, and also inhibits presynaptic neurotransmitter release; OsD enhances cAMP, causing modulation signaling; and Rq(R165L) enhances arrestin signaling, a specific pathway that has been linked to the mechanisms of psychoactive drugs; and κ-opioid receptor DREADD or KORD, which reduces or inhibits excitation of neurons and also inhibits presynaptic neurotransmitter release. (See, e.g., Kelly Rae Chi, 2015, The Scientist; and S. M. Sternson and B. L. Roth, 2014, Ann Rev Neuroscience, 37:387-407). DREADDs have been successfully expressed in nonhuman primates without apparent toxicity, and CNO-DREADDs can modulate circuitry, electrophysiology, and behavior in nonhuman primates (M. A. G. Eldridge et al., 2016, Nature Neurosci., 19:37-39), In an embodiment, the chemical actuator perlapine, an approved medication in humans may be used as a DREADD ligand for activating CNO-based DREADDs in humans. Other small molecule DREADD actuators may include salvinorin B and/or its precursor, salvinorin A. (See, e.g., B. L. Roth, 2016, Neuron, 89(4):683-694).

Orthogonal ligand-gated ion channels, called pharmacologically selective actuator molecules (PSAMs) and pharmacologically selective effector molecules (PSEMs), are other types of chemogenetic molecules that are used as optogenetic agents and in optogenetic methods, in a manner similar to the use of DREADDs. Each PSAM is exclusively activated by a PSEM cognate synthetic agonist. By way of nonlimiting example, three specific PSAM/PSEM tools have been designed, each with different ion conductance properties for controlling neuronal excitability. (See, e.g., Shapiro, M. G. et al., 2012, ACS Chem. Neurosci., 3(8):619-629). These include the cation-selective activator, PSAM^{Q79G,Q139G}-5HT3HC/PSEM^{22S}, the anion-selective silencer, PSAM^{L141F,Y115F}-GlyR/PSEM⁸⁹S, and a third Ca²⁺-selective channel, PSAM^{Q79G,L141S}-nAChR V13′T/PSEM^{9S}. (See, Ibid., and Magnus, C. J. et al., 2011, Science, 333(6047):1292-1296). Both DREADDS and PSAMs-PSEMs allow control over neuronal activity, in a temporal manner, from minutes to hours. (See, e.g., Kelly Rae Chi, 2015, The Scientist; and S. M. Sternson and B. L. Roth, 2014, Ann Rev Neuroscience, 37:387-407). By way of example, different PSAMs have been used with various ion channels and PSEMs to control neurons, e.g., E/I balance in neurons. Such PSAM-PSEM pairings include, without limitation, PSAM^{L141F, Y115F}-5HT3 HC, which is activated by the ligand PSEM^{89S} allowing cations to flow into the cell and boost excitability; PSAM^{L141F, Y115F}-GlyR, which is activated by the ligand PSEM^{89S}, silencing neurons; and PSAM^{Q79G,L141S}-nAChR V13, which is activated by the ligand PSEM9S, enhancing calcium signaling. Because there are two different PSEM ligands, PSAMs-PSEMs can also be combined in the same animal (subject).

“Detect” refers to identifying the presence, absence or amount of a molecule, compound, or agent to be detected.

By “disease” is meant any pathology, disorder, condition, and/or the symptoms thereof, that adversely affects, damages or interferes with the normal function of a cell, tissue, organ, or part of the body, such as the brain, including the cerebral cortex of the brain and brain tissues, or central nervous system (CNS). In embodiments, the disease is a developmental or neurodevelopmental disorder, a neuropathological disorder, neuropsychological disorder, a neuronal disorder, a neuromuscular disorder, a neurophysiological disorder, and/or the symptoms thereof. In an embodiment, a symptom of a disease, pathology, or disorder, such as a neuropsychiatric disorder, is seizures. Nonlimiting examples of other neurological, neurodevelopmental, neurodegenerative, neuropsychiatric, neurogenetic, or neuromuscular diseases, disorders, or pathologies include Alzheimer's disease, Parkinson's disease, Dystonia, ALS and Down Syndrome.

By “effective amount” is meant the amount of a required to ameliorate the symptoms of a disease relative to an untreated patient. The effective amount of active compound(s) used to practice the described methods for therapeutic treatment of a disease varies depending upon the manner of administration, the age, body weight, and general health of the subject. Ultimately, the attending physician, clinician, or veterinarian will decide the appropriate amount and dosage regimen. Such amount is referred to as an “effective” amount. In one embodiment, an effective amount is the amount of an rAAV vector comprising a specific enhancer sequence (e.g., such as S9E1-S9E40, (SEQ ID NOS: 1-80) as described herein) and one or more transgene sequences (e.g., a therapeutic gene sequence) inserted therein that is required to reduce, ameliorate, abate, inhibit, eliminate, or stabilize a symptom of a neurological, neurodevelopmental, neurodegenerative, neuropsychiatric, neurogenetic, or neuromuscular disease or disorder, or the severity thereof. In another embodiment, an effective amount is the amount of an rAAV vector comprising a specific enhancer sequence (e.g., S9E1-S9E40, as described herein) and one or more transgenes (e.g., a therapeutic gene, or effector genes encoding products such as Gq-DREADD or PSAM, or a clustered regularly interspaced short palindromic repeats-Cas9 (CRISPR-Cas9) protein or variant thereof, a Zinc Finger Protein, a Transcription activator-like effector nuclease (TALEN), or an engineered form thereof) sequences inserted therein required to direct, restrict, or regulate expression of the transgene in a target neuron cell type or population, such as a GABAergic interneuron cell, for example, parvalbumin (PV)-expressing interneurons, e.g., mouse and human S9E1-S9E5 (SEQ ID NOs: 1-10), Somatostatin (SST)-expressing interneurons, e.g., mouse and human S9E6-S9E10 (SEQ ID NOs: 11-20); Vaso-active Intestinal Peptide (VIP)-expressing interneurons, e.g., mouse and human S9E11-S9E15 (SEQ ID NOs: 21-30); and non-VIP/CGE-derived interneurons (ID2), e.g., mouse and human S9E16-S9E20 (SEQ ID NOs: 31-40), or of basal forebrain neurons, such as Dopamine-Receptor 1 (D1)-expressing medium-spiny neurons (Drd1 neurons), e.g., mouse and human S9E21 (SEQ ID NOs: 41 and 42), S9E25 (SEQ ID NOs: 43 and 44), S9E33 (SEQ ID NOs: 45 and 46), S9E34 (SEQ ID NOs: 47 and 48) and S9E36 (SEQ ID NOs: 49 and 50); Dopamine-Receptor 2 (D2)-expressing medium-spiny neurons (Drd2 neurons), e.g., mouse and human S9E22 (SEQ ID NOs: 51 and 52), S9E23 (SEQ ID NOs: 53 and 54), S9E24 (SEQ ID NOs: 55 and 56), S9E31 (SEQ ID NOs: 57 and 58) and S9E32 (SEQ ID NOs: 59 and 60); Cholinergic neurons, e.g., mouse and human S9E27 (SEQ ID NOs: 61 and 62), S9E28 (SEQ ID NOs: 63 and 64), S9E38 (SEQ ID NOs.: 65 and 66), and S9E39 (SEQ ID NOs: 67 and 68); Cholinergic interneurons of the striatum (Ch-IN), e.g., mouse and human S9E26 (SEQ ID NOs: 69 and 70), S9E35 (SEQ ID NOs: 71 and 72), and S9E40 (SEQ ID NOs: 73 and 74); and Cholinergic projection neurons of the basal ganglia (Ch-PN), e.g., mouse and human S9E29 (SEQ ID NOs: 75 and 76), S9E30 (SEQ ID NOs: 77 and 78), and S937 (SEQ ID NOs: 79 and 80). In an embodiment, the enhancer is one or more of mouse or human S9E1-S9E5 (SEQ ID NOs: 1-10)), as described herein, which restricts expression of a transgene (e.g., a therapeutic gene or an effector gene or polynucleotide (e.g., a gene or polynucleotide encoding Gq-DREADD or PSAM for chemogenetic modulation of PV-interneuron activity, or a clustered regularly interspaced short palindromic repeats-Cas9 (CRISPR-Cas9) protein or variant thereof, a Zinc Finger Protein, a Transcription activator-like effector nuclease (TALEN), or an engineered form thereof) to PV-interneuron cells (e.g., basket or chandelier). In an embodiment, the enhancer is one or more of mouse or human S9E6-S9E10 (SEQ ID NOs: 11-20), as described herein, which restricts expression of a transgene to SST-interneuron cells. In an embodiment, the enhancer is one or more of mouse or human S9E11-S9E15 (SEQ ID NOs: 21-30), as described herein, which restricts expression of a transgene (e.g., a therapeutic gene or an effector gene or polynucleotide) to VIP-interneuron cells. In an embodiment, the enhancer is one or more of mouse or human S9E16-S9E20 (SEQ ID NOs: 31-40), as described herein, which restricts expression of a transgene, to non-VIP/CGE-derived interneurons (ID2-interneuron cells). In an embodiment, the enhancer is one or more of mouse or human S9E21 (SEQ ID NOs: 41 and 42), S9E25 (SEQ ID NOs: 43 and 44), S9E33 (SEQ ID NOs: 45 and 46), S9E34 (SEQ ID NOs: 47 and 48) and S9E36 (SEQ ID NOs: 49 and 50), as described herein, which restricts expression of a transgene to Dopamine-Receptor 1 (D1)-expressing medium-spiny neurons (Drd1 neuron cells). In an embodiment, the enhancer is one or more of mouse or human S9E22 (SEQ ID NOs: 51 and 52), S9E23 (SEQ ID NOs: 53 and 54), S9E24 (SEQ ID NOs: 55 and 56), S9E31 (SEQ ID NOs: 57 and 58) and S9E32 (SEQ ID NOs: 59 and 60), as described herein, which restricts expression of a transgene gene to Dopamine-Receptor 2 (D2)-expressing medium-spiny neurons (Drd2 neuron cells). In an embodiment, the enhancer is one or more of mouse or human S9E26 (SEQ ID NOs: 69 and 70), S9E35 (SEQ ID NOs: 71 and 72), and S9E40 (SEQ ID NOs: 73 and 74), as described herein, which restricts expression of a transgene to Cholinergic interneurons. In an embodiment, the enhancer is one or more of mouse or human S9E26 (SEQ ID NOs: 69 and 70), S9E35 (SEQ ID NOs: 71 and 72), and S9E40 (SEQ ID NOs: 73 and 74), which restricts expression of a transgene to Cholinergic interneurons of the striatum (Ch-IN). In an embodiment, the enhancer is one or more of mouse or human S9E29 (SEQ ID NOs: 75 and 76), S9E30 (SEQ ID NOs: 77 and 78), and S937 (SEQ ID NOs: 79 and 80), which restricts expression of a transgene to Cholinergic projection neurons of the basal ganglia (Ch-PN). In embodiments, the enhancer element sequence is a mouse enhancer sequence or the human ortholog of the mouse enhancer sequence as described herein.

As used herein, the term “endogenous” describes a molecule (e.g., a polypeptide, peptide, nucleic acid, or cofactor) that is found naturally in a particular organism (e.g., a human) or in a particular location within an organism (e.g., an organ, a tissue, or a cell, such as a human cell).

As used herein, the term “exogenous” refers to a molecule (e.g., a polypeptide, peptide nucleic acid, or cofactor) that is not found naturally or endogenously in a particular organism (e.g., a human) or in a particular location within an organism (e.g., an organ, a tissue, or a cell, such as a human cell). Exogenous materials include those that are provided from an external source to an organism or to cultured matter extracted therefrom.

An “enhancer,” “enhancer element,” “enhancer sequence,” or “enhancer regulatory element or sequence,” refers to a nucleic acid or polynucleotide sequence or a region of a nucleic acid or polynucleotide sequence, e.g., DNA or RNA, of about 50-2500 nucleotides, that contains one or more binding sites that are recognized and bound by one or more binding protein(s), e.g., transcription factor(s). In general, the binding proteins function as activators to increase the likelihood that transcription of a particular target gene will occur. Enhancers can activate transcription independent of their location, distance or orientation with respect to the promoters of genes. For example, enhancer sequences may be located upstream of a gene, downstream of a gene, within the coding region of a gene, or up to one million base pairs away from the gene. Typically, and without intending to be bound by theory, the binding of a DNA binding protein(s) or transcription factor(s) to an enhancer changes or alters the conformation of the DNA, thereby allowing interactions to occur between or among the transcription factor(s) bound to the DNA.

Enhancers have been described as clusters of DNA sequences capable of binding combinations of transcription factors that then interact with components of the mediator complex or TFIID to help recruit RNA polymerase II (RNAPII). To accomplish this, enhancer-bound transcription factors loop out the intervening sequences and contact the promoter region of a gene, thus allowing enhancers to act in a distance-independent fashion. In addition, activation of eukaryotic genes requires de-compaction of the chromatin fiber, which is carried out by enhancer-bound transcription factors that can recruit histone modifying enzymes or ATP-dependent chromatin remodeling complexes to alter chromatin structure and increase the accessibility of the DNA to other proteins. (For a review of enhancer function, see, e.g., Ong, C.-T. and Corces, V. G., 2011, Nat. Rev. Genetics, 12(4):283-293).

As described herein, mouse enhancer sequences and their human counterpart sequences were identified for targeting intended neuronal cells and/or neuronal cell populations. The isolated, cloned enhancer sequences, called S9E1-S9E40 (SEQ ID NOs: 1-80) herein, were discovered to have the ability to restrict or regulate the expression of a transgene (e.g., an exogenous polynucleotide sequence encoding a target protein, such as a therapeutic protein or reporter protein) or an effector gene, within neuronal cell types, such as inhibitory GABA-ergic interneurons (e.g., parvalbumin (PV)-expressing interneurons, Somatostatin (SST)-expressing interneurons; Vaso-active Intestinal Peptide (VIP)-expressing interneurons; and non-VIP/CGE-derived interneurons (ID2 interneurons) and basal forebrain neurons (e.g., Dopamine-Receptor 1 (D1)-expressing medium-spiny neurons (Drd1 neurons); Dopamine-Receptor 2 (D2)-expressing medium-spiny neurons (Drd2 neurons); Cholinergic interneurons, namely, Cholinergic interneurons of the striatum (Ch-IN); and Cholinergic projection neurons of the basal ganglia (Ch-PN)). In an embodiment, the enhancer element is isolated from a naturally occurring environment. Such an enhancer element is molecularly cloned and used or contained in a delivery vector, e.g., a viral vector, for delivery to a cell, tissue, or region of the body, such as the brain or central nervous system (CNS).

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “functionally expressed” is meant that a gene, polynucleotide, or transgene contained in or inserted into the polynucleotide of an rAAV or rAAV vector as described herein is expressed in an infected or transduced cell and produces its encoded product, which is functional and/or active in the cell. In an embodiment, the cell is an interneuron cell. In an embodiment, the cell is a GABAergic interneuron cell. In an embodiment, the cell is a GABAergic interneuron cell. In an embodiment, the cell is a basal forebrain neuron. In an embodiment, the transgene is a therapeutic gene, which encodes a therapeutic protein, or an effector protein, such as a nuclease as described herein. In an embodiment, the transgene is a detectable reporter gene, such as d-Tomato (excitation peak: 554 nm; emission peak: 581 nm; a fluorescent dimer protein that emits orange-red light when it is excited by green-yellow light), Channelrhodopsin (ChR2), Green Fluorescent Protein (GFP), Red Fluorescent Protein (RFP), and the like. In an embodiment, the transgene encodes a Designer receptor exclusively activated by designer drugs (DREADD) or Gq-DREADD. In an embodiment, the transgene encodes PSAM. In embodiments, the transgene is a polynucleotide which encodes a clustered regularly interspaced short palindromic repeats-Cas9 (CRISPR-Cas9) protein or variant thereof, a Zinc Finger Protein, a Transcription activator-like effector nuclease (TALEN), or an engineered form thereof. Cas9 is a dual RNA-guided endonuclease enzyme associated with the Clustered Regularly Interspaced Short Palindromic Repeats (CRISPR) adaptive immune system in bacteria such as Streptococcus pyrogenes. Cas9 uses base pairing to recognize and cleave target DNAs with complementarity to the guide RNA. The programmable sequence specificity of Cas9 and RNA-guided. DNA cleavage or nicking activities of Cas9 have been harnessed for precise genome editing and gene expression control in many cells and organisms. In brief, Cas9 and a guide RNA interact to form a complex that can identify target nucleic acid sequences with high selectivity. Cas9 locates and cleaves or nicks a target DNA in CRISPR % Cas systems. It will be appreciated by those skilled in the art that a guide RNA is included in engineered CRISPR/Cas9 systems to bind to the Cas9 protein and induce a conformational change in Cas9, which promotes its effector function and activity. (M. Jinek et al., 2014, Science, 343(6176); H. Nishimasu et al., 2014, Cell, 156(5):935-949; J. A. Doudna et al, 2014; Science, 346(6213)) In an embodiment, a guide RNA is used in conjunction with Cas9 as an effector protein encoded by a polynucleotide in a vector harboring an isolated, cloned enhancer element as described herein. Zinc Finger proteins constitute transcriptional activator proteins containing a zinc finger domain structure (i.e., a structural motif characterized by the coordination of one or more zinc ions), which function to bind and interact with DNA, RNA, poly-ADP-ribose, and other proteins or molecules, and play a role in sequence specific gene regulation. (M. Cassandri et al., 2017, Cell Death Discov., 3(17071); J. H. Laity et al., 2001, Curr. Opin. Struct. Biol., 11:39-46). By way of example, engineered zinc finger arrays can be fused to a DNA cleavage domain (e.g., the DNA cleavage domain of Fok1) to generate zinc finger nucleases. Zinc finger-Fok1 fusions are useful for sequence-specific manipulation of genomes in mammalian cells and organisms. (B. Schierling et al., 2012, Nucleic Acids Research, 40(6):2623-38; J. Guo et al., 2010, J. Mol. Biol., 400(1):96-107). TALENs, which are nucleases that contain DNA binding domains, are useful as genome editing tools for targeted gene editing and genome modifications, e.g., by creating a targeted double-strand break in the genome (or cellular DNA) that stimulates cellular DNA repair through homology directed repair (HDR) or non-homologous end-joining (NHE). (J. C. Miller et al., 2010, Nature Biotechnology, 29, 143-148; D. G. Ousterout et al., 2016, Methods Mol. Biol., 1338.27-42). TALENs can be generated by fusing a (designed) DNA binding domain that recognizes a specific DNA sequence to a nonspecific DNA cleaving domain, allowing for the cleavage of DNA at a specific site with high accuracy.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The term “interneuron” refers to a neuron (nerve cell), or local circuit neuron in the central nervous system (CNS) that relays impulses between sensory neurons and motor neurons. In general, neurons are specialized cells that function primarily in the transmission of nerve impulses. Neurons have cellular processes, such as dendrites and axons. Dendrites, which are shorter processes in the cell body of a neuron, receive inputs from other neurons and conduct signals to the cell body. Axons are longer, single processes of the cell soma and relay signals toward the tip of the neuron (called the synaptic terminal). The three, main types of neurons include sensory neurons, interneurons (of the CNS), and motor neurons. In the human brain, there are about 100 billion interneurons, which receive impulses from the sensory neurons. Interneurons interpret the information received from other neurons and relay impulses to motor neurons for an appropriate response in a function called ‘integration.’ The term “neuron” may be used interchangeably herein with the term “interneuron,” which may be considered to be a neuron that exhibits a certain type of activity, as described supra.

The terms “isolated,” “purified,” or “biologically pure” refer to material that is free to varying degrees from components which normally accompany or are associated with it as found in its native state. “Isolate” denotes a degree of separation from original source or surroundings. “Purify” denotes a degree of separation that is higher than isolation. A “purified” or “biologically pure” protein or polynucleotide is sufficiently free of other materials such that any impurities do not materially affect the biological properties of the protein or polynucleotide, or cause other adverse consequences. That is, a polynucleotide (nucleic acid), polypeptide, or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid, protein, or peptide gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which flank the gene in the naturally-occurring genome of the organism from which a nucleic acid molecule, such as a nucleic acid molecule described herein, is derived. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide that has been separated from components that naturally accompany it. Typically, a polypeptide is isolated when it is at least 60%, by weight, free from the proteins and naturally-occurring organic molecules with which it is naturally associated. Preferably, the preparation is at least 75%, or at least 85%, or at least 90%, or at least 99%, by weight, a desired polypeptide. An isolated polypeptide may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression, level or activity that is associated with a disease or disorder.

The term “mutation,” as used herein, refers to a substitution of a nucleotide base or amino acid residue within a sequence, e.g., a nucleic acid or amino acid sequence, respectively, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

As used herein, “obtaining” as in “obtaining an agent” includes synthesizing, purchasing, or otherwise acquiring the agent.

By “polynucleotide” is meant a nucleic acid molecule, e.g., a double-stranded (ds) DNA polynucleotide, a single-stranded (ss) DNA polynucleotide, a dsRNA polynucleotide, or a ssRNA polynucleotide, that encodes one or more polypeptides. The term encompasses positive-sense (i.e., protein-coding) DNA polynucleotides, which are capable of being transcribed to form an RNA transcript, which can be subsequently translated to produce a polypeptide following one or more optional RNA processing events (e.g., intron excision by RNA splicing, or ligation of a 5′ cap or a 3′ polyadenyl tail). The term additionally encompasses positive-sense RNA polynucleotides, capable of being directly translated to produce a polypeptide following one or more optional RNA processing events. As used herein, a polynucleotide may be contained within a viral vector, such as a recombinant adeno-associated viral vector (rAAV).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (2′—e.g., fluororibose, ribose, 2′-deoxyribose, arabinose, and hexose); and/or modified phosphate groups (e.g., phosphorothioates and 5′-N-phosphoramidite linkages).

As used herein, the term “pharmaceutically acceptable” refers to molecular entities, biological products and compositions that are physiologically tolerable and do not typically produce an allergic or other adverse reaction, such as gastric upset, dizziness and the like, when administered to a patient (e.g., a human patient).

As used herein, the terms “prevent,” “preventing,” “prevention,” “prophylactic treatment” and the like refer to reducing the probability of developing a disorder or condition in a subject, who does not have, but who is at risk of, susceptible to, or predisposed to, developing a disorder or condition.

As used herein, the term “pseudotyped” refers to a viral vector that contains one or more foreign viral structural proteins, e.g., envelope glycoproteins. A pseudotyped virus may be one in which the envelope glycoproteins of an enveloped virus or the capsid proteins of a nonenveloped virus originate from a virus that differs from the source of the original virus genome and the genome replication apparatus. (D. A. Sanders, 2002, Curr. Opin. Biotechnol., 13:437-442). The foreign viral envelope proteins of a pseudotyped virus can be utilized to alter host tropism or to increase or decrease the stability of the virus particles. Examples of pseudotyped viral vectors include a virus that contains one or more envelope glycoproteins that do not naturally occur on the exterior of the wild-type virus. Pseudotyped viral vectors can infect cells and express and produce proteins or molecules encoded by polynucleotides, e.g., reporter or effector proteins or molecules, contained within the viral vectors.

The term “recombinant” as used herein in the context of proteins or nucleic acids refers to proteins or nucleic acids that do not occur in nature (or in a naturally occurring protein or nucleic acid sequence), but are the product of human engineering, often or typically utilizing molecular biological or molecular genetic tools and techniques practiced by the skilled practitioner in the art. For example, in some embodiments, a recombinant protein or nucleic acid molecule comprises an amino acid or nucleotide sequence that comprises at least one, at least two, at least three, at least four, at least five, at least six, at least seven, or at least eight mutations as compared to any naturally occurring sequence.

By “reduces” is meant a negative alteration of at least 5%, 10%, 25%, 50%, 75%, or 100%.

By “reference” is meant a standard or control condition. A “reference sequence” is a defined sequence used as a basis for sequence comparison. A reference sequence may be a subset of or the entirety of a specified sequence, for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, or about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, or about 100 nucleotides, or about 300 nucleotides, or any integer thereabouts or therebetween.

By “specifically binds” is meant a nucleic acid molecule, polypeptide, or complex thereof (e.g., a binding protein such as a transcription factor and its cognate nucleic acid binding region), or a compound, or molecule that recognizes and binds a given polypeptide and/or nucleic acid molecule, but which does not substantially recognize and bind other molecules in a sample, for example, a biological sample.

By “subject” is meant a mammal, including, but not limited to, a human or non-human mammal, such as a non-human primate, e.g., a marmoset, or a non-human mammal, such as a bovine, equine, canine, ovine, or feline mammal, or a sheep, goat, llama, camel, or a rodent (rat, mouse), ferret, gerbil, hamster, or zebrafish. A subject is typically a patient, such as a human patient, who receives treatment for a particular disease or condition as described herein (e.g., neurological, neurodevelopmental, neurodegenerative, neuropsychiatric, neurogenetic, or neuromuscular disease, disorder, or pathology, such as seizures or epilepsy), and/or the symptoms thereof. Examples of subjects and patients include mammals, such as humans, receiving treatment for such diseases, pathologies, or conditions, or who are at risk of, or susceptible to, having such diseases, pathologies, or conditions.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, inclusive of the first and last values.

As used herein, the term “therapeutically effective amount” refers to a quantity of a therapeutic agent that is sufficient to treat, abate, reduce, diagnose, prevent, and/or delay the onset of one or more symptoms of a disease, pathology, disorder, and/or condition upon administration to a patient in need of treatment. In some cases, a therapeutically effective amount may also refer to a quantity of a therapeutic agent that is administered prophylactically (e.g., in advance of the development of full-blown disease) to a subject who is at risk of, or susceptible to, developing a disease, pathology, or condition, or the symptoms thereof, such as a neurological, neurodevelopmental, neurodegenerative, neuropsychiatric, neurogenetic, or neuromuscular disease, pathology, disorder, or condition. In an embodiment, a symptom of a disease, pathology, or disorder, such as a neuropsychiatric disorder, is seizures. Nonlimiting examples of other neurological, neurodevelopmental, neurodegenerative, neuropsychiatric, neurogenetic, or neuromuscular diseases, disorders, or pathologies include Alzheimer's disease, Parkinson's disease, Dystonia, ALS and Down Syndrome.

A “therapeutic gene,” which may be a transgene harbored in a vector with an enhancer element sequence as described herein, refers to a polynucleotide sequence (gene) that encodes a therapeutic protein or molecule. Without limitation, a therapeutic gene (or transgene) may constitute a normal, functional gene, or functional portion thereof, for expression in a particular cell type, such as a neuronal or interneuronal cell types described herein, to correct cellular defects and ameliorate, abate, abrogate, alleviate, or eliminate (cure) a disease, such as a neurological, neurodevelopmental, neurodegenerative, neuropsychiatric, neurogenetic, or neuromuscular disease, disorder, or pathology, and/or the symptoms thereof, as described herein. (See, e.g., T. Friedman et al., 1989, Science, Vol. 22(4910):1275-1281). By way of example, in delivery vectors containing an enhancer element sequence (e.g., one or more of SEQ ID NOs: 1-40) and a therapeutic gene, the expression of a therapeutic gene is restricted (or regulated) by the presence of the enhancer element to certain neuronal or interneuronal cell types and/or populations transduced by the vector. In such cases, the expressed therapeutic gene can replace a faulty, abnormal or aberrant gene or provide a new gene or can encode a therapeutic protein whose expression restores function, restores normal function, compensates for, and/or improves or abrogates abnormal function of a neuronal or interneuronal cell-expressed protein or polypeptide, in an attempt to correct or cure a disease or disorder.

As used herein a “transgene” refers to an exogenous gene or polynucleotide that encodes a protein or polypeptide and is introduced into and expressed in a neuronal cell type or population. By way of non-limiting example, transgenes include reporter genes, therapeutic genes, effector genes such as DREADD- and PSAM-encoding “effector” genes, or another type of “effector” gene that can be used to edit a gene or a polynucleotide sequence (e.g., a protein-encoding polynucleotide sequence; a target gene or polynucleotide sequence), and/or modulate or edit the expression of a target gene or polynucleotide sequence, for example, polynucleotides encoding CRISPR-Cas9 proteins, Zinc Finger Proteins, engineered Zinc Finger Proteins, and Transcription activator-like effector nucleases (TALENs), which are restriction enzymes that can be engineered to cut specific sequences of DNA. TALENs are made by fusing a TAL effector DNA-binding domain to a DNA cleavage domain (a nuclease that cuts DNA strands). Transcription activator-like effectors (TALEs) can be engineered to bind to practically any desired DNA sequence so that when combined with a nuclease, DNA can be cut at specific locations. The restriction enzymes can be introduced into cells, e.g., for use in gene editing, gene therapy, or for genome editing in situ, e.g., employing genome or base editing with engineered nucleases. Zinc finger nucleases, CRISPR/Cas9, and variants and analogs thereof, as well as TALENs are useful in the field of genome or base editing.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. “Treat” or “treatment” may refer to therapeutic treatment, in which the object is to prevent or slow down (lessen or reduce) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder, as well as those prone to have the condition or disorder or those in whom the condition or disorder is to be prevented.

As used herein, the terms “prevent,” “preventing,” “prevention,” “prophylactic treatment” and the like, refer to inhibiting or blocking a disease state, or the full development of a disease in a subject, or reducing the probability of developing a disease, disorder or condition in a subject, who does not have, but is at risk of developing, or is susceptible to developing, a disease, disorder, or condition.

As used herein, the term “vector” refers to a nucleic acid (e.g., a DNA vector, such as a plasmid), a RNA vector, virus or other suitable replicon (e.g., viral vector). A “vector” further refers to a nucleic acid (polynucleotide) molecule into which foreign nucleic acid can be inserted without disrupting the ability of the vector to be expressed in, replicate in, and/or integrate into a host cell. A variety of vectors have been developed for the delivery of polynucleotides encoding exogenous proteins into a prokaryotic or eukaryotic cell. A vector may contain a polynucleotide sequence that includes gene of interest (e.g., a transgene, such as a therapeutic gene, a reporter gene, or an effector gene) as well as, for example, additional sequence elements capable of regulating transcription, translation, and/or the integration of these polynucleotide sequences into the genome of a cell. A vector may contain regulatory sequences, such as a promoter, e.g., a subgenomic promoter, region and an enhancer region, which direct gene transcription. A vector may contain polynucleotide sequences (enhancer sequences) that enhance the rate of translation of these genes or improve the stability or nuclear export of the mRNA that results from gene transcription. These sequence elements may include, e.g., 5′ and 3′ untranslated regions, an internal ribosomal entry site (IRES), and/or a polyadenylation signal site in order to direct efficient transcription of a gene carried on the expression vector. Vectors, such as viral vectors or the rAAV vectors described herein, may also be referred to as expression vectors. In an embodiment, the components, polynucleotides, or encoding polynucleotides contained in a vector are operably linked to allow for expression in a cell.

“Transduction” refers to a process by which DNA or polynucleotide, e.g., one or more transgenes, contained in a virus or virus vector is introduced or transferred into a cell by the virus or virus vector, wherein the DNA or polynucleotide is expressed. In an embodiment, the DNA or polynucleotide transduced into a cell by a virus vector, such as an rAAV vector as described herein, is stably expressed in the cell. In some cases, a virus or virus vector is said to infect a cell.

As used herein, the term “vehicle” refers to a solvent, diluent, or carrier component of a pharmaceutical composition.

By “virus particle” (also called a virion) is meant a virus (infectious agent) that exists as an independent particle comprising the core viral genome or genetic material (RNA or DNA); a protein coat, called the capsid, which surrounds the genetic material and protects it; and, in some cases, an envelope of lipids surrounding the capsid. A virus particle may refer to the form of a virus before it infects a cell and becomes intracellular, or to the form of the virus that infects a cell.

By “virus-like particles (VLPs)” is meant virus particles made up of one of more viral structural proteins, but lacking the viral genome. Because VLPs lack a viral genome, they are non-infectious and yield safer and potentially more-economical vaccines and vaccine products. In addition, VLPs can often be produced by heterologous expression and can be easily purified. Most VLPs comprise at least a viral core protein that drives budding and release of particles from a host cell.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, preferably at least 70%, more preferably 80% or 85%, and most preferably 90%, 95% or even 99% identical at the amino acid level or nucleic acid to the sequence used for comparison, for example, over a specified comparison window. Optimal alignment may be conducted using the homology alignment algorithm of Needleman and Wunsch, 1970, J. Mol. Biol., 48:443. An indication that two peptide or polypeptide sequences are substantially identical is that one peptide or polypeptide is immunologically reactive with specific antibodies raised against the second peptide or polypeptide, although such cross-reactivity is not required for two polypeptides to be deemed substantially identical. Thus, a peptide or polypeptide is substantially identical to a second peptide or polypeptide, for example, where the two differ only by a conservative substitution. Peptides or polypeptides that are “substantially similar” share sequences as noted above except that residue positions which are not identical may differ by conservative amino acid changes. Conservative substitutions typically include, but are not limited to, substitutions within the following groups: glycine and alanine; valine, isoleucine, and leucine; aspartic acid and glutamic acid; asparagine and glutamine; serine and threonine; lysine and arginine; and phenylalanine and tyrosine, and others as known to the skilled person in the art.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “substantially identical” is generally meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 70%, at least 75%, at least 80%, at least 85%, at least 90%, at least 93%, at least 95%, or greater, at least 98%, or greater, or at least 99%, or greater, identical at the amino acid level or nucleic acid to the sequence used for comparison.

Polynucleotides or viral nucleic acid molecules useful in the methods and compositions as described herein include any nucleic acid molecule that encodes a polypeptide, or a fragment thereof, or that encodes the components of viral vectors described herein. The polynucleotides or viral nucleic acid molecules may encode polypeptide products harbored by the viral vectors, such as recombinant adeno-associated virus (rAAV) and the like, as well as a peptide or fragment thereof. Such nucleic acid molecules need not be 100% identical with an endogenous sequence or a viral vector nucleic acid sequence, but will typically exhibit substantial identity.

Polynucleotides having substantial identity to an endogenous sequence or to a viral vector sequence are typically capable of hybridizing with at least one strand of a double-stranded nucleic acid molecule or to a viral vector nucleic acid molecule. Nucleic acid molecules useful in the described methods include any nucleic acid molecule that encodes a polypeptide as described herein, or a fragment thereof. By “hybridize” is meant pairing or the nucleic acid molecules to form a double-stranded molecule between complementary polynucleotide sequences (e.g., a gene or nucleic acid sequence described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In an embodiment, wash steps will occur at 25° C. in 30 mM NaCl, 3 mM trisodium citrate, and 0.1% SDS. In another embodiment, wash steps will occur at 42° C. in 15 mM NaCl, 1.5 mM trisodium citrate, and 0.1% SDS. In yet another embodiment, wash steps will occur at 68° C. in 15 mM NaCl, 1.5 mM trisodium citrate, and 0.1% SDS. Additional variations of these conditions will be readily apparent to those skilled in the art. Hybridization techniques are well known to those skilled in the art and are described, for example, in Benton and Davis (Science, 196:180, 1977); Grunstein and Hogness (Proc. Natl. Acad. Sci., USA, 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Nonlimiting examples of “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

By “ortholog” is meant any polypeptide or nucleic acid molecule of an organism that is highly related to a reference protein or nucleic acid sequence from another organism. The degree of relatedness may be expressed as the probability that a reference protein would identify a sequence, for example, in a blast search. The probability that a reference sequence would identify a random sequence as an ortholog is extremely low, less than e⁻¹⁰, e⁻²⁰, e⁻³⁰, e⁻⁴⁰, e⁻⁵⁰, e⁻⁷⁵, e⁻¹⁰⁰. The skilled artisan understands that an ortholog is likely to be functionally related to the reference protein or nucleic acid sequence. In other words, the ortholog and its reference molecule would be expected to fulfill similar, if not equivalent, functional roles in their respective organisms, e.g., mouse and human orthologs. In general, an ortholog is a homologous gene in which the gene sequence has diverged after a speciation event, but the gene and its main function are conserved.

It is not required that an ortholog, when aligned with a reference sequence, have a particular degree of amino acid sequence identity to the reference sequence. A protein ortholog might share significant amino acid sequence identity over the entire length of the protein, for example, or, alternatively, might share significant amino acid sequence identity over only a single functionally important domain of the protein. Such functionally important domains may be defined by genetic mutations or by structure-function assays. Orthologs may be identified using methods practiced in the art. The functional role of an ortholog may be assayed using methods well known to the skilled artisan. For example, function might be assayed in vivo or in vitro using a biochemical, immunological, or enzymatic assay; or transformation rescue. Alternatively, bioassays may be carried out in tissue culture; function may also be assayed by gene inactivation (e.g., by RNAi, siRNA, or gene knockout), or gene over-expression, as well as by other methods.

Ranges as provided herein are understood to be shorthand for all of the values within the range. For example, a range of 1 to 50 is understood to include any number, combination of numbers, or sub-range from the group 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, inclusive of the first and last values.

The amino acid sequence of the human Gq-DREADD (hM3Dq) excitatory receptor is derived from the amino-acid sequence of the unmodified human muscarinic acetylcholine receptor M3 set forth above. In the Gq-DREADD (hM3Dq) receptor amino acid sequence (590 aa), the tyrosine in position 149 is replaced by a cysteine, and the arginine in position 239 is replaced by a glycine (US Publication No. 2018/0078658), as shown below:
Met Thr Leu His Asn Asn Ser Thr Thr Ser Pro Leu Phe Pro Asn Ile Ser Ser Ser Trp Ile His Ser Pro Ser Asp Ala Gly Leu Pro Pro Gly Thr Val Thr His Phe Gly Ser Tyr Asn Val Ser Arg Ala Ala Gly Asn Phe Ser Ser Pro Asp Gly Thr Thr Asp Asp Pro Leu Gly Gly His Thr Val Trp Gln Val Val Phe Ile Ala Phe Leu Thr Gly Ile Leu Ala Leu Val Thr Ile Ile Gly Asn Ile Leu Val Ile Val Ser Phe Lys Val Asn Lys Gln Leu Lys Thr Val Asn Asn Tyr Phe Leu Leu Ser Leu Ala Cys Ala Asp Leu Ile Ile Gly Val Ile Ser Met Asn Leu Phe Thr Thr Tyr Ile Ile Met Asn Arg Trp Ala Leu Gly Asn Leu Ala Cys Asp Leu Trp Leu Ala Ile Asp Cys Val Ala Ser Asn Ala Ser Val Met Asn Leu Leu Val Ile Ser Phe Asp Arg Tyr Phe Ser Ile Thr Arg Pro Leu Thr Tyr Arg Ala Lys Arg Thr Thr Lys Arg Ala Gly Val Met Ile Gly Leu Ala Trp Val Ile Ser Phe Val Leu Trp Ala Pro Ala Ile Leu Phe Trp Gln Tyr Phe Val Gly Lys Arg Thr Val Pro Pro Gly Glu Cys Phe Ile Gln Phe Leu Ser Glu Pro Thr Ile Thr Phe Gly Thr Ala Ile Ala Gly Phe Tyr Met Pro Val Thr Ile Met Thr Ile Leu Tyr Trp Arg Ile Tyr Lys Glu Thr Glu Lys Arg Thr Lys Glu Leu Ala Gly Leu Gln Ala Ser Gly Thr Glu Ala Glu Thr Glu Asn Phe Val His Pro Thr Gly Ser Ser Arg Ser Cys Ser Ser Tyr Glu Leu Gln Gln Gln Ser Met Lys Arg Ser Asn Arg Arg Lys Tyr Gly Arg Cys His Phe Trp Phe Thr Thr Lys Ser Trp Lys Pro Ser Ser Glu Gln Met Asp Gln Asp His Ser Ser Ser Asp Ser Trp Asn Asn Asn Asp Ala Ala Ala Ser Leu Glu Asn Ser Ala Ser Ser Asp Glu Glu Asp Ile Gly Ser Glu Thr Arg Ala Ile Tyr Ser Ile Val Leu Lys Leu Pro Gly His Ser Thr Ile Leu Asn Ser Thr Lys Leu Pro Ser Ser Asp Asn Leu Gln Val Pro Glu Glu Glu Leu Gly Met Val Asp Leu Glu Arg Lys Ala Asp Lys Leu Gin Ala Gln Lys Ser Val Asp Asp Gly Gly Ser Phe Pro Lys Ser Phe Ser Lys Leu Pro Ile Gln Leu Glu Ser Ala Val Asp Thr Ala Lys Thr Ser Asp Val Asn Ser Ser Val Gly Lys Ser Thr Ala Thr Leu Pro Leu Ser Phe Lys Glu Ala Thr Leu Ala Lys Arg Phe Ala Leu Lys Thr Arg Ser Gln Ile Thr Lys Arg Lys Arg Met Ser Leu Val Lys Glu Lys Lys Ala Ala Gln Thr Leu Ser Ala Ile Leu Leu Ala Phe Ile Ile Thr Trp Thr Pro Tyr Asn Ile Met Val Leu Val Asn Thr Phe Cys Asp Ser Cys Ile Pro Lys Thr Phe Trp Asn Leu Gly Tyr Trp Leu Cys Tyr Ile Asn Ser Thr Val Asn Pro Val Cys Tyr Ala Leu Cys Asn Lys Thr Phe Arg Thr Thr Phe Lys Met Leu Leu Leu Cys Gln Cys Asp Lys Lys Lys Arg Arg Lys Gln Gln Tyr Gln Gln Arg Gln Ser Val Ile Phe His Lys Arg Ala Pro Glu Gln Ala Leu.

Unless specifically stated or obvious from context, as used herein, the term “or” is understood to be inclusive. Unless specifically stated or obvious from context, as used herein, the terms “a”, “an”, and “the” are understood to be singular or plural.

As used herein, the term “about” or “approximately” means within an acceptable error range for the type of value described and the method used to measure the value. For example, these terms can signify within 20%, more preferably within 10%, and most preferably still within 5% of a given value or range. More specifically, “about” can be understood as within 20%, 10%, 9%, 8%, 7%, 6%, 5%, 4%, 3%, 2%, 1%, 0.5%, 0.1%, 0.05%, or 0.01% of the stated value or range. Alternatively, especially in biological systems, the term “about” means within one log unit (i.e., one order of magnitude), preferably within a factor of two of a given value. Unless specifically stated or obvious from context, as used herein, the term “about” is understood as within a range of normal tolerance in the art, for example within 2 standard deviations of the mean. Unless otherwise clear from context, all numerical values provided herein are modified by the term about.

The recitation of a listing of chemical groups or component groups in any definition of a variable herein includes definitions of that variable as any single group or combination of listed groups. The recitation of an embodiment for a variable or aspect herein includes that embodiment as any single embodiment or in combination with any other embodiments or portions thereof as described in the disclosure.

Any compositions or methods provided herein can be combined with one or more of any of the other compositions and methods provided herein.

### BRIEF DESCRIPTION OF THE DRAWINGS

FIGS. 1A and 1B present charts showing the neuronal cell enhancer elements S9E1-S9E40 as described herein. FIG. 1A shows the mouse enhancer sequences (SEQ ID NOs: 1, 3, 5, 7, 9, 11, 13, 15, 17, 19, 21, 23, 25, 27, 29, 31, 33, 35, 37, 39, 41, 43, 35, 37, 49, 51, 53, 55, 57, 59, 61, 63, 65, 67, 69, 71, 73, 75, 77 and 79). FIG. 1B shows the human enhancer sequences that are counterparts of the above-mentioned mouse enhancer sequences (SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 and 80, respectively). The charts include the name of the mouse or human enhancer element; the neuronal cell population that is targeted by the enhancer element (Intended Target Population); a gene whose expression is associated with a target neuronal cell type, e.g., a marker gene, which is naturally and exclusively present in the cell type of interest (Target gene); the genome (mouse or human) associated with the enhancer element sequence; the coordinates within the mouse or human genome in which the enhancer element is located; the site that provides the sequence of the mouse or human enhancer element in the genome; and the length (bp) of the mouse or human enhancer element sequence. FIG. 1B also indicates the percent of identical bases between the mouse and human enhancer sequences. The neuronal cell populations targeted by the enhancer elements described herein include classes of inhibitory GABA-ergic neurons, such as “PV”: parvalbumin expressing interneurons (e.g., basket or chandelier type PV interneurons); “SST”: Somatostatin expressing interneurons; “VIP”: Vaso-active Intestinal Peptide expressing interneurons; and “ID2”: non-VIP/CGE-derived interneurons; and classes of basal forebrain neurons, such as “Drd1”: Dopamine-Receptor 1 expressing medium-spiny neurons; “Drd2”: Dopamine-Receptor 2 expressing medium-spiny neurons; “Cholinergic”: Cholinergic neurons (e.g., ChAT); “Ch-IN”: Cholinergic interneurons of the striatum; and “Ch-PN”: Cholinergic projection neurons of the basal ganglia.

FIG. 2 presents a schematic of a viral vector (adeno associated virus AAV) backbone which harbors at least one neuron-specific enhancer sequence described herein, i.e., S9E1-S9E40. Other components of the viral vector shown in FIG. 1 include Ori (an origin of replication), e.g., from bacteria or lambda phage; two inverted terminal repeats (ITRs), a promoter sequence (e.g., beta-globin promoter); an intron sequence (for efficient RNA maturation); a transgene, e.g., a polynucleotide encoding a therapeutic protein; a Woodchuck Hepatitis Virus Posttranscriptional Regulatory Element (WPRE) sequence that, when transcribed, creates a tertiary structure enhancing expression of the transgene delivered by the AAV vector; an SV40 pA (SV40 polyadenylation site); an F1 origin (a phage-derived origin that allows for the replication and packaging of ssDNA into phage particles), and an ampicillin resistance gene (AmpR). The two ITR sequences are 145 bases each; the DNA sequence between the ITRs is packaged into the AAV vector molecule for delivery of nucleic acids into cells.

FIGS. 3A and 3B present bar graphs showing the results of systemic injection of 10E+11 viral particles containing various enhancer element sequences (S9E1-S9E20) and reporter transgenes per adult mouse and the targeting of each enhancer element sequence to certain interneuronal cells. In FIG. 3A, the leftmost bar (of the group of three bars) for each enhancer shown on the x-axis represents targeting of the enhancer to parvalbumin-expressing (“PV”) interneurons; the middle bar for each enhancer element shown on the x-axis represents targeting of the enhancer to Somatostatin-expressing (“SST”) interneurons; and the rightmost bar for each enhancer element shown on the x-axis represents targeting of the enhancer to Vaso-active Intestinal Peptide expressing (“VIP”) interneurons. FIG. 3B shows targeting of various enhancer elements (S9E26-S9E30, S9E35 and S9E37-S9E40) to ChAT (cholinergic) interneurons, a subclass of vasoactive intestinal peptide (ChAT-VIP) neurons. Specificity is expressed as a percentage of the cellular population expressing the viral reporter that co-expressed a marker gene for the indicated target population. Arrows in black indicate enhancers with specificity of at least 70%-75% for the target neuronal cell population.

FIG. 4 presents confocal microscope images of immunohistochemical analyses related to the targeting of specific neuronal cell populations by rAAV vectors harboring a reporter gene (d-tomato, “dTom”) and a representative enhancer element sequence as described herein (“rAAV-Enhancer-dTom” vector). Adult mice were systemically injected with 10E+11 viral particles per mouse. The rAAV-Enhancer-dTomato vector expressed the reporter dTomato under the control of the Enhancer regulatory element. Brain sections of injected mice were analyzed 3 weeks post-injection by immunohistochemistry (IHC) for expression of both the reporter and the neuronal cell marker. Detection of specific PV, SST, or ChAT-expressing cells transduced by the vector is visualized. ChAT interneurons are a subclass of vasoactive intestinal peptide (ChAT-VIP) neurons, which have been reported to directly excite neighboring neurons in several layers through fast synaptic transmission of acetylcholine (ACh) in rodent medial prefrontal cortex (mPFCA), (J. Obermayer et al., 2019, Nature Commun., 10:5280; https://doi.org/10.1038/s41467-019-13244-9). Shown are the results of the specific expression of the dTom reporter in PV-expressing cells in the brain using the S9E1, S9E3, S9E4, S9E8 and S9E15 enhancer element sequences. Specific expression of the dTom reporter in SST cells in the brain using the S9E10 enhancer element, and specific expression of the dTom reporter in ChAT neurons in the brain using the S9E27 enhancer element are shown. The images illustrate the specificity of expression for the target populations as shown by immunohistochemistry for the viral reporter and the indicated marker gene for enhancers with specificity above 75%. All images were taken in S1, except S9E27, which shows staining in the striatum. The merged images reflect staining of both dTomato and the neuronal cell type.

FIG. 5 provides images showing the expression profile and specificity of the enhancer element S9E2 in mice using immunohistochemistry (IHC) analysis and PV markers. Adult mice were injected systemically with an rAAV vector construct containing the S9E2 (AAV-S9E2) enhancer element sequence and the dTomato reporter, allowing for the expression of the dTomato reporter under the control of the S9E2 enhancer element in cells of the brain. Brain tissue was analyzed 3 weeks post-injection by immunohistochemistry (IHC) for expression of both the reporter and the PV marker in interneuron cells of the brain. The middle image shows viral expression of dTomato under control of the S9E2 enhancer element (white/stippled area). The right image shows expression of PV in PV-expressing interneurons, i.e., PV+-Chandelier interneurons, targeted by the S9E2 enhancer, as detected by anti-PV antibody staining (white areas). The left image (merged) shows expression of both the dTomato and PV marker in PV-expressing interneurons (PV+-Chandelier interneurons). The high degree of colocalization and the layer specific distribution of cells expressing the viral reporter demonstrate that the S9E2 enhancer restricted expression of PV+ Chandelier interneurons.

FIGS. 6A and 6B provide images and graphic quantification showing the expression profile and specificity of the enhancer element S9E10 in mice using immunohistochemistry (IHC) analysis or RNASCOPE® with the indicated markers, namely, dTomato and SST. Adult mice were injected systemically with an rAAV vector construct containing the S9E10 (AAV-S9E10) enhancer element sequence and the dTomato reporter, allowing for the expression of the dTomato reporter under the control of the S9E10 enhancer element in cells of the brain. Brain tissue of the injected mice was analyzed as described above in FIG. 5. FIG. 6A: As described above for FIG. 5, the middle image shows viral expression of dTomato under control of the S9E10 enhancer element (white/stippled area). The right image shows expression of SST in SST-expressing interneurons targeted by the S9E10 enhancer, as detected by anti-SST antibody staining (white areas). The left image (merged) shows expression of both the dTomato and SST marker in SST-expressing interneurons. FIG. 6B: Quantification of the staining in neuronal cells shows a high degree of specificity for SST. The layer localization of the SST-expressing cells is reminiscent of the SST-HPSE subpopulation of neurons, e.g., in layer 4 of the brain neocortex. (F. Scala et al., 2019, Nature Commun., 10:4174 (doi: 10.1038/s41467-019-12058-z)). In the graph shown in FIG. 6B, the leftmost bar corresponds to S9E10_V1 and the rightmost bar corresponds to S9E10_M1.

FIG. 7 provides images and tracings showing reporter expression and electrophysiology profiling of SST interneuron cells in a macaque (Salman). Following hippocampal injection (5 weeks), SST-expressing interneurons in the macaque expressed the AAV viral reporter under the control of the S9E10 enhancer. Beneath the images are SST firing patterns upon local injection of AAV-S9E10 in the hippocampus of the animal. Electrophysiology recordings show a stereotypical signature of SST cells, thus demonstrating that the specificity of the S9E10 enhancer is maintained for SST-expressing interneurons in the hippocampus of non-human primates.

FIG. 8 provides tiled images (for imaging of the whole slide) following viral targeting of neocortical choline acetyltransferase (ChAT)-expressing neurons (cholinergic neurons) in mice. IHC with the indicated markers were used to show the expression profile and specificity of the S9E27 enhancer in mice injected systemically with AAV-S9E27. “ChAT” represents all neurons that utilize acetylcholine as a neurotransmitter and includes ChAT-IN and ChAT-PN, which are differentiated by their location in the brain (IN is located in striatum and PN is located in the basal ganglia). Adult mice were injected systemically with an rAAV vector construct containing the S9E27 enhancer element sequence and the dTomato reporter, allowing for the expression of the dTomato reporter under the control of the S9E27 enhancer element in cells of the brain. Brain tissue was analyzed 3 weeks post-injection by immunohistochemistry (IHC) for expression of both the reporter and the ChAT marker in interneuron cells of the brain. The images labeled “dTom” show viral expression of the dTomato reporter under control of the S9E27 enhancer element (white stippled areas). The images labeled “Chat” show expression of ChAT in ChAT-expressing interneurons targeted by the enhancer, as detected by anti-ChAT antibody staining (white areas). The left images labeled “merged” show expression of both the dTomato and ChAT marker in ChAT-expressing interneurons. Quantification analysis in the cortex showed that >90% of cells expressing the viral reporter co-expressed the ChAT cholinergic marker. The high degree of co-localization and the layer specific distribution of cells expressing the viral reporter demonstrate that the S9E27 enhancer restricts expression of cholinergic neurons across brain structures. Overall, the specificity of the S9E27 enhancer for targeting cholinergic neurons and expressing the dTomato reporter transgene in the specific ChAT neuron cell population is observed.

FIG. 9 provides images demonstrating the expression profile and specificity of the S9E24 and S9E36 enhancer elements in mice using IHC with the indicated markers (PV and dTomato reporter). Adult mice were injected systemically with an rAAV vector construct containing the S9E24 or the S9E36 enhancer element sequence and the dTomato reporter, allowing for the expression of the dTomato reporter under the control of these enhancer elements (enhancer regulatory elements) in cells of the brain. Brain tissue was analyzed 3 weeks post-injection by immunohistochemistry (IHC) for expression of both the reporter and the PV marker in brain neuronal cells. The staining results show the specificity of expression of the S9E24 and S9E36 enhancers and reporter expression in arkypallidal (ArkyP) projection neurons in the brain. In the merged images, PV staining (of PV-expressing cells) is more localized in the left side of the images, while reporter (dTomato) staining is more localized in the right side of the images. The lack of co-localization with PV shows that the targeted neuronal cell population corresponds to ArkyP projection neurons in the globus pallidus.

FIG. 10 provides images of brain regions following systemic injection of AAV vectors (AAVs) containing one of S9E21 to S9E24, S9E33, S9E34, or S9E36 enhancer elements in mice. The indicated enhancers were characterized for their expression profiles across brain regions. Each enhancer was found to have a unique expression profile, showing strong expression in specific subsets of brain regions, including the globus pallidus (GP), thalamic nuclei, Zona Inserta (ZI), Substantia Nigra (SNr) and subthalamic nuclei (STN). The individual images at the left and middle of FIG. 10 show sagittal sections of mouse brains injected systemically with AAVs containing the indicated enhancer. Shown at the right in FIG. 10 is a representative image of the expression profiles of these enhancers in the indicated regions of the brain, such as the globus pallidus and thalamic and subthalamic structures.

FIG. 11 illustrates the approach used in the identification and isolation of cell-type specific enhancer sequences as described herein.

FIGS. 12A-12D provide schematics, illustrations, confocal microscope images, graphs and tracings (resulting from electro-physiological studies). FIG. 12A shows a schematic depiction of a vector construct (e.g., AAV) containing, from left to right, an isolated enhancer element sequence as described herein, a promoter sequence, a nuclear localization signal (nls) for the dTom reporter gene, and the dTom reporter gene. FIG. 12B shows an illustration of brain cortical regions depicting the localization of the area and cells (somatostatin (SST)-expressing neuronal cells) targeted by the S9E10 enhancer element when the AAV construct is injected into cortical tissue. In FIG. 12C, which presents confocal microscope images of immunohistochemical staining of SST neuronal cells in mouse cerebral cortex, the leftmost image shows expression of SST marker in SST-neurons in layers of the cortical tissue. The middle image shows viral expression of dTomato marker (reporter) under control of the S9E10 enhancer element in the image. The rightmost image (merged) shows expression of both the dTomatoSST and the SST marker in SST-expressing interneurons targeted by the S9E10 enhancer. The high degree of colocalization and the layer specific distribution of cells expressing the viral reporter as evidenced by the staining demonstrate that the S9E10 enhancer restricted gene expression in SST interneurons.

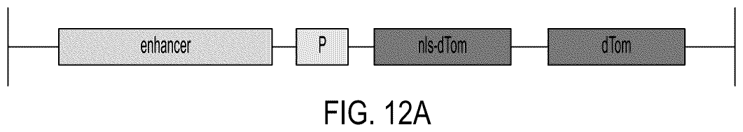

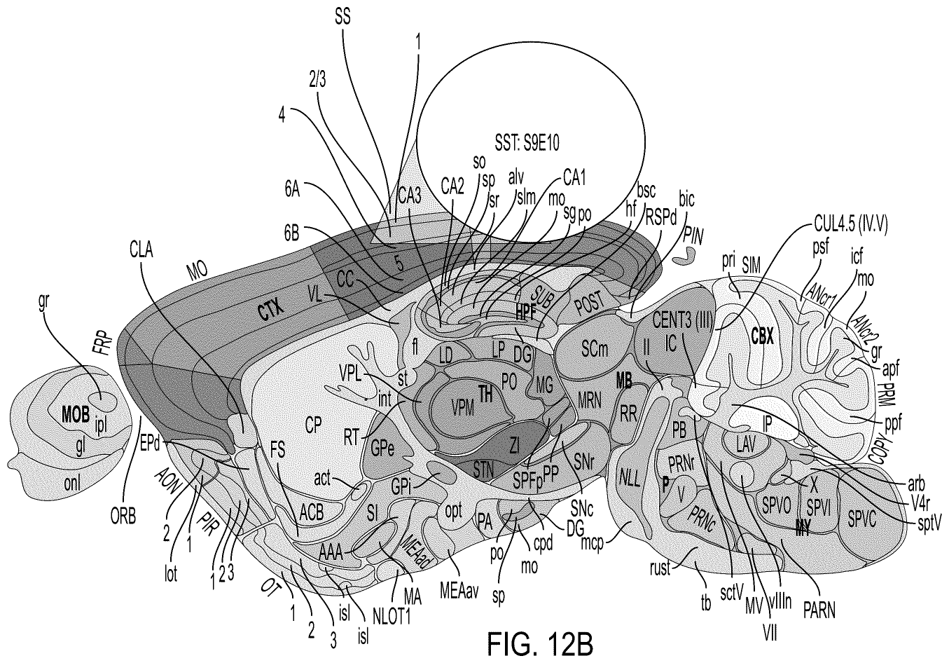

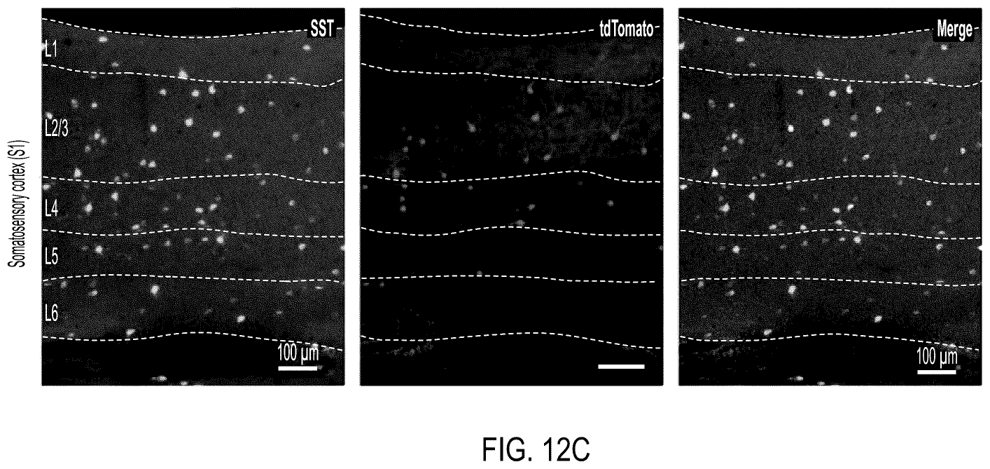

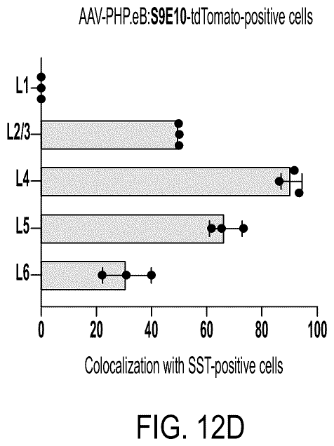

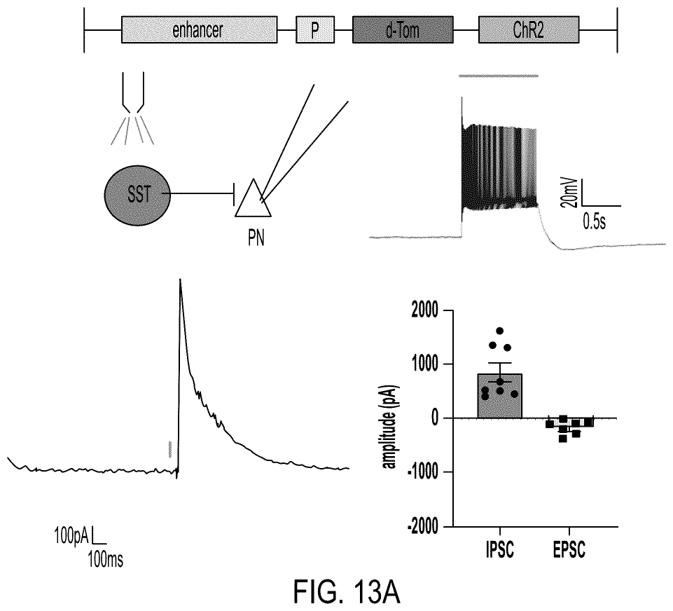

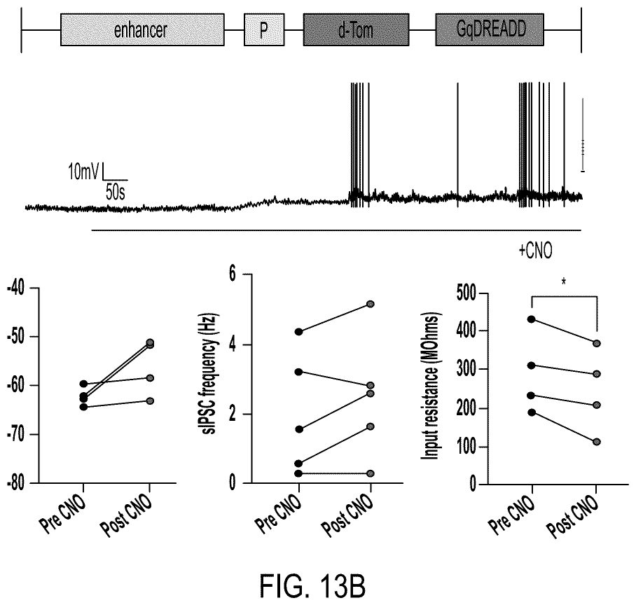

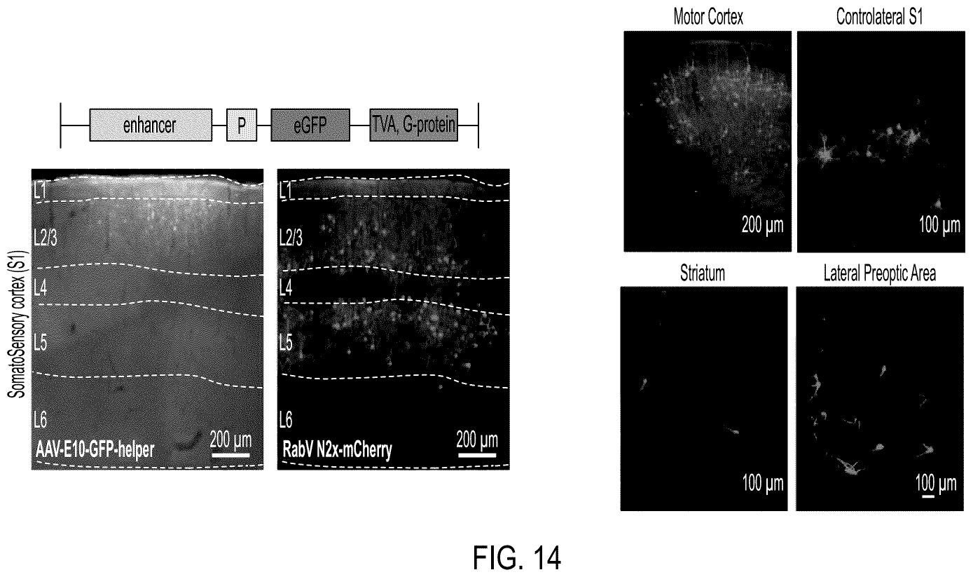

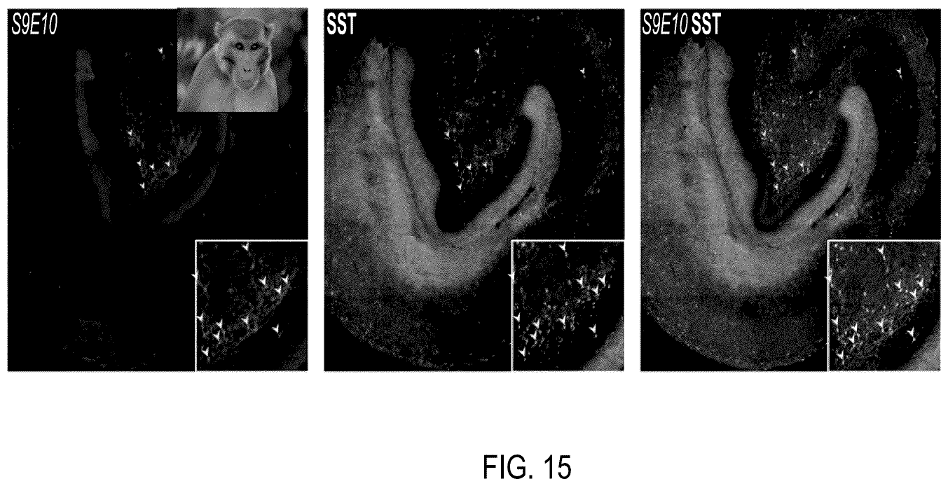

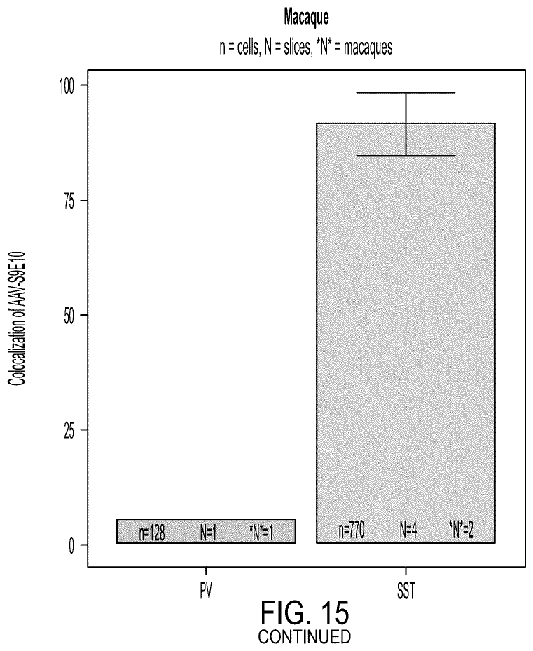

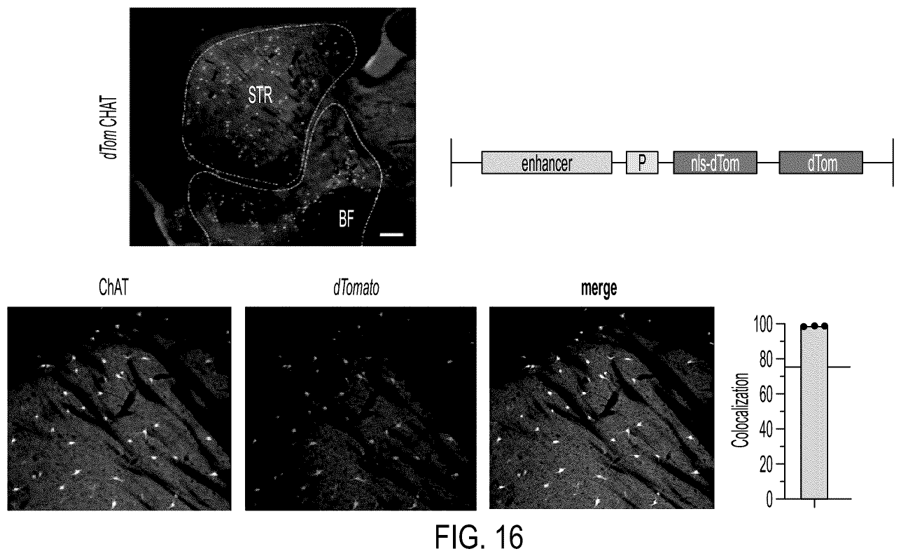

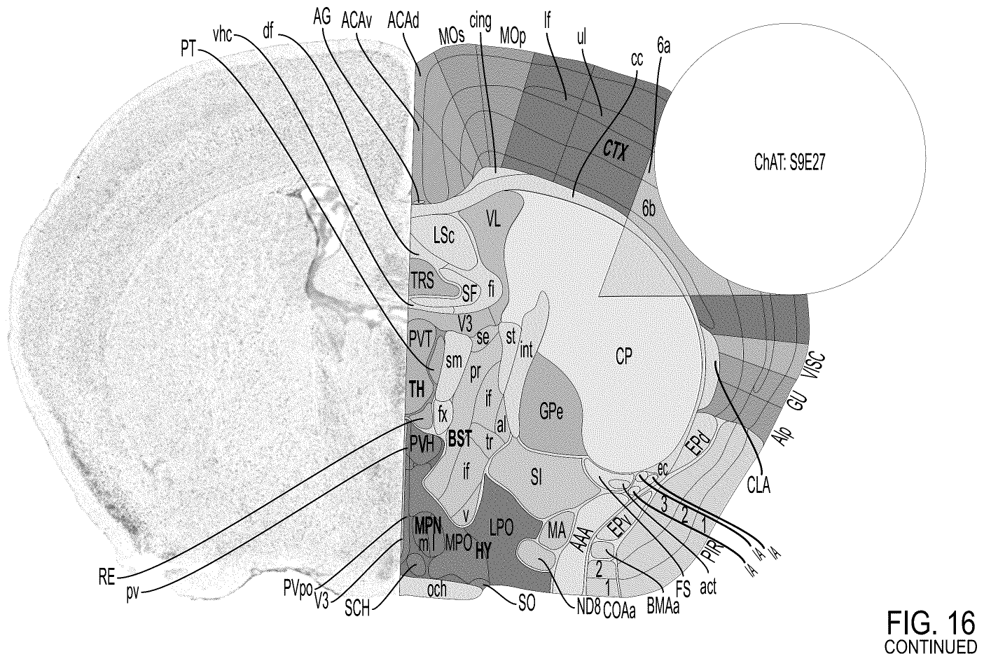

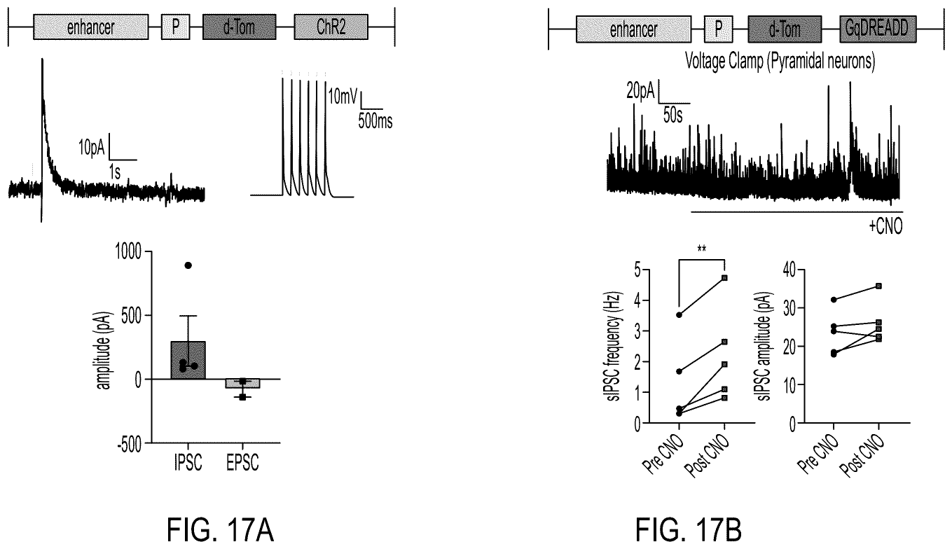

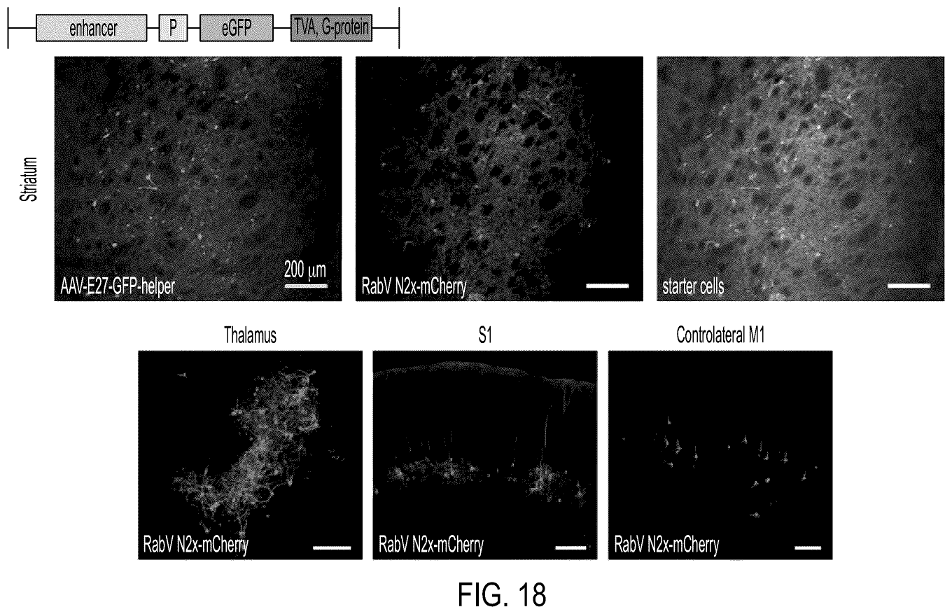

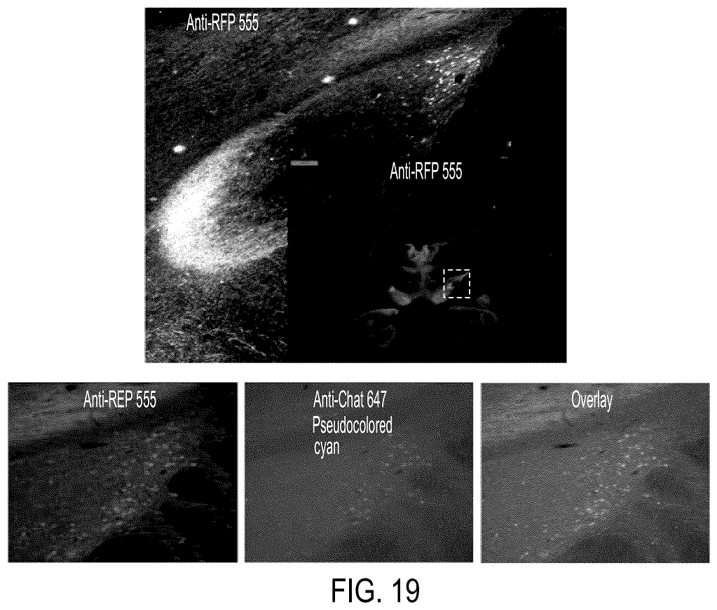

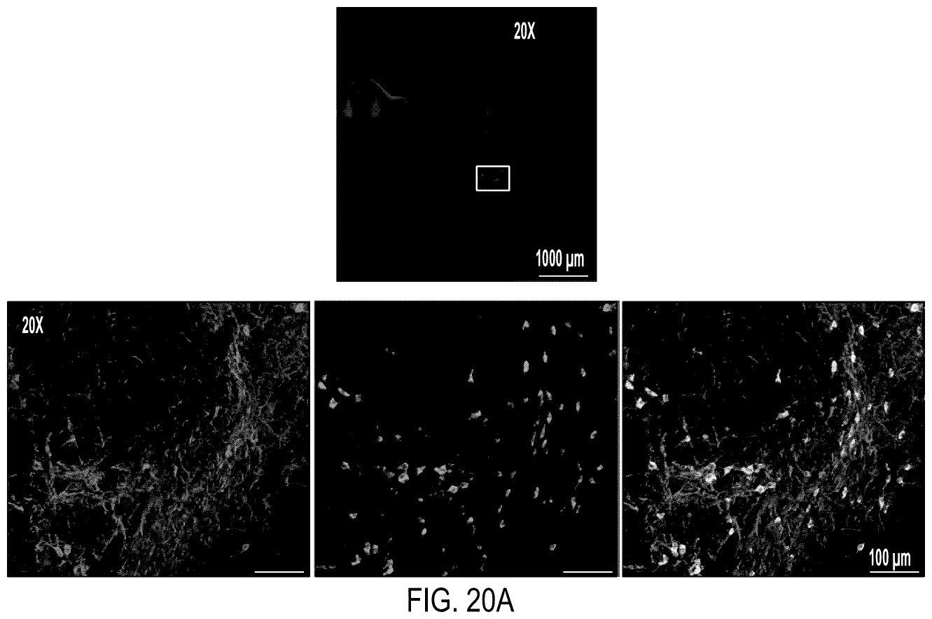

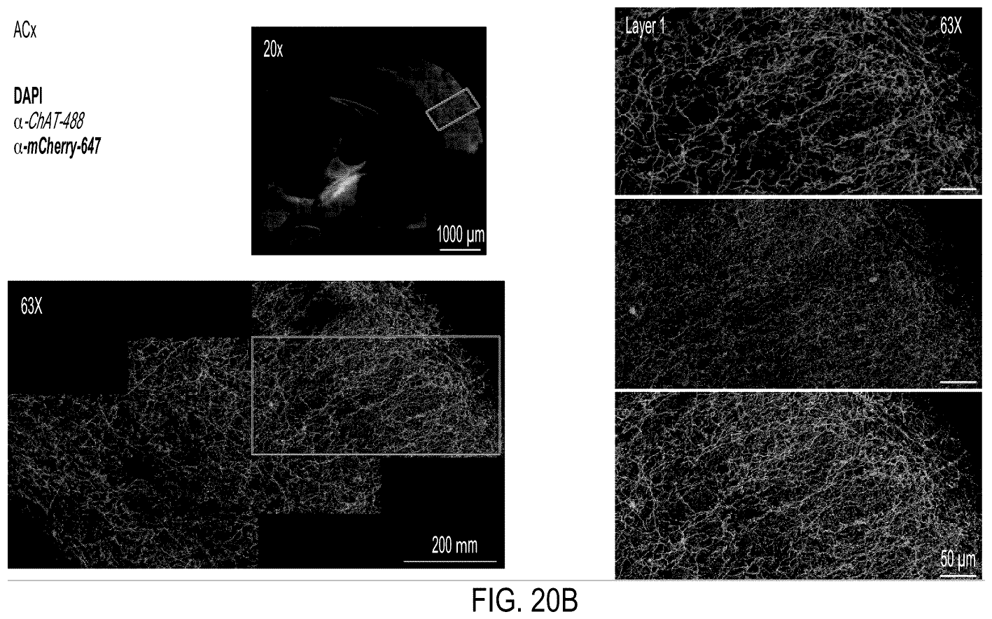

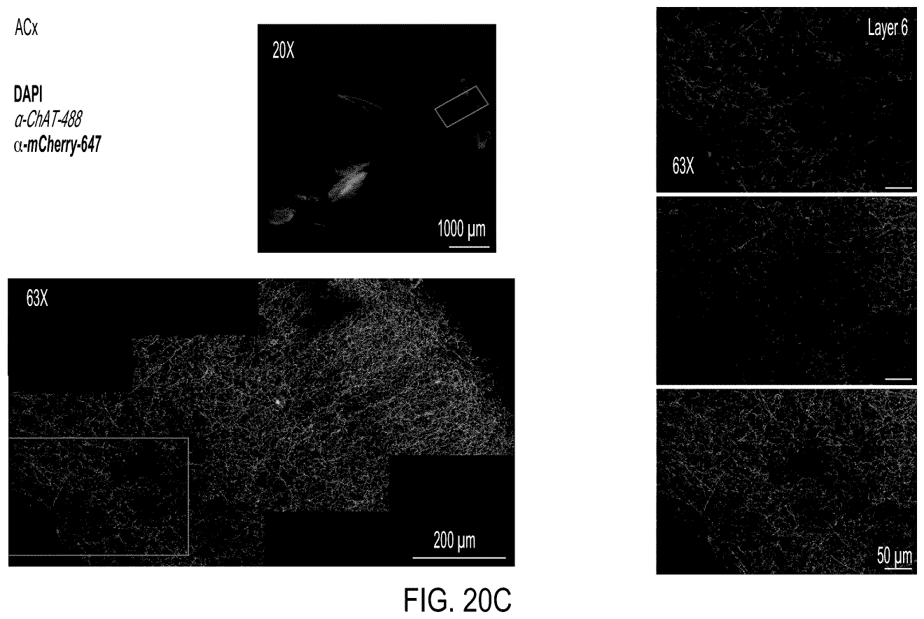

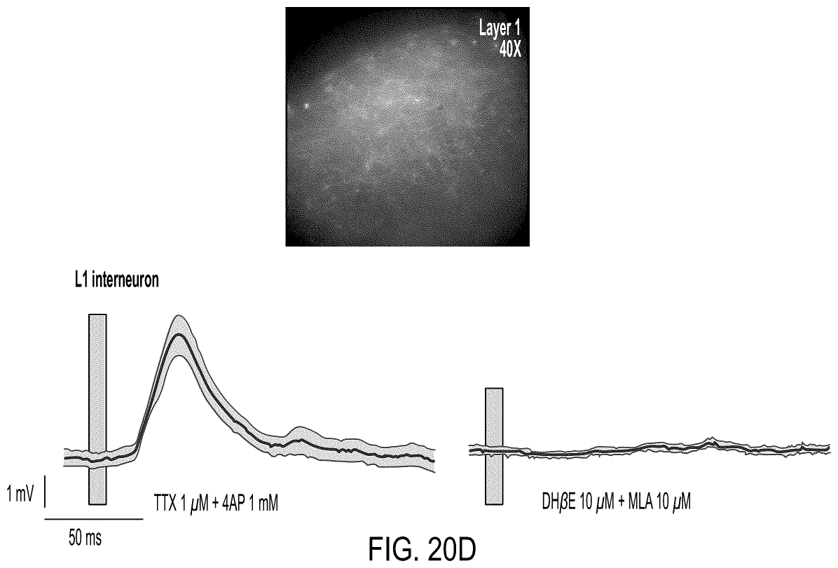

In brief, for the experiments, adult mice were injected systemically with an rAAV vector construct containing the enhancer element sequence, e.g., S9E10 (AAV-S9E10), (or another enhancer element sequence as described herein), and the dTomato reporter, allowing for the expression of the dTomato reporter under the control of the S9E10 enhancer element in cells of the brain. Brain tissue was analyzed 3 weeks post-injection by immunohistochemistry (IHC) for expression of both the reporter and the SST marker in interneuron cells of the brain. FIG. 12D presents a graph showing amounts of co-localization of dTomato reporter gene expression with SST-positive cells, as targeted by the S9E10 enhancer component of the AAV construct, in the different cortical layers (L1-L6).

FIGS. 13A and 13B provide schematics, illustrations, graphs and tracings of results from optogenetic and chemogenetic studies related to the use of viral constructs containing the isolated enhancer sequence S9E10 that targets in SST neurons and restricts the expression of genes in SST neurons. FIG. 13A shows a schematic of a vector construct (e.g., an rAAV vector construct) containing an isolated enhancer element, a promoter sequence, d-Tomato reporter gene, and ChR2 (Channelrhodopsin-2) gene. ChR2 is a nonspecific cation channel derived from the green algae Chlamydomonas reinhardtii. ChR2 absorbs blue light, causing a conformational change that allows H⁺, Na⁺, K⁺, and Ca⁺ ions to passively diffuse down their concentration gradients. Current-clamp experiments can be conducted in ChR2-expressing cells to assess the cells for light-induced depolarization when the cells are illuminated with blue light, (G. Nagel et al., 2003, Proc. Natl. Acad. Sci. USA, 100(24): 13940-13945), as shown in the illustration, tracings and graph in the figure. The graph in FIG. 13A shows inhibitory postsynaptic current (IPSC) and excitatory postsynaptic current (EPSC) versus amplitude (pA) in cells transduced with the vector construct and subjected to patch voltage-clamp recordings. FIG. 13B shows a schematic of a vector construct (e.g., an rAAV vector construct) containing an isolated enhancer element, a promoter sequence, d-Tomato reporter gene, and a GqDREADD (Designer Receptors Exclusively Activated by Designer Drugs) gene. Gq-DREADDs are modified muscarinic G-protein coupled chemogenetic receptors that are introduced into cells by viral vectors where they selectively modulate cellular activity by chemical means. Gq-DREADDs signal through the Gαq/11 G-protein and activate neuronal firing through stimulating phospholipase C, which releases intracellular calcium stores. There are currently three Gq DREADDs based on human muscarinic receptors: hM1Dq, hM3Dq, and hM5Dq, with hM3Dq being the most widely employed. Gq-DREADDs provide a chemogenetic approach that allows for selective and remote control of neuronal activity with a high degree of spatial resolution. (B. L. Roth, 2016, Neuron, 89(4): 683-694.) The muscarinic receptor-based DREADDs can be engineered to respond to nM concentrations of ligand clozapine N-oxide (CNO), a metabolite of the antipsychotic clozapine, which is pharmacologically inert and does not cross the blood-brain barrier in rodents (mice and rats). The tracing in FIG. 13B shows that the neuronal cells transduced with the GqDREADD-encoding AAV vector showed activity following transduction and contact with the CNO ligand. The graphs in FIG. 13B show spontaneous inhibitory postsynaptic current (sIPSC) frequency (middle graph) and Input resistance (rightmost graph) of neuronal cells transduced with the AAV vector pre- and post-contact with CNO. As shown by the results of the optogenetic and chemogenetic studies, the same SST-targeting, isolated enhancer sequence S9E10 can be used to activate SST neuronal cells.

FIG. 14 shows a schematic depiction of an AAV vector construct containing, from left to right, an isolated enhancer element sequence as described herein, namely, the SST-targeting S9E10 enhancer element, a promoter sequence, a green fluorescent protein (GFP) reporter sequence and TVA and Rabies virus G protein sequences (TVA, G-protein), e.g., as available from Addgene (Watertown, MA). Engineered rabies virus (RV) and AAV vectors are commonly used retrograde viral tracers because of their properties of axon terminal absorption and rapid gene expression, particularly in neuronal circuit assemblies. RV-AG pseudotyped with the avian sarcoma leucosis virus glycoprotein EnvA can specifically recognize and infect neurons that express the TVA receptor, an avian receptor protein that is absent in mammalian cells. TVA can be expressed by transgenic animals or viral vectors, and retrograde infection of the EnvA-pseudotyped virus along the axon terminal can be restricted to directly input neurons. (See, e.g., Z. Han et al., 2022, Molecular Brain, 15(13):1-13; T. J. M. Roelofs et al., 2021, Nature Scientific Reports, 11(10400), 12 pages; E. M. Callaway et al., 2015, J. Neurosci., 35(24):8979-8985). In brief, RV is targeted to cells expressing TVA. The initial RV infection is targeted to starter cells of interest by expressing TVA in those cells. The presence of GFP in the vector results in the cells infected with RV to express and be labeled with GFP. In some case, the starter cells also express a unique marker gene, for example, an RFP or a TVA-mCherry fusion protein, which allows them to be uniquely identified for subsequent analyses. Because RVdG is not able to spread between neurons in the absence of G, G also needs to be expressed in the starter cells. The G expressed in trans allows for trans-complementation and the production of G+RVdG particles that can bud out from the starter cells. These particles have rabies G in their envelope and thus have the normal infectious properties of the intact virus. They can be taken up selectively at synaptic sites by the presynaptic terminals of neurons. (Callaway et al., Ibid.). The leftmost panels of the figure show microscopic images of the staining of layers (e.g., L1, L2/3, L4, L5 and L6) of the mouse somatosensory cortex (S1) following transduction of neuronal cells in the cortex with the vector construct harboring the isolated S9E10 enhancer sequence. The rightmost panels of the figure show microscopic images of the staining of neurons in the motor cortex, striatum, contralateral S1, and lateral preoptic area following transduction of neuronal cells in the cortex with the vector construct. The layer specific distribution of cells expressing the viral reporter as evidenced by the staining demonstrate that the S9E10 enhancer targeted and restricted gene expression of rabies virus protein in neuronal cells located in specific areas of the cerebral cortex.

FIG. 15 shows confocal microscope images of tissue slices containing fluorescently-stained neurons and a graph showing colocalization of the AAV-S9E10 enhancer element-containing vector used to transduce SST-expressing neuronal cells in the hippocampus of the brain in macaque monkeys. A specialized population of SST-expressing cells in the macaque hippocampus showed staining. Co-localization of the S9E10 enhancer targeting SST-expressing cells as transduced by the AAV delivery vector harboring S9E10 and SST-expressing cells is evident.

FIG. 16 provides schematic illustrations, confocal microscope images and a graph. The illustration of the vector construct similar to that described in FIG. 12A. In the studies related to this figure, the construct contained the isolated ChAT cell-targeting enhancer sequence S9E27. Also shown is an illustration of brain cortical regions depicting the localization of the area and cortical ChAT cells (choline acetyltransferase (ChAT)-expressing neuronal cells) targeted by the S9E27 enhancer element when the AAV construct is injected into cortical tissue. ChAT is a transferase enzyme that is responsible for the synthesis of the neurotransmitter acetylcholine (ACh) by catalyzing the transfer of an acetyl group from the coenzyme acetyl-CoA to choline. The confocal images show reporter and ChAT staining of ChAT-expressing cells of the cortex that were transduced with the vector construct and targeted by the isolated enhancer element S9E27 sequence present in the construct. The top image shows dTom+ChAT staining in the delineated striatum (STR) and basal forebrain (BF) areas of the cerebral cortex. The leftmost microscope image at the bottom shows staining of ChAT in ChAT-expressing cells in the cortex; the middle image shows dTomato expression in the cells; and the rightmost image shows a merger of the ChAT and d-Tomato staining results. The graph shows colocalization of AAV-S9E27 with ChAT-positive cells. The high degree of colocalization and the specific distribution of cells expressing the viral reporter as evidenced by the staining demonstrate that the S9E27 enhancer element targeted and restricted gene expression in ChAT interneurons.

FIGS. 17A and 17B present schematic illustrations of vector constructs, electrophysiological tracings and graphs. FIG. 17A depicts the vector illustration as described in FIG. 13A. The tracings and graph below the vector illustration in FIG. 17A demonstrate the results of optogenetic studies of ChAT-expressing neuronal cells (ChAT/cholinergic neurons) targeted by the construct containing the isolated S9E27 enhancer element sequence that restricts expression of genes (e.g., ChR2) to ChAT neurons. FIG. 17B depicts the vector illustration as described in FIG. 13B. The tracings and graph below the vector illustration in FIG. 17B demonstrate the results of chemogenetic studies of ChAT-expressing neuronal cells (ChAT/cholinergic neurons) targeted by the construct containing the isolated S9E27 enhancer element sequence that restricts expression of genes (e.g., Gq-DREADD) to ChAT neurons.

FIG. 18 presents a schematic illustration of the vector construct as described in FIG. 14 and confocal microscope images. (AAV-S9E27-GFP-TVA, G-protein). The isolated enhancer sequence contained in the AAV viral vector used in this study was S9E27. Shown in the confocal microscope images is staining in the striatum, thalamus, S1 and contralateral M1 regions of the cerebral cortex following transduction of cells in these regions with the viral vector carrying the rabies virus protein. The high degree of specific distribution of cells expressing the viral reporter as evidenced by the staining patterns demonstrates that the S9E27 enhancer restricted gene expression of the rabies virus protein in ChAT-expressing neuronal cell populations in the brain regions as shown.

FIG. 19 shows the specificity of staining of ChAT-expressing neurons in the cerebral cortex of a macaque monkey following transduction of the cells in the cortex with an AAV vector containing an isolated ChAT-targeting enhancer sequence (S9E27) and a reporter gene.

FIGS. 20A-20D show confocal microscope images (20×/63×) and histology and physiology data using the isolated S9E27 enhancer element encoded by a recombinant adeno-associated virus vector with the reporters ChR2 and m-Cherry (AAVS9E27-ChR2-mCherry construct). Anatomical studies using this enhancer element show co-localization of the enhancer with ChAT-specific staining of basal forebrain neurons as well as axons in the auditory cortex in mice. In addition, cholinergic currents were recorded in auditory cortical neurons following transduction of the cells using the isolated S9E27 enhancer/enhancer construct. Adult mice were injected systemically with the rAAV vector construct containing the S9E27 enhancer element sequence and the dTomato reporter, allowing for the expression of the dTomato reporter under the control of the S9E27 enhancer element (enhancer regulatory element) in cells of the brain. Brain tissue was analyzed 3 weeks post-injection by immunohistochemistry (IHC) for expression of both the reporter and the marker in neuronal cells of the brain. FIG. 20A shows confocal images of histology staining of neuronal cells (ChAT neurons) following transduction of cortical neurons with the AAVS9E27-ChR2-mCherry construct. Injection details: AAVS9E27-ChR2-mCherry (1:10→1.3×10¹²) in GP/SI. (AP=−0.2/−0.8 mm—ML=2.5 left—DV=−4/−3.25). Top image: DAPI (4′,6-diamino-2-phenylindole) staining of cortical tissue; Left and Right images: α-mCherry-647 staining; Middle image; α-ChAT-488 staining of cholinergic neurons (ChAT). FIG. 20B shows confocal images of histology staining of neuronal cells (ChAT neurons) at 20× and 63× magnification as described for FIG. 20A. The top left image (20× magnification) shows DAPI staining of cortical brain tissue, and the region from which the images at the bottom left and the set of images on the right (Layer 1 of the cortical tissue; 63× magnification) were taken. Bottom left image: staining with DAPI, α-mCherry-647, and α-ChAT-488. Top right image: staining with DAPI and α-mCherry-647; Middle right image: staining with DAPI and α-ChAT-488; Bottom right image: staining with DAPI, α-mCherry-647, and α-ChAT-488. FIG. 20C shows staining results as described for FIG. 20B, except that the right set of images represents staining of cells in Layer 6 of the cortical tissue (63× magnification).

FIG. 20D shows an image (top portion of figure; 40× magnification) of Layer 1 (L1) of mouse cortical tissue into which the AAVS9E27-ChR2-mCherry construct was injected, as described for FIG. 20A. The bottom portion of the figure presents electrophysiological tracings demonstrating polarization and depolarization of L1 interneurons. The leftmost tracing shows the results of contacting L1 interneuron cells with the voltage-gated sodium channel blocker tetrodotoxin (TTX), (1 μM)+4AP (1 mM). Neuronal cell sodium channel activity (increased action potential) is observed following the application of TTX+4AP in the L1 interneuron cells, as the AAVS9E27 targets the reporter for expression in interneurons expressing sodium channels and exhibiting sodium channel activity. The rightmost tracing shows the results of contacting L1 interneuron cells with DHβE (10 μM), a member of the Erythrina family of alkaloids and a potent competitive antagonist of the α4β2-subtype of the neuronal nicotinic acetylcholine receptors (nAChRs), + methyllycaconitine (MLA) (10 μM). MLA is a selective and potent antagonist of the α7 neuronal nicotinic acetylcholine receptor (nAChR); its anthracite ester side-chain is important for its activity. No neuronal cell activity is observed following the application of DHβE+MLA in the L1 interneuron cells, as the AAVS9E27 does not target the reporter for expression in interneurons expressing nAChRs.

### DETAILED DESCRIPTION OF THE EMBODIMENTS

Described and provided herein are new enhancer element sequences (termed mouse or human S9E1-S9E40 herein that target, restrict, regulate, or modify the expression of a gene (e.g., a transgene) in certain neuronal cell types and/or populations. As described herein, forty (40) mouse enhancer sequences (SEQ ID NOs: 1, 3, 5, 7, 9, 11, 13, 15, 17, 19, 21, 23, 25, 27, 29, 31, 33, 35, 37, 39, 41, 43, 35, 37, 49, 51, 53, 55, 57, 59, 61, 63, 65, 67, 69, 71, 73, 75, 77 and 79) were identified as being capable of restricting the expression of a transgene to different neuronal cell types and/or neuronal cell populations. In addition, the enhancer sequences that are the human counterparts of the mouse enhancer sequences were identified (SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 and 80, respectively). (Table 1 herein).

The isolated enhancer sequences as described herein were discovered using an in-silico selection (see. e.g., D. Vormstein-Schneider et al., 2020, Nat. Neurosci., 23(12):1629-1636, incorporated herein by reference), which relies on combining chromatin accessibility data with cross-species conservation to identify putative enhancer elements in the vicinity of genes enriched in the target neuronal population. In embodiments, the application of the method led to the identification of five candidate enhancer sequences for eight specific neuronal populations, namely, four distinct classes of inhibitory GABA-ergic neurons (i.e. parvalbumin-expressing interneurons (PV interneurons); Somatostatin expressing interneurons (SST interneurons); Vaso-active Intestinal Peptide expressing interneurons (VIP interneurons) and non-VIP/CGE-derived interneurons (ID2 interneurons); and four distinct classes of basal forebrain neurons (i.e. Dopamine-Receptor 1 expressing medium-spiny neurons (D1 (or Drd1) neurons); Dopamine-Receptor 2 expressing medium-spiny neurons (D2 (or Drd2) neurons); Cholinergic interneurons of the striatum (Ch-IN interneurons); and Cholinergic projection neurons of the basal ganglia (Ch-PN neurons) in the central nervous system (CNS), including the brain. Table 1 presents the mouse and human enhancer sequences, their SEQ ID NOs., target neuronal cell type and target gene.

TABLE 1

| Mouse Enhancer ID | Human Enhancer ID | Intended Target | |
|---|---|---|---|
| SEQ ID NO | SEQ ID NO | Neuron Population | Target Gene |
| S9E1 SEQ ID NO: 1 | huS9E1 SEQ ID NO: 2 | PV_Basket | Prss23 |
| S9E2 SEQ ID NO: 3 | huS9E2 SEQ ID NO: 4 | PV_Basket | Lpl |
| S9E3 SEQ ID NO: 5 | huS9E3 SEQ ID NO: 6 | PV_Chandelier | Cntnap5b |
| S9E4 SEQ ID NO: 7 | huS9E4 SEQ ID NO: 8 | PV_Chandelier | Plcxd3 |
| S9E5 SEQ ID NO: 9 | huS9E5 SEQ ID NO: 10 | PV_Chandelier | Elf5 |
| S9E6 SEQ ID NO: 11 | huS9E6 SEQ ID NO: 12 | SST | Satb1 |
| S9E7 SEQ ID NO: 13 | huS9E7 SEQ ID NO: 14 | SST | Ccna1 |
| S9E8 SEQ ID NO: 15 | huS9E8 SEQ ID NO: 16 | SST | Calb1 |
| S9E9 SEQ ID NO: 17 | huS9E9 SEQ ID NO: 18 | SST | Smc2 |
| S9E10 SEQ ID NO: 19 | huS9E10 SEQ ID NO: 20 | SST | Hpse |
| S9E11 SEQ ID NO: 21 | huS9E11 SEQ ID NO: 22 | VIP | Prox1 |
| S9E12 SEQ ID NO: 23 | huS9E12 SEQ ID NO: 24 | VIP | Prox1 |
| S9E13 SEQ ID NO: 25 | huS9E13 SEQ ID NO: 26 | VIP | Vip |
| S9E14 SEQ ID NO: 27 | huS9E14 SEQ ID NO: 28 | VIP | Npy5r |
| S9E15 SEQ ID NO: 29 | huS9E15 SEQ ID NO: 30 | VIP | Grpr |
| S9E16 SEQ ID NO: 31 | huS9E16 SEQ ID NO: 32 | ID2 | Sv2c |
| S9E17 SEQ ID NO: 33 | huS9E17 SEQ ID NO: 34 | ID2 | Pde11a |
| S9E18 SEQ ID NO: 35 | huS9E18 SEQ ID NO: 36 | ID2 | Wt1 |
| S9E19 SEQ ID NO: 37 | huS9E19 SEQ ID NO: 38 | ID2 | Lamp5 |
| S9E20 SEQ ID NO: 39 | huS9E20 SEQ ID NO: 40 | ID2 | Lamp5 |
| S9E21 SEQ ID NO: 41 | huS9E21 SEQ ID NO: 42 | Drd1 | Slc35d3 |
| S9E25 SEQ ID NO: 43 | huS9E25 SEQ ID NO: 44 | Drd1 | Drd1 |
| S9E33 SEQ ID NO: 45 | huS9E33 SEQ ID NO: 46 | Drd1 | Chrm4 |
| S9E34 SEQ ID NO: 47 | huS9E34 SEQ ID NO: 48 | Drd1 | Chrm4 |
| S9E36 SEQ ID NO: 49 | huS9E36 SEQ ID NO: 50 | ArkyP; Drd1 | Tac1 |
| S9E22 SEQ ID NO: 51 | huS9E22 SEQ ID NO: 52 | Drd2 | Gpr6 |
| S9E23 SEQ ID NO: 53 | huS9E23 SEQ ID NO: 54 | Drd2 | Gpr6 |
| S9E24 SEQ ID NO: 55 | huS9E24 SEQ ID NO: 56 | ArkyP; Drd2 | Adora2a |
| S9E31 SEQ ID NO: 57 | huS9E31 SEQ ID NO: 58 | Drd2 | Sp9 |
| S9E32 SEQ ID NO: 59 | huS9E32 SEQ ID NO: 60 | Drd2 | Sp9 |
| S9E27 SEQ ID NO: 61 | huS9E27 SEQ ID NO: 62 | Cholinergic (ChAT) | Chat |
| S9E28 SEQ ID NO: 63 | huS9E28 SEQ ID NO: 64 | Cholinergic (ChAT) | Chat |
| S9E38 SEQ ID NO: 65 | huS9E38 SEQ ID NO: 66 | Cholinergic (ChAT) | Zic1 |
| S9E39 SEQ ID NO: 67 | huS9E39 SEQ ID NO: 68 | Cholinergic (ChAT) | Zic1 |
| S9E26 SEQ ID NO: 69 | huS9E26 SEQ ID NO: 70 | Ch-IN | Isl1 |
| S9E35 SEQ ID NO: 71 | huS9E35 SEQ ID NO: 72 | Ch-IN | Tshz2 |
| S9E40 SEQ ID NO: 73 | huS9E40 SEQ ID NO: 74 | Ch-IN | Zic1 |
| S9E29 SEQ ID NO: 75 | huS9E29 SEQ ID NO: 76 | Ch-PN | Chat |
| S9E30 SEQ ID NO: 77 | huS9E30 SEQ ID NO: 78 | Ch-PN | Pcdh9 |
| S9E37 SEQ ID NO: 79 | huS9E37 SEQ ID NO: 80 | Ch-PN | Zic1 |

The isolated enhancer sequences S9E1-S9E40 (SEQ ID NOS: 1-80) as described herein can be used to restrict or regulate therapeutic interventions, such as, without limitation, gene replacement, gene modulation, gene editing, modulation of cellular activity, to distinct neuronal populations in the CNS and across organs. Because limiting the intervention to the cellular population exclusively affected by a given pathology has the potential to greatly reduce off-target effects, the new enhancer sequences identified and described herein have the potential to substantially improve current or future therapeutic approaches for the treatment of diseases, disorders, and pathologies of the CNS and the brain. The enhancer elements as identified and described herein may be employed across species and/or in cells of different species. By way of example, the mouse and human enhancer element sequences provided here can be used to restrict the expression of genes, e.g., transgenes, in human neuron and interneuron cell types and can be utilized in other mammalian subjects.

In embodiments, the enhancer sequences (e.g., S9E1 to S9E40, SEQ ID NOs: 1-80) were individually cloned into AAV viral vectors, which were used to produce AAV particles. In an embodiment, the AAV viral vectors contained a transgene. When administered to (e.g., injected into) mice, e.g., adult mice, the viruses were capable of driving expression of the transgene harbored in the vector exclusively in target neuronal populations.

By way of representative and nonlimiting examples, enhancer element sequences, S9E10 (SEQ ID NO: 19) (huS9E10 (SEQ ID NO: 20)) and S9E27 (SEQ ID NO: 61) (huS9E27 (SEQ ID NO: 62) showed a high degree of specificity for targeting two therapeutically relevant neuronal cell populations. The S9E10 enhancer exhibited >85% specificity for SST interneurons in the cortex, which is therapeutically advantageous, particularly for clinical intervention to reduce seizures and associated neuropsychiatric disorders.

The S9E27 enhancer exhibited >95% specificity for both Cholinergic interneurons in the striatum and Cholinergic projection neurons in the basal nuclei. The ability of the enhancer sequence to target these neuronal populations is advantageous and beneficial for therapeutics and treatment for neurological and neurodegenerative disorders, including, without limitation, Alzheimer's disease, Parkinson's disease, Dystonia, amyotrophic lateral sclerosis (ALS), Down Syndrome, and/or symptoms thereof. The S9E27 enhancer may be beneficial and useful in therapy and treatment of movement disorders, including, but not limited to, ataxia, dystonia, tremors, Essential Tremor, Lewy Body dementia, motor stereotypies, Parkinson's disease, and/or symptoms thereof.

The S9E2 enhancer targets a subset of PV-interneurons called Chandelier cells that represent less than 1% of all cortical neurons and that may be associated with and/or involved in cognition and seizures. The S9E2 enhancer sequence may be useful in clinical intervention to reduce seizures and associated neuropsychiatric disorders, and/or symptoms thereof.

The S9E24 and S9E36 enhancers target the arkypallidal (ArkyP) neuronal cell population in the globus pallidus region of the brain. ArkyP neuronal cells are involved in the regulation of voluntary movement. As such, these two enhancer sequences may be useful in clinical intervention to alleviate the symptoms related to neuromuscular and movement disorders, including, but not limited to, ataxia, dystonia, tremors, Essential Tremor, Lewy Body dementia, motor stereotypies, Parkinson's disease, and/or symptoms thereof.

The S9E21-24, 33, 34, 36 enhancers show a diverse set of expression profiles in specific regions of the ventral CNS, including the globus pallidus and thalamic/subthalamic structures. Stimulation of neurons in these regions has been shown to positively affect various aspects of movement disorders, including, but not limited to, ataxia, dystonia, tremors, Essential Tremor, Lewy Body dementia, motor stereotypies, Parkinson's disease, and other conditions, such as obsessive-compulsive disorder (OCD) and epilepsy, and/or symptoms thereof.

Vectors, such as expression vector, e.g., AAV, containing the above-described enhancers and polynucleotides (e.g., transgenes; therapeutic genes) can be used to restore normal cellular function, e.g., by restoring expression of certain genes to the appropriate interneuron or neuron target cell populations, advantageously to address the cause of the disease, for example, by restoring the excitation-inhibition balance in the neuronal cell or cell population.

Specific viral-based therapeutic products, compositions, methods and approaches for treating or ameliorating neurological, neurodevelopmental, neurodegenerative, neuropsychiatric, neurogenetic, or neuromuscular diseases, disorders, and pathologies are described herein. As described, virus vectors and vehicles for gene delivery are designed and produced to contain a specific enhancer sequence (enhancer) and a polynucleotide sequence of a gene of interest, such as a transgene or reporter gene, which is functionally expressed in certain interneuron or neuron cell populations following transduction of the interneuron or neuron cells by the virus vector or vehicle. In an embodiment, a virus vector or vehicle is provided which comprises the polynucleotide of a specific enhancer sequence (enhancer), which is functionally expressed in certain interneuron or neuron cell populations following transduction of the interneuron or neuron cells by the virus vector or vehicle. In an embodiment, the enhancer harbored by the virus is capable of restricting the expression of the transgene to certain neuronal or interneuronal cells. In embodiments, the cells include inhibitory GABA-ergic neurons, namely, parvalbumin (PV)-expressing interneurons, Somatostatin (SST)-expressing interneurons; Vaso-active Intestinal Peptide (VIP)-expressing interneurons; and non-VIP/CGE-derived interneurons (ID2), or basal forebrain neurons, namely, Dopamine-Receptor 1 (D1)-expressing medium-spiny neurons (Drd1 neurons); Dopamine-Receptor 2 (D2)-expressing medium-spiny neurons (Drd2 neurons); Cholinergic neurons (e.g., ChAT), Cholinergic interneurons of the striatum (Ch-IN); and Cholinergic projection neurons of the basal ganglia (Ch-PN). In embodiments, expression of the transgene is restricted to expression in cells that are deficient for that gene, or in cells that have a nonfunctional, mutated, or aberrantly expressed gene. In an embodiment, the expression of the transgene is specifically modulated in the interneuron cell or other neuronal cell. In other embodiments, the transgene is an effector gene or a therapeutic gene.

In an embodiment, the virus vector contains a specific enhancer sequence and a transgene (e.g., a therapeutic gene or an effector gene) associated with a neurological, neurodevelopmental, neurodegenerative, neuropsychiatric, neurogenetic, or neuromuscular disease, disorder, or condition, and the enhancer is capable of restricting the expression of the transgene to an interneuron cell population that has loss-of-function for the gene, is deficient for the gene, or that expresses a mutant, variant, or defective form of the gene associated with the neurological, neurodevelopmental, neurodegenerative, neuropsychiatric, neurogenetic, or neuromuscular disease, disorder, and pathology. In embodiments, the enhancer sequence inserted in the virus vector polynucleotide is one or more of S9E1 to S9E40 (SEQ ID NOs: 1-80) as described herein. In certain embodiments, the expression of the transgene (e.g., reporter gene, therapeutic gene, or effector gene) in interneurons may be determined by the detection of markers that are specific for interneuron cells, e.g., without limitation, GABA GAD67, or PV interneuron cell markers. In an embodiment, the virus vector or vehicle is an adeno-associated virus (AAV) or a recombinant AAV (rAAV). The terms “AAV” and “rAAV” are used interchangeably herein.

A transgene refers to a gene (or genes) of interest (such as a reporter gene, a therapeutic gene, or an effector gene) contained in the rAAV vector or vehicle as described herein and is expressed and functional in a certain cell types or populations as described herein, especially by virtue of the enhancer sequence also contained in the rAAV vector, which restricts (or regulates) the expression of the gene to defined neuronal cell populations, namely, distinct classes of inhibitory GABA-ergic neurons, i.e. parvalbumin-expressing interneurons—PV; Somatostatin expressing interneurons—SST; Vaso-active Intestinal Peptide expressing interneurons—VIP and non-VIP/CGE-derived interneurons—ID2 interneurons; and distinct classes of basal forebrain neurons, i.e. Dopamine-Receptor 1 expressing medium-spiny neurons—D1 (Drd1 neurons); Dopamine-Receptor 2 expressing medium-spiny neurons—D2 (Drd2 neurons); Cholinergic neurons (e.g., ChAT), Cholinergic interneurons of the striatum—Ch-IN and Cholinergic projection neurons of the basal ganglia—Ch-PN), or subtypes thereof. In some cases, the gene of interest (e.g., a therapeutic gene) is a normal form of a gene that is expressed in the cell type transduced by rAAV and whose encoded product functions to provide a normal or normally-functioning product in the cell, such as, for example, a cell in which there is a loss of function of the same gene as the transgene. In some cases, the transgene may be a reporter gene, e.g., green fluorescent protein (GFP) or red fluorescent protein (RFP) that provides a detectable signal following transduction of a cell by the rAAV vector. In some cases, the transgene may be a clustered regularly interspaced short palindromic repeats-Cas9 (CRISPR-Cas9) protein or variant thereof, a Zinc Finger Protein, a Transcription activator-like effector nuclease (TALEN), or an engineered form thereof. In some cases, the transgene may comprise a sequence containing both a reporter gene and a gene that encodes a product whose expression and activity provide for normal cell function. The latter type of gene may be considered to be a therapeutic gene. In a particular embodiment, the rAAV contains a specific enhancer sequence as described herein, namely, SEQ ID NOS: 1-80, and a transgene, such as a therapeutic gene for expression in a particular neuronal cell type in which the function of a gene corresponding to the transgene, or the function of another gene in the neuronal cell is lacking, aberrant, mutated, silent, or otherwise defective.

The rAAV vectors and methods described herein are based, at least in part, on the discovery and demonstration that an enhancer element contained in a vector can restrict the expression of a transgene contained in the virus vector (or in another virus vector), such as a gene associated with a neurological, neurodevelopmental, neurodegenerative, neuropsychiatric, neurogenetic, or neuromuscular disease, disorder, or pathology, or a reporter gene, to interneuron cells (“interneurons”), such GABA-ergic interneurons or basal forebrain neurons in the brain where the disease-associated gene is expressed and the encoded gene (transgene) product is functional. In an embodiment, such an expressed, functional gene offsets, replaces, compensates, or substitutes for, the abnormal, aberrant, or lack of function of a gene encoding a product involved in the normal functioning of the neuronal cell type or population.

In an embodiment, a suitable viral vector, e.g., a lentiviral vector or, in particular, an adeno-associated virus (AAV) vector, or a recombinant adeno-associated virus (rAAV) vector, is used to restrict expression of a transgene in GABA-ergic PV-expressing interneurons in a mammal, in which an enhancer element as described herein, e.g., mouse or human S9E1-S9E5 (SEQ ID NOs: 1-10), provided in cis. In embodiments, the enhancer element is one of mouse or human S9E1-S9E40 (SEQ ID NOS: 1-80). In an embodiment, the enhancer element is S9E10, which has >85% specificity for SST interneurons in the cortex, and can be useful for clinical intervention with the goal of reducing seizures and associated neurological and neuropsychiatric disorders. In an embodiment, the enhancer element is S9E27, which has a >95% specificity for targeting both Cholinergic interneurons in the striatum and Cholinergic projection neurons in the basal nuclei and can be useful for clinical intervention to alleviate symptoms related to neurodegenerative disorders such as Alzheimer's disease or Parkinson's disease. As such, targeting these neuronal populations has utility and relevance for neurological, neurodevelopmental, neurodegenerative, neuropsychiatric, neurogenetic, or neuromuscular disorders, including, but not limited to, Alzheimer's disease, Parkinson's disease, Dystonia, ALS and Down Syndrome.

In an embodiment, the viral vector or rAAV vector comprising the enhancer drives the expression of a copy of a transgene in a transduced GABA-ergic neuron or a basal forebrain neuron, e.g., for the treatment and therapy of neuronal disorders, such as seizures or epilepsy. In other embodiments, the vector or rAAV vector comprising the enhancer drives the expression of effectors like Gq-DREADD or PSAM for chemogenetic modulation of PV-interneuron activity for the treatment of various forms of seizures, epilepsy, including focal and pharmacologically intractable epilepsy and/or the symptoms thereof. In another embodiment, the vector or rAAV vector comprising the enhancer drives the expression of transgene effectors such as encoded CRISPR/Cas, ZFP, or TALEN proteins, and the like, for gene editing or gene expression modulation in particular neuronal cell types.

In general, a viral vector or rAAV vector comprises a polynucleotide comprising an enhancer sequence selected from S9E1-S9E40 as described herein, and a transgene sequence, such as, a polynucleotide sequence encoding a desired transgene product. In an embodiment, the polynucleotide comprises an enhancer sequence selected from S9E10 or S9E27 as described herein. In certain embodiments, methods are provided for therapeutic and prophylactic treatments for a neurological, neurodevelopmental, neurodegenerative, neuropsychiatric, neurogenetic, or neuromuscular disease, disorder, or pathology in an individual (e.g., a human patient) in need thereof. By virtue of the enhancers described herein and contained in the delivery vectors, particular neuronal cell types associated with various neurological conditions are targeted and transgenes are introduced therein. In embodiments, interneurons, which are implicated in various neurological disorders and diseases, Medium Spiny Neurons, which are implicated in motor disfunction, and Cholinergic neurons, which are the earliest neurons to degenerate in Alzheimer's disease, can be specifically targeted and transgenes (e.g., therapeutic and/or effector transgenes) introduced into the cell types implicated in the neurological disorders, conditions and diseases, thus providing treatment and therapies for the neurological disorders, conditions and diseases. As such, the ability to target such neurons by using the enhancer elements described herein provides an advantageous therapeutic benefit for treating a variety of neurological conditions, such as, without limitation, Alzheimer's disease and Parkinson's disease.

In an certain embodiment, a method is provided in which an individual or subject in need, e.g., a patient afflicted with a neurological, neurodevelopmental, neurodegenerative, neuropsychiatric, neurogenetic, or neuromuscular disease, disorder, or pathology is administered a viral vector, such as a recombinant adeno-associated virus (rAAV) vector comprising an enhancer sequence as described herein and a transgene polynucleotide sequence encoding, for example, a therapeutic protein-encoding polynucleotide sequence; a (Gq-DREADD)-encoding polynucleotide sequence; a PSAM-encoding polynucleotide sequence; a clustered regularly interspaced short palindromic repeats-Cas9 (CRISPR-Cas9)-encoding polynucleotide sequence, or variant thereof; a Zinc Finger Protein-encoding polynucleotide sequence; or a TALEN-encoding polynucleotide sequence, (or an engineered form thereof), such that the transgene product is expressed in intended neuronal cell populations of the individual or subject as described herein. Thus, a method is provided for converting interneurons and neurons, for example, PV-expressing interneurons, in an individual or subject in need, that do not express a given protein or polypeptide or that express a protein or polypeptide having abnormal or harmful activity to interneurons and neurons that express a protein or polypeptide having normal, non-aberrant, and/or non-harmful function or activity. As such, the directed expression of the genes and encoded proteins in neuronal target cells and cell populations is linked to the presence of the enhancer element (S9E1-S9E40) as described herein that is also provided as a component of the rAAV vector genome. In an embodiment, the polynucleotide comprises an enhancer sequence selected from S9E10 or S9E27 as described herein. In certain embodiments, methods are provided for therapeutic and prophylactic treatments for a neurological, neurodevelopmental, neurodegenerative, neuropsychiatric, neurogenetic, or neuromuscular disease, disorder, or pathology in an individual (e.g., a human patient) in need thereof, involving administering to the individual or patient a viral vector, such as a recombinant adeno-associated virus (rAAV) vector, comprising an enhancer sequence as described herein, and a transgene polynucleotide sequence encoding a protein or polypeptide to achieve a therapeutic treatment or effect.

In an embodiment, a prophylactic or therapeutic treatment method is provided for prophylaxis and/or therapy for neurological, neurodevelopmental, neurodegenerative, neuropsychiatric, neurogenetic, or neuromuscular diseases, disorders, or pathologies, including but not limited to, Alzheimer's disease, Parkinson's disease, Dystonia, ALS and Down Syndrome, which comprises introducing into an individual or subject in need a viral vector or an rAAV vector which comprises the enhancer sequence S9E27 (SEQ ID NO: 61 or SEQ ID NO: 62) as described herein, and a transgene polynucleotide sequence such that the severity of the neurological, neurodevelopmental, neurodegenerative, neuropsychiatric, neurogenetic, or neuromuscular diseases or disorders and/or the symptoms experienced by the individual or subject is reduced, treated or prevented.

In an embodiment, a prophylactic or therapeutic treatment method is provided for prophylaxis and/or therapy for reducing seizures and associated neuropsychiatric disorders, which comprises introducing into an individual or subject in need a viral vector or an rAAV vector which comprises the enhancer sequence S9E10 (SEQ ID NO: 10) as described herein, and a transgene polynucleotide sequence such that the severity of the seizures or the disorder and/or the symptoms experienced by the individual or subject is reduced, treated or prevented. In an embodiment, the individual or subject in need is experiencing a seizure (e.g., an epileptic seizure) at the time of administering the vector. Following administration of the vector to the individual or subject, the severity of the seizures and/or the symptoms thereof are reduced, treated, or prevented.

In a certain embodiment, a prophylactic or therapeutic treatment method is provided for prophylaxis and/or therapy for a neurological, neurodevelopmental, neurodegenerative, neuropsychiatric, neurogenetic, or neuromuscular disease, disorder, or pathology, such as seizures or epilepsy, and/or the symptoms thereof, which comprises introducing into an individual a viral vector or an rAAV vector which comprises an enhancer sequence (mouse or human S9E1-S9E40) as described herein, and a sequence encoding an hM3Dq modified muscarinic receptor (Gq-DREADD)-encoding polynucleotide sequence, and subsequently administering to the individual an effective amount of an agonist of the Gq-DREADD such that the severity of the disease, disorder, or pathology, such as seizures, epilepsy, and/or the symptoms thereof is reduced, treated or prevented. In an embodiment, the individual or subject in need is experiencing a symptom of the neurological, neurodevelopmental, neurodegenerative, neuropsychiatric, neurogenetic, or neuromuscular disease, disorder, or pathology (e.g., seizure or epileptic seizure) at the time of administering the agonist of the Gq-DREADD receptor. Following administration of the agonist, the severity of the disease, disorder, or pathology (e.g., seizure) is reduced. In embodiments, Gq-DREADD receptor agonist is clozapine-N4-oxide (CNO), perlapine, salvinorin B, salvinorin A. or another suitable Gq-DREADD receptor agonist as known and used in the art.

In embodiments of the therapeutic and prophylactic methods described herein, the individual or subject is experiencing, or is at risk for developing, a partial seizure or a generalized seizure. In other embodiments the individual or subject has, is suspected of having, or has been diagnosed with epilepsy of any form, including, without limitation, pharmaco-resistant epilepsy. In accordance with the described methods, seizures, epilepsy, or related symptoms are inhibited, blocked, reduced, abated, or prevented.

In an embodiment, a composition comprising a viral vector or rAAV vector is administered to a subject in need thereof. In an embodiment, the administration of a composition comprising a vector (or the vector itself) comprising an enhancer element, e.g., mouse or human S9E1-S9E40, as described herein and a transgene polynucleotide facilitates conversion of neurons or interneurons (e.g., certain GABA-ergic interneurons or basal forebrain neurons as described herein) of an individual or subject that do not express the transgene into neuronal or interneuronal cells which do express the transgene, e.g., in the brain or CNS. In another embodiment, the administration of a composition comprising a vector (or the vector itself) comprising an enhancer element, e.g., mouse or human S9E1-S9E5, as described herein and a polynucleotide encoding Gq-DREADD receptor facilitates conversion of interneurons or PV-expressing interneurons of an individual or subject that do not express Gq-DREADD receptor into Gq-DREADD receptor-expressing interneurons or PV-expressing interneurons in the brain, thereby resulting in interneurons or PV-expressing interneurons that are responsive to a Gq-DREADD agonist. In another embodiment, the administration of a composition comprising a vector (or the vector itself) comprising an enhancer element, e.g., mouse or human S9E1-S9E5 (SEQ ID NOs: 1-10), as described herein and a polynucleotide encoding a PSAM facilitates conversion of interneurons or PV-expressing interneurons of an individual or subject that do not express PSAM into PSAM-expressing interneurons or PV-expressing interneurons in the brain. In embodiments, the vectors, compositions and methods as described herein are used in the prophylactic or therapeutic treatment of a neurological, neurodevelopmental, neurodegenerative, neuropsychiatric, neurogenetic, or neuromuscular disease, disorder, or pathology, e.g., Alzheimer's disease, Parkinson's disease, Dystonia, ALS and Down Syndrome. In embodiments, the vectors, compositions and methods as described herein are used in the prophylactic or therapeutic treatment of partial and/or generalized seizures. In an embodiment, the enhancer element is S9E10 (or huS9E10) or S9E27 (or huS9E27).

In an embodiment, the vectors, compositions and methods as described herein are used in the prophylactic or therapeutic treatment of a number of different neurological, neurodevelopmental, neurodegenerative, neuropsychiatric, neurogenetic, or neuromuscular diseases, disorders, conditions, or pathologies. The enhancers described herein not only target interneurons, but they also target Medium Spiny Neurons that are implicated in motor disfunction, and Cholinergic neurons that are the earliest neurons to degenerate in Alzheimer's disease. In another embodiment, the vectors, compositions and methods as described herein are used in the prophylactic or therapeutic treatment of various forms of epilepsy, including, without limitation, pharmaco-resistant epilepsy and/or may constitute a replacement of a pharmacological treatment. In embodiments, the vectors, compositions and methods as described herein are used in the prophylactic or therapeutic treatment of one or more seizure disorders, which include, but are not limited to, epilepsy, including, localization-related epilepsies, generalized epilepsies, epilepsies with both generalized and/or local seizures, and the like, seizures associated with Lennox-Gastaut syndrome, seizures as a complication of a disease or condition (such as seizures associated with encephalopathy, phenylketonuria, juvenile Gaucher's disease, Unvericht-Lundborg's progressive myoclonic epilepsy, stroke, head trauma, stress, hormonal changes, drug use or withdrawal, alcohol use or withdrawal, sleep deprivation, fever, infection, brain cancer, and the like, or chemically-induced seizure disorders.

In some embodiments, the administration of a viral vector or rAAV vector comprising an enhancer element as described herein and a transgene may occur at a time prior to the onset of the neurological, neurodevelopmental, neurodegenerative, neuropsychiatric, neurogenetic, or neuromuscular disease, disorder, or pathology, or symptom thereof, e.g., a seizure or epileptic seizure, for example, days, weeks, months, or years prior to administration. By way of example, those in the art have demonstrated that rAAV driven expression can last for at least six years in a non-human primate model (Rivera, V. M. et al., 2005, Blood, 105:1424-1430).

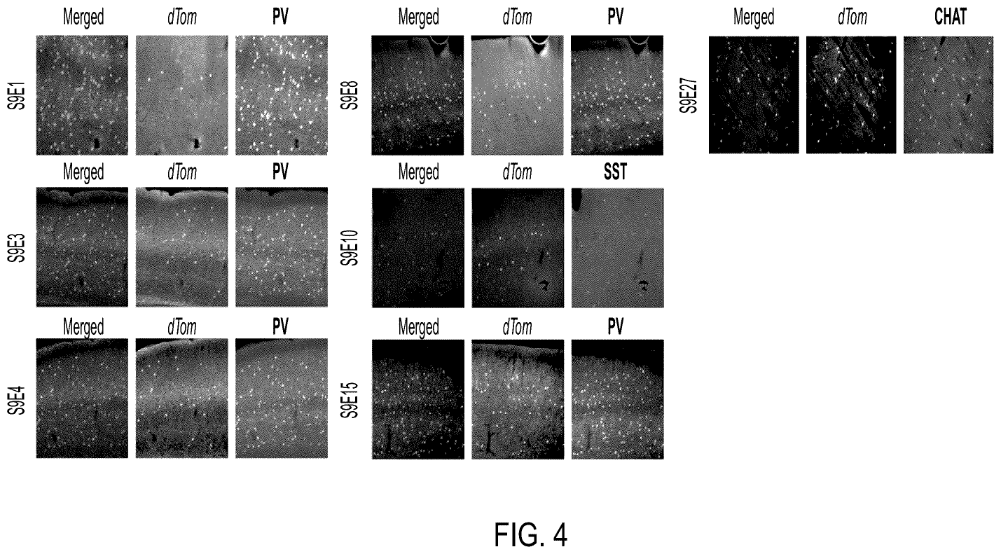

In an embodiment, the rAAV vector, which comprises an enhancer sequence as described for restricting expression of a transgene in particular neuronal populations and cell types, also comprises capsid proteins that enhance the targeting ability of the virus vector and allow the vector to specifically transduce interneuron cells, such as GABAergic interneuron cells, and/or specific subpopulations of GABAergic interneuron cells, particularly in the cerebral cortex of the brain. rAAV vectors that transduce GABAergic interneurons and rAAV vectors that comprise capsid proteins which increase the likelihood that the virus will specifically transduce GABAergic interneurons, in particular, the subpopulation of GABAergic interneurons that also expresses parvalbumin (PV), called PV-expressing interneurons, (also called PV-expressing cortical interneurons) are highly suitable for use in the compositions and methods described herein. By way of example, specific expression of a transgene in PV-expressing interneurons in the brain may be achieved using the representative S9E1, S9E3, S9E4, S9E8 and S9E15 enhancer element sequences in an rAAV vector (FIG. 4). An rAAV vector containing the S9E10 enhancer sequence can comprise capsid proteins that enhance the targeting ability of the virus vector and allow the vector to specifically transduce SST interneurons. (FIG. 4). An rAAV vector containing the S9E27 enhancer sequence can comprise capsid proteins that enhance the targeting ability of the virus vector and allow the vector to specifically transduce ChAT neurons in the brain. (FIG. 4).

Methods utilizing an AAV vector, which is designed and molecularly engineered to harbor an enhancer as described herein that restricts that expression of a transgene to certain interneuron cells, involve administering a therapeutically effective amount of the viral vector, a viral particle, a virus-like particle, or a pharmaceutical composition comprising the viral vector, particle, or virus-like particle to a subject in need, in particular, to transduce neurons or interneuron cells in the subject with the vector harboring the enhancer sequence and the transgene, express the gene in the neuron or interneuron cells and provide a functional response, e.g., the provision of a functional gene and/or gene product, or modulation of a gene or gene product, such as an increase in function of the gene or gene product, in neuron or interneuron cells of the subject following administration. By way of example, the functional expression of the transgene in the transduced neurons or interneuronal cells normalizes the excitability of neuronal or interneuron cell populations that are deficient in the gene, such as GABA-ergic interneurons, e.g., parvalbumin (PV)-expressing interneurons, Somatostatin (SST)-expressing interneurons; Vaso-active Intestinal Peptide (VIP)-expressing interneurons; and non-VIP/CGE-derived interneurons (ID2 interneurons); or basal forebrain neurons, namely, Dopamine-Receptor 1 (D1)-expressing medium-spiny neurons (Drd1 neurons); Dopamine-Receptor 2 (D2)-expressing medium-spiny neurons (Drd2 neurons); Cholinergic interneurons of the striatum (Ch-IN); and Cholinergic projection neurons of the basal ganglia (Ch-PN).

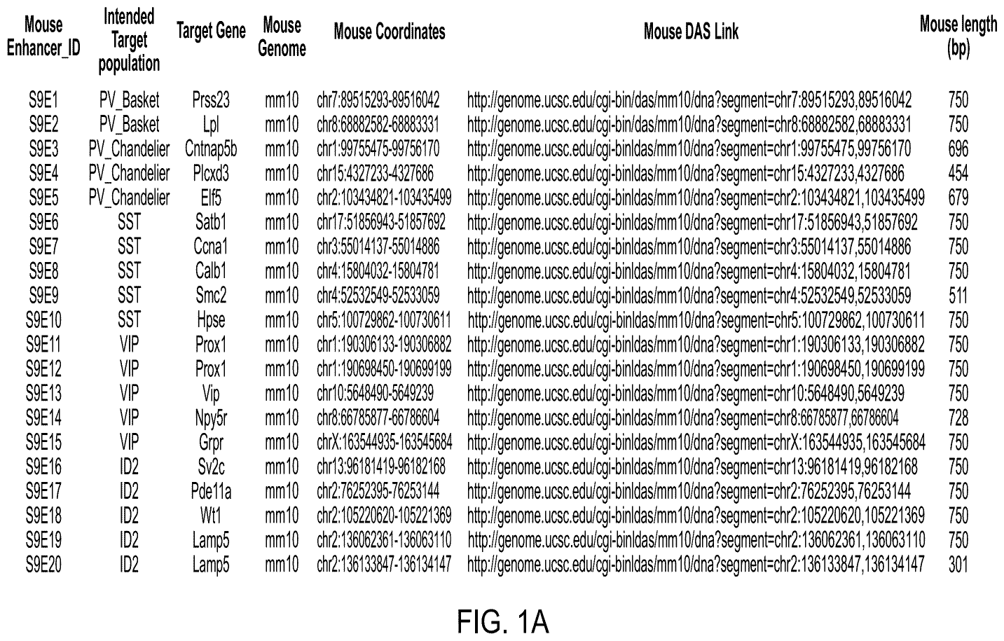

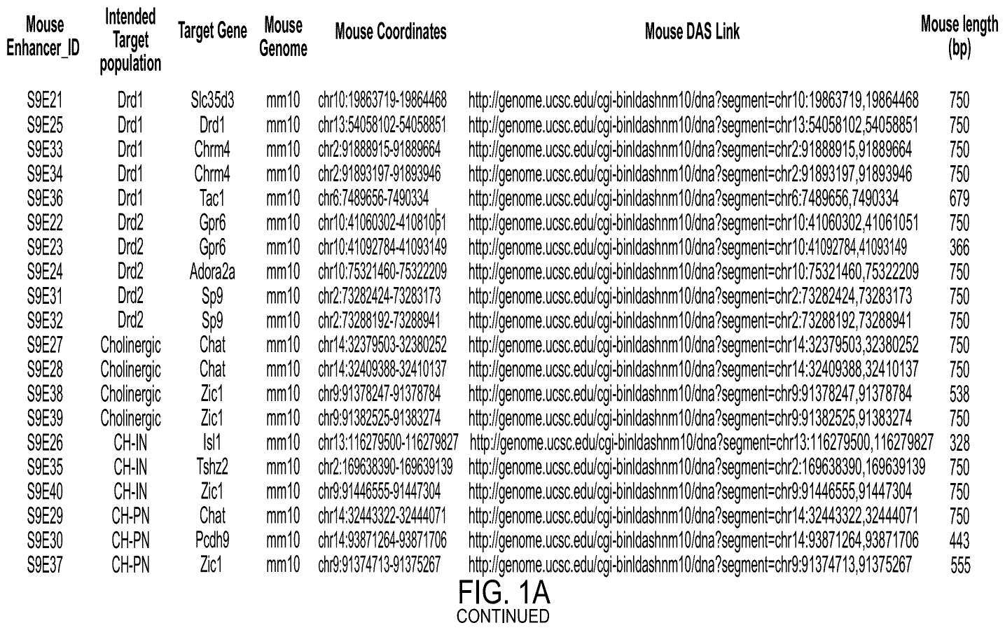

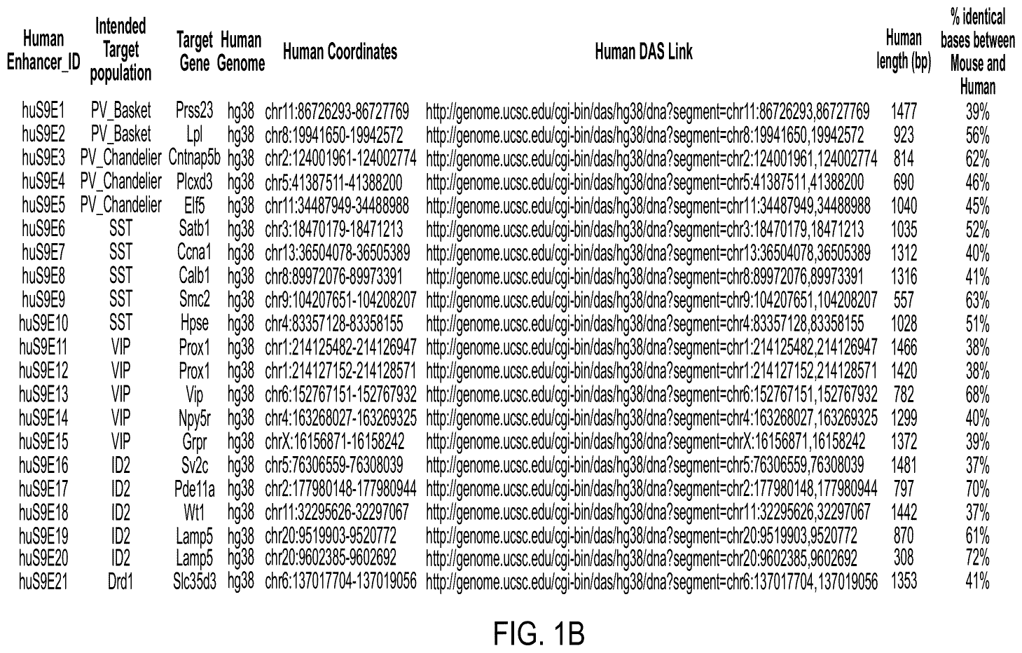

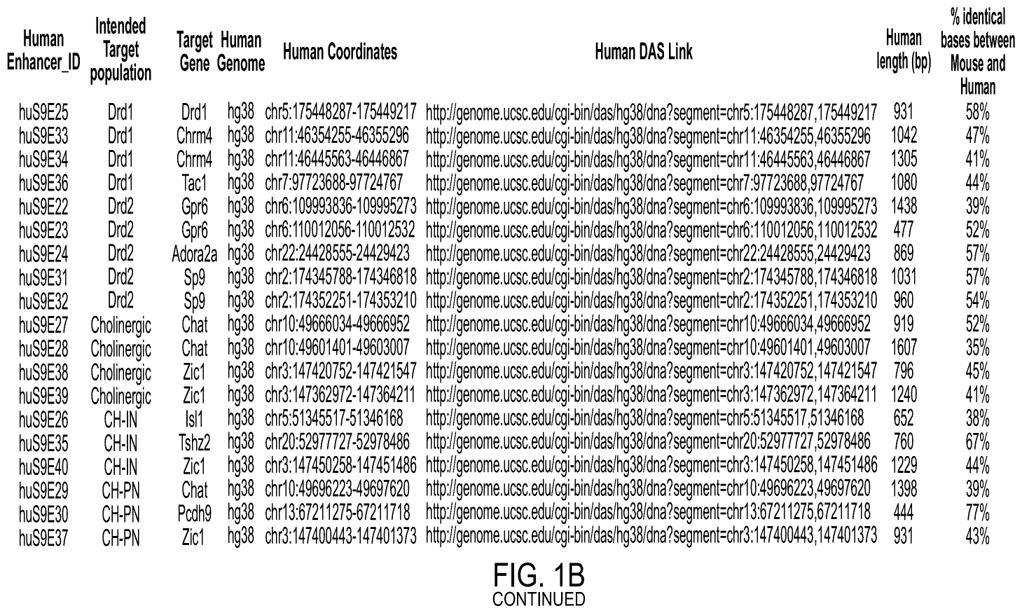

In an embodiment, the enhancer polynucleotide sequence that restricts, regulates, or modulates the expression of a transgene (e.g., a therapeutic gene) in an interneuron cell is about 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1050, 1100, 1150, 1200, 1250, 1300, 1350, 1400, 1450, 1500, 1550, 1600, 1650, 1700, 1650, 1800, 1850, 1900, 1950, 2000, 2050, or 2500 nucleotides (base pairs (bp)), or longer, e.g., greater than 2500 nucleotides (bp) in length, including the bp values at the beginning and end of the ranges, and all larger and smaller values in between these aforementioned bp lengths. The lengths (in bp) of the mouse and human enhancer polynucleotide sequences that target the different intended neuronal cell types described herein are provided in FIGS. 1A and 1B. In embodiments, the enhancer sequence for restricting, regulating, or modulating (e.g., enhancing) expression of a gene or transgene (e.g., a therapeutic gene or effector gene) in a target interneuron or neuron cell and/or population may be derived from an intronic or intergenic sequence of a genomic polynucleotide, e.g., DNA or RNA.

In an embodiment, a neuronal cell enhancer sequence comprises a nucleotide sequence which contains one or more regions of 50-500 bp or longer, 50-250 bp or longer, 100-200 bp or longer, 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M9E1,” located on chromosome 7, at start/stop positions 89515293-89516042, shown in FIG. 1A, or a human ortholog thereof.

(SEQ ID NO: 1)
ttgctacaagagaacatctagtttttcagacatctattgtctattctaca
aagtacggtttcctgtgaccacaataaaatgtaagaaagtacatgaaggt
ggtgctcatgctagacaccagttggaagagcgacaaagggtcctccatga
aatctgtagccagggacactgaacaatgaggctttgctaattccccaagc
aaatgcctacaaatgaacaaagctctgtgaatacactgaagccaagcttg
ctctcattttccagaatcacataaccccttaaggcaagacagacccagtc
acagtatggcttaagaatgatggtcttgtaaggaaacgggaggggactta
agaagcccagcaaatcaagtacaatctcatggtcacacagagcttaactt
ctctatcaggttttaggcaaaatgagccatcctagtaatcccaagacagc
ctagcagcagagcaattacagagtgctgtcaatggtgagaccatgagaaa
cgaaatccctggagttatttggaattatgcatgaataatgcccggtgcat
taactcctaaaacatccatgggagggatgctcaggagaacctgagactat
tagcacagcagacctgggccaacaatgttctcagacacactaatgggaac
tttggcacttaggccacaggcagaaaaatatacctacatccactgtctac
ctgaatagtcatcagtgagacagaaatgaaatccatattaattaccttg
g.

In an embodiment, a human neuronal cell enhancer sequence, which is the human ortholog of S9E1, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E1,” located on chromosome 11, at start/stop positions 86726293-86727769, shown in FIG. 1B.

(SEQ ID NO: 2)
ttcaagagcaccagcgagtcaccacaactctaactgctttacaggtgttt
tttctcttaatcctcatatcaaccctatgcagtagatactattatttcac
attctttccactttaagtcaagctaactgagaccaagagctgctaagtag
cccactaaaagtcccagagctagaaagttggaaagaaagaattcaaagca
ggtcatatgtgaagactacaccagtatgactccctcctctggaatcccat
gtgcaccttactcagattctggttctgtctttcaccactctcttgtgggt
aggtgtcctggtgactttcctgaccataatgttgcttagtcatctgaatt
ccctgtgtcaggtacagtgcctgatacagagtggtcacccaaccagtgtg
ttgaatgaatgaagaatgaatgaattgtgtcttcactgcagggagagtga
ttttggtggatagtcagatggaatcaggttgaagggcacctaaagcttaa
tccctgctcgtgagtctttatgtgcctttgggcaaatatcttactttcta
catgaagtttttctccttggcatgagtgaatctttcatatcttcccagat
gcattatggattcattcacgaattagtagtgtgcattaacactataagtg
ctatacttctgcttagcatatgccccacaggcaacagagagatccatccc
tttcacagcagcttcaacttccaggctactctggggtttctaaatcactt
tggagtacttggaatgtgtttaagagttaacgaaagcatggaaataggca
accaaagtgcccttctacctgtatcctagttctttttttttttgtcttgg
atttttcccaaggttcttaaaggaagaagtggttgacagccacatgtata
agcaaaggcctctgcttaggaagcaggactcggctcatttctgtctgtaa
cttgatgtatgactttgggcaatgtgctctccttccctattcatctgttt
ccttatcttgaaaatcacaaaattgccctgcatgacttctctttcagctc
taatattctttgatgcaacactggacaagatgctatgtgtggagctctgc
actggaagctgcagatacagccatgagtggaggatcggggtctgggtctg
taagcactaagagcctgagcagtgggccagtgctccaatgaagccagagg
cctcctgggtcctgacacagctggggatgggcgagtactttagtggggca
tgagatgaagggcaggaaacaaggctaaaaaaaggagaaagggggcttct
gtacctcatgaggaatttagactccatcctgtattcctgttagcccaact
ttgttcctttgtcccaccatcactgttttggccaattctgtgctatggtg
gttaatagcagggccaccagagctgcaagttcaggtttgtatctatgctt
tgctgcttactggcagcgtgaccttgg

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M92,” located on chromosome 8, at start/stop positions 68882582-68883331, shown in FIG. 1A, or a human ortholog thereof.

(SEQ ID NO: 3)
tttaaccacagcaggcctagccctttgtcccacaaatgagtttgcctagg
ggtaaaaatgtgggtgagaaaggcacagcttggagcgatcccagctcccc
tctgggctcccagtttccgtgttcactctttatttttttttttttccacg
aatagtgtgcgagtttgagaggcattgccctgtgcttcttcctggagccc
ctgcagcctctcctggtcccaggcaggaaagcagagagctaaaagagagc
atagagggtcctgctctgccaagtcccgttccttcagccacagtgctcac
ttgaggttaatatctgagttaaataactgggagatgacaagggagtgact
gattaatagcggcattgaaaggtgtggggagacaagtgggggaagagtcg
gcagtggaaagacgttcagtcctttgacagactgtatttttagcacagcc
tccctttcctgctgcgctgtagaggagactttatactccttgactgactg
actgtttctgttctgtcagattgtccagtcaagtcgatgaaaacatttca
gggggtcttgtccccttttcctctgaccatagcctgtttccacattatca
tcaccaaggctgcactgtgttgtgtgtgtggctcatctgaggaaaccgta
ttgtgatggtgactgagttcttctgagtcttcagacacttggtgcccctc
ctctgatggcattcatctgtctttagtgtcacttccgtgctgctactttt

In an embodiment, a human neuronal cell enhancer sequence, which is the human ortholog of SME, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E2,” located on chromosome 8, at start/stop positions 19941650-19942572, shown in FIG. 1B.

(SEQ ID NO: 4)
tttcactctactcacccaatatatcatgcctctcccatcacagcaacttt
ctccctctttctcctccagatgcattcatctaggaaggtaagaatttcag
ggagagaaagatgtcaccgtggtagaaagacagggatcacctcctctggg
ctcttgagtttacttattcattctggattctttctaacaagaatatgagg
acaagaggcactgtcctcaggcacttcgtcctgggagccaccaccatctc
tgcatggccccaattaggaaacgtgaagagctaggagagggagagtatgg
tcagtgcttagcagctgaagttccacttgcctggccatcgtgaatttcca
ggctgtcttctgagttgaacatgatggcaaaggagagcaaaatagcagat
gtcactgaaggagagctcagcgagggagtgattgattaatagctgtattg
aaaggtgggagtcaggtacgggggaagagcggcgatggaaaattttcgct
ttctttcagcagcttatttttaactcagctttctgttcttgctttattat
ggaggaaaaattgggccatagagtttactgccttatgccagattgttcaa
gaaaatgccttgcaacttacaatattttgcagctagtttcttccgtgacc
accacaaagactgcattgacttaaatatgaagatgttccagccatcaaaa
tgatggttggtgatgattttggatcacaaagtgtaaggaaagtattcaag
acatgagtatcatgattttttaaggtctggatgaagagaccatttggatt
tactaataaggtaaattccaacttttatggcaataaaaacaacaaaaaca
cttatcagtgtaaagctttgggatcatctatccattaaatgagtttgtca
ccacagtgaactaaatacctttt

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E3,” located on chromosome 1, at start/stop positions 99755475-99756170, shown in FIG. 1A, or a human ortholog thereof.

(SEQ ID NO: 5)
cacaggcagaggagagccatggcagagcatctgtggatctaaaggacttc
taacctcagaggactcctctgttcaaatggaagaatgacagcttccaagc
atccagaaaatgaggaaattgagttattccacctgctttttatctaaata
gtctctttgttagaagctcaaacttttgatttagttaaaagattaacagc
agtacaatctattctccatgtctcacctgatttatcctggactatagctt
gagttaccagtcacaaaacaggaatgagagaaagagttttggccaatttt
atagccagatgaatcaaagcacaactggggaaaggctgagtatagtgaat
ggagcagggatgcaaagttgaagctgctggatttctaacccagcaagaat
ctcatcaccttgttattttgggcaagtcccttctgttcttttcttatttt
tgaatgacctggttctatgaacttggctatttatgagaattatgaaaagg
attaaaaaaaataccagaactccaggacttagttaaaatagatgctctaa
atttcaaagaaagcttggaaagaccctagggtttgatctgtgccagtaaa
tttcctaattctctggatccaaggaagctgtctgcatccactgactatgc
tgaggtacaaacatgtcccagtaagggtagaaacgggctggccttc

In an embodiment, a human neuronal cell enhancer sequence, which is the human ortholog of S9E3, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E3,” located on chromosome 2, at start/stop positions 124001961-124002774, shown in FIG. 1B.

(SEQ ID NO: 6)
aataggtgttctctggggatgaacggagccacagcagaagcatctgtagg
cctgtgtattgtttctaacaatccagagccccacagatttctatcctctg
gattcctcattcaaaatggacgaattcccgcttcccggcagttatgtttt
cagcgcttcccacaaatgagggcatttaatatgtcatagtagttctcttc
cattttccactttaacgctccctttattagaggtacaagccattgattta
attaaaaggttaacaccagagcaatctaatctttcctccccacccttctc
atctgatttatccagggctgcagcttacattagaaatcacaaaaaaggga
aaaagcatcaggccagttttataactagataaatcaaagcacagctggga
agggcttagggtagtgaatagaacatgcattttgagttgggatttttaga
tttttaactcagcaagaatttcatcataatgttattttgggcatgttcct
tctcctctccagggctgttttctgatctgttgaatgactggggttttctc
aacttgactgattatgagaatcacatgagatatttatcaaaaatacagat
ttccaggacccatatcagatggaaaatctgaatcttcaaagaggataaag
tcgcccctaaacattgtttatgctcacaagttcagcatactctatgtaag
tggaccctatgggcttgtccagctctgacgtcttgtccacctctgactgt
gatctcaaagcactcttatatataagtagtagatagaaaaataatcaagg
gctctgaatttttc

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “4,” located on chromosome 15, at start/stop positions 4327233-4327686, shown in FIG. 1A, or a human ortholog thereof.

(SEQ ID NO: 7)
tattgatactcttctgaagccattgttgcttgtaagacagatttactttc
accttgtgtcaagaacatccagagagaaagactaacatcactgctgctca
gctgcctaatcgtttcagcaaaacatgtgagtgtgtttgtggacagtaac
tattaccatgggaaaggactcattcacagccttcctgattaacggcttta
ttgcttgttattatccttttcatcttacacaactataaatatataatcta
atgatcctttcactgaaatagcatctttaatgatgggattttcaaagtgc
ttctcagtcattcaataattaggcagattcaccctgcgcccccccccctt
ttttttcttttttgagctttttactgagaatcttgaagagttgggggaaa
taaaaaaagaaagaaagtcttctactgaaacagtcattttttagaggaga
caaa

In an embodiment, a human neuronal cell enhancer sequence, which is the human ortholog of S9E4, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E4,” located on chromosome 5, at start/stop positions 41387511-41388200, shown in FIG. 1B.

(SEQ ID NO: 8)
tattgcaagtattctgtaattttttagactttatgtgtaaagggatatga
atgacttgaggcaattacatcattctgattaaaggcccccagtaactgga
agtaagttagtctctctgtgtaaaaaattatatgtactactagttctatt
ttgtaaatgttttcttcaagtaaaatggtctaccaacctgatggaaagaa
atgttcttatttttaaactaatgaatttctcattcagtaaaacatttatc
aagcacctgctataagtgctgttattatgctaagtataagacaagcattg
ttctcatcctcataaatttacagcttaatatgtgatgcacatagataaat
aattataatacattttggtaatgattttttaggcactattgtgtagcatg
gggtagagaggagaaaatgtttgatcattgattattaatatcaatgggct
tatgcaaataatttttaaagaactagactaaatgcaaaagtagccttgct
ttttgggttaaatgataaatggctcttgggagccacactaaacagggtac
ataatgggaccattttcatctggctaattttgccttagtttcttgctgtg
gactagttggaaactaaaaaaaaaaatacatacaatccttgccctcaaga
gtcttataatttaattagggaagttaggcatagagacaaa

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M5E,” located on chromosome 2, at start/stop positions 103434821-103435499, shown in FIG. 1A, or a human ortholog thereof.

(SEQ ID NO: 9)
aagccagcccttccttcccgcgccaaagctgcaaatgatggtaccctaca
cttcactcgctgtctctctacagcgtcgccctgggcgaaaaactttccca
actccctatgctaataaatcatgctaattatggtcctaaattgtctaact
taggcttcctctaaagccgactgtctgtctcgctctttccctgtcctgaa
tttgttctggttgttccttaacctgcgaatgaacgcagagagttgggcag
ctcctcctgtccagtatggtatgaactttctgattctcatgtccctacct
ggagctacttcagccgtgggtatcacctccctattctgagggccctggag
gacatcagcccactgaactcaatacatccaactgggtagcaccatttgaa
agagctcccccagagtcaatggcaactctctccagctcctcagcctttct
tcccaaggcagctggccctcaagtgccctgacccacttcagacttcaaag
gagctggcaaggcctgatgcttaatagtggcctctggactttcacatcaa
gctttctgggcataggatactggctggcttcggctaggatcctaggcaaa
caggcttgataccaagcttctcctgaaagttcagtgtgtcttgtaaatga
gagacatatcaaggaaaacactgtacctt

In an embodiment, a human neuronal cell enhancer sequence, which is the human ortholog of S9E5, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E5,” located on chromosome 11, at start/stop positions 34487949-34488988, shown in FIG. 1B.

(SEQ ID NO: 10)
aagattgttccacctcgtgacctccgcactggccattcgctgcttggaac
cttttcttctctctcttttcctgggtaacttcttctcatgctttaattca
actatctcttcctccaaatagctgtccctggacccagtttaaatgcaatc
tttccccattacacacaaaacatgctgatctttccctttataatatcatc
gtgatgagcagttatgtatttatcatttgggttatttaatatctctaccc
cgaccttgcctcaatgagagcaagactcaaatagtgctcatcaccacagc
tacagtgcctagctctgggcctggcatatagtaagtgtccaataaatgtt
tgttcaaagaatgaaggaaacaggagagaaataaatgggtaaggggagca
aaggcatgaagggctttagatttagacacacctgggtcatctgttgcctt
ctaagcatgtcacctttaacctctaagcttcagtttctacccctataaaa
aggctatagcactagggcttgtctcatattgggaagattacctgacacaa
tgtctataaagatattagcacagggcctggttcataggaagcactcaata
aatgctagctgtcatcctcatccttatcctcatcatcataatcatcagca
cattgaatctaactgtggctagttatatattcaggttacacctacttcaa
aagaacttactggacacaccttctctttccattgtattagcttccttttg
tcgtgtggaaggcaggctggtaagtgattcctatttgtaggtgaagacac
tgacccctggagaaagggttggtcaggtctaggtcactactacttagaga
gtaaagcaaatgcctgcctgtggctcaagcctttggccacagtaatgtct
cccaggcttcacaggctcatttcttccaggggctttgactgggttaggtg
tggtggtaaaagctgggatttacatcccccaggctgctgcagatggatca
caagggattctaccagcaccaggaaacagtgtgtgagctt

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M96,” located on chromosome 17, at start/stop positions 51856943-51857692, shown in FIG. 1A, or a human ortholog thereof.

(SEQ ID NO: 11)
agatctgggttacctggcaggggccttgtacccttatgacacccatagat
ttctccacacaagcaatttttacaatagaaatgagttcacaagattcagc
atggtgttatggttcataccatgagggaacagagactatcagagaacttt
gggaaatgggcagaaagtctagcattatacaaatgctcattccttgcttg
cacttggttatgtaagatacaaacatcaggcaaacactctgctatctttg
taacatttctatacatataaaatgatttcaaagtaaaacgaatggaaaaa
gacccataccttgttaaaagagagggcacatctattctatgggaaaacta
ctctgccgtctcctgctttgtggcatgataaggggagcagaaagggatgg
tagaaagatggtttcctcttaatacagtttggctcttgagtcttttatct
tttacagagtaaagtgtttcttagaaagtcattgactgaatactaactca
tgttttctctacagttttgctctagaaaccttcatctctgcatcctggga
gtgggtagctctgggtagcaaaagcagctccatgtgcagaaagcagatct
gtgcaaggagaagtaagccctcatctctctgaaaatgggcccttgttgat
gtaagtacatctttttctgtttgggagaagagtaggggaaagcatatgtt
tccaagcagcacttcgtgtgtgtgtgtgtgtgtgtgtgtgtgtgtgtgtg

In an embodiment, a human neuronal cell enhancer sequence, which is the human ortholog of S9E6, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E6,” located on chromosome 3, at start/stop positions 18470179-18471213, shown in FIG. 1B.

(SEQ ID NO: 12)
atctctggggtccagaagtagaagctttttattgcctccggagtttattc
ttatacatcaaggataaacattcatgacctaacatcatctctcttgtttt
ctcccgccaaagctataaggatagtctaatcatgaaaaaacattaaacaa
actcaaaatggggacagtctataaaatacctaaccagttctcttcagaac
agttaaggtcctgaaaggcgaggagagaagaagaaacaatcacaacttgg
aggacattaatgcactaaatttaatatggtttcataagttgggctgaaaa
gaaaaggacagtagaaaagctgggatatgagaatacagtctaagtgtagt
actaatgttaagtttttaatctctaacttgatggcatggttatagaaaat
agaaacattaggcaaagctgggtgaaaggtatacaggaactctactatct
atacaacttttctgtaaacataaaattatttcaaaataaaactaatataa
gaaaaaaacatggccgacataacagatagaatggatatcttctctagtct
atgaaaaaccctgtgtcctacattcacttgctttgtgatataataaaagg
gggaaaggaagggatgatagaaacattattcctcttaataaacttggatt
ttaaaatcttttatcttttcacagcataaaacattccacttaggatgcta
tatgttgagcattaactttctctttttctttatagtctttctgtaatagt
tcaaatcctgcccaccctgagggggatgtttctagatgatgaaggaggtc
ataaacagagagtagattaatataattatctttatttcatgtctatttgc
aaatgggccattgttcacatagttgtctctctttctaaatggaagaatag
ttggagttgggggtggggaaataccagaactgagaggagtaaaggtgctt
caagacaatgcttcttaaactttggttttgtgtttgcatctcttggggat
cttgtttaaagtaggttgagattcagtatgtttg

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M97,” located on chromosome 3, at start/stop positions 55014137-55014886, shown in FIG. 1A, or a human ortholog thereof.

(SEQ ID NO: 13)
ctctgtgtcatcttgtttgaatttttaatgaacatatatttataatttct
tatataatttaaacagtgttttcaaaccataggattgcatgcaaatgtaa
tatgcccttataatataaatggcctttctcttgcttttttcttctgtcag
caagaataacaatttcctgcgggacctctggcaaaacaaatcaccctctg
tgctcggtcctgactggtggaaatacttagcatgcagtgggaatgaacag
aaatgcagggtcacctgagaacagtgttcagcatctgtaagcaatgtgag
gcaccttagatgatatgttaggaagcagctggccttgcagctccagtatt
atgtttcacgagcgtcagcatctccagtctagacagaggaagcagcagat
gagggcaggatccttaagtagtttctatatcctgagttgaaatgaaatgg
ccattgaaaatcagagctgaagacattccaaagggacacacaaagggcag
ataccaatctatctctgcattataccacagagataacctgctcatgtatg
ggtccaaatggatgccaatgttctgtctcctgtgtggctcccatatgatt
ctcacaactgtggtaggtggtggttcctcaagtgaggaaactgagatcca
taaaaggatataagctttcacaaatggagacagtggggggttggggtgga
ttgctggctagttttatgtcaacttgacacaagctagagtcaactgagag

In an embodiment, a human neuronal cell enhancer sequence, which is the human ortholog of S9E7, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E7,” located on chromosome 13, at start/stop positions 36504078-36505389, shown in FIG. 1B.

(SEQ ID NO: 14)
ctctgatttttatttttttattctgcttacattgagtttaatctactttt
ttatttctccagtttcttaaggcagaagctagggacattaatttgagacc
tttcttgttttctaatataggtatttagtgatataaacatctccttaatt
attgttttaatggaattctgacaccaactggtgtactgcaatttaaggct
aacactaactattggagttaaccttagactctaaagtctcaggggcacag
ttgaatgagactatgatcaattccatttccagctgtaaatggggtcccag
tccatctgcgcttcttaccagattcagagattccatgatcccattaggtt
tgataagttgctagaatgactcatagaattcaggaaagcactatatttat
gattatagtattatcataaaagaattagggtcagctccatagggcaaagt
tcagcaggatcctgaatgcagagcttccatgttctctctctatagaatta
ggatgcattgccctcctgtcgcatcaacatgctcaataaccaaaatgctg
aactgagcttcagtgtccagagttcttattggggtttcattacataggca
tgattgattgagtaattggccaatctccagcttccctcccttccttagtg
gtcaggctggctcaaagtcccacccatctaatcatataattggtctttct
ggtggcccatgcctataatcctgagtcatctcattttttagcataaacat
aggtgtgatctgagaggctcatgaataacagagacattactattactcaa
taaattacaaggatatcttattcttcccaggaacaagggacaaagggcag
tcaaattctgtattatacaagtggcatgtcacaaattttgatatgtttaa
atttggttttcatttagttaaaaatactttttaatttcccttttgatctc
atatttaacccatgggttatttaaaacatattatttagtttccaaatatt
tgggggattttccagagttctctctgttgttcctttctaaaaattccact
gtggtcaaaaacatactttatataacttgaatccctttaaacttatttgg
ggctgtgttacagcccagcatatgaactatctttataaatgtttggtgtg
cacctgagaagaatgtgttttctgttgaattgggggattttttctataaa
tatcaatatttaaagttggtttgcaatattatttcattattctatatcca
tattgattttctgtctgttctataaattattgagacagaagtacaaaaat
gtccactgtaag

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “SME,” located on chromosome 4, at start/stop positions 15804032-15804781, shown in FIG. 1A, or a human ortholog thereof.

(SEQ ID NO: 15)
gaaaacaagctggaaaaatagtcgtctacagaaagtcaagtacaagttta
agggcactggttgtctctctctctctctctctctctctctctctctctct
ctctctctctctctccctctttttcctttcaaatttttctcagacaccag
aattgcatgctaatggatagcaagacccacttttgccagtcattgaaaat
aactcaggtattcaaattttgatctacgagttgttcaggatccaggaact
acccagagtttaggagccagatacaagccataaagagggctttccaatgt
gttcctttttaccgagattagaagtctgcatacattttacattgagatgt
aaactgccactgcaaatgtaagtacactgcaaagtcatcacgagaaattt
tcagttcatttaaagtcactacaggaaaagtattacagtcaagagtttta
ttgtctgttttgtatttctcacattgcttgatcacaatgagcccagtaaa
gggctcaggaagcaagttttacagcaggagtttatagtgccctacgtcag
agacatgcctatgtatggggaggccttggcaggcaattctctgaaatgtt
tcctattcaacatccctgttacaagacaaatagtcatagcaattacaatg
acaatacaaagaactatgatttattaaaaaactattatgttttgcctttt
ataactccagaaggtaaacaatgtatcttgtcaaaacaacctcaagaaaa

In an embodiment, a human neuronal cell enhancer sequence, which is the human ortholog of SME, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E2,” located on chromosome 8, at start/stop positions 89972076-89973391, shown in FIG. 1B.

(SEQ ID NO: 16)
gaaattcacaacctggacaatcagtcacaaatcaagagttaactatctgc
agagatcaacatccaacgtggtaaaaacactgcagtcattaagagtcact
atccagcactttagaactaattaccttacatctttcccctggacttctcc
ttggaaacatacatatttcataaagtaaagctaagttatgattgaagagt
tacaatactttaaaatttttttgataactgatgcatgtattttaatttca
ctacaatagatgctgtaagtctgttttaaagcaatgatttctggaagctg
caatctcaaatgagttatctgcaggcaagagcagactcacttggcaggac
agaatgcaggcaactgcgttagttatggtttttctaaacaaccgacttta
aaattctatttattttacttttaaaatatacagagaacagtggaaactat
tttaactgttaacaaaaactaaatctacaataagagcactggaaagttag
tagacattcggacacataaaaaggatcaattttaatatctctttgaatcc
tttttatcctttatctcctaataaatattcccccataacttatacttctt
tcttagtgtaaaagccaactaacactttacttatatacaaataactagtt
taccaattagttcacacaataatgtaacaacactaggacagattacagag
ccagttacagacaaaaacagtaaggaaatgtttaacacatatgactgtgt
gttacagtcaaacatgactataaaactatgactgagtagatgtgagattc
ttcctgctttcccaaaacactaaagttgaatgatttttaaattatctaca
gctttctattattcacactgtcattcttaattatgccatagataacagtt
tacagtagccagggagcagctaactgtagcttagttcgatgaagtttgag
ccaggttcctaaacatatgtgtatggtagcctgttaggcctctctaatgg
ctctaccagagttctaactttattagtgtcattctttaacagctggcttc
tccactaagtgccatgagggcagaaactgtaactttttttggctcatcat
tatatccacaagtctatcttgcctgcttcactgtaggcattcaatacata
tttacaaataaactgacattacaaaatataaataaaaattcaacttaata
atttaaaagtgaataaaaagtataaacatccttcaattccctcaagtata
gatttaagtattaaaattctagaacttgaacaatgtattttggttttgtt
ttgagaacactgataa

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E9,” located on chromosome 4, at start/stop positions 52532549-52533059, shown in FIG. 1A, or a human ortholog thereof.

(SEQ ID NO: 17)
aagaggatgggatagggggtttccagaggggaaactgagaaagaggataa
catttgaaatgtaagtaaagaaaatatccaataaaaaacaaaaacacaaa
acaagaaagagcaatggctagggtgttcccaattaaatctcacactcgaa
agcacggatccaggaagctagaacagacaacaacacaccaggaagctaca
catagacgtagcatcttccagttatagaaaatcaggaatagagaaaggca
tttcctgacagaaaccataggaaaaatcacctcacttccagaagagcaca
tagaaaaattatcctcgtttctctcagaagctgtgtaagcaagaggagag
tggaatgaaatatttactgtgctgagagaataaaacatcaacctagaatt
ttgtaccctgtggaattaccctccaaaagcgaaggcaaaaatacttttca
gacaaatcgaaggaatttgttgtcaggagacgtgccttagaagaaatgtt
aaaggaagttc

In an embodiment, a human neuronal cell enhancer sequence, which is the human ortholog of S9E9, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E9,” located on chromosome 9, at start/stop positions 104207651-104208207, shown in FIG. 1B.

(SEQ ID NO: 18)
aagagaacagagactgaatggcatagaacagaatatctaagaactgtaga
acaactccaggtgtaagtagcatgcacataatagaaataccagaaggaga
agaaaagaaccgaggaaatatttgaaagagtaatgattgagaattttctc
aaagtaatatcagacacaaagccatagatccagaaagttcggggaacgcc
tgcaagataagtgacccaaaaaaccacacctgggcatatgaattttaaat
tacagaaaatcaaatataaagaaaatgtccttaagccagaaggaaaaaaa
aaaaaacccaccttacctctagaacagatataaggattacatctgatttc
ttcttagaaaccatgcaagcaagaagagagtggaataaaatatttaaagt
attgtgaggaaaaaaacccccaccagcctaaagttctgcactctgtaaaa
ttgtccttcaaaggtgaaagagaaacaaagaagttttttttttttggaca
aattgagataacttgttgtcagtagatctttcttgcaagaagtgttaaaa
gtagttc

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M9E10,” located on chromosome 5, at start/stop positions 100729862-100730611, shown in FIG. 1A, or a human ortholog thereof.

(SEQ ID NO: 19)
aggttcctgtctgcaagcatagcagggtatcattaatagtgtcaggagtt
ggctctctcccataggatgggtctcaagttggggcaatcattggttggct
gatcccttggtctctgtcccatctttattcctgcacatcttgtaggcagg
acaaattttggtttgttccaaagtgatttttaaagcattaccattttgtg
ttttgttggtttaaaacactttagctattctggtgggtgcgtgatcctgc
ctcactacggtctcagtctccatctcgatggtgactaaggatgcctccta
tgcacttatttaccatctgaatggttcctttatagaatgcatgactattc
aatttttttgctcattggacagcattttaatgtgtgaaaggattatacat
ccttcaagatatggaagaaaaagcacaaagccctaaattctgtgaattca
gatgtgtaaaaaaagacaaggaagtgcaaactatatcagcttggtgtctc
attctgggtggctgtcagagccctgtgagctgacttaagaattgggtccc
tgcaatgggtgctggcacttgcaaggggatcatgagctcaaggccatact
gagctgcataatgagaccttgtctcagtaactagactggggttggaggtc
agtactaaagggctagcctagcctaaattcccgagttccacccccagcac
tataaaaggaaaaggaggaagaggtggagggaaataaagaaaaggaaagc

In an embodiment, a human neuronal cell enhancer sequence, which is the human ortholog of S9E10, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E10,” located on chromosome 4, at start/stop positions 83357128-83358155, shown in FIG. 1B.

(SEQ ID NO: 20)
aggttacacactctttgtgttaagttctttttactcagcacagtgttttt
tgaaattagttcaggctattgaatgtatctgagtttattcatttttttct
aagtagtttccattgcgtgaatatatcatagcttgtttatctattttcct
attgatgggcatgtgaggttgttgacaagttttggtattatgaataaagc
tgctaagaacattcttatctcactctttttgtagagctatgttttcattt
ctcaagggtaaataactaggagtggaacttctgagtcataggattggtat
gaaactgccaaaagtttttccaaagtggttgtatcattttacactcccat
gaacaatgtatgagagctctcgtggttccattagaccaacacttcatgtt
gccagtttttaaaattttagccattctggtgggtgtataatccacctcat
cgtggttttctttttcatttctatgatgactaataatgttctttatgtgc
ttatttgccatttgaataggttcctttatgaaatgactgttgaatttttt
gctaattggataacattttaacatatataagggttgtatacccttcaaga
tagcaaaaatttaaaaagaattcagaattctgtgaattcagatacataaa
tatctacagagccaaagaaatgcaaagtgcattggcacaatgggccagtc
taagtggggtgtcagagttctgtaagttgctttacgaaatcccagcactt
tgggaggctgaggcgggtggatcgcgaggtcaggaggtcgagaccatcct
agctaacatggtgaaaccccatctctactaaaaatacaaaaaaatagctg
ggcatggtggcgggtgcctgtaatcccagctacttgggaggctgaggcag
aagaatggcgtgaacctgggaggtggagcttgcagtgagccgagattgca
ccactacactccaggctgggtgacagagctagactctgtctcaaaaaaaa
aaaaaaaaaaaaaataataataattagg

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence, called “S9E11”, located on chromosome 1, at start/stop positions 190306133-190306882, shown in FIG. 1A, or a human ortholog thereof.

(SEQ ID NO: 21)
gctatagcttcaaccagctgccactcaaagccccaatgtgccttcttgggtcctgcacacacatcctact
atgttttatgaatttttcgtttctctgttccaactcaatctaactaagaaaacacactgccacccattgt
ctgtgtctgaaaaaaaaatacaggaacatcaactgatgtgtgcctgactcagaagaaaagaagaccatgg
ccacagtctcactgctcagggcattcagggaagcacaattgtactcgccctgagaaacagtgcagatggt
gtaattggcatcaccagcccacatagatcagagatgcatttatttccatgactttgtttgcatcatggcc
atttttctgggctcctgctcacatctaacctcttcaagggtcatgagagagtgaagcattttgcccctgg
gattgacaggtgaggggatgtttgcatttccgtttacctgagaaatactcatttcttgcagcatcagaaa
acatttggttatgttatctcgtgggtccaggaggagcatggagtgtgccaaatttaagaggcactgccaa
atgccgtagcctgcattatgtagctgaggccgccagtaaaactggtgctaactagttgaacattccagct
cttgcctcagaagcaaaacaaccctgttagtcatttccaattgggctcccaatgctcgcctttggttaga
agacgtccttgtgtctgtgtgcaaatgctggcccctaggcacaactgatt

In an embodiment, a human neuronal cell enhancer sequence, which is the human ortholog of S9E11, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E11,”, located on chromosome 1, at start/stop positions 214125482-214126947, shown in FIG. 1B.

(SEQ ID NO: 22)
gccatcagcaacaaccattgcagccagaacagaaagcctttattaggtgtcttcggcgtcttctgcacac
agcatcaaactagatcaatgagtatcgattggtttggtcatgccccatgttccctcaagggaagacgggc
tctttaaacaaccaacccaaaatgaaacaaaacaaaacatataggctggacgaaagctccaagtttcaga
ttttcatggaaaaataaatttgcaatagattggttagaattacagaaagagtgctttaaataaactgttc
tagaaaaaaatcacagctacaactccgccatgttagagtgtacagtctacgaataacctaatagccttct
cagggaacatcaagcatgctaatgacaagataactttttgctttgttttattgtgttttaatcagagtgc
catggggtggtgaggagattttgtgccagacaaaaccgagttagaattcctgttcttttacttattaatg
tgttaccttaagcacattctgaaatttctctgtgcctcaattgtctcatctgtgaaatggggattaaaaa
cagctatcccacagagtttttgttgtatggataaaattaagtaatgtcaggctaggtgcagtggtacaca
cctgtaatcctagcactttgggaggcagaggcaggcaaacagcttgagctcaggagtttgagaatggcct
gagcaacatggtgagaccctgcctatctctacaaaaattagctgggagtggtggcacacacctgtcgtcc
cagctacttgggaggctaaggcaggaggatcgcttgaacctgggaggttgaggctgcagtgaaccaatat
catgccactgcactccagcctgggtaacaaagtgagaccctgttaaaaaaaaaaaaaaaattaagtacgt
ccataacaagcttagcatgatgcttacaaccagcaaattctcaaaaaatggttgttattggtattgggta
tttccttttaaacagcatttcctgagggccagctttgtgcaagacactgttctcagctctttgcgtgtct
tactcatttcacctcctaagaatcctgtgtggcaagcactattcttatactcattttatacatgaggaaa
ctgaggcctccttgcccacgatcacatagtaaatggcaaccccaagctgtctggctccagaagatttcct
cagaaaggaaggtcagtttgttgatctaaatagttgagctgccatttttgcctagaaaacccgagtggaa
gatttcatcagtgtccccctcatctgctgacagcagtcagtgaacatccaacatcaactggactctgcct
ccttggaaaggccccaggcttgatctggatgaatgacctctgtgtacagttacctttgacttggcctcct
acctgaaaacacaaagagggacagttacataaggtaatgctgacctatattcttgacaccctgagt

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M9E12,” located on chromosome 1, at start/stop positions 190698450-190699199, shown in FIG. 1A, or a human ortholog thereof.

(SEQ ID NO: 23)
ctacaggggaagagaggagaaaataaagagggagatgggcatgtacatatttaaattcacattatgtgtg
tgtgtttgcaagacacatataaatggcaaatagggaaacttcacaaaaaaaatgaaaagacagacagcta
gatagccccagattttaatgccacccccaaggtagaacaggtggcgaaaatataataaaacatgttgcaa
ggaacttgtcatgaattttaaaaacaaaaacgcctacagaccaaataagctgctctgacactgggctaaa
tccgaagttcccgaactattttaaatgacaaagcagaagctccctagagaaggtccaacagaaatggttt
gagggcaattgctgctaaactcatagagagaggaacttggagagaggacgaagcctgctctctctgccac
aaaggccacgtgcaaacgtccacagccagaccatgtgcaggaccaaggtgtgcccacaaaggcccctggg
agcctcccgtgctccattgttggtgggtttctcaatgcacatgtgacaggatgctgggctcaggctcatg
ggcagccactccaggaccccatgtgaaataggccagagatagaggttctagctagtacaggctgaccccc
ccaccccacgctcagcagtgcccaccctcacaccctcatctgtttggccattgcaagcaccagcctgtgc
tttgttcttggccaaccatatatgaagaagtcacttccgtggcccctccc

In an embodiment, a human neuronal cell enhancer sequence, which is the human ortholog of S9E12, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E12,” located on chromosome 1, at start/stop positions 214127152-214128571, shown in FIG. 1B.

(SEQ ID NO: 24)
atacacataggccggacaaaagcttaaagtttcagattttaatggaaaaataaatgtgtaataggcttgt
tggactaacagaagggttctttaaagaaattgcttagttttcaaatactaaacaccattagataggcaag
tactgccaaatgagtttctggagacagaatttaatgaacaagagacaaaatctttcaattatgtcattgt
gcatatatagtttttgcatagatgaagtttggttcctctaatagttaaacatcaccccctacaaacttgt
tttgataattgccttaagagatactgtctccaaataaaaaagaaaggtctgattttttaaaaaatctaaa
ttgtccgaattcgtggacaccttattagagaaggttttgggttactagtttaaaaattgttccctcgttt
tttgttttttaataggatatcaggaaaatccaccaacaaaatcatctctgaagtcattaacatgagacaa
acaaaagcggtgactgagatccagagtcacctaagccaatttaaagtcttagaatatacggcccattctt
tacaaagtcctcaaaccatcacctcagagttccttaatagaagtcagaagcccaaattcaaacacagaac
taccatggaattgtgtggtgacacccaaaacatcacctgtctggcctcagtttcctccctcatatagatg
atcaaaccacctccataagttcctcatagagatgacgatgatgatggtgatgacgatgatgatgatgatg
tcatttagcagtgttggtaatgaaaatagcagctgcttgtttattgagtcctaccatatgccaaagcact
attctaagcctttgacttgaattaactcacaatatcctcctggaaattttgcaccagggatttctcccca
aatccgtgtctaccatgctctcttaagcgaagaatattgtgacaggttccatattcaaaaagtagcctta
acagtatttttcatcttgcagaccctttatacaacgtgtttttgacatttttcccatcaagaggaggggt
ctatatttcttccacttgaacctagatgactttaccaccacggcagaagtgacactatatgacttccaag
gctgggtcataaaatgcaatacagctctttgccttctcaagctgttcaccccaacccagccacgaggctg
ggaggtatcctgggcaacaaggagagaccctatgtaaaggctgcagccaactctccagcagagttcccca
ccaacacccaacatcaatggccagatgagtgagggaaggcatctgcaggtgattcagccccagccattga
gtccccaccagccttcaagtcttcccagtggattttcccagacatcaaggtgctgtccctgctgtgggcc
cactctgaattcctgatccc

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M9E13,” located on chromosome 10, at start/stop positions 5648490-5649239, shown in FIG. 1A, or a human ortholog thereof.

(SEQ ID NO: 25)
ttaaagatggctcccaagaattgcgtaatcatatccacgaatcactaagattagaagtacacacttcagt
acatttacatatgcaaagaaagtacaccccgtcacttatctgccacttatgtgtttggtgtagccaactc
aaacagtagcctgtctgacttctgaatagcatttgtactttaaaaaaacaaacaaacaaaaacagaaaca
gaaagagggaaggacacattagagtcatctgcctcctgttaccagctttctctccctgattattagaata
tgtccccactccatctctttcacctaggtaattgcaagtcccctgcaaggtgcatcccttttcataagct
agtctttctcattctcgcacatacaattgtgctccttaggtttcttttccttggacactcgcctggtttc
ataatgtagttccttttgccttcgcttttctcctctctacgggattatgggttcattcacttgagtttcc
tgccctgagatatgcctgcctcatttgctgccgtggtaggagggctcacatagatccctcagatgtcttt
ttgtctgttagactctctctattcggcatatgatgctagggatgcatttatccatttatgttgtaattta
actatgtgataaaaattgtagactctgaagcagaagttgttcttcagtgtaagcttataattgactgcta
atgaaataattgccatgtataggtttttacagcaagaaacatctgccctg

In an embodiment, a human neuronal cell enhancer sequence, which is the human ortholog of S9E13, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E13,” located on chromosome 6, at start/stop positions 152767151-152767932, shown in FIG. 1B.

(SEQ ID NO: 26)
Tcaaagatagatactaagagttctataatcgtgtctatgagttcctaagacccttagagctgcacatttc
agtagatctagattttagaaaaatatactctaattacttatttccctattcttgtttggtgttccacact
caaatggtctcctagctggttaactgataacagctgcagtttttggaaaggagaaagagggaaaggatac
atgaaaagcatctgccttttcacaaacctctgttaacagctttttctccctattagagagaaaacatttc
cttggtccttctcttttgccttatgtaattgcaaatctctggcaagatgcatctcttttcagactttaac
ttgtctcatttttcccccacacaattgtgctctttcttttcttgttccttggaaactcgccctcgtttca
taattttgcttcttccccctttgctgttctgttctctacagagttacgggttcattctctcgggtgtctt
attctgcctcatgccctatctacatgttaacctcatctgttccagagacagaaaatggatctagatctct
ccagatttccttttgtctcttaacttttctttgcatgggatcttagacagatgcatgacacttgttaaag
tatctttctcaatttacatttcatttacctttctgctttaaaaacatagaatctttgagctagcagttgc
cattatctgtaagtttgtagttgatgtctaaggaaattactatgccattattcctaatttttcttcaaag
acatttcccctt

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E14,” located on chromosome 8, at start/stop positions 66785877-66786604, shown in FIG. 1A, or a human ortholog thereof.

(SEQ ID NO: 27)
agagcagagacatttcagcagcagtaggaaagaccaggatctgcctctcctcaattaggccaccttcttt
cttcctttgtacaataatttatctaaatctttaaaaaaaaatacctagcaacgagtcccttgatgttttt
aatcttgaaaggcaggcatggacactttgtttcaaacatcatgaaactgctcagctatccagtaatgatc
cccttatttttttagccacctgggaagtgtgggtggtgtgggctctccaattctctcttggaaaggagtc
gtgggtgtcatgccaatgttcctttggtattctctcaatgccaaacagattgtgtgtgtgcctctgttag
tcctatctctgacactgatgaattcaacccactggcattaaataaggccatgacaaatggcaggagggtt
acaacacgtggctagtacaatagaagtatgagtcagatggcagaaagctttattggtctttgattctctc
ttatactggacagtgttatctctatagtcaagttatagtgctctgatattccttttctcctgtggctcca
ctaaatattccctagcttggtcttgcaagatagttcagtgtttaaaggtgcctgccatcaaatatgacaa
tgtgaagggatgcctagactccacatgatgaaagaactgagtcccaaaagttatcttctggtccctcccc
aacacatatataacaatggcacctacac

In an embodiment, a human neuronal cell enhancer sequence, which is the human ortholog of S9E14, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E14,” located on chromosome 4, at start/stop positions 163268027-163269325, shown in FIG. 1B.

(SEQ ID NO: 28)
aggagacatggattcctgctttgcctccttcactgatgagttctgtttcttaagagcagttgtcaaactt
atttgactctcatttgcaccttctttctaagtagataagcattaagggtaaagatgctttaaatatattt
atgctactcaaatggaagctttataaacaatgcaaatgttacaggctgatcagatcaaggatgtgacaca
aattagaaaatataaagtattagtggatgccatgatttgtgagacagccgctggagtatccctcatagta
gaacttgatgtttggaatttgaatataggttctcagaccaaaagaagaaagatatggttagaacttaccc
ttggtgttctctcaatggcttacttaccattacaaatgaaaagagttaacatgtacataagagctctttg
ccaggaactgttaaagcattttacataaattatctcatttaattatcccatagccccgtacatataaata
tatcttaattttttagttgaaagtactggcaccagagatgtcagacaactatcccaaggaagcattagta
gttagaaactagagctaggattcaaactgttgttgtctgactctaaaatcagcacttttaacagcacctc
gctaaactagatgagtctcccttaatttggacacacacaaaaaatagatttgtcgtggatcacaagcagt
cacttagattttggtctaatgtgtaattctcagtttcaatgatctctgcaaacgtcatattagtagttcg
tgagttacgtaaattgaaaatatcatctatctcttgtacaaaaataaaagctttgtcaaatttatattca
cacatgttgaaacaatgcattcagagaacaatgcattcattttatgtgggaatttaaataggtccatgtg
ggataaagatctgaccggtgttctcatgcaaaaattactagagtaactggactaaagtcaagaaacctga
tttctcacaccagctctgctactaaacacctgttgttcagaggtagcaaattatctcttctttttctttg
ttttttttttttttttttttttttaacccccttataatatcacaggactagactaggtagcctccaatat
ccttttcagttctaaaattttatgcttcgataattgtgaataaatgtgttgcccatcccttgagttaaac
atgaatgatgaatcatctcaagtctgaaaaatatggctttaatcttatgtgacacctcaggctcacggta
tctcaggaaagccctaccaatcacatttggctgctatgc

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M9E15,” located on chromosome X, at start/stop positions 163544935-163545684, shown in FIG. 1A, or a human ortholog thereof.

(SEQ ID NO: 29)
ttttaattaaaggagactttatgcaaccacacacaccaggaagatttataagtgcaagaatgtaaattta
gtctatttttaaaggatgttattgacaggcacagttcaaaagacctcaccagggagcccttcgtgagaca
ctgcgtgtttcatgatgaagcttgagctttttttttcccatcatgcatattaagttgccagaatgatcct
taattaatcacatgaaagcctgacacttgaaaaactaatgtgtgagagacatggaagttcttcattaccc
tcgtctcctctgccaaggcattcaggttgagagcaatcctggctgggaaagcccacttccacacacctgg
cctctgctcattcccagaagctgctggcagcacttctgggtcactgtggctgtgggcagacacaaggtgt
atttccttccagggtggcagcatatgcctctgaaaaaccaaactagatagaacctgggatctggcagccc
aaactcctctaatagggattttataggaggatgctctatggggaactaaattgagtcttcttttgtgaga
aagagaccaatctcatcagaatccagaaagctaggcatagtagcctaagaaccattgaagtgaccaggga
aggaccccttccaaatccatatgccctgggtcactcagccaccatcttttaaaacagcccgattttgagt
ctggggctccctgtcatgagaagggaaatggaatcagactgtattttgaa

In an embodiment, a human neuronal cell enhancer sequence, which is the human ortholog of S9E15, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E15,” located on chromosome X, at start/stop positions 16156871-16158242, shown in FIG. 1B.

(SEQ ID NO: 30)
ttatattgcaggcggccaagcattaagtccagctgaatatattcagagcaatcaacaaataacacactga
gaacagacttttctcttatgagacttcacattagaaattcattcagtagaatgtatctaaatttttaaaa
agcttcaatgttgcacatatgttaaatttaagaatccctgggttagaatattactgtggaatcatgtcca
tgactaatttttttaaaaacattgcatttacctagtgttcatggtctataaagtacttttatactcatta
cctcatttcatccaagccttccctgctggccaaagagctgctttacatcctttactatggaaatgtgacc
atctaccattacattaaaaggcccatggaactatgaggctgaaacatgaaactgttaagattgggccatt
ttcgaactacaaaaaggcaatttcatctggttcagcttaatctgatttcatattgtttcgaggtgaacta
actgtacttcattcccatgggctattcagttatactgaagagcagtgctgatcatcacttctccacttta
caggatattctcttgtccccaaagtgaatctgtttaacttggcacaagggagtggacagggtcattgtgg
gctgccccaggagagacatattggtgggcacaacattcaatgccacaatgaaatggaaggctaagacaaa
gaagggtgggatccttgggagcaaagacactggccaagaaatacaccaaatttgaccaaaacctgaatgg
agtgctgtggctctggaagtgctggaaaggaaggggctcaacttcaccaaacactgacaatgtgccaagt
ccagtgctgggcacatgaagccgtggcagggagattacttactgatcctcaagtcacatggggaagttga
gtggcagagtataagtgtggagctgatctctctatgtcgatgtccattccactgcttagcattgcttccc
aaatccaacctgtgtgaaaaattaagttcacagtacttagaagaaaactccactccctattctaggtttt
gtccagagacctgaactaatgatagagtcaaagaatgtatgcaaagagcattgaggcacctagtttaagg
aattgttgagggcagaggctgaaatgcagcacaaagcgggtgagtcagaaagtcccttaggtcagttgtt
cgcaccctccttctcccctactcagagacacaattcacttgcaactggaagccctgggaagaatttccca
cctagttctgtgtctgaggagagaaaaaaacttgctaaggaccaagttagagagaaaacccaaatgagaa
cagcgaatctagccatctttctttgaatcagaattagttgga

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M9E16,” located on chromosome 13, at start/stop positions 96181419-96182168, shown in FIG. 1A, or a human ortholog thereof.

(SEQ ID NO: 31)
ctaaaatcttccctgtgattaatggggtgaaatgtactatagaacaggttcaaatgaatcacagtaaaat
atcctttcaattatagataactttctgaggaaagaagaaaaactgatgttagcagataagtaaatacata
agggaacctgacactttcccatatcacttgtcatttcatagtttcaaattaagctcagctccccaatatc
aattaaacccttacagtgtgaagcacgttgctgcgtattgggaacggtgagataaacggaaagcgagaca
aagatgggacattagagttgccgtggaagtggctgacaggatggataaaaatttctctgtatcgtggtaa
ttgaatgggggcacgagtctattaatatcagaatacaatttgtttctagaatgggcgttcatttattatg
atcagacgcttgctcttgcactgctggagatgctgcgtcggactcatgtggctcacatattcaagtcaag
aaggctggcactctttctctctgttgctcttccatatttgttttctctgtcttccctgacagtttctccc
gaaggaagacaaccagtgctcgcctatctcctatcagtctttgttctattaccatgttcttaatggaagg
tgtgtgctcacacctccatttctctcaccccatttacctttcaacatacaacagtctggattctgctctt
acaatacatcaaaaattcctctacgattgacaaatggttggcagatcacc

In an embodiment, a human neuronal cell enhancer sequence, which is the human ortholog of S9E16, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E16,” located on chromosome 5, at start/stop positions 76306559-76308039, shown in FIG. 1B.

(SEQ ID NO: 32)
ctaaaaccttagctgctagttataagcatgacctcaaaaccagaaaccatcagggagaaggttaaaagaa
ttctctgggtaaaacaaaatgtgaattccacacagcaaaaaataccataggcacatataaatgataaatt
gagggcaggagggggttgaaacagaaatgaattaataacctgaatatataaattggaaattcataaagaa
atgtcaacaaacacttagcatataaaaatgctcagcttcactaatagtaaaaattaattacaaattaaag
caattatgacatttagctttctcctattattcagagaagacaatatttaaaagatagataagatccagtg
ttctctggggcaagtgaggggttactgctgagtctgttgtgggaattctgaaaacagataccacatttgt
agagggggatgatctacagtcttaagtattttttcccaataggaataaactattgattcctctggttgct
tttcaggttttttctttgcctgccatggatttctttgggtttactatgtttggggtttgctcagcttctt
gaatctgtaggtttacagcttttgccaaatttatgaggttttcagccattatttattttgtcaaaaaaca
aaattacaacaaatttagtgtaaagagctagttgacttttatctgctattctagaagcaggcaacacctc
actctataaaatggggtgagtgctctaatgagctgagcagagaaagttggcttcataggcagaaaagctc
taaagagagcagatacagagaacaaaaagcagatagccctttcaaagtccctttctgtaaagggctaaaa
cagaggggacttccttattatgctcatttgggttggccggaatcacctgttttttggaaaactggctctt
ttcaaagttcaatttgattaggtggcacttagcctgagtgactccattctgcttaggactggtctgctgt
ggcctaggtgcaggagactagcccaaaacaatggcctcccgtaaaatttaactatttgaatactttttca
gtgctgccctttttctcttctgcctgaactccagcaacataaatattagatcttttgttatagttccgca
ggtccctgatttagtttgttttctctgttcttcactttagatagtttccattgttctgtcttccagttca
ctggttctttcctctgtctcctccattctgctgttgagcccaaccactaagctttttattttggttatca
tatttttcagttctaaaattttcatttagttcttctttatttcttctacttctttgctagggctttctat
tttttccatttttgccgtgatgctttctaaatttcatgtgtttcaagtgtgttcatagttgctcattgaa
gccttattatggctgctttaaaatattcatcagataattctaacatctctgtcatatcagtgttgccatc
tattgattgtc

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M9E17,” located on chromosome 2, at start/stop positions 76252395-76253144, shown in FIG. 1A, or a human ortholog thereof.

(SEQ ID NO: 33)
tgatatccaaaatcaggatgaagactactagatgaagaaatatcttaggggttaacaaccgactatagac
tgagagtccccagtgtcttcttcagcttttatatctcctaaatgtccaattagctgatcatttttacgta
actattagtcaacctgaaaattcctttctaacttcatcaatctataggactgtgataaacttatctcttg
gcttttttggattagagaatggagttgaactgtcaagtagaaatgaatatttataaagtagatgcttatt
ttggtattcagaaaagcagtctctactcattggtaggattattcttgcggacaaaattctgaattacaag
ttctagccctcctttcagtatctccgatatctacactgttcagtgcagatttatttagtcctgttacaaa
ggcattggttggctttctaaaaagaagttcttctgaagcagaaaattgactccatgtaaatttctcactc
tgttgcacatattaaagtacattgattttgctgatggaaagaggcagaaggacagggagagatgtggggg
aagaggccaaaaaggtcataaatttcaaatcatggagatccagagaatattatcaaccttttctcgtcag
agaaattgtttgagagccctgttatgtccctgaaaatgaaaggtagagttgccagtgaaggtcaagagta
actctcctcaggatggagagggttttggatggtatatgaatatttgaagt

In an embodiment, a human neuronal cell enhancer sequence, which is the human ortholog of S9E17, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E17,” located on chromosome 2, at start/stop positions 177980148-177980944, shown in FIG. 1B.

(SEQ ID NO: 34)
tgacatcctaaaaaaaaaacgaagaataccagctaaggagaaattttaggcattataaatcaagtctatc
ccgatattcccaattctatctctgatgagttaatagaaatctaatttgatagttttagcttttatatcct
atgactaatgtccaatcaactgtctgtattgtgtaactattggtctaaccgtaattcttttttaactgca
tcaacctgtggcattgttataaattaaacttttttcttggtttggatttttgatcagtgaatggagtcaa
actgtaacatagacataagtattagtaaaaatacggcacttactttgacctttggaagacagctgttctc
agaatgattgggttattctcactgatacaattttgggttctaagttttagcccaggttcagtgtctctga
catctacacaattcaatgcagatttatttagttttttatgaagacattggttggattttttaaaagaaat
tcttttgaagcagaaaattgactccatgcagatttttcactctgttgcacatattaaactacaacaattt
gggtgatggaaaaaagcagaaataatttgagaggcgtgggcgaaaaggccaataaggctataaaaatttc
gaattacaaagagtcaagagaatattaccaaccttttattgtcagagaaatgaagagtcttacttgtgcc
cttgaacatgaaaggtggggttgtcagtgaatttcaagagcaattctttaaatagaggtgggtggttctc
aatgagacatgagggtggtgtgaaagt

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M9E18,” located on chromosome 2, at start/stop positions 105220620-105221369, shown in FIG. 1A, or a human ortholog thereof.

(SEQ ID NO: 35)
gagacccagacagacacatacacacagagatacagagagagacacagacacacacagatacacacacaga
gacacagacacacacacacacacacatgcacacacacacacacacacacacacacacacacacacactgt
gtttggttgtgagttactgcaactttgatggatttttttctctcctgtctctgacttcctttgaaaactc
acataagccaggttttcagtgcagttggaagcagactctgttagaagaaagaggccaatagatgggaaag
cagtgacaaatgaagctgcggggagaaaatgaaaactcgagctactcgagagcagagccacgggagtcct
tgtgcagtttggccaggctgggcctgtgcagacttagtttttggttctgactcacaaccaacatttcatg
gtacgttagcatttatgaggcatttcgctcacattgccaattggcctgggttcccataaatctccgctgg
agtgtatttggtatgtataattgtagtgggacgtagtaggtattatttttggcagtctagcagtctctcc
tacatctccccctttctggaagctgactcaggcctcaccagcaacagtaccctctcacctgaacccaccg
ggacctgtcagtgtgtccccttggagctgtgatatacagctgtgtggctctgcacccacctacatcattg
ccactctctgttgtaaaagtgggagaagaagcagagctgtagaacatcacagatggagagaactccacga
cgcttttaggcccaggatcaatctttgcctggaactagatcaacggggttggctgaggtaggtgtgcctg
taaactgtgct

In an embodiment, a human neuronal cell enhancer sequence, which is the human ortholog of S9E18, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E18,” located on chromosome 11, at start/stop positions 32295626-32297067, shown in FIG. 1B.

(SEQ ID NO: 36)
gaaacccactaactcactttacaatttaaattaagctgattacac
acaaacacacacacacacacacacacacacacacacagacttatt
aatgtagggtcttcctgaatggcatctgtttcagttatctatttc
tacatagcaaaatgacacaaaattgagtggcttaaaataataaga
attggccatttctccagtttctgtactttgctgggcagttcctct
gatgccttccctaaagctcacccatcctgcaacaatcagtgggaa
gtccaagatgcctcctctctcaagcatctggcactcggtgctgga
tgttggttgtgtggcctcagttctcctccatgtctccgcttatcc
tctaataagctagagcagtttctacacatggtgctcaggacaaga
acccaaaaggactgaagtggaagctgcaacacctcctgacttgca
tagcatcacttccaccacattctgttggtcaaagcaagatacaag
gccaccagatcaaggagaggggaaacaggctccaccttgatggga
aaagcagcagaatctcattgcaaaggggcctagcaggagctggtg
cagctgtcttttaggaaatgagctgccacataggaaatgagaagc
caggcgcagactgggctgggctttcgcatatgacttagttctaag
aattaaatcatagcatctgtggttaaaaagagaaagcttgaaaac
cactgcattaagcaaaaaggccagaaaataccatgaaaataggag
tgtggagcttttttgttccttgaataaatgtcttcctggtaggaa
gcacaggattcctgaggaaatgacatttcttctccattgagcaga
cagtgaaagatcgatgtgaccaatgaagatggtgagcaagtgggt
agggcaggcggttgatctttgcaggagcaggtatctgcaatttgt
cctcatagggcagatgacaacagagagtgtgacgcttgtgtggag
acaatcaggctgtccaataagagagacagagatgcagcaaggtga
gactgagtcccctgaagtgggagggaatggcatcaaaggaagcaa
atgcagaaagaaataggagctgctcagatgccgggatgttcatca
acaggacaaaaggctgaaaagtggcccagaaggactcttccttcc
tcttccagaaagagagcagaaaaaataagaaagacgctttgtcaa
gatagcaaagccatatgaacataatcactggacaccaagcaaatg
acaggagactctatgatgctccagcaactcaaaagaagaggacaa
aggagaaagagaagagaacagagtgagactgaggtttatacagta
accaagccatctccttctaagaaacactttccagctccctctatg
tgggcaggtggacacatagaggcacacttgtatatgtggttttct
aa

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M9E19,” located on chromosome 2, at start/stop positions 136062361-136063110, shown in FIG. 1A, or a human ortholog thereof.

(SEQ ID NO: 37)
tatagtcactatgggtggagaaatcacatatggtgtacgtttaaa
tttgtgtacaaagaacaaacaactactgaataaagactagaacta
cttcttttaaaataagtgaatttcatcatatagagttagttacat
cttttctttaaaacaatttgtacccatttcaaggttgtagatggg
gaagattactcggcatacagcgagactgtaagcatacataatttc
ttagatacagacagaataggaccaggtaaattgcagtattaccca
ggcatgtcagactagtaattgtggctcgggcatcagtgtttctga
gaggaggcaggactcattcagaatcatccactagattttgtgcct
ttgtgtgcaaggtgttcttttgttcctggctcctcctgtctatgc
tttttagcatttgaccaccagtgtcttagaagaggctgttgtatt
ccattcaatggaattggaaagatccctgttgggttgccttttctg
tttccagcactgggacttccagggccttccagtatgttgtattca
tctatgtggacgtgagtgggctgtacctgccagaggggagaatgt
ctggttatatatctacacaaccactgtgtttgttgtgtgcatttt
cagatgtgtgtgagtgtgtgtgtgtgtgtggggggggggtatgtg
tgtgtgtctctctgtgtgtgtttatatttatgtttacgtgtgttt
gaaaatggccaagggcttgtagtgaaaagt

In an embodiment, a human neuronal cell enhancer sequence, which is the human ortholog of S9E19, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E19,” located on chromosome 20, at start/stop positions 9519903-9520772, shown in FIG. 1B.

(SEQ ID NO: 38)
tatagttgcctatggcctgagaaatcaaacctcatagatttttag
aatggtgtataaagtaagaataaataagtactgaataaggagcag
agctactttcctaaaaatcagtacaattcattgcatggagtaaat
ggtgtcttttttttaaaaccaattttgatccacttaaaggtccta
gatagggaaggctgctgggtagacatagagagtttgtaactacat
ataacttcctaaatgcaaatagagaagtcacaatcaaatggcact
tgtctcaccccaggcttttatggctaataattaggatgttctctc
ctgtatggtgtccccaggcttacgccttgacattcctgggaggtg
gcaagcctcattcagaatcactcactggatcagtgtgcctttgtg
tggctgcgtttttttgttcctggctcctcccttctgttcctctca
gcacctgccccgactttgttttgtaagagatcattattttcaatt
tggtggaattttaaagattccttttgggtgccttttcgggatcta
tcactgggcctttatccccagaatacaagctcaggggcctttcgg
tgtgtgcatgtgagtggagtatgcttgcatggagaaggaatatca
cgtcatatatctgcacacctactgcaggggtgtgtccgtgtgttc
gtgtgtgtgtgtgtgtgtgtgtgtgtgtttgtgtgttcatgtgtt
tggaccgtggccattgtcaagcacagcataatggtttcctgtgaa
gagtctattccctgtgtaagggggtcatacctggcactcttcaca
cctgtctactgaataggccttatgagaaaatcaaggtagtgtcca
cgaagtcggtaagt

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E20,” located on chromosome 2, at start/stop positions 136133847-136134147, shown in FIG. 1A, or a human ortholog thereof.

(SEQ ID NO: 39)
ttctgttaccatacctttggcaataaccctgctatggatccttcg
tcatggggatccaggctccttggcatcaggaatgccgccactata
catgcacagctgcactgcacaagccgattttcatggatggagcct
caggctgttttgtcagccagtttctgggaagcaaaaggttctgtt
ttgtagtacagtgtatctttatcatatcacactgcaggactgtcc
catgctaggttggttaactagcaggaagcttggtttacatgcagg
atttgacagagctgatattcaaggctactgc

In an embodiment, a human neuronal cell enhancer sequence, which is the human ortholog of S9E20, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E20,” located on chromosome 20, at start/stop positions 9602385-9602692, shown in FIG. 1B.

(SEQ ID NO: 40)
tgttgctaccatacaacctagggaataatagtgcccatggctagc
tgcattgtattcaggtggagcacagccccttggtgtctgggatgt
gcttactacagatgctcacctgtcatgaattaattatctttatgg
ctagtgcttcaggctgattcgtcagtcagtttctgagaatctaaa
agttctttttgcagatcattctattctcatcattttgcactgcag
gacttttccatgctagattagtagctagtaagaagcttggtttac
atgcaatatttgacagagctgagattcagagctgctcc

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E21,” located on chromosome 10, at start/stop positions 19863719-19864468, shown in FIG. 1A, or a human ortholog thereof.

(SEQ ID NO: 41)
ttcccaaagaaagaactgtggaagcaatgtgtagagaaacacaca
cagatctgcacttggcttacactctgtgaactaagagtcagtgag
catgcaaaagatcaggactaatgctacagagaacaaggagttcta
aagataaattaatatataaagagaattccagttatataagcggtc
atattacaggccaagtaccacagacctcacaacagaataagggct
ccccattcagacataataaggataaaggaagaaagaaaacgcaag
gactgtatgaatcatgatcagagaggaaaatatcaaagaaaaaaa
tgtcttaagtgcaggtcggtaagctagttaacaagggaagacgaa
gagctcttctcagcaattatccttaaagagtgactgcagacgttt
tatttgtcaggttttaaacctttaaagagatcttaacactgtcaa
gaagaaaggtggtgtaagaacgcctttggacctattttctcagat
ctttaagaatagagacattaagaagaatgaagactataaatactc
aattcacagataacatcagcaaggtcctgttaagatcacaaggtt
aaaaaaaaaaaaaagacatttttgagtgggtatgaaagatactgc
gagggagaggtgttttctaagagaataaaatgtgaataaagggta
aaacttcactggcttgtaatcagtggccacacgtcataagaagga
taattaaaggacatcaaaactaactttagc

In an embodiment, a human neuronal cell enhancer sequence, which is the human ortholog of S9E21, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E21,” located on chromosome 6, at start/stop positions 137017704-137019056, shown in FIG. 1B.

(SEQ ID NO: 42)
ttactaaagaatatagtaaagcaataattagaaaaggaaccaatg
aaataagacaatttatttaaataaacactgatatgttcacatagg
tgcactctaagaaaaactgtatcctagaggattaatttacagaaa
tccttgtagagaaatggtaaagaattgagaaactactaaaaactt
ggaaaaggaaaggagaggctactttcttataaaataaagcaaaga
taatccttggattaaagtaaatgaaaaaattcagtggtaatatgt
aggaatcaaaatattaatttaaaatattggaaagatcacaaagca
cggagtgataaccaatcaggttttatgaacaacttatcctgtcac
aaatatgcacattctagtcaatcaatgaacttgaaaaatataaaa
tattaatactgccaatcaaacttaggaaataaaatttagttcagt
cactgatggcaggtccaatcttggctaaggatcataattcttgag
taaagtagctgggactaatgtcagagaaaggaattatgtgtgagg
taatccttagttaagattagttctatacaatgtattgttattttg
caagccaagtgaaaagaaaaattatttactttagaattaaacttt
gtaatatcaccctgattctaagcaaaattagctctttagtgtaaa
gatggctgagtgtaggtatgcttgtgttataattgtcatgactcc
tacttttaaccttgctcatttaaaaaattttgttttattttttta
aaaaatgttacagagaagcacaatcaattgaccactgccgtgtgc
gtgtgtgtgtgtgtgtgtgtgtgtgtgtgtgtgtgttaggggtag
gggaatctcaaaaggacatagcctgcccaacatcagtctttgatt
tcttcttgtaattctttggaaccattgggtagcaagtggtgttgg
tattttctggagattacatagagtgaatatttatcaatgatttaa
tactgctttgtggtactactaggttctcaattttaatcagatttt
tgaagacaaaatagaacatgaggaggcagcacattgagtataaca
cttctcagctcttccaaaatgtggaacccatttaacataaaaatt
gccaaggatctttccagagctaaaatgctgtggcctacattttta
gatggccttaacatatgaagagtggttagcatcttatacaaaata
aaatagactgtgcattataatatcactggtggaagacattgtcaa
aacatggctttatagaaatgaaaaggaaattgaacattttggttt
gtggcaacatgagtttggagatagaaaacagaagttatcttagat
agt

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E25,” located on chromosome 13, at start/stop positions 54058102-54058851, shown in FIG. 1A, or a human ortholog thereof.

(SEQ ID NO: 43)
gcacagtgtggacatcatggggatctcaaagtttggctagaaatt
tggtttttgttttggtttctgtgcccctcccccttgggagtcaag
gtgaagcaaatcttccaatttattctttagaaatctccctacaaa
cagtaagaacatgaatataaatgcaagcagatattagcatttgat
catgctgggaatgtagacacaaacacaaaagcatctttgtctccc
aaatcagcaagtctttgtaggtacactgtgatttatgcacggtga
ggaggtaaaggttaaaaagatgttttcaataacttttttatttta
ggactgtaaacgtcagtgaatacagaaggcactgtcagacgaatg
cactgtcaagtgttctcaaaatttatacgtaggcagcttctggga
ttattaaaactggcttactaactacctaaaatggttgcttgcagt
gttagcgaagagtgttgatagcttgatcttcagcaagtgactaca
ttgctcattacagacaataaatccctaatcccatgtcctgttttc
atcttccatagctatcatacaactttaaatcaacatgtgtccctt
tgggtaaaaattggcagctagagtggccaccatatttactatggt
tctctccttcttcttcctcttttataggcttaggtgaggtttggt
ttgagttttgtcttttgtggtggtgagttaaacctgcaacctctc
agacactgagcaagcccttcaccccttagc

In an embodiment, a human neuronal cell enhancer sequence, which is the human ortholog of S9E25, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E5,” located on chromosome 5, at start/stop positions 175448287-175449217, shown in FIG. 1B.

(SEQ ID NO: 44)
gcacagagtgtgaatttcagaggaacttacagttttgctagaggt
ttggagtttttcctattttcttatttgaagctacggtgaaacaaa
gccttctattttgttctataaaaatctcccccacaaacctcaaga
acacaaatgtaaatgcaatcaggtattagatcatgctgagaatgt
aaacccaacacaaaagcacctttgtttcccaatttagtgtgtctt
ttaagctctactgtgggtgatttatgcactgcgaagaggaggatg
tcaaaatgatgcttttcaacaaatctttatttggagtatataaaa
gtcagtgaatacagaaggcacactatcaaagggaagcttaatctc
ataaaagactaaataaagcaacagaacggtcaagtatatcaaatg
atgtatttaagactctttgggattattaaagcggactttatacac
aaaatgattagcttacgatgttagtgaaaagaagataaagtttgg
ccgacctgactaagtaactagaatgcatgtttcagacaataaatg
taacttttctgaaattcctaatcccatgtcatattttcccctcct
atagttatacaattcaaaaatcaacactttaaaatttcaaaatca
atatatatcagtttctgcagtggttggtgggtagtgtaacatgca
taatgtgttttctattgttgcacctgctcttcgttccgtttttcc
cctcttttctgccttttataggcttaactgagtagtttttataat
tccgtttcatcttttttattggctaattttctatattttttgtta
ttttcatggttgctttagagtttatatcatttctctgtctatata
tatatatacacacacatacacatatatatattttacaatatacat
atgtaatatttttcaaagcctgcattcaagt

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E33,” located on chromosome 2, at start/stop positions 91888915-91889664, shown in FIG. 1A, or a human ortholog thereof.

(SEQ ID NO: 45)
Tacctggctaaaatcaaatacctacaagcaaaaacgaaggtgtct
tttgtcatgtgaggccagagctgagggaatttgtttggaggattc
tgctgggaagataggatccagttggtccactgctgtcttgctaag
gcattcaatggcagctcgctagctgtgtgcctcatgcaagcagct
gtttctgagcatgcccatctccagtgaggctttttgattctcatt
aatctgtcattaggtaagaaaactgaggtgactggaaaaatctca
tttgaaaagcattcctctcacctagaggatcaaggtatctcaaca
aagattttgctcaatcagacattcaccaactctgcttttctgggg
ttttgaggtcttgcttgttcctatattcaaagaaccaaagtgttc
agtaaatgtctctaactcactcttcagtggaagggaagccacaca
tgttgtatgagtgcacacatgcgtgtgtacatgtgtgttttgcta
ggatttaaatgccttcatgcatgcttggcagcctgagattccctc
ttttagttgtgctaggtacactgtaatgtgccatccttaggcaca
ctgtgatgtgcctgccatccagcatatccagaaattttgtcaaat
gggactgaagcatctctctgcctcggtgtctgcctatctctcttc
tctctctctctctctctctctctctctctctctctctctctctct
ctctctctctctctgtatagtttagcatag

In an embodiment, a human neuronal cell enhancer sequence, which is the human ortholog of S9E33, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E33,” located on chromosome 11, at start/stop positions 46354255-46355296, shown in FIG. 1B.

(SEQ ID NO: 46)
Tagctcactgtaatttcaaacccctaggctcaagcaatccttcca
cctcagcctcccgagtagctaggactataggtgggggcccccata
ccgggctcattttttctttctttctttcttttgtagagacgaggt
cctgctatgttgcccaggttggtctcgaactcctggccttaagtg
atcctcccaccttggcctcctgagttgctgagattacaggcctga
gccactgtgcccagcctctggagtctttttaagaaatgattttga
aatctttctaaacacattaaatatcttggcgttagaaaagtgtgc
agaataaggtgacacctactgcctagcttggttggagcttagcat
taggccatatttgctttagatagagatggaggcccctgtgtctta
ctccctggaggaaaccacaattctgattttggtgtgtgtgatgtt
cttgtgcctgtttttaaaattgtcctacataggtattcatgccta
gcaacatacagtgctgtttcgtatgattttactctggtttaaatg
gtataccaggcatattctgcatttaggtttttccctcaatattgt
tttggagatttaaccatcctaaacatgaattctcacggatccatt
ttaacctccacgtccgattccattgtgtaacagcagcttatttgc
ccaggctctgttgacggacacttgggtggtgtgcagtttttcgct
attgcaagcgatccctctgtgttctctccaccagtgcttgctcac
agggtctgtgtattttcagcttagacagatgttgccaaattgtgc
tccgaagtagttgtcctaatttttaggcctctgtgtttatttttt
tattttattttatttttttgagacagagtctcactctcgcccagg
ctagagtgcagtggtgcgatctcggctcactgccagctctgcctc
ccgggttcaggccattctcctgcctcagcctcccaagtagctggg
actacaggcgcccgccaccacgcccggctaattttttgtattttt
agtagag

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E34,” located on chromosome 2, at start/stop positions 91893197-91893946, shown in FIG. 1, or a human ortholog thereof.

(SEQ ID NO: 47)
Tcttggggaagcagatttggctttgagtgcggagttgatactgaa
attacagactctttatttttatctattggaaggataccatttctt
ctcggaaagattgggacagaagcaaggcagctttgttgtcaaaaa
tgatttacctcttatcaagattctaccctcattaatttccttaaa
gctgcctgtcagttgattaaaagctgtttccgctgcaattatctt
aaagttgacttcagtgatgtagcacaaagcatctatcctcaaaac
gttcctctcttcagaacattgcagaaaagtaggaggggcaagtta
aaagtcagaggtgtctctgatgtgatttgtcagaggaggggagaa
caagatacaaaattaaaaataacattagaactgccatcagagcca
ttcgttcaggagaatttttccatgacagtcacacagctctgatga
tcccctcacacatactcaccccacggtgtctctcttagcagaatg
gagtccatactacatgtcttctcaaaagaaaattgtgctgtgtgc
ctcctgtaggtattaaactgaaactcttaggacccagggagctct
gaattaagagtttattagactgggcatgtagttcagcaacagagt
acttacttgcctaacatgtgagcagccctagtgaatcaccagtag
tggaagggaaagtagggtatgtttcctccaccacctacagcaagg
ctagatagcagtttgaaaaatgtccctaaa

In an embodiment, a human neuronal cell enhancer sequence, which is the human ortholog of S9E34, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E34,” located on chromosome 11, at start/stop positions 46445563-46446867, shown in FIG. 1B.

(SEQ ID NO: 48)
tcttgatttttctatgcctctttgataatgaaaatcattctttag
ttacaaggtctaccacttccctggaaggaattattccatttcatt
ctgatccaggctcagttccactgagtttccgaattgtttcctggg
cttgcttatcaagatggttgacaccctcctttgaccactagtctc
tcaaattaatttagctccttatcattttctccagcttttaacaac
cccaaatgatagaaggtaagttgattttctcagtacatgatgaac
aaaatgctagttgctaagagagggtagaaaataaattgaaaaaac
aaacttcatattttaagagcttattctgggggaagggataagaac
tacaaacaggaaaacagttaaggaataataccagatgctttttga
gtactgtggatctgcaaatgggaacacaaatgattaagactccca
aatcccggctccagaggcatcctgtactgtccatgtctcaagaaa
catcttctggagaacagctgtgccataaaatcaacatgtagggtt
gccactgtgtggaagagactggggaggtgctctctgccaatggtg
ccagggtgctgaggaggaatgaagcctggcaccaaggagagtcat
tttttttgtgtcatataaacacagattagggagctaaagatgcca
gcaacagattagagtactgttaccaggttcttttcttcaagaact
gtaggccaactgagcctgtctcttggaaacaacaggaggaaaaag
cctgatgttattaaaacaaccccaaccatttagtaggcctctggc
cactaagatgaaggcaggaaatgtgccagagggggttaatggact
gagctagaaagtatttacatacgaggcacttagcctcagaaacca
aatcctgagagactgctaagggatttctaaggtttcaaggttctc
ccatgtagtaatgacagaatggaacaagctagactcagagtttct
caacctctgcactactgaaattttaggacagataattatttgtgt
gaagagtcttgtgcatcgcagaacatttagcatccctggcctcta
cccactagatgccagtagcaccccactccacatagttgagacaac
caaaaatgtctccagacactgccgaatgtcctctggggataaaaa
tcattcctagttgagaaatgctaagctagataaacatatcctaca
ttttaactccagggtaggtgctcttagcaccactactagaatgtc
tgctgctaacaagtgatgctgttatagtcatggaaagaaactaat

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E36,” located on chromosome 6, at start/stop positions 7489656-7490334, shown in FIG. 1A, or a human ortholog thereof.

(SEQ ID NO: 49)
Taggttccagggaaatgtgtaggaaattgaaacacaaaacatatt
ttataacaagagtcctgcatgtcagtagatctaaaaacccaaaga
gggtagtgtctcttcttctgttgtcttcaaactatattatggaca
tatttagacataatatcagctatagccaaataacacccagtgatt
tgaagcacaggaattgaaggcctaaactatcccttgaggggacag
ggactgcttctttgctgctgctgcggaaattcattttaatcttga
gctttagtctgtccctatctggagactgaagcatttctttttgtc
tacaatagaaaaatgacatcaatttagccctgtactttgcaatcc
cacgcttgtgcgtgtcatatttcttgaattatcacagttgtccat
caattgtgtcagacggagcaggaaatttcgctgtgggttgggttc
tgccaagatacagatttctataaaagattcatataaaaagtgctt
ccttataggatgctagcttgtgtgtgtgtgtgtgtgtgtgtgtgt
gtgtgtgagtgtgtgtgcatgtgtgtgtgcatatgccattaattt
tgggaatataagtgcttttactcacaaacacccttttgtgtttcc
ttactctcagggttttcatgatcttggaatttagtttgggtcctt
gtgc

In an embodiment, a human neuronal cell enhancer sequence, which is the human ortholog of S9E36, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E36,” located on chromosome 7, at start/stop positions 97723688-97724767, shown in FIG. 1B.

(SEQ ID NO: 50)
Tagactctccaaccagggaattaacatggaacaaggcattgcaca
aaaatctcagaggaaagagcagagacaagttattcagactgcagg
gcatcttggtcaagtcctgtgctccctctgtcactccctcaggct
aagaaagtaaagctgaacacaaaattcttagccctcagacgaggg
gaaataaatctcagatattgttggaacacaagtagtcactcccac
tctgtctgcccagagccatgcaaacaacatcaagatggagaaaat
ggcccagagactatgcaaatatattagatactgtattagtcagag
ttttccagagaaacagaaccaacaggatgtgtgtggagaaagagg
gattgattttaagaaactggctcacaaattgtggaggctggcaaa
tctaaaatgcacacagctgtctggcaggctgaaaattccagcaag
aagttgatgtcttcaggtcttaagtcccaaggcagtctggaaaca
gaattccttcatcctcaagggacctcagtctttttctcttaagac
cctcaactgattggatgaagcccacccacatcatggaaggtaatg
actttccgcaaagtctactgattgaaatattaataacatctaaaa
aaaaacttcacaagaacatgtagatgtatataatcaaataattca
gcatcatagcctaatcaaagtgacataaaaaattaagtatcacac
acaaacatgagggtgcacatacacatatccatctagaataataat
ctagaagaagaataatagttaacattcaatgcatgtgctaggtac
tgttttttgtttgtttgtttgtctgtttgtttgtttttgagatga
agttttgcttttgttgcccaggctggagtgcaatggcttaatctc
ggctcactgcaacctctgtctcctgggttcaagcgattttcctgc
ctcagcctcctgagtagctgggattgcaggtgcccaccaccatgc
ccagctaatttttgtatatttcatagagacagggtttcactgagt
tgaccaagctggtcttgaacttctgacctcgggtgatccacctgc

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E22,” located on chromosome 10, at start/stop positions 41060302-41061051, shown in FIG. 1A, or a human ortholog thereof.

(SEQ ID NO: 51)
Ataagtggaagtgcgtccacagcagtgaaaatgagatttgttgag
atggactaagacaagtgatactttcatcgttattaattctgcctt
cgttttcataattacctttgtgtgtgcagtgtttacaaaacaggc
atttaataaccttccagtaagagaaagaatactgtgtcatgctca
tttactctgtgctcctattaataacagcaatttaaaaaattaagg
gagcaattattcctttaacacagagcagactccaatattacagta
acacccaaggggttgtaaccattgttggctttggttacactgtga
cataacttaatagacattggggggtaagggaggagggctgttacc
tgcatgctcagatatagacaagtccccaaagcctacagccagggg
ctgataagtgactgaacacagaacattctctcaatggccatgaga
gaactcctgcctgctcttttcattttcaagctgccagtcaaaaaa
acgctcacagagtcagtgcccaagctgggcatattaggctgcttc
tgtgtgctgtggagtttgtaagaaacaatttctttctccaagctg
cattgtttaagggagtaatccattcttttcagagtacccttgaat
acaaactactaggagtgtagtatatccatcccagtgagcaggtct
tggtaagtagcaatgactcccaagtacatgacctgtgtgtgcggg
catgtgcatgcgtgtgtctgtctgtctgtc

In an embodiment, a human neuronal cell enhancer sequence, which is the human ortholog of S9E22, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E2,” located on chromosome 6, at start/stop positions 109993836-109995273, shown in FIG. 1B.

(SEQ ID NO: 52)
atatgcaggagagaactgtgtgccacagacctccttttgtctcag
cttaaattgtagcgtaaccaaacacaacaaagattacagtacctt
gcagtagggtgttactgtactattgaaataagccttgtatcaaaa
gaataattgcccctagattttaaaaattactattattagggaaaa
cacacagtaaatgagcatgagacagtactgttacttttactggaa
gggtgttaaatttgcattttgcaaatattataaacaggataaata
tgaaaactatggcagaaggtaaagacaactaaataataatttgag
aacattttgataaatcatatttccactattgtggatgaccatcca
ctttattactgaaatgtctgacagctgataacatcaatttttgtg
gctgaggcatttaattactatgtgacaggtgtcactgaatgggtt
ggtactaaaccagcagaatttccatgccatgcaaagtgcttgtga
aatccagtctgcctggggcttgcagattgcacttaactccaggat
cctgtgactatttgtgaccttcttcctcctgccatagggccacct
tctctatccaggcagccaactgtgtttgctgggggttttggttgt
ggcatcagagggaatcagggaggcagggaccttagaagccatctt
ttgcaactcccttgggtttccatgatatcaggtctcagagaaaga
gggacacttattcgagactgcacagctagtaaatagggaagacag
atttggaactcagtttaccagggcttcttaccacataaggtgttc
agcattctgtcagctacacaggcccaggtgtgttcttgcctgtct
ttatagcccctctctgcccctccctctgctctcagacatgtccct
tccttacatcttaggaacacagctgggccagcactccatcaccaa
ccctggtgcccaggctgagctccgagcattattaggggtggtgac
acaagcctgacctttggcaaacagggtgctgatggagctgttttc
agagcctgacactctcttctggacacaagctcagacgggatagtc
ccacagctccagagttcttgccctttgatgtgatgaggggaaggg
attgtgaaccccacgctctgcttatcattctctcaaggctcaaag
tgtggctgcctttcctctttctttcagagcttcacaagacaaaat
gatactaccaaggaggcagagtctggtggcagcttccaggtctat
tgttgtagacaactctctttggctaagcattcagggagttggtag
acaagttgagccatcatccctaacaccagagccaaactctcctga
gctctgtaatttagtccaggacctctttcatatcttgttcagtgc
attcagcatcatcttcaacaacaaaatcatgctgttggccatc

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E23,” located on chromosome 10, at start/stop positions 41092784-41093149, shown in FIG. 1A, or a human ortholog thereof.

(SEQ ID NO: 53)
Cccacttccatcccccgcctccctttctgagattccccccttcca
tcccccgcctccctttctgagatcctcccccgttccgtcccctgc
ctcctttcctttctccccattagtctaatgcagtcattacttcca
ctaaacttgcaaaagagcctcttctggctttgctggtttccacca
taatttgcctattttccagttgaaccatgttcttgttattatctc
tacctactctttatttgcggtttaatttgcttttcttcctctgag
ttctggaggtgggagcttggcttatttttaggttattgatttaaa
agctctcttcttttttctaatattagtgctcagagctatgacttt
ctcttg

In an embodiment, a human neuronal cell enhancer sequence, which is the human ortholog of S9E23, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E3,” located on chromosome 6, at start/stop positions 110012056-110012532, shown in FIG. 1B.

(SEQ ID NO: 54)
Ctcccttccatggctccgaggccccacaccatctttcctgctctc
actggcctcacctcctaccattctccccttgctccctctctccca
cctcactggccccttggctcttccttggtcccagcttgttcctac
ttcaagacctttgccacgtggcccactctgcctagatgcccccct
accgtgcccactgtgcacagacactacttcttggattggaggggt
ctgagcactaatgtcacctcttctggaaggccctttcctgaccat
ttggttcaaattggcatcccctttcctcttctctcagtcccttgt
tgaattccttcatggtgtaaaacttcttaccatttattactgttt
tatttatttgtctgttagctgagattttatttatctctctcacaa
gaatgcaaagcccattgcagtattctaagtgtttgtaatgtgtgg
ttggtgcccaacaaagacttttacata

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E24,” located on chromosome 10, at start/stop positions 75321460-75322209, shown in FIG. 1A, or a human ortholog thereof.

(SEQ ID NO: 55)
Ccatccaggacccaagctatctgaatctctaaggtggcagctgtt
attgaagaattcccacagttcttgtcttttgagacacctgcggga
gaccccaggactgaagggagcaacaggggcagctgatagagttcc
ccgtgacacagacaactaggtagctggtgaacattgtccctgtga
ggaagaggcagcctggtgtgatggcacatgccagtggaactgtca
cttagtgggtagaaatgggaggatcaagagttcaagactatgcct
ggatacatagcaagcttgaggccagcctgggctaacagggacctg
tcacaaaaaaaatggtaaggctgggattgtagaacccaggaagaa
acccactgcagctgcaatgggcagtgtgtgtactgccctgtcccc
tctgcttggccggagcaggagcagtgtgtgcaggcctgcctgtga
gctcgtagacacatctagcagggtctggagccaccacctgggcct
gtgtacacagagcttcgggtggctcaggagagatgtgggttggga
ttaaatgattttaattaaagtggctggttcaggatgggagcgaat
ggggacgaagaggcggcaatgctaaatctgaatgctgggggccga
gcggctgttccttcaaggctctcgactcatgttccacagtatgtg
cctggaggtgacctgggacagtgtctgtgctctaccctgtcccca
acagtgacccatcataaggaaatgtatt

In an embodiment, a human neuronal cell enhancer sequence, which is the human ortholog of S9E24, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E4,” located on chromosome 22 at start/stop positions 24428555-24429423, shown in FIG. 1B.

(SEQ ID NO: 56)
Ccattcctttctttgcctctttggagattctaaaggtctctgaaa
tcattattagattactgtattaccttccttcctactggaatgaat
tcacctccatgttttttattctgttgcttacctttcttagccttc
atatacctcttgcgctttgaaattgatttgcgactcctcttagct
gagccaatgttgtggagacagggagtggcctgcagtgcctgaaac
tgcagggcagcagccgcctcccaggaatgtcctgggtcctttcct
cctttgaggtcccctgccatatgctcagatggggccaggcacagc
aacgggggcacacctccctttggcccagcagttggggtgctgcca
ggcctggtggaggtacttggtaaacatttgttatggaaaagcagc
aggagggttctaaggctgggattctggagcccaggaacaggccag
gcatggctggagcgggtccgagtccgtgcactgtgtcctgtatgt
tgctcttagccgcactcaggagcagatgtgcaggtcactgcctag
aagctggtgggcacctccgtgcagcaggctctggggggtatagcc
accatctgggcatgtgtacagagggccttgctagtgcgacatatg
aggaaccagaagatgtgtgggttttgattaaatggagttttaatt
aaagtggccaggttgggggaggtgaggaggaaggcaggaagcagc
agcagttttgagtctgaccgcgtggaatcctataaggtggttctt
tcaagaggcattatgcacattccgcagcatgtgtcccctgtctgt
atggggcagcatctgccctctgatctgtccccaacaaggggtaat
cacagagaagtatt

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E31,” located on chromosome 2, at start/stop positions 73282424-73283173, shown in FIG. 1A, or a human ortholog thereof.

(SEQ ID NO: 57)
Tgggtaagattagcccttttaaaattattcccttatgttttaata
ttctcaacaggaaggattaaattagagattaaaataaaaacctta
ggggctaccttgtaaagttaagggtggtatcacattttggtgaaa
gtcaaaagtataagaaagtgtatttctttaattcattcttaatta
taggatgcatttcaacgtttttcttcatgaacactatattggttt
ggaaccctcttttaaggcagtaagtattgcataggttttgacatt
acaaaaattataaagtctaaaaaggtgtacggcaggaaataatca
ctgcaacactgatgaactcggtgtaaattagcctttatcaatttc
aaatgcatgcttttctaagtgaaggagtaggatctgaggtgtgct
gagatagtaaaggatatgggaaggagttcagctcctctctagttc
ccttcacgtgcataatcaaaggactatttacatcatagatgcagc
tacatcacaggacatctgtttgtgtgcatagagaaatattagata
tggaatagtttgaactccttccaaatttattttcttacactactt
ggaaattgtgatggctatctaaaccatttttgtactagacaaaat
aaaaaataaaaccctgatttaaaaaaaatacattcacaataggca
aaaaagttttagttgcatataatctgcattttttattgaaaattt
ccatagttttctcttcttctaatcctttgg

In an embodiment, a human neuronal cell enhancer sequence, which is the human ortholog of S9E31, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E31,”, located on chromosome 2 at start/stop positions 174345788-174346818, shown in FIG. 1B.

(SEQ ID NO: 58)
tgggtgaccaaggctgaaagccaaacaaaatgaacatcttccata
actacatggaaattttttctcagaattagatggttaaaaatattg
gagattttggcagtactttatagtttctcaatcctaacaataagg
ataattaaatcagagactaaaaaaaatctcatggccactttataa
aaagtggaccacattggccaggcatggtggttcacgcctataatc
ccagcactttgggaggttgaggcaggcagattgcttgaattcagg
agttcaagaccagcctgagcaacatagcaagatctcatctgcatt
aaaaaagtggaccacggtttgtgagtcaaatgaaaaaagtgtaag
aaataacgttttaaattcatttttaattttaggatgcatttcagc
atttctcttcatgaacactgtattgcttcagaaaccctttaaggt
agtaagtattgcataggttttgacattataaaaattataaagtct
aaaaaggcataaggtagaaaataatcactgcaacactgatgaact
caaggtgtaaattagcagtgtttaccaatttcaaatgcatgcttt
tttttaagtgaagaagggatctttgaggtgcgctgtaagttattt
aacaatactgtggaaggaattcaagttcctccccgccccaccccc
accctgcgtcttaaaaatctaaataatcaagggttgtttacctct
tcacttaagtttttcttagaggacacttgttttgtgtgtgtgcac
agacaagtcaaagacataatcagatgatgtggagtggttttactt
ctttcaaatgttactcctacagaatttatgaaattgtgactgcca
actatcaaaaccatctaatcagtatttgttttgtattaaatagca
gatggttaaaaaatatagaacactgataaaaaaacttgtgtccaa
gatgtgaagttatgttttttggaactgcatgttacttgtgatttg
taccagttctttctgtagttttcctttaccatatcctttga

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E32,” located on chromosome 2, at start/stop positions 73288192-73288941, shown in FIG. 1A, or a human ortholog thereof.

(SEQ ID NO: 59)
Gttaatttcatttatctatgtacctggaatttgacacttctagtg
actagggttggtgacaaatcacttcaggctagctctcagtgtgtg
tattttatattttctgcagactgctgttgtcacttgtgcaacaat
atcccaagctggaaattgagctgctttatggccacataatatgca
ctaatgaaatatctttaaaaatgtaaaggttggctcccttttcaa
agccttgggactcagggtaggagaaactctttcctccctcccttt
attctcatcacattttgcttctcaagcagatcacaaaccaggtga
taacacacggtctcagatgacacatctatgatagaactataatgt
ggggagagaactgctgccagcaaacatttcacaggagaaggtggg
gacagacaaagcacccagaaagagattccaagtttgttgatcaaa
ccgtttgttctttcccttccggttgggtcttctgccacttatttc
agtgctcagctacatgttactctgcaaagcagaagtcagcatggc
tcaagtctcttcactcatcacagcagttctgatgagacaatttta
ggcagactgatccctccctgtaacaggcctcctgttcttttagga
ttaccaagaactaactgtacacccgctgctctgtgttctagggat
gaggcatataatggtgggggtggggcatcacccatgactggacta
tgggggttaaaactaatttctatggatcgt

In an embodiment, a human neuronal cell enhancer sequence, which is the human ortholog of S9E32, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E32,” located on chromosome 2 at start/stop positions 174352251-174353210, shown in FIG. 1B.

(SEQ ID NO: 60)
gtaaccaccaagtactttaattaatcttgcttttattcccagatt
tcaaaatttttaattactagcttgggttgctaacaagcagtttta
ggctggctattggtatgtatgttttatatttttttacactacact
tccacttttgcaaaatctcagactgaaaagtgagctttttgtttc
ccaaatatgaactaacaataaattttaaaaattttcagctgggag
tggtgactcatgcgtgtaatcccagcattttgagaggcagaggcg
ggaggatcacttgagcccaggaggccaacttggccaacatggcaa
aaccatgtctctaaataaataaataatttttttttaaaggtttgc
cccctgccctcaaacataaggtttatgtaaggtaagagagactct
tatctcattttgtatattcctgtttcattttgttttccctgcaga
tagcaaactaatgacaacatttcatggcagctatttaaagttgcc
agcctaaatcacctacactggcttaaatgacgtactccagctgat
gtgtctgtcacttagggaagaatgagacagttactgccaaaaagg
agttcatacgggagggtagggggatagaaaaatattcagaaatga
gttctttagtttgctggttgaatactttgtctatttctttttact
gctttgttggtcttttaccacttatttgcgcactcacctgcatat
tatcatggaaagcaaaaggcaatcagttcaaatttgtttacctgt
caatagcagatgtggttaaaaatttggtgagaaaaactcagttat
aagataatatattttactggactttccaggctttaccattccatt
catattgcgaagaacaactgtaagtttgccctaggtactagggag
atgatggggaaaaactgtctattattggagtatgggggaagttat
taatgatgtcatagt

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E27,” located on chromosome 14, at start/stop positions 32379503-32380252, shown in FIG. 1A, or a human ortholog thereof.

(SEQ ID NO: 61)
Atttttcttttccccctctgaacctaggccttaggctggctaggc
aagtctccactacagccccgcccactccatctcggctgtggctcg
cgggaaaggcagccccctacagttagtctccccagccatcttctc
cggttcccctgatgctctcatggccccagccatcctagagctggt
gcgccatcgctcacctttagcattcggcctgctgggggtgagctt
ttgggttagaaggaacaaggcatcagatggggtcgtgttttttta
cattccaatcagttttcggaggggaagaacagacatttttgtcag
ctaattgcatttaattacagagacaatcgctgtttccgaacgttg
ttgtgtccactccctcatccttcaatcagttcaagcacatccggc
cccagctgggagccttttaattgcataatttggatttcacttctg
gaaagaaacttgagaaagactccgtgagggtctccattaacacaa
tcagctccccaggcacccggggctggccctaattgacccatctca
gctaatgcctctttccaaagaacgcccgcgtcatttctttggctt
gtttgtgctattgtatcctgttttagtgtcttgatcttaaacatc
tttaaagagggcactcagtggcttgtttaactggaagtggtccag
ctggtttggaaggacagtgcatcaggtgatgtaagtccaagggta
ggaatttctggcctctccttcccctgccca

In an embodiment, a human neuronal cell enhancer sequence, which is the human ortholog of S9E27, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E7,” located on chromosome 10 at start/stop positions 49666034-49666952, shown in FIG. 1B.

(SEQ ID NO: 62)
Atttcagaaattcttagtgattcaagcaggacccttaggcagctg
ggctcctttatttggagcaggctatccagggactctgacaaaaac
ccatgtggtagagcctagagcagggctctcttctggcctcaggga
cttaggggacagctggcagggagcagggctgggaggagggcacag
cctcctccagagtcagccccagccccagccccagctccagctcca
gccccagctccagctccagccccagctccagctccagcatgggca
ggacaggcaggtcagcagaggcagagttgagatgtctccagagac
tgtgatcataggagagacacaagaggcacttcctattagagcaca
tttttatgggaagtctaaagggcagagggagggagtaagaaagca
accagagaaatctacagcatagagcccgtgtgttctgctccacat
ccctccaatcctgctgcttcctgctgagcactctgctctagccca
gcaatcctgtaggtctcccttctttgtgccactgtgaaggctctc
cacacttcgacctggactgtcacaggctggcagaggtggggtggg
catttgacctttgctcagctctttgaaaacacaagcagcctcaag
aagtgagttccttgaacccctggttttagatggaggatctctatt
agatgtcatgggagatttgggcactaccctttgaacatacaaact
taagtcattggcctgagatttatgctccttccctcccacggccac
cttcctactctgcttaggaagttgcagcacaagggtcctgggttt
tctggggaaaggcagcccacctgctgtaagttggcccaaagctac
ttgtctgttctctccaagtgaccacagtgcagatctgagttctcc
tccggttcctttccctcca

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E28,” located on chromosome 14, at start/stop positions 32409388-32410137, shown in FIG. 1A, or a human ortholog thereof.

(SEQ ID NO: 63)
Tgcctctgcctcccaagtgccgggattaaaggcatgcgccaccac
tgcccagtgagttcctgtatcttaagacgtccatatttgcttctt
gaccagattggattgtgttggcatgcatgattggacatagctata
gtaataaagatgggcccattcaggtgcaaggtatctggggagaga
ccccaacccactccccagagtgtacttggccctgcttgtggagct
ggaagcttccaaggcgacttgctgctccggcctgagtcaaggctt
tccttcatctaggggccatttgaggcactgtggcaatcaggctaa
tttactaaatggcaccctggtggcacccaaccattagatggcttc
ccacagggagtaatttatgaactggaggaaatctggccgtcttct
ctgttgatttcccccagatccttaattactgtcagtactgcctgg
ctggtctgatttcctccccaaggcccgcccacccatgcacaatta
cccagcacccggcacttccaatcctgctgtcacctggaattgtgg
ttagttgtctaagatgttttgaatttagagggctaagactttgtg
atccggatcacaaaccaaaaaagtgtgtctctgaaagcatccaag
ccataaatgtcactatactgtgggaaagataccactaaacaactc
agaccccaaattcattttattagatgatttcagggataagtagaa
aggattgttgagccagtgtagtgtggggca

In an embodiment, a human neuronal cell enhancer sequence, which is the human ortholog of S9E28, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E8,” located on chromosome 10 at start/stop positions 49601401-49603007, shown in FIG. 1B.

(SEQ ID NO: 64)
tgcctcagcctcccaagtagctgggattagaggcatgcaccacca
tgcctggctaattttttgtatttttagtagagatggggtttcatc
atgttggccaggctggtcttgaactcctgaccttgtgatccgcct
gcctcggcctcccaaagtgctgggattataggtgtgagccaccat
gcccggcctggaagagctacatttctaaaggttgactcagggagc
aatttgggggatcaggagctggtgtcaacaccgggtgaggggatt
tgccaacaggctactgcaatagccccagcaagagctcttaaggac
acaggtcagaatgtctagagaaagaagagataaagggtgcccagc
cctctgtacttggggctgaaaagtcccccttacccaagggaggac
acctacattgaccctgcacacaggtcagagggactatggaaagag
aaggccacagaggaagccctcagacctccaagcaccaaagccaca
cagccaagtggatgaagaactcatgagcgtgagtcagatggcctg
tgtctcggcacattactcatccacctgtgctgccaattttcaact
gataaaatggggcccagaagaggactgacctcacaggggtgttct
gagagtgaaagctaatgacagtaaaagccttccgctgtgtctggc
accatgacatctcctgacctgtctctctcacatcagacttgccta
gacacggcggtggctgacagcctgggaagacacacttcattgaaa
ctgggggactttgcaggcgccactgctcctctctgtacccagcct
gcagtgaccatgcaagcacagtatgcgtgctcctcagggctcagc
cttcccactagatggggcttcctcctgtgtctcccccatctgcat
ccaggagcccctttcagcagaagcctgtttacctcaagatagatg
atcttgaacccatggcttcattaacaaagcacacaaggtagatgg
ttgctatctacccacccagaacaactggctcctaaacaagcttgt
ccaattgattttcactaagcccccctctgccactgtttgccaggc
actgggatatagagcagaggcatagcatttgtctccatgggcatc
cttccgaggcagtcccctgcacacagccccaccctccctctgccc
tggtgtcctaggagacccctgcaccaaacctacacttttccaata
tgaaaagggaagtgacctggctgatcagtgctgacctttctaatc
atgacaagcttcggaattcaggagagatggctgcagccttcatct
gtctccttggttttcccaacaaagccaactgactgtaaaatacag
tttcccttaaacatcttttctgcctacctgactagtcaaccaggg
tagctgtttcctggtctccccagggtgtgtctggagtatttgtca
agaatacttcaacttccttctaagtgtccctgcatccactccaaa
ctcagcagaactttggcataatgagaaagccctaagtcttttcaa
aactgacaaacaggcttgttcctccattccacagttattgattca
gggtgttctgtgccaggcagtgagcctggcca

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E38,” located on chromosome 9, at start/stop positions 91378247-91378784, shown in FIG. 1A, or a human ortholog thereof.

(SEQ ID NO: 65)
Cagtagcattggtggctgccagaccgaggtgcagcaggcggtcct
ttccgggcccagtgggacattgaaattattatatacacgtctagg
acgctttaaaatcactacctcttccaacttcaccacctccgaaat
gacccctcctatcatctccatctttggcttgggagcaccccttgg
tgttggtggacaaagtaatctggagataagggtcttacacctctg
cagcagaggccgtgcccccaggcacccacagactcagccctgctg
cctttcccacctaccccattatcgctacccctttccagcgcatgg
cgcggcaggggattcggcagaactgcagcccagccggacgaaggc
ggaggctttttgactttgcttggtctgccagcttcactgctggca
ccgaccggctcaagatcccagcaccgactgccagaaacctagatg
cggctgggcagagtgtgtccccggggtccggggaaagtgcgaagc
ccacgccaccgcttctcatgcacgttatttgtctcgacaacag

In an embodiment, a human neuronal cell enhancer sequence, which is the human ortholog of S9E38, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E38,” located on chromosome 3 at start/stop positions 147420752-147421547, shown in FIG. 1B.

(SEQ ID NO: 66)
Cagaagccactcacccctcactactcatgactaggaagaaagtgg
ccggggcatgagggttcgagttctgtcctaggtcccggaagccag
ggctcccagcgtctagtcaggggcgcagaaaccctcgttcctcca
aacccccgccacagggaccactccagaagaaacccattcgtgatt
tgggtccctttgtgtttgagtattgtggggagtgggctatctaga
ctctaaggactccaagctaacgatgacctgtgtgggccttgctct
gaacagaaaactcaaactcagcgtgggttcccgcgtcttccccaa
aaggccaaggcccaaagaacccttttcatttggcgaggggcttga
ggaagggaggggctggggcgggagagggtcggggtctgcaagcca
gcggcccggcgcagcggacttgtccggctgctcagcttggcagct
agcgtccgtgccggcaacgctgttaccctctcctgggctggagag
tggggacatcccttccccgcccctaggcgtccctggggtcggcgc
cagagcccgggagcgccggaggctctggctggatggcggcgagga
gggctctgagtcctctcccggccaggccgagcttagtatgtggag
gcggcggcgggagtgcgggcatggaccaagggaagggggacaccc
aaaccgcattggtgccttcctttccctccactcgcgccgtagacc
tccccaaattgcccgtctccggtatgactgaatctgtctgctggc
ctctcagctaccaactctgcccgaaatctag

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E39,” located on chromosome 9, at start/stop positions 91382525-91383274, shown in FIG. 1A, or a human ortholog thereof.

(SEQ ID NO: 67)
ctgattttttgcctaagccaaaggcaggttacatgcaaacctcta
tttaaatctgagcgccagtgccacggcggaatggagactgttgat
tatatcagggagtcttccctgcaaaccgggaaagaagcgatcaca
aagccaacagaggctcatcttgtgttcagtcttatgctagagaag
ttttagcacccctgatcacccccaggaccttcagttgctaagatc
acagacacctctccctttccttttcgaaacaacctcagctgagcc
agagatttagggttctggcttctgggagggtctgtggtgggagtg
ggggtcattttctcttccatagagcagcaaattcaagcagcaaga
aactcctggaccgagccttcctccaaccaatattcccagctttgt
gtgcgttctggccttcctgaattttgcctagacctgcggtatacc
cggtgggcctggcctgctcctagcagccactcacccgaagcttgc
cacagtcagggccgtgatttcaacagatttaagatccctcctggc
ctgaaccgattttcattttcatctatcagcctctgggctcctctg
gcctgcgaggtggaacaggcgggcgggtaattgtctggggaggat
catagacctggaattgtcagtttggactgagagggtcccctctac
actcgataaggtctccgcgacactgctggatccgtatattaaata
aatattaatattaaataaatattaaaatcg

In an embodiment, a human neuronal cell enhancer sequence, which is the human ortholog of S9E39, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E39,” located on chromosome 3 at start/stop positions 147362972-147364211, shown in FIG. 1B.

(SEQ ID NO: 68)
ctgtgtttttttctaagccatgtttgaacagtgtaattatttgtg
atgtacaaagaaaggaggaaaaaaattagaatgcagaattgttgc
tagggatttgcaaacatcatggaaaaagtagtacaaacccttgcc
aagatcccagaactctaatctctggatcctgatctccaaaggaag
gctagaaaattctgccttggcctaaaagtcttgagaaaatgagat
tgaaaactgttgataaaatcagctgggagaaaaatatataaatgt
tcttggcccaagtgaccaggattagtatttaaaatttatgttgtt
aagtaaattcaaatacaaaatcaaagaaaacttttgtctagcaac
atgactgcttatggtatgtgttctcttactggttaaatgtaccat
agacatatccacacgcacacacacaagaaagttccagtttgaaga
acttgaccttttctctcttatgtattttttccttcatttatttac
ctttgcagttccaaataaatttagaaaactactccctagacaatt
gataaggcagaatgttaactggccaaaagtttctgggccaccttt
tagccctcagggagaattgaagggagatatctgtgaggtctggtc
ataggcacatggaaaggaaacaggaagtaaccatagtaaccacta
cctaggctagaaaaggtgattctgtagggcctgagtggttccatc
cattttgaatgaaacttaaatgcaaaggcagagagcaagcctaca
cacacacacacacacacacacacacacacacacacacagatcccg
aggctcaaaaacaataaacctaaacagagtgactagcactttcag
tgactcaagacccagtcccctacaaattctaaacatcttagcttc
attttaaatggtcagcgagagcttaggaaaggagatgtcctaggc
acagtcttctttcttcctcccttcctctcatagaaaaagccctaa
ccttgtagaataaaacctactgtcaaagtaccttctcatttagga
catttttaggaaactgttcccatctatccactgagtgtcattctc
ctcttgaacctctgagttctatggatctccctgtgtcttctcact
gtacaaatagagtcacttcctcctgctgaacattgctcaaattgc
ctcaaaatgtggtcccagcatcacctgggaacttattagaaatgt
aaattcacataaactgttaaaatgg

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E26,” located on chromosome 13, at start/stop positions 116279500-116279827, shown in FIG. 1A, or a human ortholog thereof.

(SEQ ID NO: 69)
Gtgcactgtaaggaaaaagacaaattacccagagctattacagtc
attagtcccatcagcctgggaaggcctcccactgcattttccgtg
cagtgtatagagcggtcctggggcttatcacatcttgcagggggt
cgtcttctgccaggcatagtcacatagcttgacatttcacttaaa
aaaaaaaaagcctgggctctcatgctccagccaggcttctgtttc
ctggcttctcaaccagtggctaaaataaaggcagccagcccagac
tgaaggctgtggccattcatctaaatgcagaaatagaaatgcatc
ccttccagaggct

In an embodiment, a human neuronal cell enhancer sequence, which is the human ortholog of S9E26, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E6,” located on chromosome 5 at start/stop positions 51345517-51346168, shown in FIG. 1B.

(SEQ ID NO: 70)
Ctgcaatgcaggtatgacacttttgaaaagagacaaggaagaaat
gaggatggggaaagagcttcagactacatagcatttcggaaaaga
tcttggccaggttgatagagagcccctaagcaaaggggcctgtag
aggagtccatcattaggcaaaaatgacctgacactagttctcttg
cagtgttcagtcattggctgtgaggaactctggagagggcagcct
tggcaaaaacaccacagtcgattggatggtatggcagctgggggc
tatcagtcatctatgctctgcactaactctctccatggctgccac
agtctactcttttaatgcatgaatctacttctccacgcatcttta
agattcaggtacttcatacttctcatggacttcacctcctagggg
ccttccttcttaaaatgttctcttctattggctctgatagtgcta
gacatcattcattttcccccagccattccctggtttcctgggttg
actcatctttctctgaataagtttttaaataatgggggggatctc
ctcaggaacttcctataggtcttcctcttaaagatggagatgagt
gagacaaattaaagccctcatcaatgcaattgttctcaggacaat
aactgaaattcactatcttcct

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E35,” located on chromosome 2, at start/stop positions 169638390-169639139, shown in FIG. 1A, or a human ortholog thereof.

(SEQ ID NO: 71)
Tattgatctcatttttaatctgccatatcaggacataatatatta
accctaatcagaagattgcaagcggcaggagagagggaaggagag
ggcgccagggaactttcggcagtgaaatccctgaagcttgtttgg
gggggggggggggtcagatgcttgaggctacctttgggaagggta
aaaatgtggctcacaagtactttactcttcttttccttccacttt
gtgtttcttgctctcagccaactcggctctccttgctgccttcag
agaaactccacactccatagatacccctcctaagcagctttagat
ttgtcctactcgtgttttggctgttgtcacactctcctgcagagg
atcaggccctctcacatcctttctgacttctcactctttggggga
gtcttgggggaaactttcttggacctgggtttgtctgggttggtc
tatttctcttcccaaggcttaggaatacagctctttgctgagcag
gtcttaaagggagacttgagctggcactactatttaaggtccttg
tgatgaagcatctctggacatccctgcaactgagcataggaatat
caaccaggggggtgggggtgggggtggggagaaccaaacctgggc
tctgttcctgtttgtatgaggtctgtgagttaaacatccaaatta
tagcttcctctcaaagtatgtggaccatctacgtacttagcagta
gtttgggaagactcttactgtcaaggaagt

In an embodiment, a human neuronal cell enhancer sequence, which is the human ortholog of S9E35, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E35,” located on chromosome 20 at start/stop positions 52977727-52978486, shown in FIG. 1B.

(SEQ ID NO: 72)
Tattgatttcatttttaatctgccatatcaggacacaatacgtta
accctaatcagcagattacaagtgacagagagtttagtggaaact
ctgtaagggagcaggggctccgggaaacttcttgacaatgaaaat
cttctctgaggcttaagagtttggatgctgaagattacttttggg
gaggggagaaacattcctcagaaacagtctacatttttttcttcc
actttacatttctttatctcccagttgcccattctactgctacac
tttgggaatgcttaacacccaagaaatacctcccaagtattttta
ggaggtcggtccctgaagcagcttgacttttgtcatagtctccaa
cggacgtttatgctttcatttcatcccagctgatttctcattcat
ttgacggaatttctccaaaggataatacctgagttgatctgtttc
ttttcccagggccttgggaattgtgctgtccattgaatgggtttt
ggtcatgggggtgggggcacagactgagcctggcattttttaaag
ccattgaaatgatgcctcactgttcatcactgcagctgagcttat
caagtcaaccaagaagaaaacctaggccctggtcacgtttgtgct
atgcctgtaattagctgacacgaggtaaatgtccaaagtcgacct
tcccttccatcactgtatttatactatcaattttatttgggaatg
tcatggtctgggaagactcagccactatttcgaaggcaat

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “M940,” located on chromosome 9, at start/stop positions 91446555-91447304, shown in FIG. 1A, or a human ortholog thereof.

(SEQ ID NO: 73)
Atcttggttagttgtccctactttattaggaccaaacaaaattca
cacatgaggtaataaattatcgtgagctctgaaggctttactttt
ttttgtctaatgaattaatatactaaagaatacagtaaacgcatt
ttgtgctgctttgatcattcgcttacatgtcattaagtagtctaa
ggattttataatggaagggctctctttacaaattagaccaaagaa
cacgtttcacgcggaagatcagcatatatattgtattttacttca
gatgcacatgacacaaccgtaatattagaggcagatgattaaaag
agcattacctgagccctgagagtcactgaatgagctatgctaaat
tggttgttttctgttttgtgattaattgtgcttctaaaccatatt
ttagtgtgctttgtttttctgtccccagtcatcccatattattaa
agagaaattctgcatatctctcctggagtccaattaaggatttgc
atcatgactagatttttcactttaattactgtatataaagtaggt
tttgacttatttccatatttatcaaactatgcagaagaagcaaaa
ttcatcaaataaacagatatctgtggctataattgcagcctgcat
cgagcacgtaggaagaaaatatgagggtccaaatttcattatgta
gaacagaataaacatctgaataattaaactgtgtgtaaatcatag
tcttggcatagtaaaaagaggcgacatctt

In an embodiment, a human neuronal cell enhancer sequence, which is the human ortholog of S9E40, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E40,” located on chromosome 3 at start/stop positions 147450258-147451486, shown in FIG. 1B.

(SEQ ID NO: 74)
gtcttgaatagcttttattcattttccaaggtattttgagatgat
ggatgaaaataatttatatttaacagtagctaagaatatgaaaat
tttactcctcttagaattatttctaataagaaagtttagatatta
aaataattggaaactaaagatgaattatttgtttgcttttacaca
tgctttgtattagataagcttgcttctgaagttggggctaacaca
cttttgaggccttcttgtatggtttatggatctcagacagctgat
ttatgttattggcatcccaacaagaatggtaacttcataacactg
actgttattttcttgacttaattcaaaatcatatattatctttct
gaaaaatagatctcagaatacttcagctttcccaaaagtcacagg
acaccatagtcattaccgcttgatgaagtataaatataatgtctc
tttcttcaaaatttagtgtcagaataaatacacttacatgctttc
tcaaacctaagttaaataataattttctttaatccaaacatatta
aatatgtagcacatatacattaaaattaaccagatcaagcagtat
tttaattcagttgcttaattacatcttctggtgaataataggttt
taccagaaaaaaaattttagactacttgaaaacgatggtgtaata
catttttcttacagtatctttattaaagatacatcaatgtacaca
tatatctttgtatttttacaatagcattaactttggttctaccat
taacattttataagatcttttcctatgaaaacaaataccaattta
ctgtaatcatggttacttacagtatctaaatccattattgtaaaa
aggcaaacctgtgataggcatcacatggagaataaaaaattaaga
gactaattgtaatattaaataagcaatcttgttatatagatacat
gtaatattgcatgttgcatgtattgttactcagagtagaaagaac
atgtgagaagtttcctaacttggataagaacatcacaaatttcta
ttctcacacaaaatgccaatttactagttccctgaagattttact
tagataactgttatgcaacaccaaataaacacaagctgaatttct
gtttattaacacaatttaagtctaattccaagcttttacaggctt
ggcccatggtacataagcgtactgagttttatttctggttttaca
agaatggaaatttt

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E29,” located on chromosome 14, at start/stop positions 32443322-32444071, shown in FIG. 1A, or a human ortholog thereof.

(SEQ ID NO: 75)
Acgtccacttagatgactcaaatcaaatgtgcttggacaggatctc
tccattttcagcccactcctcatcccagtaaatgataccggcatc
cactcagtggatcaaacaagaaacccagggatctccatccttccc
cttccttcctgatgatgcttccgccctgagagagactcgcccgca
tcctttccttttctctctcagaccggctttggtcatgctgctgtc
agtagttcacttggaaacagaactcatttctgaaatggtttctct
gtttcccttccttgacggctctggggaagagggtgccactctggg
aggaattggtttcctgctgtgagagacactgccagtggtactaat
tggattaattgatttgcccaaggggaggtgtgtgtgatgcctttc
tgctgaacttcctttaaaaacagcacttccattcagaaagccaga
ccaagtgctattaacgaattaattccgattagtgctgcccaagca
ctgagaccttgaggcaaccccgttcagtcatcttgggtctggcct
gctaataaagtgaaaactatctgggcagcagaggtgattattttg
atgcctgcctccattcctggagggaaaaagagcagggtgtgcgtt
caccagtctactgagggaattaaccaccttcaaattctaagttat
taattccccaacatggcagtcatatccgtgaaaatggatttgggg
catatgtctgtgtcctctgtagccttctt

In an embodiment, a human neuronal cell enhancer sequence, which is the human ortholog of S9E29, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E9,” located on chromosome 10 at start/stop positions 49696223-49697620, shown in FIG. 1B.

(SEQ ID NO: 76)
acgtaacattttacatagagcctcttcccgcatgatttgttgact
gtctgagcattttatcttgaatcaggaaatctctcccttgagctg
tgatttctactttcatggccaaaactttttcaatgctgagaactg
aaattgctagcttcccctgtctgcatttggagtgggaacttcatc
tccaaacacagtggaatcctgtctcctctagccctgcaaccccgg
cattaaactggtaacaagtgcttccctcggcagccccatgccctc
ctccagcatccgcctcaaccccgcaggttgtatttgtgccttcag
agtagcactacaagtgaagaagacctgccctccttggcttcatga
ccagagctgaagggcaggtaaggcgagacagtacacggattctaa
gaaataccggagaagcttctgggagatgcctgtgcccagagccct
atgtttcagtttggatcagaggcaaatgatgtctgaatcagcatt
gctgggaaaaatcaccgaggtttcctctgagcagtgggattgggg
ggtgtggggggcaggtgaggtagagggaggaagtttgcagggcta
ttctgccttccgtattgtaaatgtttttagtggtggaattgattt
tcactttgtatgtaagtgtgtgttaggggtacttttcagataaaa
acttaaaatcagggccgggcactatggcttttgcctgtaatccca
gcactttgggaatccaaggtgggtggatttgtttgaacacaggag
ttcgagagcagcctggccaacatggtgaagcctcatctctacaaa
aaatacaaaaattagccaggcctggtggcatgcacctgtagtccc
agctacttgaggggctgaggcaggaggatcacctgagcccaggag
ctagaggctgcagcgaacctagatcacgccactgcactccagcct
gggtgacaaagtgagaccctgtctcagaaaataaaataaagattt
tgacatagaagccacctgtctatggccttatcctcctctgtcact
gattaagaggaactttctctatggaaatctctgaagaatgtgctg
tcatcctcctcctttgcctccctctctcgagcacgcacctccctg
cccagaagcaagagacacagcttgctccccatcaaaggcaaaagt
gccctgtgcttcaacgtgctagacattcgggcgccttcatgcttt
taaaataccactgattaaatcacattttaagttttataagatgtc
aagtcaatgtcgttatgattaacatgtaaagaaagtgtattttaa
aataaagttagaagcatagctgtgtcttaattttttttgtttttg
agacagtcttgctctgtcaccgaggctggagtgcagtggcatgat
cat

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E30,” located on chromosome 14, at start/stop positions 93871264-93871706, shown in FIG. 1A, or a human ortholog thereof.

(SEQ ID NO: 77)
gatagttgcctaagcagccacagaacagttggaaagatcctttta
gagttaggcaccagcttggtggttatcagagaagtctaattgcaa
ttaaaatagaacgttttctggcactgaaggcttttgttttttcaa
atacatttgcacaagaatgcacaggtggggacctgtagtttttct
ttttcataaagcaaagatattcactcaaatgatactataattgtg
gaacgacttgcttacataaggacatgcatccttatgtaagttagt
gtcttagaaatgaatgagttaaaattaactctatgccttgcctaa
atgtaaaacccaactgtgacacctccttctgtgatgacagaaaaa
caaagtttcagggtatgaatgttttcaacattagaataaaacata
tataatgaaggtccccttcgttttaacatttatggtat

In an embodiment, a human neuronal cell enhancer sequence, which is the human ortholog of S9E30, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E30,” located on chromosome 13 at start/stop positions 67211275-67211718, shown in FIG. 1B.

(SEQ ID NO: 78)
gatagttgcctaagcctaagcatccacagaacagttggaaagatc
cttttagatttaggcaccagcttgatggttatcacagaagtctaa
ttgcaattaaaatagtatgttttctggcaataaagatttatctcc
taaaatatgtttgtacaagaatgtgcaggtgaggcattatatgca
ttattttttaaaatacaaatttttggcttaaataaaaatttgaaa
tttggggatgaccttcctatataaacatttatccatatgtaagtt
agtgttttagaaataattgagttaaaaaaacatagtctgtcttgg
ctgtaatagccgaattatgacattttcttcgatgatgaaagaaaa
ctaaattttcaggatatgtatgtttctcaatttaggaaaacatat
atatataaaataaaggtctcttgttttcaacgtttatat

In an embodiment, a neuronal cell enhancer sequence comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following mouse polynucleotide (DNA) sequence called “S9E37,” located on chromosome 9, at start/stop positions 91374713-91375267, shown in FIG. 1A, or a human ortholog thereof.

(SEQ ID NO: 79)
Gtctctggggaaatggaccttaaaagaatgtctggtcccattgtt
atgtttagccaaccgaggtatggaccatatggctccaagaacctt
gcgagtatcaggcagcctctgtgacctgcaggtcaactgacccct
agctactcaagaaatgtctacccaggatgggtccagatgttttta
gtgcttcaagagaagtctgcatctgggaaggagctccatggggcc
aggctactgtcagtgattttggctgaatcagatgtgtttgccctt
ttgctgctggcacggaaagtcatacacatgttcctaccttttgaa
gggcaaagatagtcagactgtatactaacaagatgtgtgtataaa
accttgctctctccccctcttctcctctccctctttctctctttt
aacctttgggtacacaggacagtgcttacaattcaggaggtctgt
aaacttgaattttttttttaaatccttctctctacaacctgctaa
tggaaagttagcattttgtctcctgtgaaaatgagaagcaaacat
tttcggcatgctcac

In an embodiment, a human neuronal cell enhancer sequence, which is the human ortholog of S9E37, comprises a nucleotide sequence which contains one or more regions of 50-500 bp or longer, 50-250 bp or longer, 100-200 bp or longer, or 100 bp or longer, having at least 70% or greater, at least 75% or greater, at least 80% or greater, at least 85% or greater, at least 90% or greater, or at least 95% or greater sequence identity to the following human polynucleotide (DNA) sequence called “huS9E37,” located on chromosome 3 at start/stop positions 147400443-147401373, shown in FIG. 1B.

(SEQ ID NO: 80)
ttctcttgaaagctgaaaacatctggacccattttaggtagcttc
ttcttgcttgagtagccaggggtcagtagacctgcaggtcactga
ggttgcctgatctttgcaagttccttagagctatatggtctatat
gttggttggctaaacccagcaatgggaccagacattctcctcgaa
ggacatttccccagagacagcaataagggattacataaggatctt
caaggtatatctttgaaataagccccaaagcagtatccagttcaa
ggtgatggggcatcaacaactgagaggtatattttagtctttaga
gagatcattttgagtcttccagagcctgggaagcagggaacccag
gctacagagattatcaattgggctggctaaggaatggtgtctcct
cagtgctcttcagattttttttccagaaacatctcactatacctg
cccccaacaccaaatgcttgactgaaacattctattcctacccat
gcctggagatataacattgttaagtctacttttctgcttcatttg
tagagaaaaagagtatctcacaacccacagatctttagcacatgt
caaaatgaaacactggaagcttgttttcttggggacctatttgga
gcatattccaccactagaccccaaatatctcttgatcttcagagc
ctctgcaaaccagtgcccagaatcatgccctaagcacttttgctc
acctacccagacgaatgcagtggcataaacaagtgatttcctagt
tcttttctagaaatagaacatttggtggccctaccatctgccaga
gctccttaaatcactggtttgaatgtctgatctcactgccttgga
aagaaaaaaaaatggaggatatttcagatcaacagctgggctatt
tttccattttaatattttaagcaatgctaaa

In embodiments of the foregoing, a mouse or human neuronal cell enhancer sequence may comprise a nucleotide sequence which contains one or more regions of 50-500 bp or longer, 50-250 bp or longer, 100-200 bp or longer, 50-700 bp or longer, 50-750 bp or longer, 100-750 bp or longer, or 100 bp or longer, having at least 70% or greater, at least 75% or greater, at least 80% or greater, at least 85% or greater, at least 90% or greater, or at least 95% or greater sequence identity to the polynucleotide (DNA) sequence of a mouse or human enhancer described above.

### GABAergic Cortical Interneurons

GABAergic interneurons, which release the neurotransmitter gamma-aminobutyric acid (GABA) are inhibitory neurons of the central nervous system and are essential for regulating and maintaining neural circuitry and activity. (Kelsom, C. and Lu, W., 2013, Cell Biosci., 3:19). GABAergic interneurons of the mammalian cerebral cortex comprise several different cortical interneuron subtypes that may be categorized and classified by their expressed protein markers.

Interneurons play a key role in the wiring and neural circuitry of the developing nervous system of both invertebrate and vertebrate organisms. In general, an interneuron is a specialized type of neuron (nerve cell) whose primary role is to form a connection between other types of neurons. Interneurons, which are neither motor neurons nor sensory neurons, differ from projection neurons in that projection neurons send their signals to more distant locations, such as the brain or the spinal cord. Critically, interneurons function to modulate neural circuitry and circuit activity. A large majority of interneurons of the central nervous system are of the inhibitory type. In contrast to excitatory neurons, inhibitory cortical interneurons typically release the neurotransmitters gamma-aminobutyric acid (GABA) and glycine. Cortical interneurons are localized in the cerebral cortex, which is defined as a sheet of outer neural tissue that functions to cover the cerebrum and cerebellum structures in the brain. (Id.) GABAergic interneurons include numerous interneuron subtypes that may be categorized by the surface markers they express. Four major cortical interneuron subtypes are parvalbumin (PV)-expressing interneurons, somatostatin (SST)-expressing interneurons (which constitute a heterogeneous population), and ionotropic serotonin receptor 5HT3a (5HT3aR)-expressing interneurons. These three subtypes together account for approximately 100% of the neocortical GABAergic interneuron population in mice. Although these interneurons home to their respective layers of the cerebral cortex, they are generated in various subpallial locations and they subsequently migrate to the cerebral cortex.

Cortical circuit function is maintained by the balance between excitatory inputs and inhibitory inputs. A disruption of the balance of neural circuits is likely to contribute to the emergence of neurological, neurodevelopmental, neurodegenerative, neuropsychiatric, neurogenetic, or neuromuscular diseases, disorders, or pathologies, such as, without limitation, seizures, epilepsy, autism spectrum disorders, and intellectual disabilities.

### The Role of GABAergic Cortical Interneurons

GABAergic neurons play an inhibitory role and synaptically release the neurotransmitter GABA to regulate the firing rate of target neurons. Neurotransmitter release typically acts through postsynaptic GABA_{A} ionotropic receptors in order to trigger a neuronal signaling pathway. Interneuron role/function is typically categorized into three components: (1) afferent input, (2) intrinsic properties of the interneuron, and (3) targets of the interneuron. In general, interneurons receive input from various sources, including pyramidal cells, as well as cells from other cortical and subcortical regions. (Kelsom, C. and Lu, W., 2013, Cell Biosci., 3:19). With regard to output, cortical interneurons engage in feed-forward and feedback inhibition. Regardless of the mode of output, the cortical interneuron network is further complicated by the fact that a single cortical interneuron is capable of making multiple connections with its excitatory neuronal target(s).

### Cortical Interneuron Subtypes

It is estimated that there are over 20 different subtypes of GABAergic interneurons in the cerebral cortex. The subtypes are also distinguished from each other based upon the calcium-binding proteins they express, which serve as markers. Based on studies performed in both mouse and rat brain tissue, the calcium-binding protein, parvalbumin (PV), and the neuropeptide somatostatin (SST), are key markers found to define the most predominant interneuron subtypes within the cerebral cortex. Of particular note, the PV-expressing interneuron population is independent from the SST-expressing population, in that expression of these markers does not overlap. In addition to PV- and SST-positive GABAergic interneurons, which together comprise approximately 70% of the total GABAergic cortical interneuron population, another subgroup of interneurons that express 5HT3aR were found to comprise approximately 30% of all interneurons. These three interneuron subpopulations account for nearly or equal to 100% of all GABAergic cortical interneurons, yet each of these populations, especially the 5HT3aR-expressing population, is heterogeneous and expresses other proteins or neuropeptides that contribute to their characterization. (Kelsom, C. and Lu, W., 2013, Cell Biosci., 3:19).

### Parvalbumin (PV)-Expressing Interneurons

PV-expressing interneuron represent approximately 40% of the GABAergic cortical interneuron population. This population of interneurons possesses a fast-spiking pattern, and fire sustained high-frequency trains of brief action potentials. These interneurons also possess the lowest input resistance and the fastest membrane time constant of all interneurons.

Two types of PV-interneurons comprise the PV interneuron group: basket cells and chandelier cells. Basket cells are interneurons that make synapses at the soma and proximal dendrite of target neurons, and usually have multipolar morphology. Several studies have shown that fast-spiking basket neurons are the dominant inhibitory system in the neocortex, where they mediate the fast inhibition of target neurons, among many other functions. Such fast-spiking basket neurons likely play a large role in regulating the delicate balance between excitatory and inhibitory inputs in the cerebral cortex. Unlike basket neurons, the chandelier cell subgroup of PV-expressing interneurons targets the axon initial segment of pyramidal neurons. Both basket cells and chandelier cells are fast-spiking, but they differ in electrophysiological properties. In contrast to other interneurons, chandelier cells may be excitatory rather than inhibitory due to their depolarizing effects on membrane potential. (Kelsom, C. and Lu, W., 2013, Cell Biosci., 3:19).

Another group of PV-expressing cells that is independent from chandelier and basket neurons in the neocortex, e.g., mouse neurocortex, are called multipolar bursting cells, which differ from chandelier and basket cells in both electrophysiology and connectivity. Multipolar bursting neurons possess synapses with pyramidal cells (or other multipolar bursting cells) that demonstrate a paired-pulse facilitation; in contrast, chandelier and basket cells are usually strongly depressing. (Kelsom, C. and Lu, W., 2013, Cell Biosci., 3:19).

### Somatostatin (SST)-Expressing Interneurons

SST-expressing interneurons constitute the second-largest interneuron group in the mouse neocortex and represent approximately 30% of the total cortical interneuron population. SST GABAergic interneurons represent a heterogeneous population of cortical interneurons. SST-positive interneurons are called Martinotti cells and possess ascending axons that arborize layer I of the cerebral cortex and establish synapses onto the dendritic tufts of pyramidal neurons. Martinotti cells are also found throughout cortical layers II-VI, but are most abundant in layer V. In contrast to PV-positive interneurons, excitatory inputs onto Martinotti cells are strongly facilitating. Additional subpopulations of SST-expressing cortical interneurons show differences in firing properties, expression of molecular markers and connectivity of different neurons within this population. (Kelsom, C. and Lu, W., 2013, Cell Biosci., 3:19).

### 5HT3aR-Expressing Interneurons

The third population of GABAergic cortical interneurons is designated as the 5HT3aR interneuron group, which accounts for approximately 30% of the GABAergic cortical interneuron population. Based on mouse studies, this population of GABAergic interneurons in the cortex express the 5HTa3 receptor, but do not express either PV or SST.

5HT3aR interneurons represent a heterogeneous population. Within the 5HT3aR interneuron group are several subsets of interneurons that also express other protein or neuropeptide markers, including vasoactive intestinal peptide (VIP). VIP-expressing interneurons are localized in cortical layers II and III. The VIP-expressing interneurons do not express PV or SST, but do express the 5HTa3 receptor, accounting for approximately 40% of the 5HT3aR population. VIP interneurons generally make synapses onto dendrites; some have been observed to target other interneurons. Compared with other cortical interneurons, VIP interneurons possess a very high input resistance and are among the most excitable of interneurons.

60% of cortical interneurons in the 5HT3aR-expressing population do not express VIP. Of this VIP-negative 5HT3aR group, nearly 80% express the interneuron marker reelin. In this latter category of cortical interneurons, the neurogliaform cell population, called spiderweb cells, express neuropeptide Y (NPY), and exhibit multiple dendrites radiating from a round soma. Neurogliaform interneurons can form synaptic connections with each other as well as with other interneuron types, in contrast to other types of interneurons that can only make synapses onto homologous neurons. Thus, neurogliaform cells play an important role in regulating neural circuitry and function by activating slow GABA_{A} and GABA_{B} receptors in order to provoke long-lasting inhibitory postsynaptic potentials onto pyramidal neurons and other interneurons.

### Non VIP/Caudal Ganglionic Eminence (CGE) Neurons

The caudal ganglionic eminence (CGE), which is a fusion of the rostral medial and lateral ganglionic eminence, begins at the mid to caudal thalamus and is the second largest source of cortical interneurons, contributing approximately 30% of all cortical interneurons (G. Miyoshi et al., 2010, J. Neuroscience, 30.1582-1594). CGE-derived cells include GABAergic interneurons, spiny interneurons, mossy cells, pyramidal and granule neurons, and even oligodendrocyte and astrocyte glial cells. Recently, it has been shown that CGE-derived interneurons specifically express the serotonin receptor 5HT3a, while Nkx6.2 and CoupTF1/2 are widely, but not selectively, expressed within the CGE (R. Batista-Brito et al., 2013, In: Patterning and Cell Type Specification in the Developing CNSa and PNS, Eds. J. L. R. Rubenstein and P. Rakic, Elsevier Inc., Academic Press; L. Lim et al., 2018, Neuron, Review, doi.org/10,1016/j.neuron.2018.10.009).

### Dopamine-Receptor 1 (D1)-Expressing Medium-Spiny Neurons (Drd1) and Dopamine-Receptor 2 (D2)-Expressing Medium-Spiny Neurons (Drd2)

Medium spiny neurons (MSNs) use γ-aminobutyric acid (GABA) as a neurotransmitter and represent approximately 90-95% of the striatal neuronal population in rodents. MSNs can be divided into two subpopulations based on their neurochemical content and axonal projection sites. Roughly half of the MSNs express dopamine (DA) receptor of the D1 type (Drd1) and contain the neuropeptides substance P (SP) and dynorphin (DYN). They innervate mainly the substantia nigra pars reticulata and the entopeduncular nucleus (rodent homologue of primate internal pallidum) and form the so-called “direct pathway”. The other half of the MSNs expresses DA receptor of the D2 type (Drd2) and contains the neuropeptide enkephalin (ENK). Their axon arborizes principally in the pallidum (rodent homologue of primate external pallidum) and forms the first segment of the so-called “indirect pathway. It has been shown that both D1 and D2 MSNs located in the nucleus accumbens (Acb) can either inhibit or disinhibit thalamic activity depending on their projection pattern and not on their genetic characteristics. Dopamine modulates cortical and thalamic glutamatergic signals impinging upon principal MSNs of the striatum. Drd1 signaling enhances dendritic excitability and glutamatergic signaling in striatonigral MSNs, while Drd2 signaling exerts the opposite effect in striatopallidal MSNs. The functional antagonism between these two major striatal dopamine receptors extends to the regulation of synaptic plasticity; however, a balanced activity between D1 and D2 MSNs is required to ensure correct motor and cognitive behaviors.

### Cholinergic Interneurons of the Striatum (Ch-IN)

The striatum is a brain region containing high levels of acetylcholine (ACh), muscarinic receptors, and other ACh-related markers. Cholinergic interneurons of the striatum (Ch-IN) are significant regulators of striatal network activity and output. While they comprise a small fraction of cells in the striatum, they are critical modulators of neuronal excitability, synaptic transmission, and synaptic plasticity within the striatal circuitry, primarily through activation of muscarinic receptors. In the striatum, a decrease in cholinergic markers is a phenotypic consequence of Parkinson's and Huntington's diseases Ch-INs are the main source of acetylcholine in the striatum and are believed to play an important role in basal ganglia physiology and pathophysiology. The increase of Ch-IN firing by the neurotransmitter corticotropin-releasing factor (CRF), e.g., in response to stress, results in the activation of muscarinic acetylcholine receptors type 5, which mediate potentiation of dopamine transmission in the striatum. Cholinergic interneurons are tonically active in primates and rodents, and display various rhythmic and irregular firing patterns that are generated autonomously Cholinergic interneurons receive extrinsic input from various brain regions, notably glutamatergic input from cortex and thalamus, dopaminergic input from the substantia nigra pars compacta, and GABAergic input primarily of striatal origin. In addition, Ch-IN neurons are the first types of neurons to be altered in individuals suffering from Parkinson's disease. Thus, the ability to target certain types of neurons by using the enhancer elements described herein provides an advantageous therapeutic benefit for treating neurological conditions such as Parkinson's disease.

### Cholinergic Interneurons of the Basal Ganglia (Ch-PN)

Cholinergic neurons are highly integral for fine tuning brain function and maintaining the balance between excitation and inhibition within neural circuits. Cholinergic neurons are also found in the basal forebrain, which is classically segregated into four main regions: the Medial Septal Nucleus (MSN), the vertical and horizontal limbs of the Diagonal Band of Broca (DB), and the Nucleus Basalis (NB) of Meynert. Within the brainstem, cholinergic neurons are found in the pedunculopontine nucleus (PPN) and the laterodorsal tegmentum. In addition to these groups, smaller cholinergic populations are located in the medial habenula, parabigeminal nucleus, cerebral cortex, hypothalamus, and olfactory bulb (N. Ahmed et al., 2019, Front. Mol. Neurosci., Vol. 12, Article 201).

Changes in the activity of striatal cholinergic interneurons (Ch-INs) have been shown to play a critical role in motor control, as well as behavioral flexibility, memory, and social behavior. Alterations to the cholinergic system can lead to severe dysfunction of neuronal circuits. For example, cholinergic neuron loss from the forebrain causes cognitive deficits associated with Parkinson's disease (PD) and Alzheimer's disease (AD). Cholinergic neuron populations are also linked to neuropsychiatric and neurodevelopmental pathologies. By way of example, the activity of Ch-INs, through the expression and function of the hyperpolarization-activated cyclic nucleotide-gated channel 2 (HCN2), is decreased in the Nucleus Accumbens (NAc) in stress and depression) A link between the cholinergic system and autism spectrum disorders has also been established, whereby autism is associated with decreased cholinergic tone and reduced neurite arbonzation. Genetic alterations of the choline transporter cause attention deficit hyperactivity disorder (ADHD) and result in the promotion of ACh synthesis. In addition, elevating ACh levels can relieve cognitive and social symptoms in a mouse model of autism. Aberrant cholinergic signaling has also been reported in schizophrenia. (N. Ahmed et al., 2019, Front. Mol. Neurosci., Vol. 12, Article 201).

### Cortical ChAT Cholinergic Neurons

The mouse cerebral cortex contains neurons that express choline acetyltransferase (ChAT or CHAT) and are a potential local source of acetylcholine. ChAT is a transferase enzyme that is responsible for the synthesis of the neurotransmitter acetylcholine. A nerve terminal proteins, ChAT is produced in the body of a neuron and is transported to the nerve terminal, where its concentration is highest. The expression of ChAT by neurons classifies the neurons as cholinergic neurons, which provide the primary source of acetylcholine to the cerebral cortex. ChAT neurons promote cortical activation during wakefulness and rapid eye movement (REM) sleep. While the neurotransmitters released by cortical ChAT⁺ neurons and their synaptic connectivity are unknown, it has been reported that nearly all cortical ChAT⁺ neurons in mice are specialized VIP⁺ interneurons that release GABA strongly onto other inhibitory interneurons and acetylcholine sparsely onto layer 1 interneurons and other VIP⁺/ChAT⁺ interneurons. (A. Granger et al., 2020, eLife, 9, e57749; pp 1-29). This differential transmission of ACh and GABA based on the postsynaptic target neuron is reflected in VIP⁺/ChAT⁺ interneuron presynaptic terminals, as quantitative molecular analysis shows that only a subset of these are specialized to release acetylcholine. In addition, a separate, sparse population of non-VIP ChAT⁺ neurons has been identified in the medial prefrontal cortex with a distinct developmental origin that release acetylcholine in layer 1 (Ibid). Thus, both cortex-region heterogeneity in cortical ChAT⁺ interneurons and target-specific co-release of acetylcholine and GABA have been determined.

### Adeno-Associated Virus (AAV)

AAV is a small (25 nm), nonenveloped virus that contains a linear single-stranded DNA genome packaged into the viral capsid. It belongs to the family Parvoviridae and is of the genus Dependovirus, because productive infection by AAV occurs in the presence of either an adenovirus or herpesvirus helper virus. In the absence of helper virus, AAV (serotype 2) can establish latency after transduction into a cell by specific but rare integration into chromosome 19q13.4. Accordingly, AAV is the only mammalian DNA virus known to be capable of site-specific integration. (Daya, S. and Berns, K. I., 2008, Clin. Microbiol. Rev., 21(4):583-593).

There are two stages to the AAV life cycle after successful infection: a lytic stage and a lysogenic stage. In the presence of adenovirus or herpesvirus helper virus, the lytic stage persists. During this period, AAV undergoes productive infection characterized by genome replication, viral gene expression, and virion production. The adenoviral genes that provide helper functions for AAV gene expression include E1a, E1b, E2a, E4, and VA RNA. While adenovirus and herpesvirus provide different sets of genes for helper function, they both regulate cellular gene expression and provide a permissive intracellular milieu for a productive AAV infection. Herpesvirus aids in AAV gene expression by providing viral DNA polymerase and helicase as well as the early functions necessary for HSV transcription.

In the absence of adenovirus or herpesvirus, AAV replication is limited; viral gene expression is repressed; and the AAV genome can establish latency by integrating into a 4-kb region on chromosome 19 (q13.4), called AAVS1. The AAVS1 locus is near several muscle-specific genes, TNNT1 and TNNI3. The AAVS1 region itself is an upstream part of the gene MBS85 whose product has been shown to be involved in actin organization. Tissue culture experiments suggest that the AAVS1 locus is a safe integration site.

Recombinant AAV (rAAV) as a Vector for Gene Delivery and Therapeutic Treatment

AAVs are well suited for use as vectors and vehicles for gene transfer to the nervous system, as they enable gene expression and knockdown, gene editing, circuit modulation, in vivo imaging, disease model development, and the assessment of therapeutic candidates for the treatment of neurological, neurodevelopmental, neurodegenerative, neuropsychiatric, neurogenetic, or neuromuscular diseases, disorders, or pathologies. AAVs provide safe, long-term expression in the nervous system. Most of the foregoing applications rely on local AAV injections into the adult brain to bypass the blood-brain barrier (BBB) and to restrict transgene expression temporally and spatially.

AAV vectors have been highly successful in fulfilling all of the features desired for a delivery vehicle, such as the ability to attach to and enter the target cell, successful transfer to the nucleus, the ability to be expressed in the nucleus for a sustained period of time, and a general lack of pathogenicity and toxicity. Recombinant AAV (rAAV) is advantageous as a delivery vector, particularly for delivery to interneurons in brain tissue, as it is focally injectable; it exhibits stable expression over time; and it is both non-pathogenic and non-integrative into the genome of the cell into which it is transduced. Twelve human serotypes of AAV (AAV serotype 1 (AAV-1) to AAV-12) and more than 100 serotypes from nonhuman primates have been reported to date. (Daya, S. and Berns, K. I., 2008, Clin. Microbiol. Rev., 21(4):583-593). In addition, rAAV has been approved by the FDA for use as a vector in at least 38 protocols for several different human clinical trials. AAV's lack of pathogenicity, persistence and its many available serotypes have increased the potential of the virus as a delivery vehicle for a gene therapy application in accordance with the described compositions and methods.

Recombinant AAV (rAAV) vectors have been constructed that do not encode the replication (Rep) proteins and that lack the cis-active, 38 base pair integration efficiency element (IEE), which is required for frequent site-specific integration. The inverted terminal repeats (ITRs) are retained because they are the cis signals required for packaging. Thus, current recombinant AAV (rAAV) vectors persist primarily as extrachromosomal elements.

Recombinant AAV (rAAV) vectors for gene therapy have been based mostly on the AAV-2 serotype. AAV-2-based rAAV vectors can transduce muscle, liver, brain, retina, and lungs, requiring several weeks for optimal expression. The efficiency of rAAV transduction is dependent on the efficiency at each step of AAV infection, i.e., virus binding, entry, trafficking, nuclear entry, uncoating, and second-strand synthesis.

Several novel AAV vector technologies have been developed to either increase the genome capacity for AAV or enhance gene expression. Trans-splicing AAV vectors have been used to increase the capacity of the vector for harboring heterologous polynucleotides by taking advantage of AAV's ability to form head-to-tail concatemers via recombination in the ITRs. In this approach, the transgene cassette is split between two rAAV vectors containing adequately placed splice donor and acceptor sites. Transcription from recombined AAV molecules, followed by the correct splicing of the mRNA transcript, results in a functional gene product. While somewhat less efficient than rAAV vectors, trans-splicing AAV vectors permit delivery of therapeutic genes up to 9 kb in size and have been successfully used for gene expression in the retina, lung and muscle.

Polynucleotides encoding rAAVs as described herein comprise an enhancer polynucleotide sequence of any one of SEQ ID NOS: 1-80. Because of its nature as an enhancer, the orientation of the enhancer polynucleotide sequence, i.e., 5′-3′ or 3′-5′, is not material to its function. Accordingly, the enhancer sequences as described herein may be used in a reverse orientation and may be used as reverse-complementary sequences. By way of example, a “PV-specific enhancer” refers to the enhancer sequences described herein that target and restrict, regulate, or modify the expression of a gene (transgene) in PV-expressing cortical interneurons (PV-cINs) as described herein.

Moreover, the enhancer sequence need not be specifically spaced relative to other sequences, such as a transgene, e.g., therapeutic gene, reporter gene, or effector gene coding sequence in a vector. In addition, the vector (e.g., rAAV) polynucleotides may include additional elements, for example, a sequence encoding a reporter or a detectable marker, such as a fluorescent protein, or an element such as a Woodchuck Hepatitis Virus Post-transcriptional Regulatory Element (WPRE), which may increase RNA stability and protein yield. A vector (e.g., an rAAV) polynucleotide may also comprise a promoter to drive transcription of one or more polynucleotides (genes) which are inserted between inverted terminal repeats (ITRs). A polyadenylation signal, such as bovine growth hormone polyadenylation signal and/or SV40 polyomavirus simian virus 40 polyadenylation signal, may be included as elements in the vector (e.g., rAAV) polynucleotide. The vector (e.g., rAAV) polynucleotide can comprise a minimal promoter, e.g., a human beta-globin minimal promoter (phog) and a chimeric intron sequence (Hermeming et al., 2004, J Virol Methods, 122(1):73-77). Without wishing to be bound by theory, ITRs may aid in concatamer formation in the nucleus after the single-stranded, AAV vector DNA (e.g., rAAV) is converted into double stranded (ds) DNA by host cell DNA polymerase complexes. Thus, the administration of the described rAAVs may form episomal concatemers in the nucleus of interneuron cells into which they are transduced. In non-dividing cells, such as adult interneurons, concatemers may remain intact in these cells for the lifetime of the interneurons. Advantageously, integration of vector (e.g., rAAV) polynucleotides into host chromosomes is likely to be negligible or absent and will not alter or affect the expression or regulation of any other human gene.

Recombinant AAV vectors can be made using standard and practiced techniques in the art and employing commercially available reagents. It will be appreciated by the skilled practitioner that rAAV vectors that been used in several clinical trials that have yielded promising results. By way of example, rAAV based therapy received marketing approval by the European Union in 2012, as reported by Kotterman, M. A. et al., 2014, Nat. Rev. Genet., 15:445-451. In some embodiments, plasmid vectors may encode all or some of the well-known replication (rep), capsid (cap) and adeno-helper components. The rep component comprises four overlapping genes encoding Rep proteins required for the AAV life cycle (e.g., Rep78, Rep68, Rep52 and Rep40). The cap component comprises overlapping nucleotide sequences of capsid proteins VP1, VP2 and VP3, which interact together to form a capsid of an icosahedral symmetry. A second plasmid that encodes helper components and provides helper function for the AAV vector may also be co-transfected into cells. The helper components comprise the adenoviral genes E2A, E4orf6, and VA RNAs for viral replication.

In an embodiment, a method of making rAAVs for the products, compositions, and uses described herein involves culturing cells that comprise an rAAV polynucleotide expression vector as described; culturing the cells to allow for expression of the polynucleotides to produce the rAAVs within the cell, and separating or isolating the rAAVs from cells in the cell culture and/or from the cell culture medium. Such methods are known and practiced by those having skill in the art. The rAAVs can be purified from the cells and cell culture medium to any desired degree of purity using conventional techniques.

In an embodiment, the rAAV vector contains an enhancer polynucleotide sequence as described herein and a chemogenetic DREADD (‘Designer receptor exclusively activated by designer drug’)-encoding sequence, e.g., a Gq-DREADD receptor (Hu, J. et al., 2016, J Biol Chem, 291:7809-7820). The amino acid sequence of the Gq-DREADD receptor has been reported by Armbruster et al. (2007, Proc Natl Acad Sci USA, 104:5163-5168). The amino acid sequence of the Gq-DREADD receptor is a derivative of the amino-acid sequence of the human muscarinic acetylcholine receptor, M3, in which the tyrosine in position 149 is replaced by a cysteine, and the arginine in position 239 is replaced by a glycine. The unmodified human sequence is provided under NCBI accession no. NP 000731.1. In an embodiment, the polynucleotide sequence that encodes the Gq-DREADD receptor in the rAAV vector can be modified, for example, by including optimized codons for expression of the Gq-DREADD receptor in human interneurons.

In an embodiment, the rAAV vector contains an enhancer polynucleotide sequence as described herein and a chemogenetic PSAM-encoding sequence.

Recombinant AAV vectors, which have a genome of small size (about 5 kb), can be engineered to package and contain larger genomes (transgenes), e.g., those that are greater than 4.7 kb. By way of example, two approaches developed to package larger amounts of genetic material (genes, polynucleotides, nucleic acid) include split AAV vectors and fragment AAV (fAAV) genome reassembly (Hirsch, M. L. et al., 2010, Mol Ther 18(1):6-8; Hirsch, M. L. et al., 2016, Methods Mol Biol. 1382:21-39). Split rAAV vector applications were developed to take advantage of the fact that rAAV genomes naturally concatamerize in the cell post-transduction and are substrates for enhanced homologous recombination (HR) (Hirsch, M. L. et al., 2016, Methods Mol Biol. 1382:21-39). This approach comprises “splitting” a large transgene into two separate vectors and upon co-transduction, intracellular large gene reconstruction via vector genome concatemerization occurs via HR or nonhomologous end joining (NHEJ). In general, three strategies exist within the split rAAV approaches: overlapping, trans-splicing, and hybrid trans-splicing.

Fragment AAV (fAAV) as an approach for AAV-mediated large gene delivery was developed based on reports that attempted encapsidation of transgenic cassettes exceeding the packaging capacity of the AAV capsid resulted in the packaging of heterogeneous single-strand genome fragments (<5 kb) of both polarities. After transduction by multiple fAAV particles, the genome fragments can undergo opposite strand annealing, followed by host-mediated DNA synthesis to reconstruct the intended oversized genome within the cell. (Hirsch, M. L. et al., 2016, Methods Mol Biol. 1382:21-39).

An advantage and benefit of the vectors, compositions and methods described herein is the identification and use of sufficiently small enhancer elements (cis-acting elements) that are capable of restricting or regulating gene expression in a defined population of cells, e.g., interneuron and neuron cell populations as described herein. In embodiments, the enhancer element is at least one of the S9E1-S9E40 enhancer sequences as described herein, which restrict gene expression to interneuronal and neuronal cell populations, such as, for example, inhibitory GABA-ergic neurons, such as parvalbumin (PV)-expressing interneurons, Somatostatin (SST)-expressing interneurons; Vaso-active Intestinal Peptide (VIP)-expressing interneurons; and non-VIP/CGE-derived interneurons (ID2); or basal forebrain neurons, namely, Dopamine-Receptor 1 (D1)-expressing medium-spiny neurons; Dopamine-Receptor 2 (D2)-expressing medium-spiny neurons; Cholinergic interneurons of the striatum (Ch-IN); and Cholinergic projection neurons of the basal ganglia (Ch-PN), see. e.g., FIGS. 1A and 1B.

The genes (transgenes) delivered by the rAAV vectors described herein are active and functional in the specific cells in which they are expressed, i.e., the products that they encode are produced, and are functionally expressed by the cells. By way of specific example, an rAAV vector as described herein which is engineered to contain an enhancer sequence that restricts expression of a transgene, e.g., reporter gene, to a GABAergic neuron (e.g., parvalbumin (PV)-expressing interneurons, Somatostatin (SST)-expressing interneurons; Vaso-active Intestinal Peptide (VIP)-expressing interneurons; and non-VIP/CGE-derived interneurons (ID2)), transduces these neuronal cell types, and the encoded reporter protein is functionally expressed in the GABAergic neuron cell type. By way of another specific example, an rAAV vector as described herein is engineered to contain an enhancer sequence that restricts expression of a transgene, e.g., a therapeutic gene or a reporter gene, or an effector gene, to basal forebrain neuron cells, (e.g., Dopamine-Receptor 1 (D1)-expressing medium-spiny neurons; Dopamine-Receptor 2 (D2)-expressing medium-spiny neurons; Cholinergic neurons (ChAT), Cholinergic interneurons of the striatum (Ch-IN); and Cholinergic projection neurons of the basal ganglia (Ch-PN)).

As another advantage, the described enhancer elements S9E1-S9E40 that restrict, regulate, control, or modulate expression of genes in certain neuronal cell populations are of a size/length (kb), e.g., less than approximately 2 kb, to allow for their insertion in a rAAV vector along with other polynucleotide sequences, e.g., a transgene, effector gene, reporter polynucleotides, polynucleotides encoding DREADDs, and other effector proteins as described herein, e.g., CRISPR/Cas (e.g., Cas9), ZFPs, TALENS, etc. By way of example, given the obligate minimal size of reporter elements (e.g., Enhanced green fluorescent protein (EGFP), orange fluorescent protein (dTomato)), alone or in combination with effector or reporter elements, (e.g. Channelrhodopsin (ChR2), DREADDs), which average about 700 bp to 2 kb, respectively, a maximum of ˜2 kb in packaging capacity remains for the insertion of a cis-acting DNA control element such as an enhancer sequence into an rAAV vector. The enhancer element sequences identified and described herein are capable of restricting expression to defined populations of neuronal cells and are sufficiently small elements to allow for additional nucleic acid sequences, reporter elements and transgenes, to also be cloned into the delivery vector, e.g., an AAV (rAAV) vector.

### Cell-Specific AAV Capsids

The rational design of AAV vectors that display selective tissue/organ targeting has broadened the applications of AAV as vector/vehicle for gene therapy. Both direct and indirect targeting approaches have been used to enhance AAV vector cell targeting specificity and retargeting. By way of example, in direct targeting, AAV vector targeting to certain cell types is mediated by small peptides or ligands that have been directly inserted into the viral capsid sequence. This approach has been successfully employed to target endothelial cells. Direct targeting requires detailed knowledge of the capsid structure such that peptides or ligands are positioned at sites that are exposed to the capsid surface; the insertion does not significantly affect capsid structure and assembly; and the native tropism is ablated to maximize targeting to a specific cell type. In indirect targeting, AAV vector targeting is mediated by an associating molecule that interacts with both the viral surface and the specific cell surface receptor. Such associating molecules for AAV vectors may include bispecific antibodies and biotin. The advantages of indirect targeting are that different adaptors can be coupled to the capsid without resulting in significant changes in the capsid structure, and the native tropism can be easily ablated. A disadvantage of using adaptors for targeting involves a potential for decreased stability of the capsid-adaptor complex in vivo.

In addition, AAV vectors may be produced that comprise capsids that allow for the increased transduction of cells and gene transfer to the central nervous system and the brain via the vasculature. (Chan, K. Y. et al., 2017, Nat. Neurosci., 20(8):1172-1179). Such vectors facilitate robust transduction of neuronal cells, including interneurons. When used with enhancers and cell-type specific promoters, such AAVs provide targeted gene expression in neuronal cells of the nervous system.

For applications that do not require high expression levels per cell, the amount of virus used, i.e., the viral dose, could be lowered. Lowering the viral load used for systemic gene delivery can reduce cost and production burden and minimize a potential risk for adverse reactions to viral components.

### Delivery of Recombinant Adeno-Associated Viral Vectors and Treatment Approaches

In general, the delivery of a transgene, e.g., a therapeutic gene, to treat a neurological, neurodevelopmental, neurodegenerative, neuropsychiatric, neurogenetic, or neuromuscular disease, disorder, or pathology at the genetic level, e.g., by modifying or correcting gene expression, such as by gene therapy, may be achieved using appropriate and effective vectors, such as viral or virus vectors, e.g., AAV or rAAV. By way of example, the use of a rAAV vector provides efficient delivery of therapeutic genes to a cell where the genes are expressed. While other methods and approaches for delivering genes to cells involve, for example, the use of purified DNA under hydrodynamic pressure, a shotgun approach using DNA adhering to gold particles, or lipid-DNA complexes, such methods and approaches frequently do not provide efficient gene delivery and result in gene expression that is lower than that required for therapeutic efficacy. Moreover, such methods are not applicable to human use. Viruses, on the other hand, represent natural vectors for the delivery and expression of exogenous genes in host cells in vivo.

An advantage associated with the use of rAAV as a viral vector is that rAAV transgene expression typically persists for years or for a lifetime, as has been demonstrated in animal models. This stands in contrast to non-rAAV viral vectors, which often lead to an initial burst of transgene expression that commonly disappears after a relatively short time, e.g., weeks.

To achieve enhanced therapy or treatment, the dose of rAAV vector that is required for a therapeutic response may be reduced, e.g., by using certain rAAV serotypes. Alternatively, the surface of the rAAV vector capsid may be altered to include specific ligands for attachment to target tissues and cells as described above. Another approach takes into consideration the trafficking of the virus particle from the endocytoplasmic vesicle to the nucleus. (Zhao, W. et al., 2007, Gene Ther., 14:545-550; Daya, S. and Berns, K. I., 2008, Clin. Microbiol. Rev., 21(4):583-593). Typically, the virus particle-to-infectivity ratio of rAAV vector preparations ranges from 10:1 to 100:1. The high ratios reflect incomplete or empty vector particles, as well as trafficking from the endocytoplasmic vesicle to the nucleus. During trafficking, the vector particle may become ubiquitinated and directed to a proteasome for degradation, rather than to the nucleus where the transgene may be expressed. It was found that ubiquitination and direction to the proteasome require phosphorylation of tyrosine residues on the surface of the rAAV vector capsid. When the seven tyrosine residues on the surface of the AAV-2 capsid were replaced phenylalanine residues, the multiplicity of infection (MOI) required for the detection of transgene expression was greatly reduced both in cell culture and in several mouse models of transduction of cells in the liver and eye. Consequently, the ability to increase transgene expression to therapeutic levels in the treatment of diseases may be enhanced.

For direct delivery to the brain, rAAV vectors may be administered by open neurosurgical procedure or by focal injection in order to bypass the blood-brain barrier, to restrict transgene expression temporally and spatially, and to target specific areas of the brain, e.g., basal forebrain and brain tissue, comprising specific populations of neuron and interneuron cells.

Systemic rAAV delivery (by intravenous injection) provides a non-invasive alternative for broad gene delivery to the nervous system; however, the high viral load required and relatively low transduction efficiency have limited wide adoption of this method. Several groups have developed rAAV capsids that enhance gene transfer to the CNS and certain tissues and cell populations after intravenous delivery. By way of example, AAV-AS capsid18 utilizes a polyalanine N-terminal extension to the AAV9.4719 VP2 capsid protein to provide higher neuronal transduction, particularly in the striatum. The AAV-BR1 capsid20, based on AAV2, may be useful for more efficient and selective transduction of brain endothelial cells. Another AAV capsid, AAV-PHP.B, comprises a capsid that transduces the majority of neurons and astrocytes across many regions of the adult mouse brain and spinal cord after intravenous injection. In an embodiment, rAAV comprises a capsid which specifically transduced interneurons, including PV interneurons, in the cerebral cortex (brain).

Other modes of rAAV vector administration may include lipid-mediated vector delivery, hydrodynamic delivery, and a gene gun. In a particular embodiment, the rAAV vectors comprise a capsid that increases the likelihood of directly infecting or transducing interneuron cells, such as GABAergic interneuron cells, such as PV-expressing interneurons, Somatostatin (SST)-expressing interneurons, Vaso-active Intestinal Peptide (VIP)-expressing interneurons, and non-VIP/CGE-derived interneurons (ID2), Dopamine-Receptor 1 (D1)-expressing medium-spiny neurons (Drd1 neurons), Dopamine-Receptor 2 (D2)-expressing medium-spiny neurons (Drd2 neurons), cholinergic neurons/interneurons, e.g., ChAT neurons, Cholinergic interneurons of the striatum (Ch-IN), or Cholinergic projection neurons of the basal ganglia (Ch-PN), and brain tissue comprising these cells.

### Treatment and Therapy for Neurological Diseases, Pathologies and Conditions

The enhancer element sequences described herein provide advantages and benefits for targeting the expression of genes in particular neuronal cell types, especially when the enhancer element sequence is a component of a delivery vector, such as a lentivirus vector, an adeno-associated virus (AAV) vector, or a recombinant adeno-associated virus (rAAV) vector, which targets a particular neuronal cell type in which the gene is expressed and functional. In an embodiment, the expressed gene is a therapeutic gene, which may treat, ameliorate, improve, reduce, abate, diminish, resolve, or eliminate a disease, pathology, or condition, or a symptom thereof, when expressed in a particular neuronal cell type, which expresses an aberrant or mutated form of the gene, or in which the gene is nonfunctional or not expressed due to loss, mutation, silencing, non- or dysfunction, and the like. In an embodiment, the genes are those associated with or causative of a particular disease, pathology, or condition, such as a neuronal, neurological, neurodevelopmental, neurodegenerative, neuropathological, neurogenetic, neuropsychiatric, or neuromuscular disease, pathology or condition, and/or symptoms thereof. Nonlimiting examples of such diseases, pathologies, or conditions include seizures, Alzheimer's disease, Parkinson's disease, Dystonia, Amyotrophic lateral Sclerosis (ALS), and Down Syndrome.

In an embodiment, the enhancer element is S9E1, which targets a PV interneuron, and a vector expressing the S9E1 (or huS9E1) enhancer element targets PV interneuron cells expressing the target gene Prss23 (serine protease 23, a member of the trypsin family of serine proteases). Examples of diseases associated with Prss23 include Coats Disease, an idiopathic disorder characterized by retinal telangiectasia with deposition of intraretinal or subretinal exudates, which can lead to retinal detachment and unilateral blindness; and exudative vitreoretinopathy, which is an inherited disorder characterized by the incomplete development of the retinal vasculature.

In an embodiment, the enhancer element is S9E2, which targets a PV interneuron, and a vector expressing the S9E2 (or huS9E2) enhancer element targets PV interneuron cells expressing the target gene Lpl (lipoprotein lipase). The Lpl gene product has the dual functions of triglyceride hydrolase and ligand/bridging factor for receptor-mediated lipoprotein uptake. Severe mutations that cause Lpl deficiency result in type I hyperlipoproteinemia, while less extreme mutations in Lpl are linked to many disorders of lipoprotein metabolism.

In an embodiment, the enhancer element is S9E3, which targets a PV interneuron, and a vector expressing the S9E3 (or huS9E3) enhancer element targets PV interneuron cells expressing the target gene Cntnap5b (Contactin associated protein family member 5). The Cntnap5b gene product belongs to the neurexin family, members of which function in the vertebrate nervous system as cell adhesion molecules and receptors. Diseases associated with CNTNAP5 include Posterior Cortical Atrophy, a rare neurologic disease characterized by impairment of higher visual processing skills and other posterior cortical functions without any evidence of ocular abnormalities, relatively intact memory and language in the early stages, and atrophy of posterior brain regions; and Dyslexia, a brain-based type of learning disability that specifically impairs a person's ability to read. Cntnap5b may play a role in the correct development and proper functioning of the peripheral and central nervous system and be involved in cell adhesion and intercellular communication.

In an embodiment, the enhancer element is S9E4, which targets a PV interneuron, and a vector expressing the S9E4 (or huS9E4) enhancer element targets PV interneuron cells expressing the target gene Plcxd3 (Phosphatidylinositol Specific Phospholipase C X Domain Containing 3). The Plcxd3 gene product is one of a group of enzymes that hydrolyze phospholipids into fatty acids and other lipophilic molecules. Diseases associated with PLCXD3 include Creutzfeldt-Jakob Disease (CJD), a rare, degenerative, fatal brain disorder. The three major categories of CJD include sporadic (the most common form, in which people do not have any known risk factors for the disease); hereditary (in which the person has a family member with the disease and tests positive for a genetic mutation associated with CJD), and acquired (in which the disease is transmitted by exposure to brain and nervous system tissue, usually through certain medical procedures). A variant CJD form can be acquired by eating meat from cattle affected by a disease similar to CJD, called bovine spongiform encephalopathy (commonly called “mad cow” disease).

In an embodiment, the enhancer element is S9E5, which targets a PV interneuron, and a vector expressing the S9E5 (or huS9E5) enhancer element targets PV interneuron cells expressing the target gene Elf5 (E74-like Factor 5, Ets domain transcription factor). The Elf5 gene product is a member of an epithelium-specific subclass of the Ets transcription factor family. Diseases associated with ELF5 include Down Syndrome and Isolated Growth Hormone Deficiency, Type Ii.

In an embodiment, the enhancer element is S9E6, which targets an SST interneuron, and a vector expressing the S9E6 (or huS9E6) enhancer element targets SST interneuron cells expressing the target gene Satb1 (Special AT-Rich Sequence Binding Protein 1 (Binds To Nuclear Matrix/Scaffold-Associating DNA)). The Satb1 gene product is a matrix protein which binds nuclear matrix and scaffold-associating DNAs. Diseases associated with SATB1 include Developmental Delay With Dysmorphic Facies And Dental Anomalies (DEFDA), characterized by generally mild global developmental delay with variably impaired intellectual development, and Kohlschutter-Tonz Syndrome-Like (KTZSL). KTZSL is characterized by global developmental delay with moderately to severely impaired intellectual development, poor or absent speech, and delayed motor skills. Early-onset epilepsy is common and may be refractory to treatment, leading to epileptic encephalopathy.

In an embodiment, the enhancer element is S9E7, which targets an SST interneuron, and a vector expressing the S9E7 (or huS9E7) enhancer element targets SST interneuron cells expressing the target gene Ccna1 (Cyclin A1). The Ccna1 gene product is a belongs to the highly conserved cyclin family, whose members are characterized by a periodicity in protein abundance through the cell cycle. The cyclin encoded by Ccna1 is expressed in testis and brain. Diseases associated with Ccna1 include myeloid leukemia and testicular cancer.

In an embodiment, the enhancer element is S9E8, which targets an SST interneuron, and a vector expressing the S9E8 (or huS9E8) enhancer element targets SST interneuron cells expressing the target gene Calb1 (Calbidin 1), a Vitamin D-dependent calcium-binding protein. The Calb1 gene product is a member of the calcium-binding protein superfamily that includes calmodulin and troponin C. The Calb1 product plays a role in buffering entry of calcium upon stimulation of glutamate receptors Depletion of this protein was noted in patients with Huntington disease. Diseases associated with Calb1 include Cerebellar Disease (ataxias, dysarthria and cerebellar cognitive affective syndrome) and Temporal Lobe Epilepsy.

In an embodiment, the enhancer element is S9E9, which targets an SST interneuron, and a vector expressing the S9E9 (or huS9E9) enhancer element targets SST interneuron cells expressing the target gene Smc2 (Structural Maintenance of Chromosomes 2). The Smc2 gene product is a central component of the condensin complex, required for conversion of interphase chromatin into mitotic-like condense chromosomes. Diseases associated with SMC2 include Pleural Empyema (a rare pulmonary condition characterized by accumulation of pus in the pleural cavity, often as a consequence of pneumonia) and Progeroid Syndrome (rare genetic disorders that mimic premature aging).

In an embodiment, the enhancer element is S9E10, which targets an SST interneuron, and a vector expressing the S9E10 (or huS9E10) enhancer element targets SST interneuron cells expressing the target gene Hpse (Heparinase). The Hpse gene product is an endoglycosidase that cleaves heparan sulfate proteoglycans (HSPGs) into heparin sulfate side chains and core proteoglycans and participates in extracellular matrix (ECM) degradation and remodeling. Diseases associated with HPSE include Urofacial Syndrome 1 (ochoa syndrome, a rare condition that causes unusual facial expressions and problems with urination) and Gastric Signet Ring Cell Adenocarcinoma.

In embodiments, the enhancer element is S9E11 (or huS9E11) or S9E12 (or huS9E12), which target a VIP interneuron, and a vector expressing the S9E11 (or huS9E11) or S9E12 (or huS9E12) enhancer element targets VIP interneuron cells expressing the target gene Prox1 ((Prospero Homeobox 1)). The Prox1 gene product is a member of the homeobox transcription factor family and plays a critical role in embryonic development and functions as a key regulatory protein in neurogenesis and the development of the heart, eye lens, liver, pancreas and the lymphatic system. Involved in the regulation of the circadian rhythm.

In an embodiment, the enhancer element is S9E13, which targets a VIP interneuron, and a vector expressing the S9E13 (or huS9E13) enhancer element targets VIP interneuron cells expressing the target gene Vip (vasoactive intestinal peptide). The Vip gene product is a member of the glucagon family. Diseases associated with VIP include Vipoma (a rare cancer caused by a type of pancreatic neuroendocrine tumor, which secretes VIP, a hormone that stimulates the secretion (and inhibits the absorption) of sodium, chloride, potassium and water within the small intestine) and Pancreatic Cholera, a disease related to related to secretory diarrhea and diarrhea. VIP causes vasodilation, lowers arterial blood pressure, stimulates myocardial contractility, increases glycogenolysis and relaxes the smooth muscle of trachea, stomach and gall bladder.

In an embodiment, the enhancer element is S9E14, which targets a VIP interneuron, and a vector expressing the S9E14 (or huS9E14) enhancer element targets VIP interneuron cells expressing the target gene NpySr (Neuropeptide Y Receptor Type 5). The NpySr gene product is a receptor for neuropeptide Y and peptide YY. The encoded protein appears to be involved in regulating food intake, with defects in this gene being associated with eating disorders. The encoded protein is also involved in a pathway that protects neuroblastoma cells from chemotherapy-induced cell death, providing a possible therapeutic target against neuroblastoma. Diseases associated with NPY5R include Cocaine Dependence and Panic Disorder. Cocaine Dependence is related to cocaine abuse and personality disorder. An important gene associated with Cocaine Dependence is DRD2 (Dopamine Receptor D2), and among its related pathways/superpathways are Peptide ligand-binding receptors and Transmission across Chemical Synapses. The drugs caffeine and Lamotrigine have been mentioned in the context of this disorder. Affiliated tissues include brain, prefrontal cortex and cortex, and related phenotypes are behavior/neurological and homeostasis/metabolism. Panic disorders relate to a group of mental illnesses that involve long-term patterns of thoughts and behaviors that are unhealthy and inflexible.

In an embodiment, the enhancer element is S9E15, which targets a VIP interneuron, and a vector expressing the S9E15 (or huS9E15) enhancer element targets VIP interneuron cells expressing the target gene Grpr (Gastrin-Releasing Peptide Receptor). The Grpr gene product regulates numerous functions of the gastrointestinal and central nervous systems, including release of gastrointestinal hormones, smooth muscle cell contraction, and epithelial cell proliferation and is a potent mitogen for neoplastic tissues. Diseases associated with GRPR include agoraphobia and prostate cancer.

In an embodiment, the enhancer element is S9E16, which targets an ID2 interneuron, and a vector expressing the S9E16 (or huS9E16) enhancer element targets ID2 interneuron cells expressing the target gene Sv2c (Synaptic Vesicle Protein 2C). The Sv2c gene product plays a role in the control of regulated secretion in neural and endocrine cells, enhancing selectively low-frequency neurotransmission. Positively regulates vesicle fusion by maintaining the readily releasable pool of secretory vesicles.

In an embodiment, the enhancer element is S9E17, which targets an ID2 interneuron, and a vector expressing the S9E17 (or huS9E17) enhancer element targets ID2 interneuron cells expressing the target gene Pde11a (Phosphodiesterase 11A). Phosphodiesterases (PDEs) are a family of phosphohydrolyases that catalyze the hydrolysis of 3′ cyclic phosphate bonds in adenosine and/or guanine 3′,5′ cyclic monophosphate (cAMP and/or cGMP). PDEs regulate the second messengers by controlling their degradation. Diseases associated with PDE11A include Pigmented Nodular Adrenocortical Disease, Primary, 2 (a rare bilateral adrenal defect causing ACTH-independent Cushing syndrome. Macroscopic appearance of the adrenals is characteristic with small pigmented micronodules observed in the cortex) and Primary Pigmented Nodular Adrenocortical Disease (a form of bilateral adrenocortical hyperplasia that is often associated with adrenocorticotrophin hormone (ACTH) independent Cushing syndrome and is characterized by small to normal sized adrenal glands containing multiple small cortical pigmented nodules (less than 1 cm in diameter)).

In an embodiment, the enhancer element is S9E18, which targets an ID2 interneuron, and a vector expressing the S9E18 (or huS9E18) enhancer element targets ID2 interneuron cells expressing the target gene Wt1 (Wt1 transcription factor). The Wt1 gene product is a tumor suppressor transcription factor that contains four zinc-finger motifs at the C-terminus and a proline/glutamine-rich DNA-binding domain at the N-terminus. It has an essential role in the normal development of the urogenital system, and it is mutated in a small subset of patients with Wilms tumor.

In an embodiment, the enhancer element is S9E19 or S9E20, which target an ID2 interneuron, and a vector expressing the S9E19 (or huS9E19) or S9E20 (or huS9E20) enhancer element targets ID2 interneuron cells expressing the target gene Lamp5 (Lysosomal Associated Membrane Protein Family Member 5). The Lamp5 gene product plays a role in short-term synaptic plasticity in a subset of GABAergic neurons in the brain.

In an embodiment, the enhancer element is S9E21, which targets a Drd1 neuron, and a vector expressing the S9E21 (or huS9E21) enhancer element targets Drd1 neuron cells expressing the target gene Slc35d3 (Solute Carrier Family 35 Member D3). Diseases associated with SLC35D3 include Thrombocytopenia With Beta-Thalassemia, X-Linked (an unusual form of thrombocytopenia associated with beta-thalassemia) and Arthrogryposis, Renal Dysfunction, And Cholestasis 1, or arc syndrome, which is a multisystem disorder, characterized by neurogenic arthrogryposis multiplex congenita, renal tubular dysfunction and neonatal cholestasis with bile duct hypoplasia and low gamma glutamyl transpeptidase activity. Platelet dysfunction is common.

In an embodiment, the enhancer element is S9E25, which targets a Drd1 neuron, and a vector expressing the S9E25 (or huS9E25) enhancer element targets Drd1 neuron cells expressing the target gene Drd1 (Dopamine D1 Receptor). The Drd1 gene product is the D1 subtype of the dopamine receptor and is the most abundant dopamine receptor in the central nervous system. A G-protein coupled receptor, the DRD1 product stimulates adenylyl cyclase and activates cyclic AMP-dependent protein kinases. D1 receptors regulate neuronal growth and development, mediate some behavioral responses, and modulate dopamine receptor D2-mediated events. Diseases associated with DRD1 include cerebral meningioma (related to meningioma, familial and intracranial meningioma, with symptoms including seizures and headache) and pathological or compulsive gambling.

In an embodiment, the enhancer element is S9E33 or S9E34, which target a Drd1 neuron, and a vector expressing the S9E33 (or huS9E33) or S9E34 (or huS9E34) enhancer element targets Drd1 neuron cells expressing the target gene Chrm4 (Cholinergic Receptor Muscarinic 4). The Chrm4 gene product is belongs to the family of muscarinic acetylcholine receptors, which mediate various cellular responses, including inhibition of adenylate cyclase, breakdown of phosphoinositides and modulation of potassium channels through the action of G proteins. Muscarinic receptors are predominantly expressed in the parasympathetic nervous system; they influence many effects of acetylcholine in the central and peripheral nervous system where they exert both inhibitory and excitatory effects.

In an embodiment, the enhancer element is S9E36, which targets a Drd1 neuron, and a vector expressing the S9E36 (or huS9E36) enhancer element targets Drd1 neuron cells expressing the target gene Tac1 (Tachykinin Precursor 1). The Tac1 gene product is a Tachykinin, a member of a family of active peptide hormones that excite neurons, evoke behavioral responses, are potent vasodilators and secretagogues, and contract many smooth muscles both directly and indirectly. Diseases associated with TAC1 include Complex Regional Pain Syndrome, which is related to reflex sympathetic dystrophy and causalgia, and has symptoms including severe prolonged chronic pain (such as back pain), seizures, and tremor; and Vasomotor Rhinitis, a rhinitis which involves a hypersensitive reaction to various potentially irritating stimuli, such as changes in weather, temperature, or barometric pressure; chemical irritants; psychological stress and emotional shocks; medications; alcohol; and certain foods.

In an embodiment, the enhancer element is S9E22 or S9E23, which target a Drd2 neuron, and a vector expressing the S9E22 (or huS9E22) or S9E23 (or huS9E23) enhancer element targets Drd2 neuron cells expressing the target gene Gpr6 (G Protein-Coupled Receptor 6). The Gpr6 gene product is an orphan receptor with constitutive G(s) signaling activity that activates cyclic AMP, promotes neurite outgrowth, and blocks myelin inhibition in neurons.

In an embodiment, the enhancer element is S9E24, which targets a Drd2 neuron, and a vector expressing the S9E24 (or huS9E24) enhancer element targets Drd2 neuron cells expressing the target gene Adora2a (Adenosine Receptor A2a). The Adora2a gene product is a member of the guanine nucleotide-binding protein (G protein)-coupled receptor (GPCR) superfamily. The Adenosine Receptor A2a protein, an adenosine receptor of A2A subtype, uses adenosine as the preferred endogenous agonist and preferentially interacts with the G(s) and G(olf) family of G proteins to increase intracellular cAMP levels. It plays an role in many biological functions, such as cardiac rhythm and circulation, cerebral and renal blood flow, immune function, pain regulation, and sleep and has been implicated in pathophysiological conditions, such as inflammatory diseases and neurodegenerative disorders. Diseases associated with ADORA2A include Acute Encephalopathy with Biphasic Seizures and Late Reduced Diffusion, as well as Intermittent Asthma.

In an embodiment, the enhancer element is S9E31 or S9E32, which target a Drd2 neuron, and a vector expressing the S9E31 (or huS9E31) or S9E32 (or huS9E32) enhancer element targets Drd2 neuron cells expressing the target gene Sp9 (Sp9 transcription factor). The Sp9 gene product plays a role in limb development; it positively regulates FGF8 expression in the apical ectodermal ridge (AER) and contributes to limb outgrowth in embryos.

In an embodiment, the enhancer element is S9E27 or S9E28, which target a cholinergic neuron, and a vector expressing the S9E27 (or huS9E27) or S9E28 (or huS9E28) enhancer element targets cholinergic neuron cells expressing the target gene Chat (Choline O-Acetyltransferase). The Chat gene product, choline acetyltransferase is an enzyme that catalyzes the biosynthesis of the neurotransmitter acetylcholine. The Choline acetyltransferase enzyme is a characteristic feature of cholinergic neurons. Changes in these neurons are associated with certain symptoms of Alzheimer's disease. Polymorphisms in the Chat gene have been associated with Alzheimer's disease and mild cognitive impairment. Mutations in the Chat gene are associated with congenital myasthenic syndrome associated with episodic apnea, having symptoms including apnea, ophthalmoparesis and respiratory distress. Diseases associated with the Chat gene and its gene product include Myasthenic syndrome, congenital, 6, presynaptic. A form of congenital myasthenic syndrome, which are a group of disorders characterized by failure of neuromuscular transmission, including pre-synaptic, synaptic, and post-synaptic disorders that are not of autoimmune origin.

In an embodiment, the enhancer element is S9E38 or S9E39, which target a cholinergic neuron, and a vector expressing the S9E38 (or huS9E38) or S9E39 (or huS9E39) enhancer element targets cholinergic neuron cells expressing the target gene Zic1 (Zic Family Member 1). The Zic1 gene product is a member of the ZIC family of C21-12-type zinc finger proteins. Members of this family are important during development. Aberrant expression of the Zic/gene is seen in medulloblastoma, a childhood brain tumor. The Zic1 gene is closely linked to the gene encoding zinc finger protein of the cerebellum 4, a related family member on chromosome 3 and encodes a transcription factor that can bind and transactivate the apolipoprotein E gene Diseases associated with ZIC1 include craniosynostosis 6 and Structural Brain Anomalies with Impaired Intellectual Development and Craniosynostosis. Craniosynostosis is a primary abnormality of skull growth involving premature fusion of the cranial sutures such that the growth velocity of the skull often cannot match that of the developing brain. This produces skull deformity and, in some cases, raises intracranial pressure. Affiliated tissues include brain and pons, and related phenotypes are scoliosis (abnormal lateral curvature of the spine) and ptosis (drooping upper eyelid).

In an embodiment, the enhancer element is S9E26, which targets a Ch-IN neuron, and a vector expressing the S9E26 (or huS9E26) enhancer element targets Ch-IN neuron cells expressing the target gene Isl1 (ISL LIM Homeobox 1). The Isl1 gene product is a member of the LIM/homeodomain family of transcription factors. The encoded protein binds to the enhancer region of the insulin gene, among others, and may play an important role in regulating insulin gene expression. The encoded protein is central to the development of pancreatic cell lineages and may also be required for motor neuron generation. Diseases associated with ISL1 include bladder exstrophy and epispadias complex (an anterior midline developmental defect with variable expression involving the infraumbilical abdominal wall including the pelvis, urinary tract, and external genitalia) and bladder exstrophy (characterized by an evaginated bladder plate, epispadias and an anterior defect of the pelvis, pelvic floor and abdominal wall).

In an embodiment, the enhancer element is S9E35, which targets a Ch-IN neuron, and a vector expressing the S9E35 (or huS9E35) enhancer element targets Ch-IN neuron cells expressing the target gene Tshz2 (Teashirt Zinc Finger Homeobox 2). The Tshz2 gene product is a member of the teashirt C2H2-type zinc-finger protein family of transcription factors. This gene encodes a protein with five C21-12-type zinc fingers, a homeobox DNA-binding domain and a coiled-coil domain. This nuclear protein is predicted to act as a transcriptional repressor. This gene is thought to play a role in the development and progression of breast and other types of cancer. Diseases associated with TSHZ2 include autosomal recessive Alport Syndrome, a genetic condition characterized by kidney disease, hearing loss, and eye abnormalities.

In an embodiment, the enhancer element is S9E40, which targets a Ch-IN neuron, and a vector expressing the S9E40 (or huS9E40) enhancer element targets Ch-IN neuron cells expressing the target gene Zic1.

In an embodiment, the enhancer element is S9E29, which targets a Ch-PN neuron, and a vector expressing the S9E29 (or huS9E29) enhancer element targets Ch-PN neuron cells expressing the target gene Chat.

In an embodiment, the enhancer element is S9E30, which targets a Ch-PN neuron, and a vector expressing the S9E30 (or huS9E30) enhancer element targets Ch-PN neuron cells expressing the target gene Pcdh9 (Protocadherin 9). The Pcdh9 gene product is a member of the protocadherin family, and cadherin superfamily, of transmembrane proteins containing cadherin domains, which mediate cell adhesion in neural tissues in the presence of calcium. The protocadherin 9 protein may be involved in signaling at neuronal synaptic junctions. Diseases associated with PCDH9 include Auditory Neuropathy, Autosomal Dominant, 1, which is a form of sensorineural hearing loss with absent or severely abnormal auditory brainstem response, in the presence of normal cochlear outer hair cell function and normal otoacoustic emissions. Auditory neuropathies result from a lesion in the area including the inner hair cells, connections between the inner hair cells and the cochlear branch of the auditory nerve, the auditory nerve itself and auditory pathways of the brainstem.

In an embodiment, the enhancer element is S9E37, which targets a Ch-PN neuron, and a vector expressing the S9E37 (or huS9E37) enhancer element targets Ch-PN neuron cells expressing the target gene Zic1.

### Pharmacogenetic Approaches

Pharmacogenetic approaches are contemplated for use with the virus vectors, rAAV vectors, compositions thereof, and methods described herein. Such approaches deliver either Gq-DREADD receptor or PSAM into PV-interneurons specifically using a viral vector, such as a rAAV vector comprising an enhancer element (e.g., S9E1-S9E5) as described herein and a polynucleotide encoding a Gq-DREADD receptor or PSAM. The targeted PV-neurons, either in a specific region upon focal injection or throughout the cortex upon systemic injection, as dictated by the type of pathology being treated, stably express the receptor (Gq-DREADD or PSAM). Thereafter, an individual (patient) is administered the drug that activates the receptor (e.g. CNO or PSEM, respectively). This approach results in a controlled alteration of the excitability of the PV-interneurons expressing the receptor and allows for a dose-dependent and time-dependent modulation of the excitation/inhibition (E/I) balance in neurons (interneurons and PV-expressing interneurons), resulting in a normalization of brain activity. Pharmacogenetic approaches using the enhancer element sequences described herein are also contemplated for regulating or modulating gene expression and function in other types of neurons, such as SST, VIP, ID2 interneurons, or Drd1, Drd2, or cholinergic (e.g., ChAT, Ch-IN, or Ch-PN) neurons.

### Pharmaceutical Compositions

Provided also are pharmaceutical compositions or formulations for treating subjects who are afflicted with, or who are at risk of developing, a neurological, neurodevelopmental, neurodegenerative, neuropsychiatric, neurogenetic, or neuromuscular disease, disorder, or pathology. In an embodiment, the pharmaceutical composition includes an AAV vector or virus particle, such as one containing an enhancer sequence as described herein (as active agent) and a pharmaceutically acceptable carrier, excipient, or diluent. When formulated in a pharmaceutical composition, an rAAV vector as therapeutic compound or product can be admixed with a pharmaceutically acceptable carrier, diluent, or excipient.

The therapeutic agent(s) may be contained in any appropriate amount in any suitable carrier substance, and is/are generally present in an amount of 1-95% by weight of the total weight of the composition. The composition may be provided in a dosage form that is suitable for a parenteral (e.g., subcutaneous, intravenous, intramuscular, or intraperitoneal) administration route, such that the agent, such as a viral vector described herein, is systemically delivered. In an embodiment, systemic injection of an rAAV vector as described herein allows for the characterization of specificity of expression across brain regions, particularly when a reporter product is also encoded by the vector. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions may be formulated to release the active agent substantially immediately upon administration or at any predetermined time or time after administration. The latter types of compositions are generally known as controlled release formulations, which include (i) formulations that create a substantially constant concentration of the agent within the body over an extended period of time; (ii) formulations that after a predetermined lag time create a substantially constant concentration of the drug within the body over an extended period of time; (iii) formulations that sustain action during a predetermined time period by maintaining a relatively constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with a target site or location, e.g., in a region of a tissue or organ; (v) formulations that allow for convenient dosing, such that doses are administered, for example, once every one, two, or several weeks; and (vi) formulations that target a specific tissue or cell type using carriers, chemical derivatives, or specifically designed vectors (e.g., comprising a certain capsid composition) to deliver the therapeutic agent, e.g., to interneurons or PV-expressing GABAergic interneurons, or pyramidal neurons, e.g., glutamatergic pyramidal neurons. For some applications, controlled release formulations obviate the need for frequent dosing during the day to sustain the plasma level of the administered agent at a therapeutic level.

Methods by which to obtain controlled release in which the rate of release outweighs the rate of metabolism of the agent in question are not meant to be limiting. By way of example, controlled release is obtained by appropriate selection of various formulation parameters and ingredients, including, e.g., various types of controlled release compositions and coatings. Thus, the therapeutic agent is formulated with appropriate excipients into a pharmaceutical composition that, upon administration, releases the agent in a controlled manner. Examples include single or multiple unit tablet or capsule compositions, oil solutions, suspensions, emulsions, microcapsules, microspheres, molecular complexes, nanoparticles, patches, and liposomes.

The administration of a composition comprising a combination of agents for the treatment or therapy of a neurological, neurodevelopmental, neurodegenerative, neuropsychiatric, neurogenetic, or neuromuscular disease, disorder, pathology, or condition may be by any suitable means that results in a concentration of the therapeutic that, combined with other components, is effective in ameliorating, abating, reducing, decreasing, or stabilizing seizures in a subject. The composition may be administered systemically, for example, formulated in a pharmaceutically-acceptable buffer such as physiological saline. In an embodiment, systemic injection of an rAAV vector as described herein allows for the characterization of specificity of expression across brain regions, particularly when a reporter product is also encoded by the vector.

Routes of administration include, for example, intracranial, parenteral, subcutaneous (s.c.), intravenous (i.v.), intraperitoneal (i.p.), intramuscular (i.m.), or intradermal administration, e.g., by injection, that optimally provide continuous, sustained levels of the agent in the patient. The amount of the therapeutic agent to be administered varies depending upon the manner of administration, the age, physical condition and body weight of the patient, and with the clinical symptoms of the neurological, neurodevelopmental, neurodegenerative, neuropsychiatric, neurogenetic, or neuromuscular disease, disorder, or pathology. Generally, amounts will be in the range of those used for other viral vector-based agents employed in the treatment of neurological, neurodevelopmental, neurodegenerative, neuropsychiatric, neurogenetic, or neuromuscular diseases, disorders, pathologies and conditions, particularly in the brain, although in certain instances lower amounts are needed if the agent exhibits increased specificity. A composition is administered at a dosage that shows a therapeutic effect, such as, for example, ameliorating, abating, reducing, decreasing, or stabilizing seizures in a patient, as determined by methods known to one skilled in the art.

The pharmaceutical composition may be administered parenterally by injection, infusion or implantation (subcutaneous, intravenous, intramuscular, intraperitoneal, intracranial, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation, and can be found, for example, in Remington: The Science and Practice of Pharmacy, supra. In particular embodiments, administration is systemic and parenteral, such as by injection or intravenous delivery.

Compositions for parenteral delivery and administration may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent (e.g., viral vector or particle comprising enhancer sequences and polynucleotides encoding a transgene, e.g., a therapeutic gene or an effector gene, and associated regulatory sequences, as described herein), the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising the active therapeutic(s) (i.e., viral vector or particle described herein) is formulated for intravenous delivery. As noted above, the pharmaceutical compositions according to the described embodiments may be in the form suitable for sterile injection. To prepare such a composition, the suitable therapeutic(s) are dissolved or suspended in a parenterally acceptable liquid vehicle. Acceptable vehicles and solvents that may be employed include water, water adjusted to a suitable pH by addition of an appropriate amount of hydrochloric acid, sodium hydroxide or a suitable buffer, 1,3-butanediol, Ringer's solution, isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Methods of Administration and Delivery

Administration of a viral vector or pharmaceutical composition as described herein to a subject, e.g., a patient having, or at risk of having, a neurological, neurodevelopmental, neurodegenerative, neuropsychiatric, neurogenetic, or neuromuscular, disease, disorder, or pathology, and/or the symptoms thereof. In embodiments, the viral vector, viral particle, or pharmaceutical composition may be delivered to a cell (e.g., a target cell such as an interneuron or a brain layer comprising interneurons) in any manner such that the viral vector, particle or composition is functional and active to express the sequences contained in the vector or virus particle. Illustratively, rAAV comprising an enhancer element as described herein and a transgene or polynucleotide sequence (e.g., a therapeutic gene) may be delivered to interneuron or neuron cells or tissue comprising interneuron or neuron cells to provide for targeted expression of the gene (and the encoded gene product) in the interneurons or neurons. Thus, viral vectors or viral particles are delivered to a cell by contacting the cell with a composition comprising the viral vectors, or viral particles and by heterologous expression of the polynucleotides harbored by the viral vector or viral particles in the cell. The polynucleotides harbored by the rAAV vector must be delivered to the cells of a subject in a form in which they can be taken up so that therapeutically effective levels of the encoded products can be produced.

Transducing rAAV vectors are used for the delivery and expression of genes encoding desired proteins, polypeptides, or peptides to cells, especially because of their high efficiency of infection and stable integration and expression (see, e.g., Cayouette et al., Human Gene Therapy, 8:423-430, 1997; Kido et al., Current Eye Research, 15:833-844, 1996; Bloomer et al., Journal of Virology, 71:6641-6649, 1997; Naldini et al., Science, 272:263-267, 1996; and Miyoshi et al., Proc. Natl. Acad. Sci. U.S.A., 94:10319, 1997). By way of example, rAAV is engineered to contain a polynucleotide encoding a specific enhancer nucleic acid sequence as described herein that preferentially directs gene expression in interneuron cell types and is used to direct and restrict the expression of a gene in GABAergic interneuron target cells or in basal forebrain target cells. In an embodiment, expression of the gene can be driven from any suitable promoter, such as a promoter specific for the target cells. In an embodiment, the rAAV vector is administered systemically. In an embodiment, systemic injection of an rAAV vector as described herein allows for the characterization of specificity of expression across brain regions, particularly, for example, when a reporter product is also encoded by the vector.

Gene transfer can also be achieved using in vitro transfection methods. Such methods include the use of calcium phosphate, DEAE dextran, electroporation, and protoplast fusion. Liposomes can also be potentially beneficial for delivery of DNA into a cell.

### Treatment Methods and Protocols

Provided are methods of administering a therapeutic agent to a subject in need, such as a subject having, undergoing, having experienced, and/or at risk of experiencing a neurological, neurodevelopmental, neurodegenerative, neuropsychiatric, neurogenetic, or neuromuscular disease, disorder, or pathology, more particularly, a seizure or epilepsy, and who also may be diagnosed with, or be suspected of having, or having symptoms of, a seizure disorder, or who is identified as being in need of such treatment, in which an effective amount of a viral vector or viral particle as described herein, or a composition described herein, is administered to the subject to produce a therapeutic effect.

According to the described methods, a therapeutic effect includes, without limitation, that amount of rAAV that is introduced into a sufficient number of interneurons so as to inhibit, reduce, or ameliorate one or more symptoms of the neurological, neurodevelopmental, neurodegenerative, neuropsychiatric, neurogenetic, or neuromuscular disease, disorder, or pathology, e.g., a seizure or epilepsy, or to prevent one or more symptoms subsequent to the administration of the rAAV vector product or composition to the subject. The amount of rAAV that is administered may be determined by the skilled practitioner in the art, such as a medical or clinical practitioner, and, as appreciated by one skilled in the art, is based on factors such as the size of the epileptic focus, the titer of the virus preparation and from data acquired in non-human primates (e.g., Colle, M.-A. et al., 2010, Hum. Mol. Genet., 19:147-158). By way of example, from 10¹⁰ to 10¹² rAAV particles may be used to transduce rAAV vectors or particles thereof to a therapeutically relevant number of interneurons. Identifying a subject in need of such treatment can be in the judgment of a subject or a health care professional and can be subjective (e.g. opinion) or objective (e.g. measurable by a test or diagnostic method).

The therapeutic methods (which include prophylactic treatment) in general comprise administration of a therapeutically effective amount of the agents described herein, such as an rAAV vector, a viral particle, or composition containing the aforementioned agents, to a subject (e.g., animal, human) in need thereof, including a mammal, particularly a human. Such treatment will be suitably administered to subjects, particularly humans or infant humans, suffering from, having, susceptible to, or at risk for a neurological, neurodevelopmental, neurodegenerative, neuropsychiatric, neurogenetic, or neuromuscular disease, disorder, or pathology, such as seizures and/or epilepsy. Determination of those subjects “at risk” can be made by any objective or subjective determination by a diagnostic test or opinion of a subject or health care provider (e.g., genetic test, enzyme or protein marker or biomarker, family history, and the like).

Viral vectors and pharmaceutical compositions as described can be used therapeutically to treat patients suffering from neurological, neurodevelopmental, neurodegenerative, neuropsychiatric, neurogenetic, or neuromuscular diseases or disorders, e.g., seizures, epilepsy, etc., or prophylactically to provide advanced treatment or protection to patients at risk for certain neurological, neurodevelopmental, neurodegenerative, neuropsychiatric, neurogenetic, or neuromuscular diseases, disorders, or pathologies, such as a prophylactic vaccination to reduce, diminish, abate, or ward off one or more symptoms of the disease, disorder, or pathology, and/or the severity thereof. A prophylactically effective amount of the rAAV vectors as described herein are not intended to be limiting herein, and may range between about 10² TU (transducing units) per kilogram body weight of the recipient and about 10² TU kilogram body weight of the recipient, or any TUs in between those values. Mouse models of disease or disorders, e.g., seizures, can be used to optimize dosages and regimens.

The therapeutic vectors as described herein may be administered to a subject in need thereof in an effective amount to normalize the excitability of certain genes associated with particular types of interneurons or neurons that may be deficient in a functioning gene to alleviate a neurological, neurodevelopmental, neurodegenerative, neuropsychiatric, neurogenetic, or neuromuscular disease, disorder, pathology, or condition and/or the symptoms thereof. The vectors and methods described herein may be of therapeutic value for an individual, e.g., a human infant, child or adult, who experiences or is at risk for experiencing one or more symptoms of the disease, disorder, or pathology. In an embodiment, an rAAV or a composition comprising an rAAVs as described herein is administered to an individual whose interneurons do not express or exhibit loss of function or expression, at the time of administration, of a certain neuronal- or interneuronal cell gene, which is dependent for expression on an enhancer element, such as S9E1-S9E40 described herein. In an embodiment, the expression of a therapeutic gene in interneuron or neuron cells transduced by the described rAAV vectors containing an enhancer sequence that restricts expression of the gene in certain cell types normalizes the excitability of interneurons or neurons that are deficient in, or have abnormal expression of, the gene. In an embodiment, a composition comprising an rAAV vector as described herein is administered to an individual whose interneurons no longer express a given gene. In an embodiment, a composition comprising an rAAV vector as described herein is administered to an individual of any age, e.g., infant to adult.

Subjects, e.g., mammalian subjects, and human patients to whom the rAAV vectors as described herein are administered may also benefit from adjunct or additional treatments, therapeutic compounds, drugs and/or surgical techniques, as are well known to those having skill in the art, to assist in or augment the therapy and treatment of the disease, disorder, or pathology.

### Kits

Also provided are kits for preventing or treating a neurological, neurodevelopmental, neurodegenerative, neuropsychiatric, neurogenetic, or neuromuscular disease, disorder, pathology, or condition, and/or the symptoms thereof in a subject in need thereof, including humans and non-human mammals. In one embodiment, the kit provides a therapeutic or prophylactic composition containing an effective amount of a rAAV vector or viral particle as described herein, which comprises an enhancer polynucleotide sequence specific for a given gene that restricts the expression of the gene, e.g., contained in the virus vector, to interneuron or neuron cells of certain types as described herein, including GABAergic interneuron cells in the brain (e.g., in the telecephalon), or to basal forebrain cells in the brain cortex. In an embodiment, the enhancer element is any one of mouse or human S9E1-S9E40 (SEQ ID NOs: 1-80). In embodiments, the enhancer is one or more of mouse or human S9E1-S9E5 (SEQ ID NO: 1-10, respectively), as described herein, which restricts expression of a transgene (e.g., a therapeutic gene, reporter gene, or an effector gene, e.g., a polynucleotide encoding a Gq-DREADD or PSAM for chemogenetic modulation of PV-interneuron activity, or genes or polynucleotides encoding CRISPR-Cas9, Zinc Finger Protein, TALENs, or engineered or variant forms thereof, to PV-interneuron cells (e.g., basket or chandelier PV interneurons). In an embodiment, the enhancer is one or more of mouse or human S9E6-S9E10 (SEQ ID NO: 11-20, respectively), as described herein, which restricts expression of a transgene to SST-interneuron cells. In an embodiment, the enhancer is one or more of mouse or human S9E11-S9E15 (SEQ ID NO: 21039, respectively), as described herein, which restricts expression of a transgene to VIP-interneuron cells. In an embodiment, the enhancer is one or more of S9E16-S9E20 (SEQ ID NO: 31-40, respectively), as described herein, which restricts expression of a transgene to non-VIP/CGE-derived interneurons (ID2-interneuron cells). In an embodiment, the enhancer is one or more of mouse or human S9E21 (SEQ ID NOs: 41 and 42), S9E25 (SEQ ID NOs: 43 and 44), S9E33 (SEQ ID NOs: 45 and 46), S9E34 (SEQ ID NOs: 47 and 48), and S9E36 (SEQ ID NOs: 49 and 50) as described herein, which restricts expression of a transgene to Dopamine-Receptor 1 (D1)-expressing medium-spiny neurons (Drd1 neuron cells). In an embodiment, the enhancer is one or more of mouse or human S9E22 (SEQ ID NOs: 51 and 52), S9E23 (SEQ ID NOs: 53 and 54), S9E24 (SEQ ID NOs: 55 and 56), S9E31 (SEQ ID NOs: 57 and 58), and S9E32 (SEQ ID NOs: 59 and 60), as described herein, which restricts expression of a transgene to Dopamine-Receptor 2 (D2)-expressing medium-spiny neurons (Drd2 neuron cells). In an embodiment, the enhancer is one or more of mouse or human S9E27 (SEQ ID NOs: 61 and 62), S9E28 (SEQ ID NOs: 63 and 64), S9E38 (SEQ ID NOs: 65 and 66), and S9E39 (SEQ ID NOs: 67 and 68), as described herein, which restricts expression of a transgene to Cholinergic interneurons. In an embodiment, the enhancer is one or more of mouse or human S9E26 (SEQ ID NOs: 69 and 70), S9E35 (SEQ ID NOs: 71 and 72), and S9E40 (SEQ ID NOs: 73 and 74), which restricts expression of a transgene to Cholinergic interneurons of the striatum (Ch-IN). In an embodiment, the enhancer is one or more of mouse or human S9E29 (SEQ ID NOs: 75 and 76), S9E30 (SEQ ID NOs: 77 and 78), and S9E37 (SEQ ID NOs: 79 and 80), which restricts expression of a transgene to Cholinergic projection neurons of the basal ganglia (Ch-PN). In an embodiment, the enhancer is S9E10 (SEQ ID NO: 19), which exhibited >85% specificity for SST interneurons in the cortex, or huS9E10 (SEQ ID NO: 20). In an embodiment, the enhancer is S9E27 (SEQ ID NO: 61), which exhibited >95% specificity for both Cholinergic interneurons in the striatum and Cholinergic projection neurons in the basal nuclei of the brain, or huS9E27 (SEQ ID NO: 62).

In some embodiments, the kit comprises a sterile container which contains the therapeutic or prophylactic composition; such containers can be boxes, ampoules, bottles, vials, tubes, bags, pouches, blister-packs, or other suitable container forms known in the art. The containers can be made of plastic, glass, laminated paper, metal foil, or other materials suitable for holding medicaments.

A composition comprising an rAAV vector comprising at least an enhancer polynucleotide sequence as described herein is provided together with instructions for administering the composition to a subject having or at risk of developing a neurological, neurodevelopmental, neurodegenerative, neuropsychiatric, neurogenetic, or neuromuscular disease, disorder, pathology, or condition, and/or the symptoms thereof. In an embodiment, the rAAV vector comprises a transgene (e.g., a therapeutic transgene) for expression in interneuron cells including inhibitory GABAergic interneurons and in neurons of the basal forebrain. The instructions will generally include information about the use of the composition for the treatment or prevention of the disease, disorder, pathology, and/or the symptoms thereof. In other embodiments, the instructions include at least one of the following: description of the therapeutic agent (rAAV comprising an enhancer polynucleotide sequence, etc.); dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Further Embodiments and Advantages Thereof

In other embodiments, the enhancers identified and described herein provide access to particular cell populations with distinct clinical relevance. By way of example, these enhancers be used to alleviate the debilitating aspects of a number of neurological, neurodevelopmental, neurodegenerative, neuropsychiatric, neurogenetic, or neuromuscular diseases, disorders, or pathologies, either through gene therapy or via modulation of neuronal activity, e.g., via optogenetic or chemogenetic approaches. (See, e.g., Walker, M. C. et al., 2019, Neuropharmacology, 107751. doi: 10.1016/j.neuropharm.2019.107751. Review. PMID: 31494141). As described and demonstrated herein, local and systemic injections can be used for effective viral delivery to the brain, thus providing delivery and administration methods for clinical interventions. By way of example, local injections (e.g., of recombinant virus carrying an enhancer element and target polynucleotide) may be employed to alleviate focal epilepsy, prefrontal cortex dysfunction or hippocampal memory disorders. Systemic administration or delivery of virus may be employed in contexts where global interventions are necessary, for example, to correct generalized seizures or for psychiatric and neurodegenerative disorders. As provided by the embodiments described and exemplified herein, the rigorous identification of enhancer elements (enhancer regulatory elements) allows for accessing target neuronal cell types. Such elements are advantageous for use in both experimental and therapeutic procedures and methods.

The practice of the described embodiments employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides, viral vectors and viral particles and, as such, may be considered in making and practicing the embodiments described herein. Particularly useful techniques for particular embodiments will be discussed in the sections that follow.

The following examples are put forth so as to provide those of ordinary skill in the art with a complete disclosure and description of how to make and use the products, compositions and therapeutic methods as described herein, and are not intended to limit the scope of what is described and exemplified herein.

### EXAMPLES

### Example 1—Identification of Cis-Regulatory Sequences that Restrict Expression of Reporter and Transgenes to Target Neuron and Interneuron Cell Populations

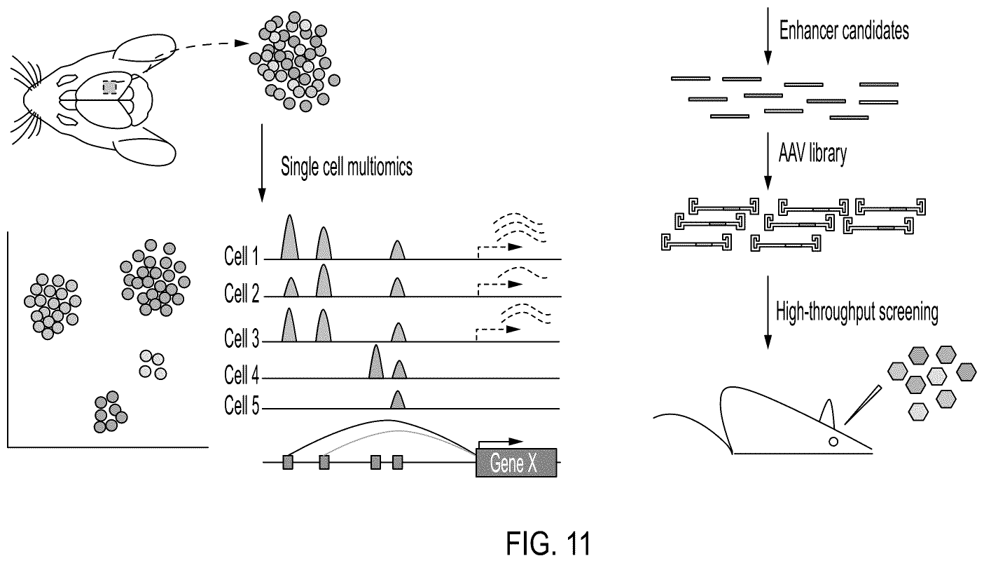

The 40 mouse and human enhancer sequences (SEQ ID NOS: 1-80) described herein were discovered and identified using an in-silico selection (D. Vormstein-Schneider et al., 2020, Nat. Neurosci., 23(12):1629-1636, incorporated by reference herein), which relies on combining chromatin accessibility data with cross-species conservation to identify putative enhancer regulatory elements in the vicinity of genes enriched in the target neuronal population. (FIG. 11). The application of the method led to the identification of candidate enhancer sequences for targeting at least eight neuronal cell populations, namely, four distinct classes of neurons and interneurons, i.e. parvalbumin-expressing interneurons (PV interneurons), e.g., PV+ Chandelier interneurons, Somatostatin expressing interneurons (SST interneurons), Vaso-active Intestinal Peptide expressing interneurons (VIP interneurons), and non-VIP/CGE-derived interneurons (ID2 interneurons); and four distinct classes of basal forebrain neurons (i.e. Dopamine-Receptor 1 expressing medium-spiny neurons (D1 or Drd1 neurons), Dopamine-Receptor 2 expressing medium-spiny neurons (D2 or Drd2 neurons), Cholinergic neurons, (e.g., ChAT neurons), Cholinergic interneurons of the striatum (Ch-IN interneurons), and Cholinergic projection neurons of the basal ganglia (Ch-PN neurons) in the central nervous system (CNS), including the brain. In addition, enhancer sequences that target the arkypallidal (ArkyP) neuronal cell population in the globus pallidus and that target neuronal cells in specific regions of the ventral CNS, including the global pallidus and thalamic and subthalamic structures were identified. The identified enhancer sequences were isolated and cloned into vectors for expression in target cells, such as neuronal cells, in particular, certain types, populations, or classes of neuronal cells as described supra.

To examine the ability of the candidate enhancers to target certain neuronal cell populations, the enhancer element sequences were individually inserted into a viral vector (rAAV)-backbone containing a minimal promoter upstream of a red fluorescent reporter transgene, e.g., d-Tomato to generate the rAAV vector called prAAV-Enhancer Sequence(E[x])-dTomato. From these constructs, rAAV particles were then produced with the PHPeB capsid (Chan, K. Y. et al., Nat. Neurosci., 20:1172-1179 (2017)). The ability of the enhancer sequences to restrict expression of the reporter gene (transgene) to target neurons and interneurons in brain was assessed by injecting each enhancer-containing rAAV vector systemically into animals (adult mice) and analyzing the co-localization between the expressed reporter across brain structures including the cortex. After 3 weeks, all virus vectors showed strong and sparse expression within the cortex, as well as across multiple brain regions, and drove expression of the transgene reporter harbored in the virus vector exclusively in the target neuronal/interneuronal populations.

Each of the enhancer elements was nominated in-silico as a candidate for an intended target neuronal cell population, e.g., S9E13 was nominated as a candidate to restrict expression to VIP interneurons. In some cases upon testing the enhancers in vivo, the quantification revealed that some enhancer elements also showed specificity for another neuronal or interneuronal cell population. By way of example, S9E13 showed specificity for PV rather than for VIP interneurons. Notwithstanding, all of the enhancers in FIGS. 1A and 1B are of interest in that they target different neuronal (or interneuronal) cells and cell populations. Among the forty mouse and human enhancer elements listed in FIGS. 1A and 1B, seven enhancer sequences in particular exhibited a high specificity for the intended target neuronal cell population, for example, S9E1-S9E5 exhibited specificity for PV interneurons; S9E10 exhibited specificity for SST interneurons; and S9E27 exhibited specificity for ChAT neurons. The products, compositions and methods described herein embrace the use of both mouse and human enhancer elements for targeting a gene (and/or regulating or modulating the expression of a gene and/or its encoded product) in an associated intended target cell or cell population.

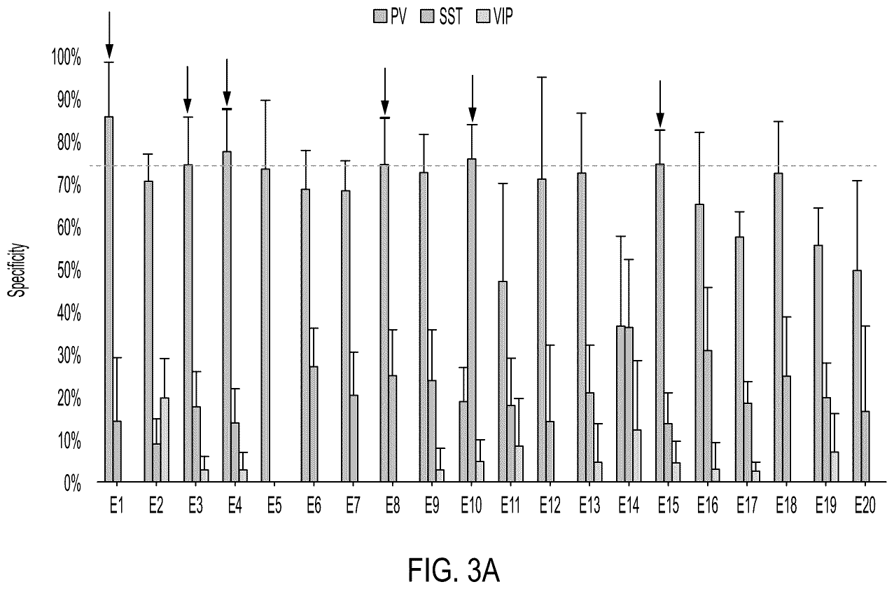

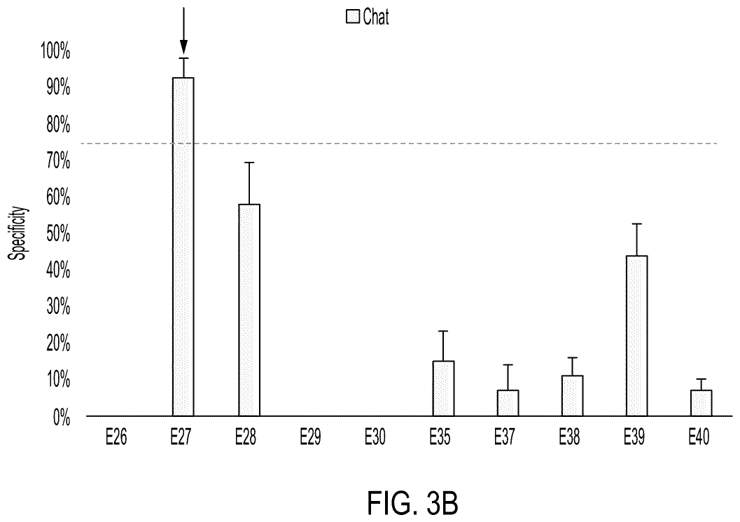

Quantification of the degree of specificity of expression of the reporter gene in PV-expressing interneurons, SST interneurons, or VIP interneurons is demonstrated graphically in FIG. 3A. Quantification of the degree of specificity of expression of the reporter gene in cholinergic/ChAT neurons in the cortex is demonstrated graphically in FIG. 3B. The expression of the reporter gene is controlled by the enhancer elements contained in rAAV vectors. The specificity was quantified as the proportion of cells expressing the viral reporter dTomato co-expressing the PV, SST, or VIP interneuron markers, or ChAT marker, assessed by immunohistochemistry on brain sections following systemic in vivo injection of each of the pAAV-[enhancer sequence]-dTomato vectors into an animal (mouse). Bar graphs represent mean+/−standard error of the mean. FIG. 3A shows the specificity of reporter gene expression in PV-expressing, SST-expressing, or VIP interneurons (GABA-ergic interneurons of brain cortex. FIG. 3B shows the specificity of reporter gene expression in ChAT Cholinergic neurons, such as Cholinergic interneurons of the striatum (Ch-IN) and Cholinergic projection neurons of the basal ganglia (Ch-PN). The results shown in FIGS. 3A and 3B were generated by systemic injection of 10E+11 viral particles containing an rAAV vector harboring the enhancer sequence and the transgene per adult mouse. Specificity is expressed as a percentage of the cellular population expressing the viral reporter that co-expressed a marker gene for the indicated target population. Arrows in black indicate enhancers with specificity of at least 75% for the target population. FIGS. 12A-12D demonstrate that the S9E10 enhancer harbored in an AAV delivery vector in conjunction at least one other gene, e.g., a reporter gene and/or an effector gene, e.g., SST, ChAT, TVA and Rabies virus G protein sequences, restricted gene expression in SST interneurons in cortical tissue.

Images showing the results of immunohistochemical (IHC) staining analysis for detection of the dTomato reporter expressed in brain sections following systemic in vivo injection of the pAAV-S9E1-dTomato vector, pAAV-S9E3-dTomato vector, pAAV-S9E4-dTomato vector, pAAV-S9E8-dTomato vector, or pAAV-S9E15-dTomato vector into an animal (mouse) allowing for detection of specific cells transduced by the vector The results are presented in FIG. 4, which demonstrates that the enhancers in the expression vectors specifically targeted and restricted expression of the dTomato reporter gene in interneurons expressing PV (PV-interneurons). Shown in FIG. 4 are images generated following the injection of vectors containing the S9E1 (SEQ ID NO: 1), S9E3 (SEQ ID NO: 5), S9E4 (SEQ ID NO: 7), S9E8 (SEQ ID NO: 15), S9E10 (SEQ ID NO: 19), S9E15 (SEQ ID NO: 29) and S9E27 (SEQ ID NO: 61) enhancer element sequences and the dTomato transgene reporter. In particular, FIG. 4 shows the results of systemic injection of 10E+11 viral particles per adult mouse. The images illustrate the specificity of expression for the target populations as show by immunohistochemistry for the viral reporter and the indicated marker gene for enhancers with specificity above 75%. All images show representative expression of the viral reporter encoded by the vector containing the enhancer sequence in the somatosensory cortex S1, except for the vector containing the S9E27 enhancer sequence and reporter-encoding polynucleotide, which shows staining in the striatum.

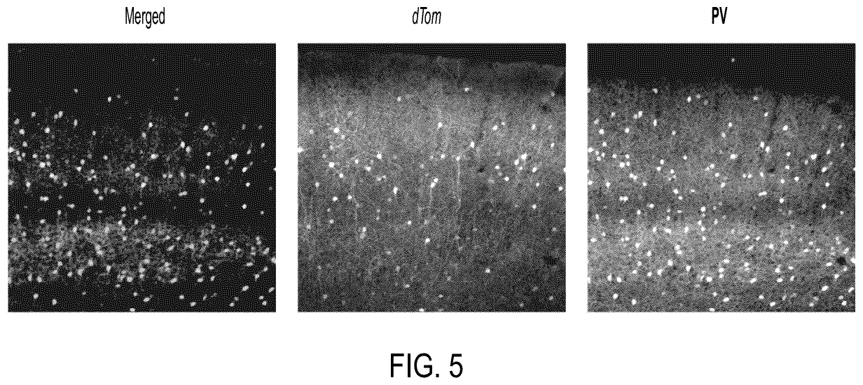

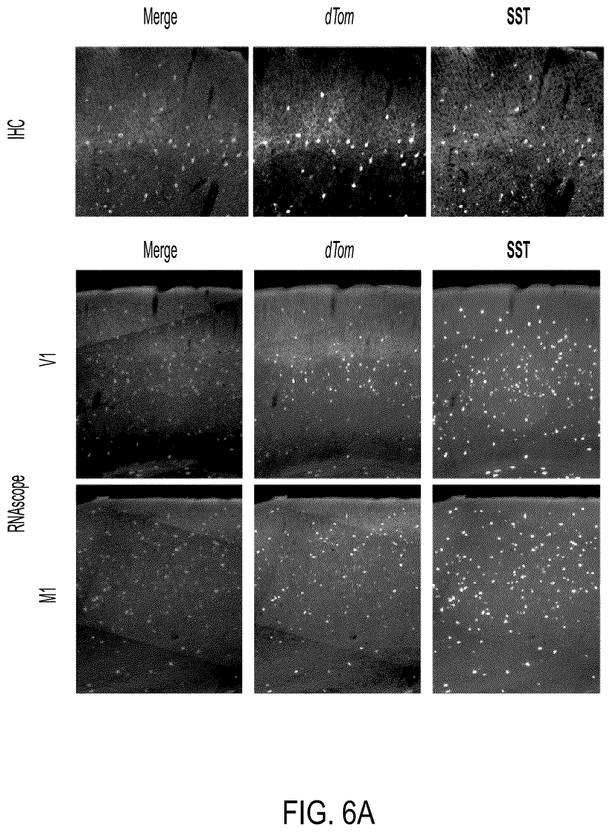

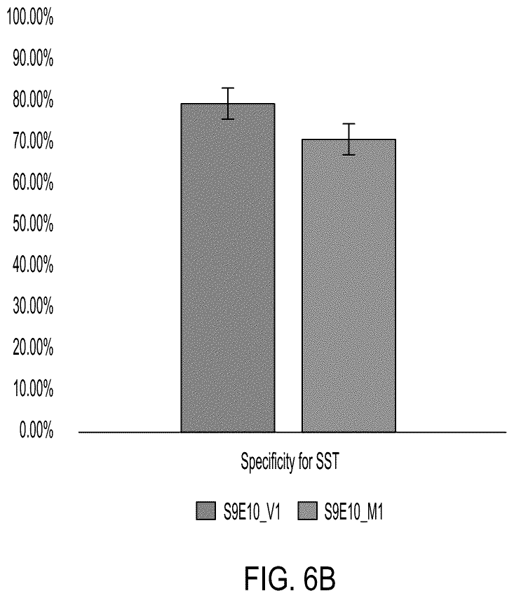

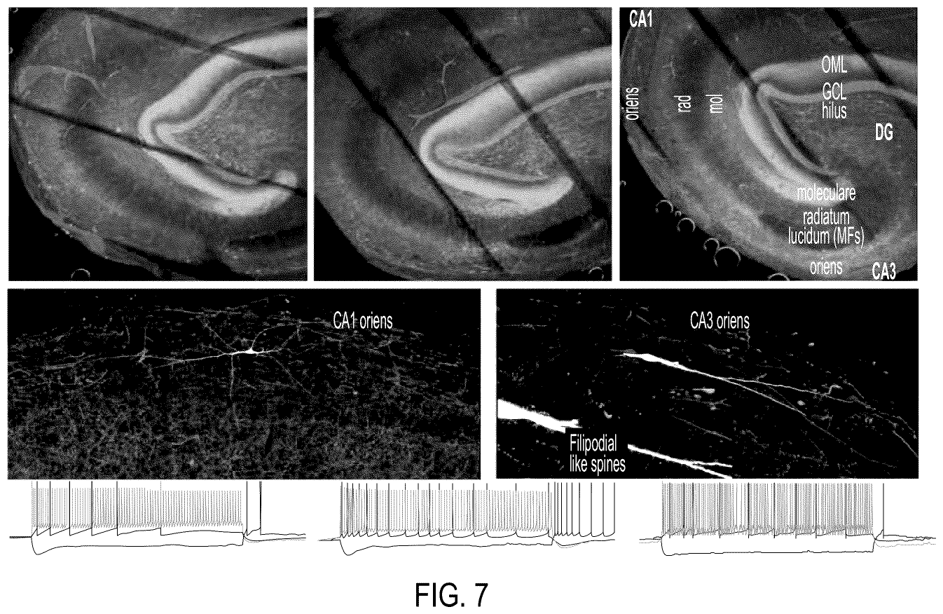

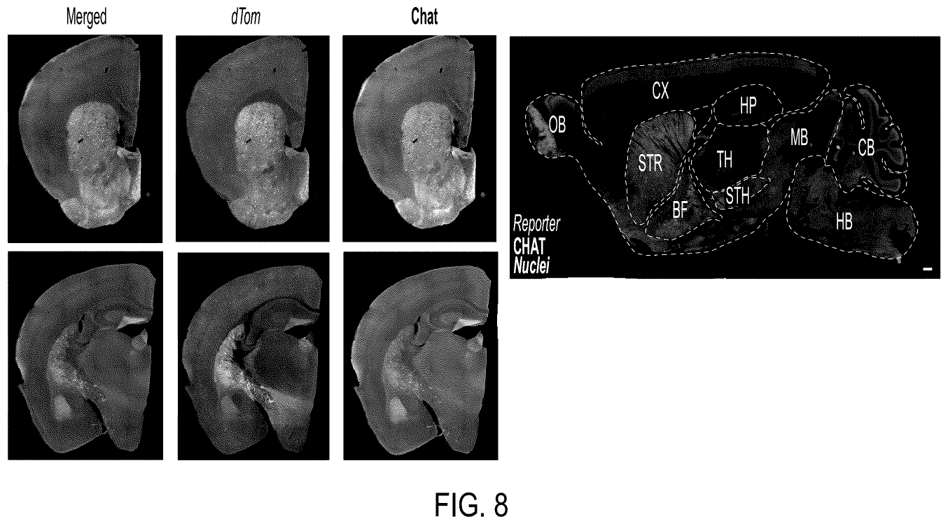

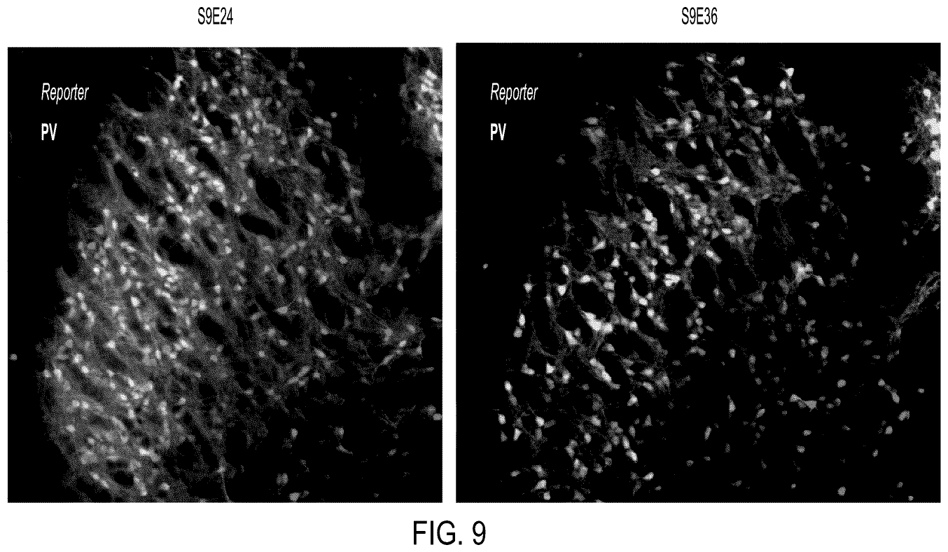

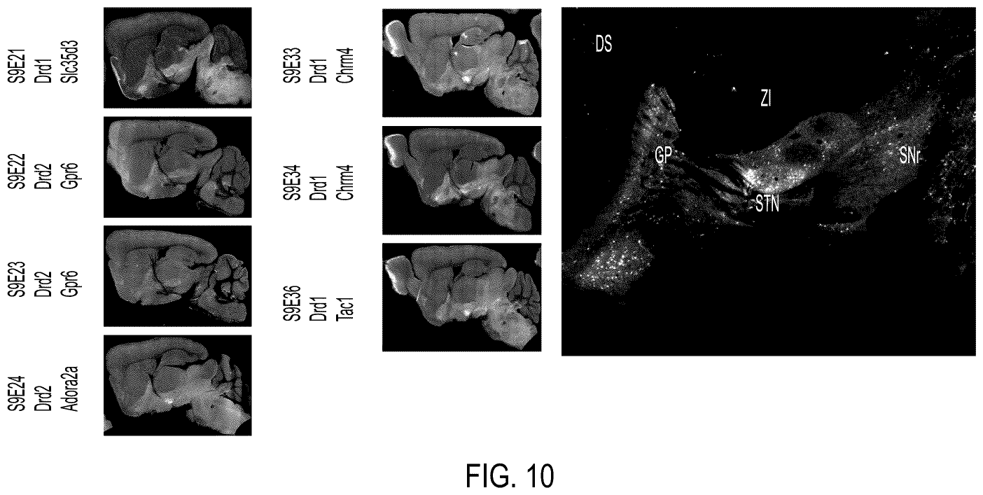

FIG. 5 shows the expression profile and specificity of the representative enhancer S9E2 in mice injected systemically with AAV-S9E2 using IHC with PV and dTomato markers. The high degree of colocalization and the layer specific distribution of cells expressing the viral reporter demonstrate that S9E2 is restricting expression of the reporter in PV+ Chandelier interneurons. FIGS. 6A and 6B show the expression profile and specificity of the representative enhancer S9E10 in mice injected systemically with AAV-S9E10 using IHC or RNAscope detection methods with the indicated markers. Quantification shows a high degree of specificity for SST-expressing interneurons (FIG. 6B). FIG. 7 shows reporter expression and electrophysiology profiling of cells expressing a viral vector (AAV) and reporter under the control of the S9E10 enhancer and shows SST firing patterns upon local injection of AAV-S9E10 in the hippocampus of a macaque. Electrophysiology recordings show stereotypical signature of SST cells, demonstrating that the specificity of the S9E10 is maintained for SST in non-human primates. FIG. 8 shows the expression profile and specificity of the representative S9E27 enhancer in mice injected systemically with AAV-S9E27 using IHC with the indicated markers. Quantification analysis in the cortex showed that >90% of cells expressing the viral reporter co-expressed the cholinergic marker ChAT. The high degree of colocalization and the layer specific distribution of cells expressing the viral reporter demonstrate that the S9E27 enhancer is restricting expression of the reporter in cholinergic neurons across brain structures. FIG. 9 shows the expression profile and specificity of the representative S9E24 and S9E36 enhancers in mice injected systemically with AAV-S9E24 or AAV-S9E36 using IHC with the indicated markers. The lack of co-localization of the reporter with PV shows that the targeted population corresponds to ArkyP projection neurons in the globus pallidus. FIG. 10 characterizes the indicated enhancers for their expression profiles across brain regions. Each enhancer can be observed to have a unique expression profile, showing strong expression in specific subsets of brain regions including the globus pallidus (GP), thalamic nuclei, Zona Inserta (ZI), Substantia Nigra (SNr) and subthalamic nuclei (STN). At left and center in FIG. 10, sagittal sections of mouse brains injected systemically with AAVs containing the indicated enhancers show the neuronal cell types, e.g., Drd1 or Drd2, and the associated genes expressed in these cell types as described supra. At right in FIG. 10 is a representative image of the expression profiles of these enhancers in the indicated regions (e.g., dorsal striatum (DS), globus pallidus (GP), thalamic nuclei, zona inserta (ZI), substantia nigra (SNr) and subthalamic nuclei (STN)).

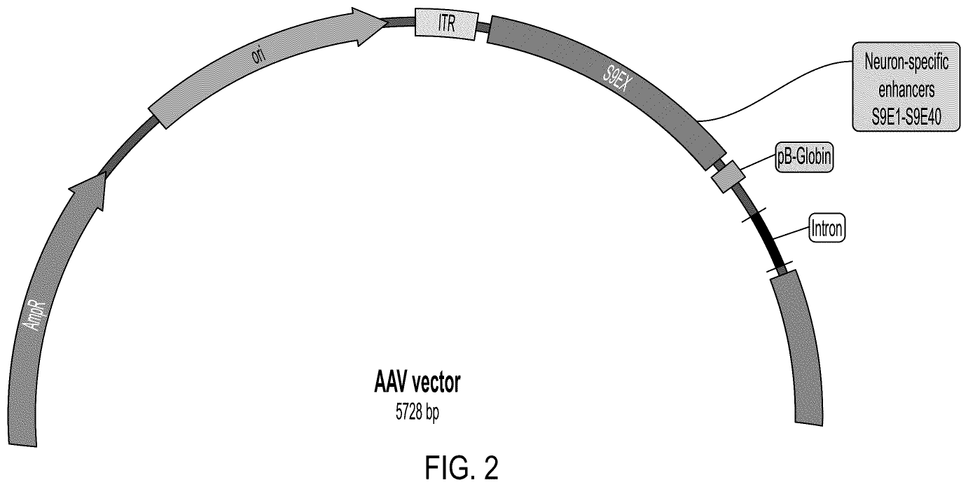

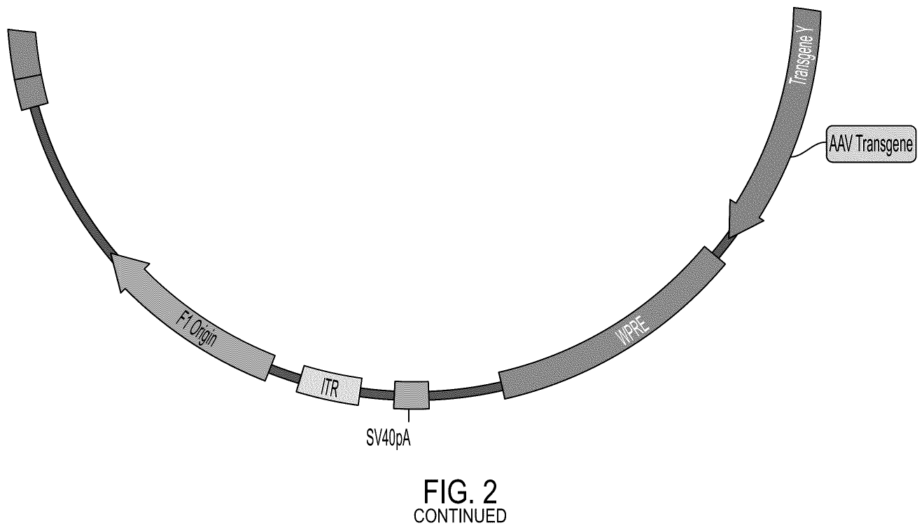

These enhancer regulatory elements account for largely non-overlapping expression in populations of interneurons and neurons with distinct functions and developmental origins. The viral tools developed as described herein provide a means for dissecting neuronal subtypes and can be advantageously used to study their normal function, as well as abnormalities in diseased cortex. The enhancer element sequences can be used to restrict expression of transgenes in particular neuronal and interneuronal cell types and/or populations as described herein. In embodiments, viral vectors are molecularly created to contain the enhancer elements and transgenes, among other functional sequences, for expression in the particular cell types. See, e.g., FIG. 2.

### Example 2—Viral Targeting of Cortical Neurons and Interneurons in Mice

The enhancer elements that target PV-expressing interneurons (e.g., mouse and human S9E1-S9E5) provide agents for use in targeting fast-spiking neurons (e.g., basket and chandelier cells), which collectively constitute 40% of all cortical (GABAergic) interneurons. These neurons exert a strong level of inhibition over local networks, and their dysfunction has been directly implicated in neurological and neuropsychiatric disorders, such as neuropsychiatric disorders and the symptoms thereof, e.g., seizures, schizophrenia, or bipolar disorders. Other enhancer elements are provided that target other types of neurons and interneurons, for example, as shown in FIGS. 1A and 1B.

Adult mice systemically-injected with rAAV-Enhancer Element-dTomato vectors showed detectable expression of the viral reporter after one week and reached a high and stable level of expression after 3 weeks. In particular, the S9E2 enhancer element was shown to have specificity for targeting PV-expressing Chandelier interneurons. (FIG. 5). In addition, the S9E10 enhancer element was shown to have a high (e.g., >70%) specificity for targeting SST interneurons in the brain cortex of both mice and macaques. (FIGS. 6A, 6B; FIG. 7; and FIGS. 12A-12D; FIG. 14 and FIG. 15). The results of optogenetic and chemogenetic studies using constructs containing the isolated SST-expressing neuronal cell enhancer S9E10 sequence and other genes are shown in FIG. 13A (optogenetic studies using the ChR2 gene in the viral construct) and FIG. 13B (chemogenetic studies using a Gq-DREADD gene in the viral construct). The S9E10 enhancer element sequence may be advantageous for clinical intervention to reduce seizures and associated neuropsychiatric disorders, e.g., schizophrenia.

In addition, as shown by quantification analysis in the cortex, in mice systemically injected with AAV containing the S9E27 enhancer and reporter gene, >90% of cells expressing the viral reporter co-expressed the cholinergic marker ChAT. (FIG. 8). The high degree of colocalization and the layer specific distribution of cells expressing the viral reporter showed that S9E27 restricted expression of cholinergic neurons across brain structures, as observed in the sagittal section of FIG. 8. FIGS. 16, 17A and 17B, 18, and 19 demonstrate the specificity of the isolated enhancer sequence S9E27 in targeting and restricting gene expression in ChAT-expressing neurons in mice and macaques, in particular, when the ChAT-expressing neurons are transduced with a viral vector containing the isolated S9E27 enhancer sequence and one or more polynucleotide (gene) sequences as shown. ChAT-expressing neurons targeted by the isolated S9E27 enhancer element sequence in vectors containing other polynucleotide (gene) sequences, e.g., ChR2 and Gq-DREADD, show responsiveness to stimuli in optogenetic and chemogenetic studies requiring expression of such polynucleotide sequences in the ChAT neurons. (FIGS. 17A and 17B). In addition, FIGS. 20A-20D show results of anatomical studies in mice using the isolated S9E27 enhancer element as a component of a recombinant AAV construct (AAVS9E27-ChR2-mCherry) in which co-localization of the enhancer with ChAT-specific staining of basal forebrain neurons as well as axons in the auditory cortex in mice were shown. In addition, cholinergic currents were recorded in auditory cortical neurons following transduction of the ChAT neurons targeted via the use of the isolated S9E27 enhancer sequence/enhancer construct.

The targeting of SST interneurons in the cortex by the S9E10 enhancer and targeting PV+ Chandelier interneurons by the S9E2 enhancer, by way of nonlimiting example, may provide beneficial uses for clinical intervention to reduce seizures and associated neuropsychiatric disorders, as well as to be therapeutically effective in cognitive disorders and cognition. Targeting neuronal cell populations, such as cholinergic interneurons in the striatum and cholinergic projection neurons in the basal nuclei by the S9E27 enhancer, for example, offers therapeutic and clinical intervention for treating neurological and neurodegenerative disorders, including, but not limited to, Alzheimer's disease, Parkinson's disease, Dystonia, ALS and Down Syndrome, and/or the symptoms thereof. This enhancer may also be beneficial in the context of treatment and therapy for movement disorders, including, without limitation, dystonia, tremors, ataxia, Essential Tremor, Lewy Body dementia, and motor stereotypies, and/or the symptoms thereof.

In addition, the S9E24 and S9E36 enhancer elements were shown to have specificity for targeting arkypallidal (ArkyP) projection neurons in the globus pallidus following systemic injection in mice. (FIG. 9). Furthermore, the S9E21, S9E22, S9E23, S9E24, S9E33, S9E34 and S9E36 enhancer elements, by way of nonlimiting example, were shown to have unique expression and targeting profiles in the globus pallidus and in thalamic/subthalamic structures in the brain. (FIG. 10). These enhancer elements may be advantageous for clinical intervention to alleviate neuronal disorders and movement disorders, including, but not limited to, ataxia, dystonia, tremors, Essential Tremor, Lewy Body dementia, motor stereotypies and Parkinson's Disease, as well as obsessive-compulsive disorder (OCD), epilepsy, and/or the symptoms thereof.

### Example 3—Materials and Methods of the Above-Described Examples

Methodology overview. The identification and isolation of cell-type specific enhancers was carried out as depicted in FIG. 11. Briefly, cells isolated from the mouse cerebral cortex were subjected to single cell multiomics to identify candidate enhancer element sequences in regulatory regions of genes expressed in neuronal cells, or specific types of neuronal cells. The various enhancer element candidates were cloned into an adeno-associated virus vector library and subjected to high throughput screening to identify those enhancer sequences that were specifically expressed in certain types of neuronal cells.

Enhancer selection. All enhancers presented herein were selected based on the co-presence of ATACseq data (for DNA accessibility) and conservation across species (using UCSC genome browser vertebrate conservation track). The genomic coordinates for mouse and human enhancer sequences are presented in FIGS. 1A and 1B.

For Selection, candidate enhancer regulatory elements were manually curated from a list of elements generated by intersecting the “context” region with both the “ATAseq peak union” file and the “Phastcons 60-way” file—see below. Accessibility. ATAC-seq data (Mo et al., 2015, Neuron, 86:1369-1384) were downloaded on the GEO repository and discretized as peaks using MACS2 ran with default parameters (https://github.com/taoliu/MACS). Using a custom R script, a file containing the union of all peaks across datasets was generated and used for enhancer selection as described below. The final selection relied on the inspection of the peaks for individual cell-types rather than on the union of all peaks. Methylation. Mouse mCH levels for non-overlapping 100 kb bins across the entire genome for mouse (Luo et al., 2018, Nat Commun, 9(1):3824) were downloaded from Brainome portal (http://brainome.org). These data were used as a confirmation for the positioning of the candidates selected using the ATAC-seq dataset described above. Conservation. The “phascons 60-way” track was downloaded from the UCSC portal (https://genome.ucsc.edu) in BED file format and filtered using a custom R script to remove any element smaller than 10 bp and fuse any element separated by less than 50 bp using Bedtools/Intersect.

rAAV cloning and viral production. All viral constructs were generated using standard cloning methods and protocols in molecular biology. The plasmid pAAV-mDlx-GFP (Addgene #83900; Addgene, Watertown, MA), (Dimidschstein, J. et al., 2016, Nat. Neuroscience, 19(12):1743-1749) was used to create a standard backbone containing the elements necessary for the production of AAVs (internal terminal repeats, minimal promoter, woodchuck posttranscriptional response element).

The enhancer sequences (necessary for restricting expression to intended neuronal cell types) were synthesized de novo by Genewiz (Cambridge, MA) and the reporters and effectors were amplified by PCR. In particular, the enhancer sequences were amplified by PCR from mouse genomic DNA using primers. By way of example, the primers for the enhancer element sequences contained the first and last 20 base pairs of each of the mouse or human enhancer sequences described herein. (FIGS. 1A and B). The enhancers, reporters and effectors were cloned using the Gibson Cloning Assembly Kit (NEB-E5510S) following standard procedures.

Final plasmids were assembled using the Gibson Assembly® Cloning Kit (NEB-E5510S), (New England BioLabs, Ipswich, MA), following the manufacturer's instructions and standard protocol. The rAAVs were produced using standard production methods. Polyethylenimine (PEI) was used for transfection (see. e.g., Longo, P. A. et al., 2013, Methods Enzymol., 529:227-240) and OptiPrep™ density gradient (Sigma-Aldrich, St. Louis, MO) was used for viral particle purification and isolation. Serotype 1 was used to produce the AAVs for local injections in mice. Serotype PHPeB was used for systemic injections in mice. Viral titer was estimated by qPCR with primers annealing via the WPRE sequence that is common to all constructs. All batches produced were in the range of 10¹⁰ to 10¹² viral genomes per ml. In particular, Woodchuck Hepatitis Virus (WHP) Posttranscriptional Regulatory Element (WPRE) is a DNA sequence, which, when transcribed, creates a tertiary structure enhancing expression. WPRE, a tripartite regulatory element with gamma, alpha, and beta components, is commonly used in molecular biology to increase expression of genes delivered by viral vectors, e.g., rAAV-dTomato. (see, e.g., Choi, J.-H. et al., 2014, Mol. Brain, 7:17). All rAAV batches produced were in the range of 10¹⁰ to 10¹² viral genomes per ml.

Animals. Mice: Female C57BL/6J mice (Mus musculus; 10 weeks old) were obtained from Jackson Labs (Bar Harbor, ME—stock #000664). All animals were maintained in a 12 light/12 dark cycle with a maximum of five animals per cage for mice and one animal per cage for rats. All animal maintenance and experimental procedures were performed according to the guidelines established by the Institutional Animal Care and Use Committee at the Broad Institute of MIT and Harvard (mice),

Mouse systemic injection. For systemic injection in adult mice, approximately 10¹¹ viral particles were injected in the retro-orbital sinus per animal. Post-operative monitoring was performed for five days post injection.

Surgery. For retro-orbital vein injection, animals were anesthetized under isoflurane (1-3% in oxygen) and placed on a temperature-controlled heating pad. Intravenous (IV) injections were performed in the retro-orbital plexus. In particular, the animal (mouse) was placed in a funnel-shaped nose cone connected to a non-rebreathing apparatus (Surgivet, Dublin, OH) and the needle was inserted, bevel down, at the medial canthus, into the retroorbital sinus. Up to 150 μL of supernatant containing replication-defective rAAV vectors were injected into the tail vein or retro-orbital plexus. Following injection, the eye was held shut for a minimum of 30 seconds to ensure homeostasis.

Immunohistochemistry (IHC). Animals injected with the virus were euthanized with Euthasol (Virbac, USA) and transcardially perfused with 4% paraformaldehyde (PFA). The brains were placed in 4% PFA overnight, and then were sectioned at 50-60 μm (in particular, 50 μm) using a Leica VTS1000 vibrosector. Floating brain sections were permeabilized with 0.1% Triton X-100 and phosphate buffered saline (PBS) for 30 minutes, washed three times with PBS, and incubated in blocking buffer (5% normal donkey serum in PBS) for 30 minutes. The sections were then incubated overnight in blocking buffer with the indicated combinations of the following primary antibodies at 4° C.: chicken anti-GFP at 1:1,000 (Abcam USA, ab13970); rabbit anti-DsRed at 1:1000 (Clontech USA 632496); goat anti-PV at 1:1,000 (Swant USA, PVG-213); guinea-pig anti-PV at 1:1,000 (Swant USA, GP-72); rabbit anti-SST at 1:2000 (Peninsula USA, T-4103.0050); anti-ChAT antibody at 1:1000 (AB144P; Millipore). The sections were then washed three times with PBS incubated with Alexa Fluor-conjugated secondary antibodies at 1:1000 (Invitrogen, USA), counterstained with DAPI (Sigma, USA) and mounted on glass slides using Fluoromount-G (Sigma, USA). Images of brain regions were acquired using a Zeiss LSM800 confocal microscope or a Zeiss Axioimager A1 epifluorescence microscope.

In situ hybridization. RNASCOPE® in situ hybridization (ISH) technology was used for the detection of target RNA within intact tissues or cultured cells with single-cell resolution. RNASCOPE® allows for the visualization of expression of allele specific oligonucleotides (ASO), miRNA, siRNA, and other nucleic acid targets between 17-50 nucleotides. The in-situ hybridization probes (Gadi; product #400951, Pvalb; product #421931, VIP; product #415961) used in the studies described herein were designed by Advanced Cell Diagnostics (Newark, CA, USA). The reagents in the RNASCOPE® Multiplex Fluorescent Reagent Kit v2 (product #323100), RNASCOPE® Probe Diluent (product #300041), HYBEZ™ oven (product #321710/321720), humidity control tray (product #310012), and HYBEZ Humidifying Paper (product #310025) were also from Advanced Cell Diagnostics. TSA Plus Fluorescein, TSA Plus Cyanine 3, and TSA Plus Cyanine 5 from PerkinElmer (#NEL741. #NEL744, and #NEL745). Brain tissue was processed as mentioned in the immunohistochemistry section supra. Brain sections were washed one time in PBS followed by three washes in 0.1% Triton X-100 and PBS, mounted on Superfrost Plus glass slides (Fisher Scientific, 12-550-15) and baked at 60° C. in the HYBEZ oven for 25 minutes. The slides were then submerged in 4% PFA for 30 minutes then washed 3 times in H₂O. RNASCOPE® H₂O₂ was applied to each section for 5 minutes at room temperature. The slides were then washed 3 times in H₂O before being submerged in pre-warmed 90° C. H₂O for 15 seconds, followed by pre-warmed 90° C. RNASCOPE® Target Retrieval for 15 minutes. Slides were washed 3 times in H₂O before RNASCOPE®?; Protease III was applied onto each section and then incubated for 15 minutes at 40° C. in the HYBEZ oven. Slides were washed 3 times in H₂O and then were incubated with probe solution diluted to 1:50 with probe diluent for 2 hours at 40° C. in HYBEZ oven. Next, the sections were washed three times in RNASCOPE® wash buffer followed by fluorescence amplification. Of note, probes against the RNA of the reporter revealed a non-specific staining that was likely attributed to the viral DNA. To reveal the viral reporter, the RNASCOPE® protocol was performed with an IHC amplification of the dTomato. The sections were incubated in blocking solution (0.3% Triton X-100 plus 5% normal horse serum in PBS) for 30 minutes. Following this, sections were incubated in antibody solution (0.1% Triton X-100 plus 5% normal horse serum in PBS) with rabbit anti-DsRed at 1:250 (Clontech USA 632496) at 4° C. overnight. The sections were then washed three times with PBS, incubated with Alexa Fluor-conjugated secondary antibodies at 1:500 (Invitrogen, USA), counterstained with DAPI (Sigma, USA) and mounted on glass slides using Fluoromount-G (Sigma, USA).

Quantification and statistics. For strength of expression, fluorescence images were taken at a standardized magnification and exposure time. The average pixel intensity of the cell bodies of each cell expressing the viral reporter was recorded and reported as an average over all cells per enhancer. For quantification of co-localization, cells expressing the indicated reporter were counted using only the corresponding color channel, and then, among these cells, the number of cells co-expressing the marker of interest was counted. A cell was considered to be positive for a given marker if the corresponding signal was above background fluorescence. The ratio of cells co-expressing both markers over the total number of cells expressing only the reporter was then calculated, reported herein as mean±s.e.m (represented as bar plots in figures herein, for example). Quantifications were performed using a minimum of two independent biological replicates. Several sections from the same animal were used when indicated. Data collection and analysis were not performed blind to the conditions of the experiments, but experimenters from different research groups performed the quantifications. No statistical methods were used to predetermine sample sizes, but the sample sizes described were similar to those reported in previous publications.

### OTHER EMBODIMENTS

From the foregoing description, it will be apparent that variations and modifications may be made to the embodiments described herein to adopt them to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof, such as described in one or more sections herein.

All patents and publications mentioned in this specification are herein incorporated by reference in their entireties to the same extent as if each independent patent and publication was specifically and individually indicated to be incorporated by reference.

What is claimed is:

1. An isolated, cloned enhancer element comprising a polynucleotide sequence having at least 75%, 85%, 90, 95% identity to a polynucleotide sequence set forth in any one of SEQ ID NOs: 3, 4, 19, 20, 41, 42, 45, 46, 47, 48, 49, 50, 51, 52, 53, 54, 55, 56, 61, or 62, wherein the enhancer element regulates or restricts expression of a gene in a neuronal cell type or population.

2. The enhancer element of claim 1, wherein the neuronal cell type or population is selected from inhibitory GABA-ergic neurons or basal forebrain neurons.

3. The enhancer element of claim 1, wherein inhibitory GABA-ergic neurons are selected from parvalbumin (PV)-expressing Chandelier interneurons, arkypallidal (ArkyP) neurons, or Somatostatin (SST)-expressing interneurons; and basal forebrain neurons are selected from Dopamine-Receptor 1 (Drd1)-expressing neurons; Dopamine-Receptor 2 (Drd2)-expressing neurons; or cholinergic (ChAT) neurons.

4. The enhancer element of claim 1, wherein the enhancer element comprises any one of S9E10 (SEQ ID NO: 19) or huS9E10 (SEQ ID NO: 20) having specificity for somatostatin (SST) neurons; S9E36 (SEQ ID NO: 49) or huS9E36 (SEQ ID NO: 50) having specificity for Arkypalladial (ArkyP) neuronal cell populations in the globus pallidus; S9E24 (SEQ ID NO: 55) or huS9E24 (SEQ ID NO: 56) having specificity for Arkypalladial (ArkyP) neuronal cell populations in the globus pallidus; S9E2 (SEQ ID NO: 3) or huS9E2 (SEQ ID NO: 4) having specificity for PV-expressing Chandelier interneurons; or
wherein the enhancer element comprises any one of S9E21 (SEQ ID NO: 41) or huS9E21 (SEQ ID NO: 42); S9E33 (SEQ ID NO: 45) or huS9E33 (SEQ ID NO: 46); or S9E34 (SEQ ID NO: 47) or huS9E34 (SEQ ID NO: 48) having specificity for Dopamine-Receptor 1 (Drd1)-expressing neurons; or S9E22 (SEQ ID NO: 51) or huS9E22 (SEQ ID NO: 52); S9E23 (SEQ ID NO: 53) or huS9E23 (SEQ ID NO: 54) having specificity for Dopamine-Receptor 2 (Drd2)-expressing neurons; or S9E27 (SEQ ID NO: 61) or huS9E27 (SEQ ID NO: 62) having specificity for cholinergic (ChAT) neurons.

5. An isolated, cloned enhancer element comprising:
a polynucleotide sequence of S9E27 (SEQ ID NO: 61) or huS9E27 (SEQ ID NO: 62), which targets ChAT cholinergic neurons in the brain;
a polynucleotide sequence of S9E10 (SEQ ID NO: 19) or huS9E10 (SEQ ID NO: 20) which targets SST interneurons in the brain;
a polynucleotide sequence of S9E36 (SEQ ID NO: 49) or huS9E36 (SEQ ID NO: 50); or
S9E24 (SEQ ID NO: 55) or huS9E24 (SEQ ID NO: 56), which targets Arkypalladial (ArkyP) neuronal cell populations in the globus pallidus;
a polynucleotide sequence of S9E21 (SEQ ID NO: 41) or huS9E21 (SEQ ID NO: 42);
S9E33 (SEQ ID NO: 45) or huS9E33 (SEQ ID NO: 46); or S9E34 (SEQ ID NO: 47) or huS9E34 (SEQ ID NO: 48), which targets Dopamine-Receptor 1 (Drd1)-expressing neurons;
a polynucleotide sequence of S9E22 (SEQ ID NO: 51) or huS9E22 (SEQ ID NO: 52); or
S9E23 (SEQ ID NO: 53) or huS9E23 (SEQ ID NO: 54) which targets Dopamine-Receptor 2 (Drd2)-expressing neurons; or
a polynucleotide sequence of S9E2 (SEQ ID NO: 3) or huS9E2 (SEQ ID NO: 4) which targets PV-expressing Chandelier interneurons in the brain cortex.

6. A viral vector comprising the enhancer element of claim 1.

7. The viral vector of claim 6, wherein the enhancer element comprises:
S9E10 (SEQ ID NO: 19) or huS9E10 (SEQ ID NO: 20) having at least 85% specificity for SST interneurons in the brain cortex;
S9E27 (SEQ ID NO: 61) or huS9E27 (SEQ ID NO: 62) having at least 90% specificity for CHAT cholinergic interneurons in the striatum of the brain and cholinergic projection neurons in the basal nuclei of the brain;
S9E36 (SEQ ID NO: 49) or huS9E36 (SEQ ID NO: 50); or S9E24 (SEQ ID NO: 55) or huS9E24 (SEQ ID NO: 56), which targets Arkypalladial (ArkyP) neuronal cell populations in the globus pallidus of the brain;
S9E2 (SEQ ID NO: 3) or huS9E2 (SEQ ID NO: 4) having at least 70% specificity for parvalbumin (PV)-expressing Chandelier interneurons in the brain;

8. The viral vector of claim 7, wherein the viral vector comprises a transgene that is a reporter gene, a therapeutic gene encoding a therapeutically or enzymatically active polypeptide, or an effector gene or polynucleotide.

9. The viral vector of claim 7, wherein the viral vector is a lentivirus vector, an adeno-associated virus (AAV) vector, or a recombinant adeno-associated virus (rAAV) vector.

10. A viral particle or virus-like particle comprising the viral vector of claim 7.

11. A cell comprising the viral vector of claim 7.

12. A pharmaceutical composition comprising the viral vector of claim 7, and a pharmaceutically acceptable vehicle, carrier, or diluent.

13. A method of restoring normal levels of target gene expression in GABA-ergic neuronal cells in which expression levels of the gene are deficient or defective, the method comprising contacting the cells with an effective amount of the viral vector of claim 7 or a pharmaceutical composition thereof, to restore normal levels of expression of the target gene in the GABAergic neuronal cells.

14. A method of restoring normal levels of target gene expression in CHAT Cholinergic (ChAT) neurons, Cholinergic interneurons in the striatum, and Cholinergic projection neurons in which expression levels of the gene are deficient or defective, the method comprising contacting the cells with an effective amount of the viral vector of claim 7, a viral particle, a virus-like particle, or a pharmaceutical composition thereof, to restore normal levels of expression of the target gene in the Cholinergic neurons (Chat neurons), Cholinergic interneurons in the striatum and Cholinergic projection neurons.

15. A method of delivering a transgene for restricted expression in an inhibitory GABA-ergic neuronal cell, the method comprising: contacting a neuronal cell with a recombinant adeno-associated virus (rAAV) vector, a viral particle, a virus-like particle, or a pharmaceutical composition thereof, comprising the transgene polynucleotide sequence, or a functional portion thereof, and an enhancer element polynucleotide sequence selected from S9E10 (SEQ ID NO: 19) or huS9E10 (SEQ ID NO: 20); S9E36 (SEQ ID NO: 49) or huS9E36 (SEQ ID NO: 50); S9E24 (SEQ ID NO: 55) or huS9E24 (SEQ ID NO: 56); or S9E2 (SEQ ID NO: 3) or huS9E2 (SEQ ID NO: 4) that restricts expression of the transgene in target GABA-ergic neuron cells of the cerebral cortex of the subject, thereby delivering the transgene to the GABA-ergic neuronal cell in the subject.

16. A method of delivering a transgene for restricted expression in a basal forebrain neuron, the method comprising: contacting the neuron with a recombinant adeno-associated virus (rAAV) vector, a viral particle, a virus-like particle, or a pharmaceutical composition thereof, comprising the transgene polynucleotide sequence, or a functional portion thereof, and an enhancer element polynucleotide sequence that restricts expression of the transgene in basal forebrain neurons of the subject, thereby delivering the transgene to basal forebrain neurons in the subject.

17. The method of claim 16, wherein the basal forebrain neuron is selected from a Dopamine-Receptor 1 (D1)-expressing medium-spiny neuron (Drd1), a Dopamine-Receptor 2 (D2)-expressing medium-spiny neuron (Drd2), or a Cholinergic (ChAT) neuron (CHAT) and the enhancer element is selected from at least one of S9E27 (SEQ ID NO: 61) or huS9E27 (SEQ ID NO: 62); S9E21 (SEQ ID NO: 41) or huS9E21 (SEQ ID NO: 42); S9E33 (SEQ ID NO: 45) or huS9E33 (SEQ ID NO: 46); or S9E34 (SEQ ID NO: 47) or huS9E34 (SEQ ID NO: 48); or S9E22 (SEQ ID NO: 51) or huS9E22 (SEQ ID NO: 52); or S9E23 (SEQ ID NO: 53) or huS9E23 (SEQ ID NO: 54).

18. A viral vector comprising an enhancer polynucleotide sequence selected from S9E10 (SEQ ID NO: 19) or huS9E10 (SEQ ID NO: 20); S9E36 (SEQ ID NO: 49) or huS9E36 (SEQ ID NO: 50); S9E24 (SEQ ID NO: 55) or huS9E24 (SEQ ID NO: 56); or S9E2 (SEQ ID NO: 3) or huS9E2 (SEQ ID NO: 4), or a functional portion thereof, and a transgene for expression in GABA-ergic interneurons of the brain cortex, wherein the vector specifically targets the GABA-ergic interneurons of the brain cortex and delivers the transgene thereto, wherein the enhancer polynucleotide sequence is S9E10 (SEQ ID NO: 19) or huS9E10 (SEQ ID NO: 20) or a functional portion thereof.

19. A viral vector comprising isolated enhancer polynucleotide sequence selected from S9E27 (SEQ ID NO: 61) or huS9E27 (SEQ ID NO: 62), or a functional portion thereof, and a transgene for expression in Cholinergic neurons, Cholinergic interneurons in the striatum, or Cholinergic projection neurons in the basal nuclei, wherein the vector specifically targets the Cholinergic neurons, Cholinergic interneurons in the striatum, or Cholinergic projection neurons in the basal nuclei and delivers the transgene thereto.

20. A method of treating, abating, or ameliorating a neurological, neurodevelopmental, neurodegenerative, neuropsychiatric, neurogenetic, or neuromuscular, disease, disorder, or pathology, and/or the symptoms thereof, in a subject, comprising administering to a subject in need thereof an effective amount a viral vector comprising an enhancer of claim 1, and a transgene.

21. The method of claim 20, wherein the disease, disorder, or pathology is one or more of a neuropsychiatric disorder, cognition, seizure, ataxia, dystonia, tremors, Essential Tremor, Lewy Body dementia, motor stereotypies and Parkinson's Disease, obsessive-compulsive disorder

22. An isolated, cloned enhancer element comprising:
a polynucleotide sequence of SEQ ID NO: 19 or 20, or a viral vector comprising the enhancer element, or a functional portion thereof, wherein the enhancer element targets SST interneurons expressing Hpse;
a polynucleotide sequence of SEQ ID NO: 41 or 42, or a viral vector comprising the enhancer element, or a functional portion thereof, wherein the enhancer element targets Dopamine-Receptor 1 (D1)-expressing medium-spiny neurons (Drd1 neurons) expressing Slc35d3;
a polynucleotide sequence of any one of SEQ ID NOs: 45-48, or a viral vector comprising the enhancer element, or a functional portion thereof, wherein the enhancer element targets Dopamine-Receptor 1 (D1)-expressing medium-spiny neurons (Drd1 neurons) expressing Chrm4;
a polynucleotide sequence of SEQ ID NO: 49 or 50, or a viral vector comprising the enhancer element, or a functional portion thereof, wherein the enhancer element targets Dopamine-Receptor 1 (D1)-expressing medium-spiny neurons (Drd1 neurons) expressing Tac1.
a polynucleotide sequence of SEQ ID NO: 55 or 56, or a viral vector comprising the enhancer element, or a functional portion thereof, wherein the enhancer element targets Dopamine-Receptor 2 (D2)-expressing medium-spiny neurons (Drd2 neurons) expressing Adora2a.
a polynucleotide sequence of SEQ ID NO: 61 or 62, or a viral vector comprising the enhancer element, or a functional portion thereof, wherein the enhancer element targets Cholinergic neurons expressing Chat.
a polynucleotide sequence of any one of SEQ ID NOs: 51-54, or a viral vector comprising the enhancer element, or a functional portion thereof, wherein the enhancer element targets Dopamine-Receptor 2 (D2)-expressing medium-spiny neurons (Drd2 neurons) expressing Gpr6; or
a polynucleotide sequence of SEQ ID NO: 3 or 4, or a viral vector comprising the enhancer element, or a functional portion thereof, wherein the enhancer element targets PV interneurons expressing Lpl.


SEQUENCE LISTING (81 sequences)

SEQ ID NO: 1 - DNA, 750 bp, Mus sp.
  source 1..750: mol_type = genomic DNA
ttgctacaagagaacatctagtttttcagacatctattgtctattctacaaagtacggtttcctgtgaccacaataaaatgtaagaaagtacatgaaggtggtgctcatgctagacaccagttggaagagcgacaaagggtcctccatgaaatctgtagccagggacactgaacaatgaggctttgctaattccccaagcaaatgcctacaaatgaacaaagctctgtgaatacactgaagccaagcttgctctcattttccagaatcacataaccccttaaggcaagacagacccagtcacagtatggcttaagaatgatggtcttgtaaggaaacgggaggggacttaagaagcccagcaaatcaagtacaatctcatggtcacacagagcttaacttctctatcaggttttaggcaaaatgagccatcctagtaatcccaagacagcctagcagcagagcaattacagagtgctgtcaatggtgagaccatgagaaacgaaatccctggagttatttggaattatgcatgaataatgcccggtgcattaactcctaaaacatccatgggagggatgctcaggagaacctgagactattagcacagcagacctgggccaacaatgttctcagacacactaatgggaactttggcacttaggccacaggcagaaaaatatacctacatccactgtctacctgaatagtcatcagtgagacagaaatgaaatccatattaattaccttgg

SEQ ID NO: 2 - DNA, 1477 bp, Homo sapiens
  source 1..1477: mol_type = genomic DNA
ttcaagagcaccagcgagtcaccacaactctaactgctttacaggtgttttttctcttaatcctcatatcaaccctatgcagtagatactattatttcacattctttccactttaagtcaagctaactgagaccaagagctgctaagtagcccactaaaagtcccagagctagaaagttggaaagaaagaattcaaagcaggtcatatgtgaagactacaccagtatgactccctcctctggaatcccatgtgcaccttactcagattctggttctgtctttcaccactctcttgtgggtaggtgtcctggtgactttcctgaccataatgttgcttagtcatctgaattccctgtgtcaggtacagtgcctgatacagagtggtcacccaaccagtgtgttgaatgaatgaagaatgaatgaattgtgtcttcactgcagggagagtgattttggtggatagtcagatggaatcaggttgaagggcacctaaagcttaatccctgctcgtgagtctttatgtgcctttgggcaaatatcttactttctacatgaagtttttctccttggcatgagtgaatctttcatatcttcccagatgcattatggattcattcacgaattagtagtgtgcattaacactataagtgctatacttctgcttagcatatgccccacaggcaacagagagatccatccctttcacagcagcttcaacttccaggctactctggggtttctaaatcactttggagtacttggaatgtgtttaagagttaacgaaagcatggaaataggcaaccaaagtgcccttctacctgtatcctagttctttttttttttgtcttggatttttcccaaggttcttaaaggaagaagtggttgacagccacatgtataagcaaaggcctctgcttaggaagcaggactcggctcatttctgtctgtaacttgatgtatgactttgggcaatgtgctctccttccctattcatctgtttccttatcttgaaaatcacaaaattgccctgcatgacttctctttcagctctaatattctttgatgcaacactggacaagatgctatgtgtggagctctgcactggaagctgcagatacagccatgagtggaggatcggggtctgggtctgtaagcactaagagcctgagcagtgggccagtgctccaatgaagccagaggcctcctgggtcctgacacagctggggatgggcgagtactttagtggggcatgagatgaagggcaggaaacaaggctaaaaaaaggagaaagggggcttctgtacctcatgaggaatttagactccatcctgtattcctgttagcccaactttgttcctttgtcccaccatcactgttttggccaattctgtgctatggtggttaatagcagggccaccagagctgcaagttcaggtttgtatctatgctttgctgcttactggcagcgtgaccttgg

SEQ ID NO: 3 - DNA, 750 bp, Mus sp.
  source 1..750: mol_type = genomic DNA
tttaaccacagcaggcctagccctttgtcccacaaatgagtttgcctaggggtaaaaatgtgggtgagaaaggcacagcttggagcgatcccagctcccctctgggctcccagtttccgtgttcactctttatttttttttttttccacgaatagtgtgcgagtttgagaggcattgccctgtgcttcttcctggagcccctgcagcctctcctggtcccaggcaggaaagcagagagctaaaagagagcatagagggtcctgctctgccaagtcccgttccttcagccacagtgctcacttgaggttaatatctgagttaaataactgggagatgacaagggagtgactgattaatagcggcattgaaaggtgtggggagacaagtgggggaagagtcgcagtggaaagacgttcagtgcctttgacagactgtatttttagcacagcctccctttcctgctgcgctgtagaggagactttatactccttgactgactgactgtttctgttctgtcagattgtccagtcaagtcgatgaaaacatttcagggggtcttgtccccttttcctctgaccatagcctgtttccacattatcatcaccaaggctgcactgtgttgtgtgtgtggctcatctgaggaaaccgtattgtgatggtgactgagttcttctgagtcttcagacacttggtgcccctcctctgatggcattcatctgtctttagtgtcacttccgtgctgctactttt

SEQ ID NO: 4 - DNA, 923 bp, Homo sapiens
  source 1..923: mol_type = genomic DNA
tttcactctactcacccaatatatcatgcctctcccatcacagcaactttctccctctttctcctccagatgcattcatctaggaaggtaagaatttcagggagagaaagatgtcaccgtggtagaaagacagggatcacctcctctgggctcttgagtttacttattcattctggattctttctaacaagaatatgaggacaagaggcactgtcctcaggcacttcgtcctgggagccaccaccatctctgcatggccccaattaggaaacgtgaagagctaggagagggagagtatggtcagtgcttagcagctgaagttccacttgcctggccatcgtgaatttccaggctgtcttctgagttgaacatgatggcaaaggagagcaaaatagcagatgtcactgaaggagagctcagcgagggagtgattgattaatagctgtattgaaaggtgggagtcaggtacgggggaagagcggcgatggaaaattttcgctttctttcagcagcttatttttaactcagctttctgttcttgctttattatggaggaaaaattgggccatagagtttactgccttatgccagattgttcaagaaaatgccttgcaacttacaatattttgcagctagtttcttccgtgaccaccacaaagactgcattgacttaaatatgaagatgttccagccatcaaaatgatggttggtgatgattttggatcacaaagtgtaaggaaagtattcaagacatgagtatcatgattttttaaggtctggatgaagagaccatttggatttactaataaggtaaattccaacttttatggcaataaaaacaacaaaaacacttatcagtgtaaagctttgggatcatctatccattaaatgagtttgtcaccacagtgaactaaatacctttt

SEQ ID NO: 5 - DNA, 696 bp, Mus sp.
  source 1..696: mol_type = genomic DNA
cacaggcagaggagagccatggcagagcatctgtggatctaaaggacttctaacctcagaggactcctctgttcaaatggaagaatgacagcttccaagcatccagaaaatgaggaaattgagttattccacctgctttttatctaaatagtctctttgttagaagctcaaacttttgatttagttaaaagattaacagcagtacaatctattctccatgtctcacctgatttatcctggactatagcttgagttaccagtcacaaaacaggaatgagagaaagagttttggccaattttatagccagatgaatcaaagcacaactggggaaaggctgagtatagtgaatggagcagggatgcaaagttgaagctgctggatttctaacccagcaagaatctcatcaccttgttattttgggcaagtcccttctgttcttttcttatttttgaatgacctggttctatgaacttggctatttatgagaattatgaaaaggattaaaaaaaataccagaactccaggacttagttaaaatagatgctctaaatttcaaagaaagcttggaaagaccctagggtttgatctgtgccagtaaatttcctaattctctggatccaaggaagctgtctgcatccactgactatgctgaggtacaaacatgtcccagtaagggtagaaacgggctggccttc

SEQ ID NO: 6 - DNA, 814 bp, Homo sapiens
  source 1..814: mol_type = genomic DNA
aataggtgttctctggggatgaacggagccacagcagaagcatctgtaggcctgtgtattgtttctaacaatccagagccccacagatttctatcctctggattcctcattcaaaatggacgaattcccgcttcccggcagttatgttttcagcgcttcccacaaatgagggcatttaatatgtcatagtagttctcttccattttccactttaacgctccctttattagaggtacaagccattgatttaattaaaaggttaacaccagagcaatctaatctttcctccccacccttctcatctgatttatccagggctgcagcttacattagaaatcacaaaaaagggaaaaagcatcaggccagttttataactagataaatcaaagcacagctgggaagggcttagggtagtgaatagaacatgcattttgagttgggatttttagatttttaactcagcaagaatttcatcataatgttattttgggcatgttccttctcctctccagggctgttttctgatctgttgaatgactggggttttctcaacttgactgattatgagaatcacatgagatatttatcaaaaatacagatttccaggacccatatcagatggaaaatctgaatcttcaaagaggataaagtcgcccctaaacattgtttatgctcacaagttcagcatactctatgtaagtggaccctatgggcttgtccagctctgacgtcttgtccacctctgactgtgatctcaaagcactcttatatataagtagtagatagaaaaataatcaagggctctgaatttttc

SEQ ID NO: 7 - DNA, 454 bp, Mus sp.
  source 1..454: mol_type = genomic DNA
tattgatactcttctgaagccattgttgcttgtaagacagatttactttcaccttgtgtcaagaacatccagagagaaagactaacatcactgctgctcagctgcctaatcgtttcagcaaaacatgtgagtgtgtttgtggacagtaactattaccatgggaaaggactcattcacagccttcctgattaacggctttattgcttgttattatccttttcatcttacacaactataaatatataatctaatgatcctttcactgaaatagcatctttaatgatgggattttcaaagtgcttctcagtcattcaataattaggcagattcaccctgcgccccccccccttttttttcttttttgagctttttactgagaatcttgaagagttgggggaaataaaaaaagaaagaaagtcttctactgaaacagtcattttttagaggagacaaa

SEQ ID NO: 8 - DNA, 690 bp, Homo sapiens
  source 1..690: mol_type = genomic DNA
tattgcaagtattctgtaattttttagactttatgtgtaaagggatatgaatgacttgaggcaattacatcattctgattaaaggcccccagtaactggaagtaagttagtctctctgtgtaaaaaattatatgtactactagttctattttgtaaatgttttcttcaagtaaaatggtctaccaacctgatggaaagaaatgttcttatttttaaactaatgaatttctcattcagtaaaacatttatcaagcacctgctataagtgctgttattatgctaagtataagacaagcattgttctcatcctcataaatttacagcttaatatgtgatgcacatagataaataattataatacattttggtaatgattttttaggcactattgtgtagcatggggtagagaggagaaaatgtttgatcattgattattaatatcaatgggcttatgcaaataatttttaaagaactagactaaatgcaaaagtagccttgctttttgggttaaatgataaatggctcttgggagccacactaaacagggtacataatgggaccattttcatctggctaattttgccttagtttcttgctgtggactagttggaaactaaaaaaaaaaatacatacaatccttgccctcaagagtcttataatttaattagggaagttaggcatagagacaaa

SEQ ID NO: 9 - DNA, 679 bp, Mus sp.
  source 1..679: mol_type = genomic DNA
aagccagcccttccttcccgcgccaaagctgcaaatgatggtaccctacacttcactcgctgtctctctacagcgtcgccctgggcgaaaaactttcccaactccctatgctaataaatcatgctaattatggtcctaaattgtctaacttaggcttcctctaaagccgactgtctgtctcgctctttccctgtcctgaatttgttctggttgttccttaacctgcgaatgaacgcagagagttgggcagctcctcctgtccagtatggtatgaactttctgattctcatgtccctacctggagctacttcagccgtgggtatcacctccctattctgagggccctggaggacatcagcccactgaactcaatacatccaactgggtagcaccatttgaaagagctcccccagagtcaatggcaactctctccagctcctcagcctttcttcccaaggcagctggccctcaagtgccctgacccacttcagacttcaaaggagctggcaaggcctgatgcttaatagtggcctctggactttcacatcaagctttctgggcataggatactggctggcttcggctaggatcctaggcaaacaggcttgataccaagcttctcctgaaagttcagtgtgtcttgtaaatgagagacatatcaaggaaaacactgtacctt

SEQ ID NO: 10 - DNA, 1040 bp, Homo sapiens
  source 1..1040: mol_type = genomic DNA
aagattgttccacctcgtgacctccgcactggccattcgctgcttggaaccttttcttctctctcttttcctgggtaacttcttctcatgctttaattcaactatctcttcctccaaatagctgtccctggacccagtttaaatgcaatctttccccattacacacaaaacatgctgatctttccctttataatatcatcgtgatgagcagttatgtatttatcatttgggttatttaatatctctaccccgaccttgcctcaatgagagcaagactcaaatagtgctcatcaccacagctacagtgcctagctctgggcctggcatatagtaagtgtccaataaatgtttgttcaaagaatgaaggaaacaggagagaaataaatgggtaaggggagcaaaggcatgaagggctttagatttagacacacctgggtcatctgttgccttctaagcatgtcacctttaacctctaagcttcagtttctacccctataaaaaggctatagcactagggcttgtctcatattgggaagattacctgacacaatgtctataaagatattagcacagggcctggttcataggaagcactcaataaatgctagctgtcatcctcatccttatcctcatcatcataatcatcagcacattgaatctaactgtggctagttatatattcaggttacacctacttcaaaagaacttactggacacaccttctctttccattgtattagcttccttttgtcgtgtggaaggcaggctggtaagtgattcctatttgtaggtgaagacactgacccctggagaaagggttggtcaggtctaggtcactactacttagagagtaaagcaaatgcctgcctgtggctcaagcctttggccacagtaatgtctcccaggcttcacaggctcatttcttccaggggctttgactgggttaggtgtggtggtaaaagctgggatttacatcccccaggctgctgcagatggatcacaagggattctaccagcaccaggaaacagtgtgtgagctt

SEQ ID NO: 11 - DNA, 750 bp, Mus sp.
  source 1..750: mol_type = genomic DNA
agatctgggttacctggcaggggccttgtacccttatgacacccatagatttctccacacaagcaatttttacaatagaaatgagttcacaagattcagcatggtgttatggttcataccatgagggaacagagactatcagagaactttgggaaatgggcagaaagtctagcattatacaaatgctcattccttgcttgcacttggttatgtaagatacaaacatcaggcaaacactctgctatctttgtaacatttctatacatataaaatgatttcaaagtaaaacgaatggaaaaagacccataccttgttaaaagagagggcacatctattctatgggaaaactactctgccgtctcctgctttgtggcatgataaggggagcagaaagggatggtagaaagatggtttcctcttaatacagtttggctcttgagtcttttatcttttacagagtaaagtgtttcttagaaagtcattgactgaatactaactcatgttttctctacagttttgctctagaaaccttcatctctgcatcctgggagtgggtagctctgggtagcaaaagcagctccatgtgcagaaagcagatctgtgcaaggagaagtaagccctcatctctctgaaaatgggcccttgttgatgtaagtacatctttttctgtttgggagaagagtaggggaaagcatatgtttccaagcagcacttcgtgtgtgtgtgtgtgtgtgtgtgtgtgtgtgtgtg

SEQ ID NO: 12 - DNA, 1035 bp, Homo sapiens
  source 1..1035: mol_type = genomic DNA
atctctggggtccagaagtagaagctttttattgcctccggagtttattcttatacatcaaggataaacattcatgacctaacatcatctctcttgttttctcccgccaaagctataaggatagtctaatcatgaaaaaacattaaacaaactcaaaatggggacagtctataaaatacctaaccagttctcttcagaacagttaaggtcctgaaaggcgaggagagaagaagaaacaatcacaacttggaggacattaatgcactaaatttaatatggtttcataagttgggctgaaaagaaaaggacagtagaaaagctgggatatgagaatacagtctaagtgtagtactaatgttaagtttttaatctctaacttgatggcatggttatagaaaatagaaacattaggcaaagctgggtgaaaggtatacaggaactctactatctatacaacttttctgtaaacataaaattatttcaaaataaaactaatataagaaaaaaacatggccgacataacagatagaatggatatcttctctagtctatgaaaaaccctgtgtcctacattcacttgctttgtgatataataaaagggggaaaggaagggatgatagaaacattattcctcttaataaacttggattttaaaatcttttatcttttcacagcataaaacattccacttaggatgctatatgttgagcattaactttctctttttctttatagtctttctgtaatagttcaaatcctgcccaccctgagggtggatgtttctagatgatgaaggaggtcataaacagagagtagattaatataattatctttatttcatgtctatttgcaaatgggccattgttcacatagttgtctctctttctaaatggaagaatagttggagttgggggtggggaaataccagaactgagaggagtaaaggtgcttcaagacaatgcttcttaaactttggttttgtgtttgcatctcttggggatcttgtttaaagtaggttgagattcagtatgtttg

SEQ ID NO: 13 - DNA, 750 bp, Mus sp.
  source 1..750: mol_type = genomic DNA
ctctgtgtcatcttgtttgaatttttaatgaacatatatttataatttcttatataatttaaacagtgttttcaaaccataggattgcatgcaaatgtaatatgcccttataatataaatggcctttctcttgcttttttcttctgtcagcaagaataacaatttcctgcgggacctctggcaaaacaaatcaccctctgtgctcggtcctgactggtggaaatacttagcatgcagtgggaatgaacagaaatgcagggtcacctgagaacagtgttcagcatctgtaagcaatgtgaggcaccttagatgatatgttaggaagcagctggccttgcagctccagtattatgtttcacgagcgtcagcatctccagtctagacagaggaagcagcagatgagggcaggatccttaagtagtttctatatcctgagttgaaatgaaatggccattgaaaatcagagctgaagacattccaaagggacacacaaagggcagataccaatctatctctgcattataccacagagataacctgctcatgtatgggtccaaatggatgccaatgttctgtctcctgtgtggctcccatatgattctcacaactgtggtaggtggtggttcctcaagtgaggaaactgagatccataaaaggatataagctttcacaaatggagacagtggggggttggggtggattgctggctagttttatgtcaacttgacacaagctagagtcaactgagag

SEQ ID NO: 14 - DNA, 1312 bp, Homo sapiens
  source 1..1312: mol_type = genomic DNA
ctctgatttttatttttttattctgcttacattgagtttaatctacttttttatttctccagtttcttaaggcagaagctagggacattaatttgagacctttcttgttttctaatataggtatttagtgatataaacatctccttaattattgttttaatggaattctgacaccaactggtgtactgcaatttaaggctaacactaactattggagttaaccttagactctaaagtctcaggggcacagttgaatgagactatgatcaattccatttccagctgtaaatggggtcccagtccatctgcgcttcttaccagattcagagattccatgatcccattaggtttgataagttgctagaatgactcatagaattcaggaaagcactatatttatgattatagtattatcataaaagaattagggtcagctccatagggcaaagttcagcaggatcctgaatgcagagcttccatgttctctctctatagaattaggatgcattgccctcctgtcgcatcaacatgctcaataaccaaaatgctgaactgagcttcagtgtccagagttcttattggggtttcattacataggcatgattgattgagtaattggccaatctccagcttccctcccttccttagtggtcaggctggctcaaagtcccacccatctaatcatataattggtctttctggtggcccatgcctataatcctgagtcatctcattttttagcataaacataggtgtgatctgagaggctcatgaataacagagacattactattactcaataaattacaaggatatcttattcttcccaggaacaagggacaaagggcagtcaaattctgtattatacaagtggcatgtcacaaattttgatatgtttaaatttggttttcatttagttaaaaatactttttaatttcccttttgatctcatatttaacccatgggttatttaaaacatattatttagtttccaaatatttgggggattttccagagttctctctgttgttcctttctaaaaattccactgtggtcaaaaacatactttatataacttgaatccctttaaacttatttggggctgtgttacagcccagcatatgaactatctttataaatgtttggtgtgcacctgagaagaatgtgttttctgttgaattgggggattttttctataaatatcaatatttaaagttggtttgcaatattatttcattattctatatccatattgattttctgtctgttctataaattattgagacagaagtacaaaaatgtccactgtaag

SEQ ID NO: 15 - DNA, 750 bp, Mus sp.
  source 1..750: mol_type = genomic DNA
gaaaacaagctggaaaaatagtcgtctacagaaagtcaagtacaagtttaagggcactggttgtctctctctctctctctctctctctctctctctctctctctctctctctctccctctttttcctttcaaatttttctcagacaccagaattgcatgctaatggatagcaagacccacttttgccagtcattgaaaataactcaggtattcaaattttgatctacgagttgttcaggatccaggaactacccagagtttaggagccagatacaagccataaagagggctttccaatgtgttcctttttaccgagattagaagtctgcatacattttacattgagatgtaaactgccactgcaaatgtaagtacactgcaaagtcatcacgagaaattttcagttcatttaaagtcactacaggaaaagtattacagtcaagagttttattgtctgttttgtatttctcacattgcttgatcacaatgagcccagtaaagggctcaggaagcaagttttacagcaggagtttatagtgccctacgtcagagacatgcctatgtatggggaggccttggcaggcaattctctgaaatgtttcctattcaacatccctgttacaagacaaatagtcatagcaattacaatgacaatacaaagaactatgatttattaaaaaactattatgttttgccttttataactccagaaggtaaacaatgtatcttgtcaaaacaacctcaagaaaa

SEQ ID NO: 16 - DNA, 1316 bp, Homo sapiens
  source 1..1316: mol_type = genomic DNA
gaaattcacaacctggacaatcagtcacaaatcaagagttaactatctgcagagatcaacatccaacgtggtaaaaacactgcagtcattaagagtcactatccagcactttagaactaattaccttacatctttcccctggacttctccttggaaacatacatatttcataaagtaaagctaagttatgattgaagagttacaatactttaaaatttttttgataactgatgcatgtattttaatttcactacaatagatgctgtaagtctgttttaaagcaatgatttctggaagctgcaatctcaaatgagttatctgcaggcaagagcagactcacttggcaggacagaatgcaggcaactgcgttagttatggtttttctaaacaaccgactttaaaattctatttattttacttttaaaatatacagagaacagtggaaactattttaactgttaacaaaaactaaatctacaataagagcactggaaagttagtagacattcggacacataaaaaggatcaattttaatatctctttgaatcctttttatcctttatctcctaataaatattcccccataacttatacttctttcttagtgtaaaagccaactaacactttacttatatacaaataactagtttaccaattagttcacacaataatgtaacaacactaggacagattacagagccagttacagacaaaaacagtaaggaaatgtttaacacatatgactgtgtgttacagtcaaacatgactataaaactatgactgagtagatgtgagattcttcctgctttcccaaaacactaaagttgaatgatttttaaattatctacagctttctattattcacactgtcattcttaattatgccatagataacagtttacagtagccagggagcagctaactgtagcttagttcgatgaagtttgagccaggttcctaaacatatgtgtatggtagcctgttaggcctctctaatggctctaccagagttctaactttattagtgtcattctttaacagctggcttctccactaagtgccatgagggcagaaactgtaactttttttggctcatcattatatccacaagtctatcttgcctgcttcactgtaggcattcaatacatatttacaaataaactgacattacaaaatataaataaaaattcaacttaataatttaaaagtgaataaaaagtataaacatccttcaattccctcaagtatagatttaagtattaaaattctagaacttgaacaatgtattttggttttgttttgagaacactgataa

SEQ ID NO: 17 - DNA, 511 bp, Mus sp.
  source 1..511: mol_type = genomic DNA
aagaggatgggatagggggtttccagaggggaaactgagaaagaggataacatttgaaatgtaagtaaagaaaatatccaataaaaaacaaaaacacaaaacaagaaagagcaatggctagggtgttcccaattaaatctcacactcgaaagcacggatccaggaagctagaacagacaacaacacaccaggaagctacacatagacgtagcatcttccagttatagaaaatcaggaatagagaaaggcatttcctgacagaaaccataggaaaaatcacctcacttccagaagagcacatagaaaaattatcctcgtttctctcagaagctgtgtaagcaagaggagagtggaatgaaatatttactgtgctgagagaataaaacatcaacctagaattttgtaccctgtggaattaccctccaaaagcgaaggcaaaaatacttttcagacaaatcgaaggaatttgttgtcaggagacgtgccttagaagaaatgttaaaggaagttc

SEQ ID NO: 18 - DNA, 557 bp, Homo sapiens
  source 1..557: mol_type = genomic DNA
aagagaacagagactgaatggcatagaacagaatatctaagaactgtagaacaactccaggtgtaagtagcatgcacataatagaaataccagaaggagaagaaaagaaccgaggaaatatttgaaagagtaatgattgagaattttctcaaagtaatatcagacacaaagccatagatccagaaagttcggggaacgcctgcaagataagtgacccaaaaaaccacacctgggcatatgaattttaaattacagaaaatcaaatataaagaaaatgtccttaagccagaaggaaaaaaaaaaaaacccaccttacctctagaacagatataaggattacatctgatttcttcttagaaaccatgcaagcaagaagagagtggaataaaatatttaaagtattgtgaggaaaaaaacccccaccagcctaaagttctgcactctgtaaaattgtccttcaaaggtgaaagagaaacaaagaagttttttttttttggacaaattgagataacttgttgtcagtagatctttcttgcaagaagtgttaaaagtagttc

SEQ ID NO: 19 - DNA, 750 bp, Mus sp.
  source 1..750: mol_type = genomic DNA
aggttcctgtctgcaagcatagcagggtatcattaatagtgtcaggagttggctctctcccataggatgggtctcaagttggggcaatcattggttggctgatcccttggtctctgtcccatctttattcctgcacatcttgtaggcaggacaaattttggtttgttccaaagtgatttttaaagcattaccattttgtgttttgttggtttaaaacactttagctattctggtgggtgcgtgatcctgcctcactacggtctcagtctccatctcgatggtgactaaggatgcctcctatgcacttatttaccatctgaatggttcctttatagaatgcatgactattcaatttttttgctcattggacagcattttaatgtgtgaaaggattatacatccttcaagatatggaagaaaaagcacaaagccctaaattctgtgaattcagatgtgtaaaaaaagacaaggaagtgcaaactatatcagcttggtgtctcattctgggtggctgtcagagccctgtgagctgacttaagaattgggtccctgcaatgggtgctggcacttgcaaggggatcatgagctcaaggccatactgagctgcataatgagaccttgtctcagtaactagactggggttggaggtcagtactaaagggctagcctagcctaaattcccgagttccacccccagcactataaaaggaaaaggaggaagaggtggagggaaataaagaaaaggaaagc

SEQ ID NO: 20 - DNA, 1028 bp, Homo sapiens
  source 1..1028: mol_type = genomic DNA
aggttacacactctttgtgttaagttctttttactcagcacagtgttttttgaaattagttcaggctattgaatgtatctgagtttattcatttttttctaagtagtttccattgcgtgaatatatcatagcttgtttatctattttcctattgatgggcatgtgaggttgttgacaagttttggtattatgaataaagctgctaagaacattcttatctcactctttttgtagagctatgttttcatttctcaagggtaaataactaggagtggaacttctgagtcataggattggtatgaaactgccaaaagtttttccaaagtggttgtatcattttacactcccatgaacaatgtatgagagctctcgtggttccattagaccaacacttcatgttgccagtttttaaaattttagccattctggtgggtgtataatccacctcatcgtggttttctttttcatttctatgatgactaataatgttctttatgtgcttatttgccatttgaataggttcctttatgaaatgactgttgaattttttgctaattggataacattttaacatatataagggttgtatacccttcaagatagcaaaaatttaaaaagaattcagaattctgtgaattcagatacataaatatctacagagccaaagaaatgcaaagtgcattggcacaatgggccagtctaagtggggtgtcagagttctgtaagttgctttacgaaatcccagcactttgggaggctgaggcgggtggatcgcgaggtcaggaggtcgagaccatcctagctaacatggtgaaaccccatctctactaaaaatacaaaaaaatagctgggcatggtggcgggtgcctgtaatcccagctacttgggaggctgaggcagaagaatggcgtgaacctgggaggtggagcttgcagtgagccgagattgcaccactacactccaggctgggtgacagagctagactctgtctcaaaaaaaaaaaaaaaaaaaaaataataataattagg

SEQ ID NO: 21 - DNA, 750 bp, Mus sp.
  source 1..750: mol_type = genomic DNA
gctatagcttcaaccagctgccactcaaagccccaatgtgccttcttgggtcctgcacacacatcctactatgttttatgaatttttcgtttctctgttccaactcaatctaactaagaaaacacactgccacccattgtctgtgtctgaaaaaaaaatacaggaacatcaactgatgtgtgcctgactcagaagaaaagaagaccatggccacagtctcactgctcagggcattcagggaagcacaattgtactcgccctgagaaacagtgcagatggtgtaattggcatcaccagcccacatagatcagagatgcatttatttccatgactttgtttgcatcatggccatttttctgggctcctgctcacatctaacctcttcaagggtcatgagagagtgaagcattttgcccctgggattgacaggtgaggggatgtttgcatttccgtttacctgagaaatactcatttcttgcagcatcagaaaacatttggttatgttatctcgtgggtccaggaggagcatggagtgtgccaaatttaagaggcactgccaaatgccgtagcctgcattatgtagctgaggccgccagtaaaactggtgctaactagttgaacattccagctcttgcctcagaagcaaaacaaccctgttagtcatttccaattgggctcccaatgctcgcctttggttagaagacgtccttgtgtctgtgtgcaaatgctggcccctaggcacaactgatt

SEQ ID NO: 22 - DNA, 1466 bp, Homo sapiens
  source 1..1466: mol_type = genomic DNA
gccatcagcaacaaccattgcagccagaacagaaagcctttattaggtgtcttcggcgtcttctgcacacagcatcaaactagatcaatgagtatcgattggtttggtcatgccccatgttccctcaagggaagacgggctctttaaacaaccaacccaaaatgaaacaaaacaaaacatataggctggacgaaagctccaagtttcagattttcatggaaaaataaatttgcaatagattggttagaattacagaaagagtgctttaaataaactgttctagaaaaaaatcacagctacaactccgccatgttagagtgtacagtctacgaataacctaatagccttctcagggaacatcaagcatgctaatgacaagataactttttgctttgttttattgtgttttaatcagagtgccatggggtggtgaggagattttgtgccagacaaaaccgagttagaattcctgttcttttacttattaatgtgttaccttaagcacattctgaaatttctctgtgcctcaattgtctcatctgtgaaatggggattaaaaacagctatcccacagagtttttgttgtatggataaaattaagtaatgtcaggctaggtgcagtggtacacacctgtaatcctagcactttgggaggcagaggcaggcaaacagcttgagctcaggagtttgagaatggcctgagcaacatggtgagaccctgcctatctctacaaaaattagctgggagtggtggcacacacctgtcgtcccagctacttgggaggctaaggcaggaggatcgcttgaacctgggaggttgaggctgcagtgaaccaatatcatgccactgcactccagcctgggtaacaaagtgagaccctgttaaaaaaaaaaaaaaaattaagtacgtccataacaagcttagcatgatgcttacaaccagcaaattctcaaaaaatggttgttattggtattgggtatttccttttaaacagcatttcctgagggccagctttgtgcaagacactgttctcagctctttgcgtgtcttactcatttcacctcctaagaatcctgtgtggcaagcactattcttatactcattttatacatgaggaaactgaggcctccttgcccacgatcacatagtaaatggcaaccccaagctgtctggctccagaagatttcctcagaaaggaaggtcagtttgttgatctaaatagttgagctgccatttttgcctagaaaacccgagtggaagatttcatcagtgtccccctcatctgctgacagcagtcagtgaacatccaacatcaactggactctgcctccttggaaaggccccaggcttgatctggatgaatgacctctgtgtacagttacctttgacttggcctcctacctgaaaacacaaagagggacagttacataaggtaatgctgacctatattcttgacaccctgagt

SEQ ID NO: 23 - DNA, 750 bp, Mus sp.
  source 1..750: mol_type = genomic DNA
ctacaggggaagagaggagaaaataaagagggagatgggcatgtacatatttaaattcacattatgtgtgtgtgtttgcaagacacatataaatggcaaatagggaaacttcacaaaaaaaatgaaaagacagacagctagatagccccagattttaatgccacccccaaggtagaacaggtggcgaaaatataataaaacatgttgcaaggaacttgtcatgaattttaaaaacaaaaacgcctacagaccaaataagctgctctgacactgggctaaatccgaagttcccgaactattttaaatgacaaagcagaagctccctagagaaggtccaacagaaatggtttgagggcaattgctgctaaactcatagagagaggaacttggagagaggacgaagcctgctctctctgccacaaaggccacgtgcaaacgtccacagccagaccatgtgcaggaccaaggtgtgcccacaaaggcccctgggagcctcccgtgctccattgttggtgggtttctcaatgcacatgtgacaggatgctgggctcaggctcatgggcagccactccaggaccccatgtgaaataggccagagatagaggttctagctagtacaggctgacccccccaccccacgctcagcagtgcccaccctcacaccctcatctgtttggccattgcaagcaccagcctgtgctttgttcttggccaaccatatatgaagaagtcacttccgtggcccctccc

SEQ ID NO: 24 - DNA, 1420 bp, Homo sapiens
  source 1..1420: mol_type = genomic DNA
atacacataggccggacaaaagcttaaagtttcagattttaatggaaaaataaatgtgtaataggcttgttggactaacagaagggttctttaaagaaattgcttagttttcaaatactaaacaccattagataggcaagtactgccaaatgagtttctggagacagaatttaatgaacaagagacaaaatctttcaattatgtcattgtgcatatatagtttttgcatagatgaagtttggttcctctaatagttaaacatcaccccctacaaacttgttttgataattgccttaagagatactgtctccaaataaaaaagaaaggtctgattttttaaaaaatctaaattgtccgaattcgtggacaccttattagagaaggttttgggttactagtttaaaaattgttccctcgttttttgttttttaataggatatcaggaaaatccaccaacaaaatcatctctgaagtcattaacatgagacaaacaaaagcggtgactgagatccagagtcacctaagccaatttaaagtcttagaatatacggcccattctttacaaagtcctcaaaccatcacctcagagttccttaatagaagtcagaagcccaaattcaaacacagaactaccatggaattgtgtggtgacacccaaaacatcacctgtctggcctcagtttcctccctcatatagatgatcaaaccacctccataagttcctcatagagatgacgatgatgatggtgatgacgatgatgatgatgatgtcatttagcagtgttggtaatgaaaatagcagctgcttgtttattgagtcctaccatatgccaaagcactattctaagcctttgacttgaattaactcacaatatcctcctggaaattttgcaccagggatttctccccaaatccgtgtctaccatgctctcttaagcgaagaatattgtgacaggttccatattcaaaaagtagccttaacagtatttttcatcttgcagaccctttatacaacgtgtttttgacatttttcccatcaagaggaggggtctatatttcttccacttgaacctagatgactttaccaccacggcagaagtgacactatatgacttccaaggctgggtcataaaatgcaatacagctctttgccttctcaagctgttcaccccaacccagccacgaggctgggaggtatcctgggcaacaaggagagaccctatgtaaaggctgcagccaactctccagcagagttccccaccaacacccaacatcaatggccagatgagtgagggaaggcatctgcaggtgattcagccccagccattgagtccccaccagccttcaagtcttcccagtggattttcccagacatcaaggtgctgtccctgctgtgggcccactctgaattcctgatccc

SEQ ID NO: 25 - DNA, 750 bp, Mus sp.
  source 1..750: mol_type = genomic DNA
ttaaagatggctcccaagaattgcgtaatcatatccacgaatcactaagattagaagtacacacttcagtacatttacatatgcaaagaaagtacaccccgtcacttatctgccacttatgtgtttggtgtagccaactcaaacagtagcctgtctgacttctgaatagcatttgtactttaaaaaaacaaacaaacaaaaacagaaacagaaagagggaaggacacattagagtcatctgcctcctgttaccagctttctctccctgattattagaatatgtccccactccatctctttcacctaggtaattgcaagtcccctgcaaggtgcatcccttttcataagctagtctttctcattctcgcacatacaattgtgctccttaggtttcttttccttggacactcgcctggtttcataatgtagttccttttgccttcgcttttctcctctctacgggattatgggttcattcacttgagtttcctgccctgagatatgcctgcctcatttgctgccgtggtaggagggctcacatagatccctcagatgtctttttgtctgttagactctctctattcggcatatgatgctagggatgcatttatccatttatgttgtaatttaactatgtgataaaaattgtagactctgaagcagaagttgttcttcagtgtaagcttataattgactgctaatgaaataattgccatgtataggtttttacagcaagaaacatctgccctg

SEQ ID NO: 26 - DNA, 782 bp, Homo sapiens
  source 1..782: mol_type = genomic DNA
tcaaagatagatactaagagttctataatcgtgtctatgagttcctaagacccttagagctgcacatttcagtagatctagattttagaaaaatatactctaattacttatttccctattcttgtttggtgttccacactcaaatggtctcctagctggttaactgataacagctgcagtttttggaaaggagaaagagggaaaggatacatgaaaagcatctgccttttcacaaacctctgttaacagctttttctccctattagagagaaaacatttccttggtccttctcttttgccttatgtaattgcaaatctctggcaagatgcatctcttttcagactttaacttgtctcatttttcccccacacaattgtgctctttcttttcttgttccttggaaactcgccctcgtttcataattttgcttcttccccctttgctgttctgttctctacagagttacgggttcattctctcgggtgtcttattctgcctcatgccctatctacatgttaacctcatctgttccagagacagaaaatggatctagatctctccagatttccttttgtctcttaacttttctttgcatgggatcttagacagatgcatgacacttgttaaagtatctttctcaatttacatttcatttacctttctgctttaaaaacatagaatctttgagctagcagttgccattatctgtaagtttgtagttgatgtctaaggaaattactatgccattattcctaatttttcttcaaagacatttcccctt

SEQ ID NO: 27 - DNA, 728 bp, Mus sp.
  source 1..728: mol_type = genomic DNA
agagcagagacatttcagcagcagtaggaaagaccaggatctgcctctcctcaattaggccaccttctttcttcctttgtacaataatttatctaaatctttaaaaaaaaatacctagcaacgagtcccttgatgtttttaatcttgaaaggcaggcatggacactttgtttcaaacatcatgaaactgctcagctatccagtaatgatccccttatttttttagccacctgggaagtgtgggtggtgtgggctctccaattctctcttggaaaggagtcgtgggtgtcatgccaatgttcctttggtattctctcaatgccaaacagattgtgtgtgtgcctctgttagtcctatctctgacactgatgaattcaacccactggcattaaataaggccatgacaaatggcaggagggttacaacacgtggctagtacaatagaagtatgagtcagatggcagaaagctttattggtctttgattctctcttatactggacagtgttatctctatagtcaagttatagtgctctgatattccttttctcctgtggctccactaaatattccctagcttggtcttgcaagatagttcagtgtttaaaggtgcctgccatcaaatatgacaatgtgaagggatgcctagactccacatgatgaaagaactgagtcccaaaagttatcttctggtccctccccaacacatatataacaatggcacctacac

SEQ ID NO: 28 - DNA, 1299 bp, Homo sapiens
  source 1..1299: mol_type = genomic DNA
aggagacatggattcctgctttgcctccttcactgatgagttctgtttcttaagagcagttgtcaaacttatttgactctcatttgcaccttctttctaagtagataagcattaagggtaaagatgctttaaatatatttatgctactcaaatggaagctttataaacaatgcaaatgttacaggctgatcagatcaaggatgtgacacaaattagaaaatataaagtattagtggatgccatgatttgtgagacagccgctggagtatccctcatagtagaacttgatgtttggaatttgaatataggttctcagaccaaaagaagaaagatatggttagaacttacccttggtgttctctcaatggcttacttaccattacaaatgaaaagagttaacatgtacataagagctctttgccaggaactgttaaagcattttacataaattatctcatttaattatcccatagccccgtacatataaatatatcttaattttttagttgaaagtactggcaccagagatgtcagacaactatcccaaggaagcattagtagttagaaactagagctaggattcaaactgttgttgtctgactctaaaatcagcacttttaacagcacctcgctaaactagatgagtctcccttaatttggacacacacaaaaaatagatttgtcgtggatcacaagcagtcacttagattttggtctaatgtgtaattctcagtttcaatgatctctgcaaacgtcatattagtagttcgtgagttacgtaaattgaaaatatcatctatctcttgtacaaaaataaaagctttgtcaaatttatattcacacatgttgaaacaatgcattcagagaacaatgcattcattttatgtgggaatttaaataggtccatgtgggataaagatctgaccggtgttctcatgcaaaaattactagagtaactggactaaagtcaagaaacctgatttctcacaccagctctgctactaaacacctgttgttcagaggtagcaaattatctcttctttttctttgttttttttttttttttttttttttaacccccttataatatcacaggactagactaggtagcctccaatatccttttcagttctaaaattttatgcttcgataattgtgaataaatgtgttgcccatcccttgagttaaacatgaatgatgaatcatctcaagtctgaaaaatatggctttaatcttatgtgacacctcaggctcacggtatctcaggaaagccctaccaatcacatttggctgctatgc

SEQ ID NO: 29 - DNA, 750 bp, Mus sp.
  source 1..750: mol_type = genomic DNA
ttttaattaaaggagactttatgcaaccacacacaccaggaagatttataagtgcaagaatgtaaatttagtctatttttaaaggatgttattgacaggcacagttcaaaagacctcaccagggagcccttcgtgagacactgcgtgtttcatgatgaagcttgagctttttttttcccatcatgcatattaagttgccagaatgatccttaattaatcacatgaaagcctgacacttgaaaaactaatgtgtgagagacatggaagttcttcattaccctcgtctcctctgccaaggcattcaggttgagagcaatcctggctgggaaagcccacttccacacacctggcctctgctcattcccagaagctgctggcagcacttctgggtcactgtggctgtgggcagacacaaggtgtatttccttccagggtggcagcatatgcctctgaaaaaccaaactagatagaacctgggatctggcagcccaaactcctctaatagggattttataggaggatgctctatggggaactaaattgagtcttcttttgtgagaaagagaccaatctcatcagaatccagaaagctaggcatagtagcctaagaaccattgaagtgaccagggaaggaccccttccaaatccatatgccctgggtcactcagccaccatcttttaaaacagcccgattttgagtctggggctccctgtcatgagaagggaaatggaatcagactgtattttgaa

SEQ ID NO: 30 - DNA, 1372 bp, Homo sapiens
  source 1..1372: mol_type = genomic DNA
ttatattgcaggcggccaagcattaagtccagctgaatatattcagagcaatcaacaaataacacactgagaacagacttttctcttatgagacttcacattagaaattcattcagtagaatgtatctaaatttttaaaaagcttcaatgttgcacatatgttaaatttaagaatccctgggttagaatattactgtggaatcatgtccatgactaatttttttaaaaacattgcatttacctagtgttcatggtctataaagtacttttatactcattacctcatttcatccaagccttccctgctggccaaagagctgctttacatcctttactatggaaatgtgaccatctaccattacattaaaaggcccatggaactatgaggctgaaacatgaaactgttaagattgggccattttcgaactacaaaaaggcaatttcatctggttcagcttaatctgatttcatattgtttcgaggtgaactaactgtacttcattcccatgggctattcagttatactgaagagcagtgctgatcatcacttctccactttacaggatattctcttgtccccaaagtgaatctgtttaacttggcacaagggagtggacagggtcattgtgggctgccccaggagagacatattggtgggcacaacattcaatgccacaatgaaatggaaggctaagacaaagaagggtgggatccttgggagcaaagacactggccaagaaatacaccaaatttgaccaaaacctgaatggagtgctgtggctctggaagtgctggaaaggaaggggctcaacttcaccaaacactgacaatgtgccaagtccagtgctgggcacatgaagccgtggcagggagattacttactgatcctcaagtcacatggggaagttgagtggcagagtataagtgtggagctgatctctctatgtcgatgtccattccactgcttagcattgcttcccaaatccaacctgtgtgaaaaattaagttcacagtacttagaagaaaactccactccctattctaggttttgtccagagacctgaactaatgatagagtcaaagaatgtatgcaaagagcattgaggcacctagtttaaggaattgttgagggcagaggctgaaatgcagcacaaagcgggtgagtcagaaagtcccttaggtcagttgttcgcaccctccttctcccctactcagagacacaattcacttgcaactggaagccctgggaagaatttcccacctagttctgtgtctgaggagagaaaaaaacttgctaaggaccaagttagagagaaaacccaaatgagaacagcgaatctagccatctttctttgaatcagaattagttgga

SEQ ID NO: 31 - DNA, 750 bp, Mus sp.
  source 1..750: mol_type = genomic DNA
ctaaaatcttccctgtgattaatggggtgaaatgtactatagaacaggttcaaatgaatcacagtaaaatatcctttcaattatagataactttctgaggaaagaagaaaaactgatgttagcagataagtaaatacataagggaacctgacactttcccatatcacttgtcatttcatagtttcaaattaagctcagctccccaatatcaattaaacccttacagtgtgaagcacgttgctgcgtattgggaacggtgagataaacggaaagcgagacaaagatgggacattagagttgccgtggaagtggctgacaggatggataaaaatttctctgtatcgtggtaattgaatgggggcacgagtctattaatatcagaatacaatttgtttctagaatgggcgttcatttattatgatcagacgcttgctcttgcactgctggagatgctgcgtcggactcatgtggctcacatattcaagtcaagaaggctggcactctttctctctgttgctcttccatatttgttttctctgtcttccctgacagtttctcccgaaggaagacaaccagtgctcgcctatctcctatcagtctttgttctattaccatgttcttaatggaaggtgtgtgctcacacctccatttctctcaccccatttacctttcaacatacaacagtctggattctgctcttacaatacatcaaaaattcctctacgattgacaaatggttggcagatcacc

SEQ ID NO: 32 - DNA, 1481 bp, Homo sapiens
  source 1..1481: mol_type = genomic DNA
ctaaaaccttagctgctagttataagcatgacctcaaaaccagaaaccatcagggagaaggttaaaagaattctctgggtaaaacaaaatgtgaattccacacagcaaaaaataccataggcacatataaatgataaattgagggcaggagggggttgaaacagaaatgaattaataacctgaatatataaattggaaattcataaagaaatgtcaacaaacacttagcatataaaaatgctcagcttcactaatagtaaaaattaattacaaattaaagcaattatgacatttagctttctcctattattcagagaagacaatatttaaaagatagataagatccagtgttctctggggcaagtgaggggttactgctgagtctgttgtgggaattctgaaaacagataccacatttgtagagggggatgatctacagtcttaagtattttttcccaataggaataaactattgattcctctggttgcttttcaggttttttctttgcctgccatggatttctttgggtttactatgtttggggtttgctcagcttcttgaatctgtaggtttacagcttttgccaaatttatgaggttttcagccattatttattttgtcaaaaaacaaaattacaacaaatttagtgtaaagagctagttgacttttatctgctattctagaagcaggcaacacctcactctataaaatggggtgagtgctctaatgagctgagcagagaaagttggcttcataggcagaaaagctctaaagagagcagatacagagaacaaaaagcagatagccctttcaaagtccctttctgtaaagggctaaaacagaggggacttccttattatgctcatttgggttggccggaatcacctgttttttggaaaactggctcttttcaaagttcaatttgattaggtggcacttagcctgagtgactccattctgcttaggactggtctgctgtggcctaggtgcaggagactagcccaaaacaatggcctcccgtaaaatttaactatttgaatactttttcagtgctgccctttttctcttctgcctgaactccagcaacataaatattagatcttttgttatagttccgcaggtccctgatttagtttgttttctctgttcttcactttagatagtttccattgttctgtcttccagttcactggttctttcctctgtctcctccattctgctgttgagcccaaccactaagctttttattttggttatcatatttttcagttctaaaattttcatttagttcttctttatttcttctacttctttgctagggctttctattttttccatttttgccgtgatgctttctaaatttcatgtgtttcaagtgtgttcatagttgctcattgaagccttattatggctgctttaaaatattcatcagataattctaacatctctgtcatatcagtgttgccatctattgattgtc

SEQ ID NO: 33 - DNA, 750 bp, Mus sp.
  source 1..750: mol_type = genomic DNA
tgatatccaaaatcaggatgaagactactagatgaagaaatatcttaggggttaacaaccgactatagactgagagtccccagtgtcttcttcagcttttatatctcctaaatgtccaattagctgatcatttttacgtaactattagtcaacctgaaaattcctttctaacttcatcaatctataggactgtgataaacttatctcttggcttttttggattagagaatggagttgaactgtcaagtagaaatgaatatttataaagtagatgcttattttggtattcagaaaagcagtctctactcattggtaggattattcttgcggacaaaattctgaattacaagttctagccctcctttcagtatctccgatatctacactgttcagtgcagatttatttagtcctgttacaaaggcattggttggctttctaaaaagaagttcttctgaagcagaaaattgactccatgtaaatttctcactctgttgcacatattaaagtacattgattttgctgatggaaagaggcagaaggacagggagagatgtgggggaagaggccaaaaaggtcataaatttcaaatcatggagatccagagaatattatcaaccttttctcgtcagagaaattgtttgagagccctgttatgtccctgaaaatgaaaggtagagttgccagtgaaggtcaagagtaactctcctcaggatggagagggttttggatggtatatgaatatttgaagt

SEQ ID NO: 34 - DNA, 797 bp, Homo sapiens
  source 1..797: mol_type = genomic DNA
tgacatcctaaaaaaaaaacgaagaataccagctaaggagaaattttaggcattataaatcaagtctatcccgatattcccaattctatctctgatgagttaatagaaatctaatttgatagttttagcttttatatcctatgactaatgtccaatcaactgtctgtattgtgtaactattggtctaaccgtaattcttttttaactgcatcaacctgtggcattgttataaattaaacttttttcttggtttggatttttgatcagtgaatggagtcaaactgtaacatagacataagtattagtaaaaatacggcacttactttgacctttggaagacagctgttctcagaatgattgggttattctcactgatacaattttgggttctaagttttagcccaggttcagtgtctctgacatctacacaattcaatgcagatttatttagttttttatgaagacattggttggattttttaaaagaaattcttttgaagcagaaaattgactccatgcagatttttcactctgttgcacatattaaactacaacaatttgggtgatggaaaaaagcagaaataatttgagaggcgtgggcgaaaaggccaataaggctataaaaatttcgaattacaaagagtcaagagaatattaccaaccttttattgtcagagaaatgaagagtcttacttgtgcccttgaacatgaaaggtggggttgtcagtgaatttcaagagcaattctttaaatagaggtgggtggttctcaatgagacatgagggtggtgtgaaagt

SEQ ID NO: 35 - DNA, 851 bp, Mus sp.
  source 1..851: mol_type = genomic DNA
gagacccagacagacacatacacacagagatacagagagagacacagacacacacagatacacacacagagacacagacacacacacacacacacatgcacacacacacacacacacacacacacacacacacacactgtgtttggttgtgagttactgcaactttgatggatttttttctctcctgtctctgacttcctttgaaaactcacataagccaggttttcagtgcagttggaagcagactctgttagaagaaagaggccaatagatgggaaagcagtgacaaatgaagctgcggggagaaaatgaaaactcgagctactcgagagcagagccacgggagtccttgtgcagtttggccaggctgggcctgtgcagacttagtttttggttctgactcacaaccaacatttcatggtacgttagcatttatgaggcatttcgctcacattgccaattggcctgggttcccataaatctccgctggagtgtatttggtatgtataattgtagtgggacgtagtaggtattatttttggcagtctagcagtctctcctacatctccccctttctggaagctgactcaggcctcaccagcaacagtaccctctcacctgaacccaccgggacctgtcagtgtgtccccttggagctgtgatatacagctgtgtggctctgcacccacctacatcattgccactctctgttgtaaaagtgggagaagaagcagagctgtagaacatcacagatggagagaactccacgacgcttttaggcccaggatcaatctttgcctggaactagatcaacggggttggctgaggtaggtgtgcctgtaaactgtgct

SEQ ID NO: 36 - DNA, 1442 bp, Homo sapiens
  source 1..1442: mol_type = genomic DNA
gaaacccactaactcactttacaatttaaattaagctgattacacacaaacacacacacacacacacacacacacacacacagacttattaatgtagggtcttcctgaatggcatctgtttcagttatctatttctacatagcaaaatgacacaaaattgagtggcttaaaataataagaattggccatttctccagtttctgtactttgctgggcagttcctctgatgccttccctaaagctcacccatcctgcaacaatcagtgggaagtccaagatgcctcctctctcaagcatctggcactcggtgctggatgttggttgtgtggcctcagttctcctccatgtctccgcttatcctctaataagctagagcagtttctacacatggtgctcaggacaagaacccaaaaggactgaagtggaagctgcaacacctcctgacttgcatagcatcacttccaccacattctgttggtcaaagcaagatacaaggccaccagatcaaggagaggggaaacaggctccaccttgatgggaaaagcagcagaatctcattgcaaaggggcctagcaggagctggtgcagctgtcttttaggaaatgagctgccacataggaaatgagaagccaggcgcagactgggctgggctttcgcatatgacttagttctaagaattaaatcatagcatctgtggttaaaaagagaaagcttgaaaaccactgcattaagcaaaaaggccagaaaataccatgaaaataggagtgtggagcttttttgttccttgaataaatgtcttcctggtaggaagcacaggattcctgaggaaatgacatttcttctccattgagcagacagtgaaagatcgatgtgaccaatgaagatggtgagcaagtgggtagggcaggcggttgatctttgcaggagcaggtatctgcaatttgtcctcatagggcagatgacaacagagagtgtgacgcttgtgtggagacaatcaggctgtccaataagagagacagagatgcagcaaggtgagactgagtcccctgaagtgggagggaatggcatcaaaggaagcaaatgcagaaagaaataggagctgctcagatgccgggatgttcatcaacaggacaaaaggctgaaaagtggcccagaaggactcttccttcctcttccagaaagagagcagaaaaaataagaaagacgctttgtcaagatagcaaagccatatgaacataatcactggacaccaagcaaatgacaggagactctatgatgctccagcaactcaaaagaagaggacaaaggagaaagagaagagaacagagtgagactgaggtttatacagtaaccaagccatctccttctaagaaacactttccagctccctctatgtgggcaggtggacacatagaggcacacttgtatatgtggttttctaa

SEQ ID NO: 37 - DNA, 750 bp, Mus sp.
  source 1..750: mol_type = genomic DNA
tatagtcactatgggtggagaaatcacatatggtgtacgtttaaatttgtgtacaaagaacaaacaactactgaataaagactagaactacttcttttaaaataagtgaatttcatcatatagagttagttacatcttttctttaaaacaatttgtacccatttcaaggttgtagatggggaagattactcggcatacagcgagactgtaagcatacataatttcttagatacagacagaataggaccaggtaaattgcagtattacccaggcatgtcagactagtaattgtggctcgggcatcagtgtttctgagaggaggcaggactcattcagaatcatccactagattttgtgcctttgtgtgcaaggtgttcttttgttcctggctcctcctgtctatgctttttagcatttgaccaccagtgtcttagaagaggctgttgtattccattcaatggaattggaaagatccctgttgggttgccttttctgtttccagcactgggacttccagggccttccagtatgttgtattcatctatgtggacgtgagtgggctgtacctgccagaggggagaatgtctggttatatatctacacaaccactgtgtttgttgtgtgcattttcagatgtgtgtgagtgtgtgtgtgtgtgtggggggggggtatgtgtgtgtgtctctctgtgtgtgtttatatttatgtttacgtgtgtttgaaaatggccaagggcttgtagtgaaaagt

SEQ ID NO: 38 - DNA, 870 bp, Homo sapiens
  source 1..870: mol_type = genomic DNA
tatagttgcctatggcctgagaaatcaaacctcatagatttttagaatggtgtataaagtaagaataaataagtactgaataaggagcagagctactttcctaaaaatcagtacaattcattgcatggagtaaatggtgtcttttgttttaaaaccaattttgatccacttaaaggtcctagatagggaaggctgctgggtagacatagagagtttgtaactacatataacttcctaaatgcaaatagagaagtcacaatcaaatggcacttgtctcaccccaggcttttatggctaataattaggatgttctctcctgtatggtgtccccaggcttacgccttgacattcctgggaggtggcaagcctcattcagaatcactcactggatcagtgtgcctttgtgtggctgcgtttttttgttcctggctcctcccttctgttcctctcagcacctgccccgactttgttttgtaagagatcattattttcaatttggtggaattttaaagattccttttgggtgccttttcgggatctatcactgggcctttatccccagaatacaagctcaggggcctttcggtgtgtgcatgtgagtggagtatgcttgcatggagaaggaatatcacgtcatatatctgcacacctactgcaggggtgtgtccgtgtgttcgtgtgtgtgtgtgtgtgtgtgtgtgtgtttgtgtgttcatgtgtttggaccgtggccattgtcaagcacagcataatggtttcctgtgaagagtctattccctgtgtaagggggtcatacctggcactcttcacacctgtctactgaataggccttatgagaaaatcaaggtagtgtccacgaagtcggtaagt

SEQ ID NO: 39 - DNA, 301 bp, Mus sp.
  source 1..301: mol_type = genomic DNA
ttctgttaccatacctttggcaataaccctgctatggatccttcgtcatggggatccaggctccttggcatcaggaatgccgccactatacatgcacagctgcactgcacaagccgattttcatggatggagcctcaggctgttttgtcagccagtttctgggaagcaaaaggttctgttttgtagtacagtgtatctttatcatatcacactgcaggactgtcccatgctaggttggttaactagcaggaagcttggtttacatgcaggatttgacagagctgatattcaaggctactgc

SEQ ID NO: 40 - DNA, 308 bp, Homo sapiens
  source 1..308: mol_type = genomic DNA
tgttgctaccatacaacctagggaataatagtgcccatggctagctgcattgtattcaggtggagcacagccccttggtgtctgggatgtgcttactacagatgctcacctgtcatgaattaattatctttatggctagtgcttcaggctgattcgtcagtcagtttctgagaatctaaaagttctttttgcagatcattctattctcatcattttgcactgcaggacttttccatgctagattagtagctagtaagaagcttggtttacatgcaatatttgacagagctgagattcagagctgctcc

SEQ ID NO: 41 - DNA, 750 bp, Mus sp.
  source 1..750: mol_type = genomic DNA
ttcccaaagaaagaactgtggaagcaatgtgtagagaaacacacacagatctgcacttggcttacactctgtgaactaagagtcagtgagcatgcaaaagatcaggactaatgctacagagaacaaggagttctaaagataaattaatatataaagagaattccagttatataagcggtcatattacaggccaagtaccacagacctcacaacagaataagggctccccattcagacataataaggataaaggaagaaagaaaacgcaaggactgtatgaatcatgatcagagaggaaaatatcaaagaaaaaaatgtcttaagtgcaggtcggtaagctagttaacaagggaagacgaagagctcttctcagcaattatccttaaagagtgactgcagacgttttatttgtcaggttttaaacctttaaagagatcttaacactgtcaagaagaaaggtggtgtaagaacgcctttggacctattttctcagatctttaagaatagagacattaagaagaatgaagactataaatactcaattcacagataacatcagcaaggtcctgttaagatcacaaggttaaaaaaaaaaaaaagacatttttgagtgggtatgaaagatactgcgagggagaggtgttttctaagagaataaaatgtgaataaagggtaaaacttcactggcttgtaatcagtggccacacgtcataagaaggataattaaaggacatcaaaactaactttagc

SEQ ID NO: 42 - DNA, 1353 bp, Homo sapiens
  source 1..1353: mol_type = genomic DNA
ttactaaagaatatagtaaagcaataattagaaaaggaaccaatgaaataagacaatttatttaaataaacactgatatgttcacataggtgcactctaagaaaaactgtatcctagaggattaatttacagaaatccttgtagagaaatggtaaagaattgagaaactactaaaaacttggaaaaggaaaggagaggctactttcttataaaataaagcaaagataatccttggattaaagtaaatgaaaaaattcagtggtaatatgtaggaatcaaaatattaatttaaaatattggaaagatcacaaagcacggagtgataaccaatcaggttttatgaacaacttatcctgtcacaaatatgcacattctagtcaatcaatgaacttgaaaaatataaaatattaatactgccaatcaaacttaggaaataaaatttagttcagtcactgatggcaggtccaatcttggctaaggatcataattcttgagtaaagtagctgggactaatgtcagagaaaggaattatgtgtgaggtaatccttagttaagattagttctatacaatgtattgttattttgcaagccaagtgaaaagaaaaattatttactttagaattaaactttgtaatatcaccctgattctaagcaaaattagctctttagtgtaaagatggctgagtgtaggtatgcttgtgttataattgtcatgactcctacttttaaccttgctcatttaaaaaattttgttttatttttttaaaaaatgttacagagaagcacaatcaattgaccactgccgtgtgcgtgtgtgtgtgtgtgtgtgtgtgtgtgtgtgtgtgttaggggtaggggaatctcaaaaggacatagcctgcccaacatcagtctttgatttcttcttgtaattctttggaaccattgggtagcaagtggtgttggtattttctggagattacatagagtgaatatttatcaatgatttaatactgctttgtggtactactaggttctcaattttaatcagatttttgaagacaaaatagaacatgaggaggcagcacattgagtataacacttctcagctcttccaaaatgtggaacccatttaacataaaaattgccaaggatctttccagagctaaaatgctgtggcctacatttttagatggccttaacatatgaagagtggttagcatcttatacaaaataaaatagactgtgcattataatatcactggtggaagacattgtcaaaacatggctttatagaaatgaaaaggaaattgaacattttggtttgtggcaacatgagtttggagatagaaaacagaagttatcttagatagt

SEQ ID NO: 43 - DNA, 750 bp, Mus sp.
  source 1..750: mol_type = genomic DNA
gcacagtgtggacatcatggggatctcaaagtttggctagaaatttggtttttgttttggtttctgtgcccctcccccttgggagtcaaggtgaagcaaatcttccaatttattctttagaaatctccctacaaacagtaagaacatgaatataaatgcaagcagatattagcatttgatcatgctgggaatgtagacacaaacacaaaagcatctttgtctcccaaatcagcaagtctttgtaggtacactgtgatttatgcacggtgaggaggtaaaggttaaaaagatgttttcaataacttttttattttaggactgtaaacgtcagtgaatacagaaggcactgtcagacgaatgcactgtcaagtgttctcaaaatttatacgtaggcagcttctgggattattaaaactggcttactaactacctaaaatggttgcttgcagtgttagcgaagagtgttgatagcttgatcttcagcaagtgactacattgctcattacagacaataaatccctaatcccatgtcctgttttcatcttccatagctatcatacaactttaaatcaacatgtgtccctttgggtaaaaattggcagctagagtggccaccatatttactatggttctctccttcttcttcctcttttataggcttaggtgaggtttggtttgagttttgtcttttgtggtggtgagttaaacctgcaacctctcagacactgagcaagcccttcaccccttagc

SEQ ID NO: 44 - DNA, 931 bp, Homo sapiens
  source 1..931: mol_type = genomic DNA
gcacagagtgtgaatttcagaggaacttacagttttgctagaggtttggagtttttcctattttcttatttgaagctacggtgaaacaaagccttctattttgttctataaaaatctcccccacaaacctcaagaacacaaatgtaaatgcaatcaggtattagatcatgctgagaatgtaaacccaacacaaaagcacctttgtttcccaatttagtgtgtcttttaagctctactgtgggtgatttatgcactgcgaagaggaggatgtcaaaatgatgcttttcaacaaatctttatttggagtatataaaagtcagtgaatacagaaggcacactatcaaagggaagcttaatctcataaaagactaaataaagcaacagaacggtcaagtatatcaaatgatgtatttaagactctttgggattattaaagcggactttatacacaaaatgattagcttacgatgttagtgaaaagaagataaagtttggccgacctgactaagtaactagaatgcatgtttcagacaataaatgtaacttttctgaaattcctaatcccatgtcatattttcccctcctatagttatacaattcaaaaatcaacactttaaaatttcaaaatcaatatatatcagtttctgcagtggttggtgggtagtgtaacatgcataatgtgttttctattgttgcacctgctcttcgttccgtttttcccctcttttctgccttttataggcttaactgagtagtttttataattccgtttcatcttttttattggctaattttctatattttttgttattttcatggttgctttagagtttatatcatttctctgtctatatatatatatacacacacatacacatatatatattttacaatatacatatgtaatatttttcaaagcctgcattcaagt

SEQ ID NO: 45 - DNA, 750 bp, Mus sp.
  source 1..750: mol_type = genomic DNA
tacctggctaaaatcaaatacctacaagcaaaaacgaaggtgtcttttgtcatgtgaggccagagctgagggaatttgtttggaggattctgctgggaagataggatccagttggtccactgctgtcttgctaaggcattcaatggcagctcgctagctgtgtgcctcatgcaagcagctgtttctgagcatgcccatctccagtgaggctttttgattctcattaatctgtcattaggtaagaaaactgaggtgactggaaaaatctcatttgaaaagcattcctctcacctagaggatcaaggtatctcaacaaagattttgctcaatcagacattcaccaactctgcttttctggggttttgaggtcttgcttgttcctatattcaaagaaccaaagtgttcagtaaatgtctctaactcactcttcagtggaagggaagccacacatgttgtatgagtgcacacatgcgtgtgtacatgtgtgttttgctaggatttaaatgccttcatgcatgcttggcagcctgagattccctcttttagttgtgctaggtacactgtaatgtgccatccttaggcacactgtgatgtgcctgccatccagcatatccagaaattttgtcaaatgggactgaagcatctctctgcctcggtgtctgcctatctctcttctctctctctctctctctctctctctctctctctctctctctctctctctctctctctctgtatagtttagcatag

SEQ ID NO: 46 - DNA, 1042 bp, Homo sapiens
  source 1..1042: mol_type = genomic DNA
tagctcactgtaatttcaaacccctaggctcaagcaatccttccacctcagcctcccgagtagctaggactataggtgggggcccccataccgggctcattttttctttctttctttcttttgtagagacgaggtcctgctatgttgcccaggttggtctcgaactcctggccttaagtgatcctcccaccttggcctcctgagttgctgagattacaggcctgagccactgtgcccagcctctggagtctttttaagaaatgattttgaaatctttctaaacacattaaatatcttggcgttagaaaagtgtgcagaataaggtgacacctactgcctagcttggttggagcttagcattaggccatatttgctttagatagagatggaggcccctgtgtcttactccctggaggaaaccacaattctgattttggtgtgtgtgatgttcttgtgcctgtttttaaaattgtcctacataggtattcatgcctagcaacatacagtgctgtttcgtatgattttactctggtttaaatggtataccaggcatattctgcatttaggtttttccctcaatattgttttggagatttaaccatcctaaacatgaattctcacggatccattttaacctccacgtccgattccattgtgtaacagcagcttatttgcccaggctctgttgacggacacttgggtggtgtgcagtttttcgctattgcaagcgatccctctgtgttctctccaccagtgcttgctcacagggtctgtgtattttcagcttagacagatgttgccaaattgtgctccgaagtagttgtcctaatttttaggcctctgtgtttatttttttattttattttatttttttgagacagagtctcactctcgcccaggctagagtgcagtggtgcgatctcggctcactgccagctctgcctcccgggttcaggccattctcctgcctcagcctcccaagtagctgggactacaggcgcccgccaccacgcccggctaattttttgtatttttagtagag

SEQ ID NO: 47 - DNA, 750 bp, Mus sp.
  source 1..750: mol_type = genomic DNA
tcttggggaagcagatttggctttgagtgcggagttgatactgaaattacagactctttatttttatctattggaaggataccatttcttctcggaaagattgggacagaagcaaggcagctttgttgtcaaaaatgatttacctcttatcaagattctaccctcattaatttccttaaagctgcctgtcagttgattaaaagctgtttccgctgcaattatcttaaagttgacttcagtgatgtagcacaaagcatctatcctcaaaacgttcctctcttcagaacattgcagaaaagtaggaggggcaagttaaaagtcagaggtgtctctgatgtgatttgtcagaggaggggagaacaagatacaaaattaaaaataacattagaactgccatcagagccattcgttcaggagaatttttccatgacagtcacacagctctgatgatcccctcacacatactcaccccacggtgtctctcttagcagaatggagtccatactacatgtcttctcaaaagaaaattgtgctgtgtgcctcctgtaggtattaaactgaaactcttaggacccagggagctctgaattaagagtttattagactgggcatgtagttcagcaacagagtacttacttgcctaacatgtgagcagccctagtgaatcaccagtagtggaagggaaagtagggtatgtttcctccaccacctacagcaaggctagatagcagtttgaaaaatgtccctaaa

SEQ ID NO: 48 - DNA, 1305 bp, Homo sapiens
  source 1..1305: mol_type = genomic DNA
tcttgatttttctatgcctctttgataatgaaaatcattctttagttacaaggtctaccacttccctggaaggaattattccatttcattctgatccaggctcagttccactgagtttccgaattgtttcctgggcttgcttatcaagatggttgacaccctcctttgaccactagtctctcaaattaatttagctccttatcattttctccagcttttaacaaccccaaatgatagaaggtaagttgattttctcagtacatgatgaacaaaatgctagttgctaagagagggtagaaaataaattgaaaaaacaaacttcatattttaagagcttattctgggggaagggataagaactacaaacaggaaaacagttaaggaataataccagatgctttttgagtactgtggatctgcaaatgggaacacaaatgattaagactcccaaatcccggctccagaggcatcctgtactgtccatgtctcaagaaacatcttctggagaacagctgtgccataaaatcaacatgtagggttgccactgtgtggaagagactggggaggtgctctctgccaatggtgccagggtgctgaggaggaatgaagcctggcaccaaggagagtcattttttttgtgtcatataaacacagattagggagctaaagatgccagcaacagattagagtactgttaccaggttcttttcttcaagaactgtaggccaactgagcctgtctcttggaaacaacaggaggaaaaagcctgatgttattaaaacaaccccaaccatttagtaggcctctggccactaagatgaaggcaggaaatgtgccagagggggttaatggactgagctagaaagtatttacatacgaggcacttagcctcagaaaccaaatcctgagagactgctaagggatttctaaggtttcaaggttctcccatgtagtaatgacagaatggaacaagctagactcagagtttctcaacctctgcactactgaaattttaggacagataattatttgtgtgaagagtcttgtgcatcgcagaacatttagcatccctggcctctacccactagatgccagtagcaccccactccacatagttgagacaaccaaaaatgtctccagacactgccgaatgtcctctggggataaaaatcattcctagttgagaaatgctaagctagataaacatatcctacattttaactccagggtaggtgctcttagcaccactactagaatgtctgctgctaacaagtgatgctgttatagtcatggaaagaaactaat

SEQ ID NO: 49 - DNA, 679 bp, Mus sp.
  source 1..679: mol_type = genomic DNA
taggttccagggaaatgtgtaggaaattgaaacacaaaacatattttataacaagagtcctgcatgtcagtagatctaaaaacccaaagagggtagtgtctcttcttctgttgtcttcaaactatattatggacatatttagacataatatcagctatagccaaataacacccagtgatttgaagcacaggaattgaaggcctaaactatcccttgaggggacagggactgcttctttgctgctgctgcggaaattcattttaatcttgagctttagtctgtccctatctggagactgaagcatttctttttgtctacaatagaaaaatgacatcaatttagccctgtactttgcaatcccacgcttgtgcgtgtcatatttcttgaattatcacagttgtccatcaattgtgtcagacggagcaggaaatttcgctgtgggttgggttctgccaagatacagatttctataaaagattcatataaaaagtgcttccttataggatgctagcttgtgtgtgtgtgtgtgtgtgtgtgtgtgtgtgtgagtgtgtgtgcatgtgtgtgtgcatatgccattaattttgggaatataagtgcttttactcacaaacacccttttgtgtttccttactctcagggttttcatgatcttggaatttagtttgggtccttgtgc

SEQ ID NO: 50 - DNA, 1080 bp, Homo sapiens
  source 1..1080: mol_type = genomic DNA
tagactctccaaccagggaattaacatggaacaaggcattgcacaaaaatctcagaggaaagagcagagacaagttattcagactgcagggcatcttggtcaagtcctgtgctccctctgtcactccctcaggctaagaaagtaaagctgaacacaaaattcttagccctcagacgaggggaaataaatctcagatattgttggaacacaagtagtcactcccactctgtctgcccagagccatgcaaacaacatcaagatggagaaaatggcccagagactatgcaaatatattagatactgtattagtcagagttttccagagaaacagaaccaacaggatgtgtgtggagaaagagggattgattttaagaaactggctcacaaattgtggaggctggcaaatctaaaatgcacacagctgtctggcaggctgaaaattccagcaagaagttgatgtcttcaggtcttaagtcccaaggcagtctggaaacagaattccttcatcctcaagggacctcagtctttttctcttaagaccctcaactgattggatgaagcccacccacatcatggaaggtaatgactttccgcaaagtctactgattgaaatattaataacatctaaaaaaaaacttcacaagaacatgtagatgtatataatcaaataattcagcatcatagcctaatcaaagtgacataaaaaattaagtatcacacacaaacatgagggtgcacatacacatatccatctagaataataatctagaagaagaataatagttaacattcaatgcatgtgctaggtactgttttttgtttgtttgtttgtctgtttgtttgtttttgagatgaagttttgcttttgttgcccaggctggagtgcaatggcttaatctcggctcactgcaacctctgtctcctgggttcaagcgattttcctgcctcagcctcctgagtagctgggattgcaggtgcccaccaccatgcccagctaatttttgtatatttcatagagacagggtttcactgagttgaccaagctggtcttgaacttctgacctcgggtgatccacctgc

SEQ ID NO: 51 - DNA, 750 bp, Mus sp.
  source 1..750: mol_type = genomic DNA
ataagtggaagtgcgtccacagcagtgaaaatgagatttgttgagatggactaagacaagtgatactttcatcgttattaattctgccttcgttttcataattacctttgtgtgtgcagtgtttacaaaacaggcatttaataaccttccagtaagagaaagaatactgtgtcatgctcatttactctgtgctcctattaataacagcaatttaaaaaattaagggagcaattattcctttaacacagagcagactccaatattacagtaacacccaaggggttgtaaccattgttggctttggttacactgtgacataacttaatagacattggggggtaagggaggagggctgttacctgcatgctcagatatagacaagtccccaaagcctacagccaggggctgataagtgactgaacacagaacattctctcaatggccatgagagaactcctgcctgctcttttcattttcaagctgccagtcaaaaaaacgctcacagagtcagtgcccaagctgggcatattaggctgcttctgtgtgctgtggagtttgtaagaaacaatttctttctccaagctgcattgtttaagggagtaatccattcttttcagagtacccttgaatacaaactactaggagtgtagtatatccatcccagtgagcaggtcttggtaagtagcaatgactcccaagtacatgacctgtgtgtgcgggcatgtgcatgcgtgtgtctgtctgtctgtc

SEQ ID NO: 52 - DNA, 1438 bp, Homo sapiens
  source 1..1438: mol_type = genomic DNA
atatgcaggagagaactgtgtgccacagacctccttttgtctcagcttaaattgtagcgtaaccaaacacaacaaagattacagtaccttgcagtagggtgttactgtactattgaaataagccttgtatcaaaagaataattgcccctagattttaaaaattactattattagggaaaacacacagtaaatgagcatgagacagtactgttacttttactggaagggtgttaaatttgcattttgcaaatattataaacaggataaatatgaaaactatggcagaaggtaaagacaactaaataataatttgagaacattttgataaatcatatttccactattgtggatgaccatccactttattactgaaatgtctgacagctgataacatcaatttttgtggctgaggcatttaattactatgtgacaggtgtcactgaatgggttggtactaaaccagcagaatttccatgccatgcaaagtgcttgtgaaatccagtctgcctggggcttgcagattgcacttaactccaggatcctgtgactatttgtgaccttcttcctcctgccatagggccaccttctctatccaggcagccaactgtgtttgctgggggttttggttgtggcatcagagggaatcagggaggcagggaccttagaagccatcttttgcaactcccttgggtttccatgatatcaggtctcagagaaagagggacacttattcgagactgcacagctagtaaatagggaagacagatttggaactcagtttaccagggcttcttaccacataaggtgttcagcattctgtcagctacacaggcccaggtgtgttcttgcctgtctttatagcccctctctgcccctccctctgctctcagacatgtcccttccttacatcttaggaacacagctgggccagcactccatcaccaaccctggtgcccaggctgagctccgagcattattaggggtggtgacacaagcctgacctttggcaaacagggtgctgatggagctgttttcagagcctgacactctcttctggacacaagctcagacgggatagtcccacagctccagagttcttgccctttgatgtgatgaggggaagggattgtgaaccccacgctctgcttatcattctctcaaggctcaaagtgtggctgcctttcctctttctttcagagcttcacaagacaaaatgatactaccaaggaggcagagtctggtggcagcttccaggtctattgttgtagacaactctctttggctaagcattcagggagttggtagacaagttgagccatcatccctaacaccagagccaaactctcctgagctctgtaatttagtccaggacctctttcatatcttgttcagtgcattcagcatcatcttcaacaacaaaatcatgctgttggccatc

SEQ ID NO: 53 - DNA, 366 bp, Mus sp.
  source 1..366: mol_type = genomic DNA
cccacttccatcccccgcctccctttctgagattccccccttccatcccccgcctccctttctgagatcctcccccgttccgtcccctgcctcctttcctttctccccattagtctaatgcagtcattacttccactaaacttgcaaaagagcctcttctggctttgctggtttccaccataatttgcctattttccagttgaaccatgttcttgttattatctctacctactctttatttgcggtttaatttgcttttcttcctctgagttctggaggtgggagcttggcttatttttaggttattgatttaaaagctctcttcttttttctaatattagtgctcagagctatgactttctcttg

SEQ ID NO: 54 - DNA, 477 bp, Homo sapiens
  source 1..477: mol_type = genomic DNA
ctcccttccatggctccgaggccccacaccatctttcctgctctcactggcctcacctcctaccattctccccttgctccctctctcccacctcactggccccttggctcttccttggtcccagcttgttcctacttcaagacctttgccacgtggcccactctgcctagatgcccccctaccgtgcccactgtgcacagacactacttcttggattggaggggtctgagcactaatgtcacctcttctggaaggccctttcctgaccatttggttcaaattggcatcccctttcctcttctctcagtcccttgttgaattccttcatggtgtaaaacttcttaccatttattactgttttatttatttgtctgttagctgagattttatttatctctctcacaagaatgcaaagcccattgcagtattctaagtgtttgtaatgtgtggttggtgcccaacaaagacttttacata

SEQ ID NO: 55 - DNA, 750 bp, Mus sp.
  source 1..750: mol_type = genomic DNA
ccatccaggacccaagctatctgaatctctaaggtggcagctgttattgaagaattcccacagttcttgtcttttgagacacctgcgggagaccccaggactgaagggagcaacaggggcagctgatagagttccccgtgacacagacaactaggtagctggtgaacattgtccctgtgaggaagaggcagcctggtgtgatggcacatgccagtggaactgtcacttagtgggtagaaatgggaggatcaagagttcaagactatgcctggatacatagcaagcttgaggccagcctgggctaacagggacctgtcacaaaacaaaatggtaaggctgggattgtagaacccaggaagaaacccactgcagctgcaatgggcagtgtgtgtactgccctgtcccctctgcttggccggagcaggagcagtgtgtgcaggcctgcctgtgagctcgtagacacatctagcagggtctggagccaccacctgggcctgtgtacacagagcttcgggtggctcaggagagatgtgggttgggattaaatgattttaattaaagtggctggttcaggatgggagcgaatggggacgaagaggcggcaatgctaaatctgaatgctgggcggccgagcggctgttccttcaaggctctcgactcatgttccacagtatgtgcctggaggtgacctgggacagtgtctgtgctctaccctgtccccaacagtgacccatcataaggaaatgtatt

SEQ ID NO: 56 - DNA, 869 bp, Homo sapiens
  source 1..869: mol_type = genomic DNA
ccattcctttctttgcctctttggagattctaaaggtctctgaaatcattattagattactgtattaccttccttcctactggaatgaattcacctccatgttttttattctgttgcttacctttcttagccttcatatacctcttgcgctttgaaattgatttgcgactcctcttagctgagccaatgttgtggagacagggagtggcctgcagtgcctgaaactgcagggcagcagccgcctcccaggaatgtcctgggtcctttcctcctttgaggtcccctgccatatgctcagatggggccaggcacagcaacgggggcacacctccctttggcccagcagttggggtgctgccaggcctggtggaggtacttggtaaacatttgttatggaaaagcagcaggagggttctaaggctgggattctggagcccaggaacaggccaggcatggctggagcgggtccgagtccgtgcactgtgtcctgtatgttgctcttagccgcactcaggagcagatgtgcaggtcactgcctagaagctggtgggcacctccgtgcagcaggctctggggggtatagccaccatctgggcatgtgtacagagggccttgctagtgcgacatatgaggaaccagaagatgtgtgggttttgattaaatggagttttaattaaagtggccaggttgggggaggtgaggaggaaggcaggaagcagcagcagttttgagtctgaccgcgtggaatcctataaggtggttctttcaagaggcattatgcacattccgcagcatgtgtcccctgtctgtatggggcagcatctgccctctgatctgtccccaacaaggggtaatcacagagaagtatt

SEQ ID NO: 57 - DNA, 750 bp, Mus sp.
  source 1..750: mol_type = genomic DNA
tgggtaagattagcccttttaaaattattcccttatgttttaatattctcaacaggaaggattaaattagagattaaaataaaaaccttaggggctaccttgtaaagttaagggtggtatcacattttggtgaaagtcaaaagtataagaaagtgtatttctttaattcattcttaattataggatgcatttcaacgtttttcttcatgaacactatattggtttggaaccctcttttaaggcagtaagtattgcataggttttgacattacaaaaattataaagtctaaaaaggtgtacggcaggaaataatcactgcaacactgatgaactcggtgtaaattagcctttatcaatttcaaatgcatgcttttctaagtgaaggagtaggatctgaggtgtgctgagatagtaaaggatatgggaaggagttcagctcctctctagttcccttcacgtgcataatcaaaggactatttacatcatagatgcagctacatcacaggacatctgtttgtgtgcatagagaaatattagatatggaatagtttgaactccttccaaatttattttcttacactacttggaaattgtgatggctatctaaaccatttttgtactagacaaaataaaaaataaaaccctgatttaaaaaaaatacattcacaataggcaaaaaagttttagttgcatataatctgcattttttattgaaaatttccatagttttctcttcttctaatcctttgg

SEQ ID NO: 58 - DNA, 1031 bp, Homo sapiens
  source 1..1031: mol_type = genomic DNA
tgggtgaccaaggctgaaagccaaacaaaatgaacatcttccataactacatggaaattttttctcagaattagatggttaaaaatattggagattttggcagtactttatagtttctcaatcctaacaataaggataattaaatcagagactaaaaaaaatctcatggccactttataaaaagtggaccacattggccaggcatggtggttcacgcctataatcccagcactttgggaggttgaggcaggcagattgcttgaattcaggagttcaagaccagcctgagcaacatagcaagatctcatctgcattaaaaaagtggaccacggtttgtgagtcaaatgaaaaaagtgtaagaaataacgttttaaattcatttttaattttaggatgcatttcagcatttctcttcatgaacactgtattgcttcagaaaccctttaaggtagtaagtattgcataggttttgacattataaaaattataaagtctaaaaaggcataaggtagaaaataatcactgcaacactgatgaactcaaggtgtaaattagcagtgtttaccaatttcaaatgcatgcttttttttaagtgaagaagggatctttgaggtgcgctgtaagttatttaacaatactgtggaaggaattcaagttcctccccgccccacccccaccctgcgtcttaaaaatctaaataatcaagggttgtttacctcttcacttaagtttttcttagaggacacttgttttgtgtgtgtgcacagacaagtcaaagacataatcagatgatgtggagtggttttacttctttcaaatgttactcctacagaatttatgaaattgtgactgccaactatcaaaaccatctaatcagtatttgttttgtattaaatagcagatggttaaaaaatatagaacactgataaaaaaacttgtgtccaagatgtgaagttatgttttttggaactgcatgttacttgtgatttgtaccagttctttctgtagttttcctttaccatatcctttga

SEQ ID NO: 59 - DNA, 750 bp, Mus sp.
  source 1..750: mol_type = genomic DNA
gttaatttcatttatctatgtacctggaatttgacacttctagtgactagggttggtgacaaatcacttcaggctagctctcagtgtgtgtattttatattttctgcagactgctgttgtcacttgtgcaacaatatcccaagctggaaattgagctgctttatggccacataatatgcactaatgaaatatctttaaaaatgtaaaggttggctcccttttcaaagccttgggactcagggtaggagaaactctttcctccctccctttattctcatcacattttgcttctcaagcagatcacaaaccaggtgataacacacggtctcagatgacacatctatgatagaactataatgtggggagagaactgctgccagcaaacatttcacaggagaaggtggggacagacaaagcacccagaaagagattccaagtttgttgatcaaaccgtttgttctttcccttccggttgggtcttctgccacttatttcagtgctcagctacatgttactctgcaaagcagaagtcagcatggctcaagtctcttcactcatcacagcagttctgatgagacaattttaggcagactgatccctccctgtaacaggcctcctgttcttttaggattaccaagaactaactgtacacccgctgctctgtgttctagggatgaggcatataatggtgggggtggggcatcacccatgactggactatgggggttaaaactaatttctatggatcgt

SEQ ID NO: 60 - DNA, 960 bp, Homo sapiens
  source 1..960: mol_type = genomic DNA
gtaaccaccaagtactttaattaatcttgcttttattcccagatttcaaaatttttaattactagcttgggttgctaacaagcagttttaggctggctattggtatgtatgttttatatttttttacactacacttccacttttgcaaaatctcagactgaaaagtgagctttttgtttcccaaatatgaactaacaataaattttaaaaattttcagctgggagtggtgactcatgcgtgtaatcccagcattttgagaggcagaggcgggaggatcacttgagcccaggaggccaacttggccaacatggcaaaaccatgtctctaaataaataaataatttttttttaaaggtttgccccctgccctcaaacataaggtttatgtaaggtaagagagactcttatctcattttgtatattcctgtttcattttgttttccctgcagatagcaaactaatgacaacatttcatggcagctatttaaagttgccagcctaaatcacctacactggcttaaatgacgtactccagctgatgtgtctgtcacttagggaagaatgagacagttactgccaaaaaggagttcatacgggagggtagggggatagaaaaatattcagaaatgagttctttagtttgctggttgaatactttgtctatttctttttactgctttgttggtcttttaccacttatttgcgcactcacctgcatattatcatggaaagcaaaaggcaatcagttcaaatttgtttacctgtcaatagcagatgtggttaaaaatttggtgagaaaaactcagttataagataatatattttactggactttccaggctttaccattccattcatattgcgaagaacaactgtaagtttgccctaggtactagggagatgatggggaaaaactgtctattattggagtatgggggaagttattaatgatgtcatagt

SEQ ID NO: 61 - DNA, 750 bp, Mus sp.
  source 1..750: mol_type = genomic DNA
atttttcttttccccctctgaacctaggccttaggctggctaggcaagtctccactacagccccgcccactccatctcggctgtggctcgcgggaaaggcagccccctacagttagtctccccagccatcttctccggttcccctgatgctctcatggccccagccatcctagagctggtgcgccatcgctcacctttagcattcggcctgctgggggtgagcttttgggttagaaggaacaaggcatcagatggggtcgtgtttttttacattccaatcagttttcggaggggaagaacagacatttttgtcagctaattgcatttaattacagagacaatcgctgtttccgaacgttgttgtgtccactccctcatccttcaatcagttcaagcacatccggccccagctgggagccttttaattgcataatttggatttcacttctggaaagaaacttgagaaagactccgtgagggtctccattaacacaatcagctccccaggcacccggggctggccctaattgacccatctcagctaatgcctctttccaaagaacgcccgcgtcatttctttggcttgtttgtgctattgtatcctgttttagtgtcttgatcttaaacatctttaaagagggcactcagtggcttgtttaactggaagtggtccagctggtttggaaggacagtgcatcaggtgatgtaagtccaagggtaggaatttctggcctctccttcccctgccca

SEQ ID NO: 62 - DNA, 919 bp, Homo sapiens
  source 1..919: mol_type = genomic DNA
atttcagaaattcttagtgattcaagcaggacccttaggcagctgggctcctttatttggagcaggctatccagggactctgacaaaaacccatgtggtagagcctagagcagggctctcttctggcctcagggacttaggggacagctggcagggagcagggctgggaggagggcacagcctcctccagagtcagccccagccccagccccagctccagctccagccccagctccagctccagccccagctccagctccagcatgggcaggacaggcaggtcagcagaggcagagttgagatgtctccagagactgtgatcataggagagacacaagaggcacttcctattagagcacatttttatgggaagtctaaagggcagagggagggagtaagaaagcaaccagagaaatctacagcatagagcccgtgtgttctgctccacatccctccaatcctgctgcttcctgctgagcactctgctctagcccagcaatcctgtaggtctcccttctttgtgccactgtgaaggctctccacacttcgacctggactgtcacaggctggcagaggtggggtgggcatttgacctttgctcagctctttgaaaacacaagcagcctcaagaagtgagttccttgaacccctggttttagatggaggatctctattagatgtcatgggagatttgggcactaccctttgaacatacaaacttaagtcattggcctgagatttatgctccttccctcccacggccaccttcctactctgcttaggaagttgcagcacaagggtcctgggttttctggggaaaggcagcccacctgctgtaagttggcccaaagctacttgtctgttctctccaagtgaccacagtgcagatctgagttctcctccggttcctttccctcca

SEQ ID NO: 63 - DNA, 750 bp, Mus sp.
  source 1..750: mol_type = genomic DNA
tgcctctgcctcccaagtgccgggattaaaggcatgcgccaccactgcccagtgagttcctgtatcttaagacgtccatatttgcttcttgaccagattggattgtgttggcatgcatgattggacatagctatagtaataaagatgggcccattcaggtgcaaggtatctggggagagaccccaacccactccccagagtgtacttggccctgcttgtggagctggaagcttccaaggcgacttgctgctccggcctgagtcaaggctttccttcatctaggggccatttgaggcactgtggcaatcaggctaatttactaaatggcaccctggtggcacccaaccattagatggcttcccacagggagtaatttatgaactggaggaaatctggccgtcttctctgttgatttcccccagatccttaattactgtcagtactgcctggctggtctgatttcctccccaaggcccgcccacccatgcacaattacccagcacccggcacttccaatcctgctgtcacctggaattgtggttagttgtctaagatgttttgaatttagagggctaagactttgtgatccggatcacaaaccaaaaaagtgtgtctctgaaagcatccaagccataaatgtcactatactgtgggaaagataccactaaacaactcagaccccaaattcattttattagatgatttcagggataagtagaaaggattgttgagccagtgtagtgtggggca

SEQ ID NO: 64 - DNA, 1607 bp, Homo sapiens
  source 1..1607: mol_type = genomic DNA
tgcctcagcctcccaagtagctgggattagaggcatgcaccaccatgcctggctaattttttgtatttttagtagagatggggtttcatcatgttggccaggctggtcttgaactcctgaccttgtgatccgcctgcctcggcctcccaaagtgctgggattataggtgtgagccaccatgcccggcctggaagagctacatttctaaaggttgactcagggagcaatttgggggatcaggagctggtgtcaacaccgggtgaggggatttgccaacaggctactgcaatagccccagcaagagctcttaaggacacaggtcagaatgtctagagaaagaagagataaagggtgcccagccctctgtacttggggctgaaaagtcccccttacccaagggaggacacctacattgaccctgcacacaggtcagagggactatggaaagagaaggccacagaggaagccctcagacctccaagcaccaaagccacacagccaagtggatgaagaactcatgagcgtgagtcagatggcctgtgtctcggcacattactcatccacctgtgctgccaattttcaactgataaaatggggcccagaagaggactgacctcacaggggtgttctgagagtgaaagctaatgacagtaaaagccttccgctgtgtctggcaccatgacatctcctgacctgtctctctcacatcagacttgcctagacacggcggtggctgacagcctgggaagacacacttcattgaaactgggggactttgcaggcgccactgctcctctctgtacccagcctgcagtgaccatgcaagcacagtatgcgtgctcctcagggctcagccttcccactagatggggcttcctcctgtgtctcccccatctgcatccaggagcccctttcagcagaagcctgtttacctcaagatagatgatcttgaacccatggcttcattaacaaagcacacaaggtagatggttgctatctacccacccagaacaactggctcctaaacaagcttgtccaattgattttcactaagcccccctctgccactgtttgccaggcactgggatatagagcagaggcatagcatttgtctccatgggcatccttccgaggcagtcccctgcacacagccccaccctccctctgccctggtgtcctaggagacccctgcaccaaacctacacttttccaatatgaaaagggaagtgacctggctgatcagtgctgacctttctaatcatgacaagcttcggaattcaggagagatggctgcagccttcatctgtctccttggttttcccaacaaagccaactgactgtaaaatacagtttcccttaaacatcttttctgcctacctgactagtcaaccagggtagctgtttcctggtctccccagggtgtgtctggagtatttgtcaagaatacttcaacttccttctaagtgtccctgcatccactccaaactcagcagaactttggcataatgagaaagccctaagtcttttcaaaactgacaaacaggcttgttcctccattccacagttattgattcagggtgttctgtgccaggcagtgagcctggcca

SEQ ID NO: 65 - DNA, 538 bp, Mus sp.
  source 1..538: mol_type = genomic DNA
cagtagcattggtggctgccagaccgaggtgcagcaggcggtcctttccgggcccagtgggacattgaaattattatatacacgtctaggacgctttaaaatcactacctcttccaacttcaccacctccgaaatgacccctcctatcatctccatctttggcttgggagcaccccttggtgttggtggacaaagtaatctggagataagggtcttacacctctgcagcagaggccgtgcccccaggcacccacagactcagccctgctgcctttcccacctaccccattatcgctacccctttccagcgcatggcgcggcaggggattcggcagaactgcagcccagccggacgaaggcggaggctttttgactttgcttggtctgccagcttcactgctggcaccgaccggctcaagatcccagcaccgactgccagaaacctagatgcggctgggcagagtgtgtccccggggtccggggaaagtgcgaagcccacgccaccgcttctcatgcacgttatttgtctcgacaacag

SEQ ID NO: 66 - DNA, 796 bp, Homo sapiens
  source 1..796: mol_type = genomic DNA
cagaagccactcacccctcactactcatgactaggaagaaagtggccggggcatgagggttcgagttctgtcctaggtcccggaagccagggctcccagcgtctagtcaggggcgcagaaaccctcgttcctccaaacccccgccacagggaccactccagaagaaacccattcgtgatttgggtccctttgtgtttgagtattgtggggagtgggctatctagactctaaggactccaagctaacgatgacctgtgtgggccttgctctgaacagaaaactcaaactcagcgtgggttcccgcgtcttccccaaaaggccaaggcccaaagaacccttttcatttggcgaggggcttgaggaagggaggggctggggcgggagagggtcggggtctgcaagccagcggcccggcgcagcggacttgtccggctgctcagcttggcagctagcgtccgtgccggcaacgctgttaccctctcctgggctggagagtggggacatcccttccccgcccctaggcgtccctggggtcggcgccagagcccgggagcgccggaggctctggctggatggcggcgaggagggctctgagtcctctcccggccaggccgagcttagtatgtggaggcggcggcgggagtgcgggcatggaccaagggaagggggacacccaaaccgcattggtgccttcctttccctccactcgcgccgtagacctccccaaattgcccgtctccggtatgactgaatctgtctgctggcctctcagctaccaactctgcccgaaatctag

SEQ ID NO: 67 - DNA, 750 bp, Mus sp.
  source 1..750: mol_type = genomic DNA
ctgattttttgcctaagccaaaggcaggttacatgcaaacctctatttaaatctgagcgccagtgccacggcggaatggagactgttgattatatcagggagtcttccctgcaaaccgggaaagaagcgatcacaaagccaacagaggctcatcttgtgttcagtcttatgctagagaagttttagcacccctgatcacccccaggaccttcagttgctaagatcacagacacctctccctttccttttcgaaacaacctcagctgagccagagatttagggttctggcttctgggagggtctgtggtgggagtgggggtcattttctcttccatagagcagcaaattcaagcagcaagaaactcctggaccgagccttcctccaaccaatattcccagctttgtgtgcgttctggccttcctgaattttgcctagacctgcggtatacccggtgggcctggcctgctcctagcagccactcacccgaagcttgccacagtcagggccgtgatttcaacagatttaagatccctcctggcctgaaccgattttcattttcatctatcagcctctgggctcctctggcctgcgaggtggaacaggcgggcgggtaattgtctggggaggatcatagacctggaattgtcagtttggactgagagggtcccctctacactcgataaggtctccgcgacactgctggatccgtatattaaataaatattaatattaaataaatattaaaatcg

SEQ ID NO: 68 - DNA, 1240 bp, Homo sapiens
  source 1..1240: mol_type = genomic DNA
ctgtgtttttttctaagccatgtttgaacagtgtaattatttgtgatgtacaaagaaaggaggaaaaaaattagaatgcagaattgttgctagggatttgcaaacatcatggaaaaagtagtacaaacccttgccaagatcccagaactctaatctctggatcctgatctccaaaggaaggctagaaaattctgccttggcctaaaagtcttgagaaaatgagattgaaaactgttgataaaatcagctgggagaaaaatatataaatgttcttggcccaagtgaccaggattagtatttaaaatttatgttgttaagtaaattcaaatacaaaatcaaagaaaacttttgtctagcaacatgactgcttatggtatgtgttctcttactggttaaatgtaccatagacatatccacacgcacacacacaagaaagttccagtttgaagaacttgaccttttctctcttatgtattttttccttcatttatttacctttgcagttccaaataaatttagaaaactactccctagacaattgataaggcagaatgttaactggccaaaagtttctgggccaccttttagccctcagggagaattgaagggagatatctgtgaggtctggtcataggcacatggaaaggaaacaggaagtaaccatagtaaccactacctaggctagaaaaggtgattctgtagggcctgagtggttccatccattttgaatgaaacttaaatgcaaaggcagagagcaagcctacacacacacacacacacacacacacacacacacacacacagatcccgaggctcaaaaacaataaacctaaacagagtgactagcactttcagtgactcaagacccagtcccctacaaattctaaacatcttagcttcattttaaatggtcagcgagagcttaggaaaggagatgtcctaggcacagtcttctttcttcctcccttcctctcatagaaaaagccctaaccttgtagaataaaacctactgtcaaagtaccttctcatttaggacatttttaggaaactgttcccatctatccactgagtgtcattctcctcttgaacctctgagttctatggatctccctgtgtcttctcactgtacaaatagagtcacttcctcctgctgaacattgctcaaattgcctcaaaatgtggtcccagcatcacctgggaacttattagaaatgtaaattcacataaactgttaaaatgg

SEQ ID NO: 69 - DNA, 328 bp, Mus sp.
  source 1..328: mol_type = genomic DNA
gtgcactgtaaggaaaaagacaaattacccagagctattacagtcattagtcccatcagcctgggaaggcctcccactgcattttccgtgcagtgtatagagcggtcctggggcttatcacatcttgcagggggtcgtcttctgccaggcatagtcacatagcttgacatttcacttaaaaaaaaaaaagcctgggctctcatgctccagccaggcttctgtttcctggcttctcaaccagtggctaaaataaaggcagccagcccagactgaaggctgtggccattcatctaaatgcagaaatagaaatgcatcccttccagaggct

SEQ ID NO: 70 - DNA, 652 bp, Homo sapiens
  source 1..652: mol_type = genomic DNA
ctgcaatgcaggtatgacacttttgaaaagagacaaggaagaaatgaggatggggaaagagcttcagactacatagcatttcggaaaagatcttggccaggttgatagagagcccctaagcaaaggggcctgtagaggagtccatcattaggcaaaaatgacctgacactagttctcttgcagtgttcagtcattggctgtgaggaactctggagagggcagccttggcaaaaacaccacagtcgattggatggtatggcagctgggggctatcagtcatctatgctctgcactaactctctccatggctgccacagtctactcttttaatgcatgaatctacttctccacgcatctttaagattcaggtacttcatacttctcatggacttcacctcctaggggccttccttcttaaaatgttctcttctattggctctgatagtgctagacatcattcattttcccccagccattccctggtttcctgggttgactcatctttctctgaataagtttttaaataatgggggggatctcctcaggaacttcctataggtcttcctcttaaagatggagatgagtgagacaaattaaagccctcatcaatgcaattgttctcaggacaataactgaaattcactatcttcct

SEQ ID NO: 71 - DNA, 750 bp, Mus sp.
  source 1..750: mol_type = genomic DNA
tattgatctcatttttaatctgccatatcaggacataatatattaaccctaatcagaagattgcaagcggcaggagagagggaaggagagggcgccagggaactttcggcagtgaaatccctgaagcttgtttgggggggggggggggtcagatgcttgaggctacctttgggaagggtaaaaatgtggctcacaagtactttactcttcttttccttccactttgtgtttcttgctctcagccaactcggctctccttgctgccttcagagaaactccacactccatagatacccctcctaagcagctttagatttgtcctactcgtgttttggctgttgtcacactctcctgcagaggatcaggccctctcacatcctttctgacttctcactctttgggggagtcttgggggaaactttcttggacctgggtttgtctgggttggtctatttctcttcccaaggcttaggaatacagctctttgctgagcaggtcttaaagggagacttgagctggcactactatttaaggtccttgtgatgaagcatctctggacatccctgcaactgagcataggaatatcaaccaggggggtgggggtgggggtggggagaaccaaacctgggctctgttcctgtttgtatgaggtctgtgagttaaacatccaaattatagcttcctctcaaagtatgtggaccatctacgtacttagcagtagtttgggaagactcttactgtcaaggaagt

SEQ ID NO: 72 - DNA, 760 bp, Homo sapiens
  source 1..760: mol_type = genomic DNA
tattgatttcatttttaatctgccatatcaggacacaatacgttaaccctaatcagcagattacaagtgacagagagtttagtggaaactctgtaagggagcaggggctccgggaaacttcttgacaatgaaaatcttctctgaggcttaagagtttggatgctgaagattacttttggggaggggagaaacattcctcagaaacagtctacatttttttcttccactttacatttctttatctcccagttgcccattctactgctacactttgggaatgcttaacacccaagaaatacctcccaagtatttttaggaggtcggtccctgaagcagcttgacttttgtcatagtctccaacggacgtttatgctttcatttcatcccagctgatttctcattcatttgacggaatttctccaaaggataatacctgagttgatctgtttcttttcccagggccttgggaattgtgctgtccattgaatgggttttggtcatgggggtgggggcacagactgagcctggcattttttaaagccattgaaatgatgcctcactgttcatcactgcagctgagcttatcaagtcaaccaagaagaaaacctaggccctggtcacgtttgtgctatgcctgtaattagctgacacgaggtaaatgtccaaagtcgaccttcccttccatcactgtatttatactatcaattttatttgggaatgtcatggtctgggaagactcagccactatttcgaaggcaat

SEQ ID NO: 73 - DNA, 750 bp, Mus sp.
  source 1..750: mol_type = genomic DNA
atcttggttagttgtccctactttattaggaccaaacaaaattcacacatgaggtaataaattatcgtgagctctgaaggctttacttttttttgtctaatgaattaatatactaaagaatacagtaaacgcattttgtgctgctttgatcattcgcttacatgtcattaagtagtctaaggattttataatggaagggctctctttacaaattagaccaaagaacacgtttcacgcggaagatcagcatatatattgtattttacttcagatgcacatgacacaaccgtaatattagaggcagatgattaaaagagcattacctgagccctgagagtcactgaatgagctatgctaaattggttgttttctgttttgtgattaattgtgcttctaaaccatattttagtgtgctttgtttttctgtccccagtcatcccatattattaaagagaaattctgcatatctctcctggagtccaattaaggatttgcatcatgactagatttttcactttaattactgtatataaagtaggttttgacttatttccatatttatcaaactatgcagaagaagcaaaattcatcaaataaacagatatctgtggctataattgcagcctgcatcgagcacgtaggaagaaaatatgagggtccaaatttcattatgtagaacagaataaacatctgaataattaaactgtgtgtaaatcatagtcttggcatagtaaaaagaggcgacatctt

SEQ ID NO: 74 - DNA, 1229 bp, Homo sapiens
  source 1..1229: mol_type = genomic DNA
gtcttgaatagcttttattcattttccaaggtattttgagatgatggatgaaaataatttatatttaacagtagctaagaatatgaaaattttactcctcttagaattatttctaataagaaagtttagatattaaaataattggaaactaaagatgaattatttgtttgcttttacacatgctttgtattagataagcttgcttctgaagttggggctaacacacttttgaggccttcttgtatggtttatggatctcagacagctgatttatgttattggcatcccaacaagaatggtaacttcataacactgactgttattttcttgacttaattcaaaatcatatattatctttctgaaaaatagatctcagaatacttcagctttcccaaaagtcacaggacaccatagtcattaccgcttgatgaagtataaatataatgtctctttcttcaaaatttagtgtcagaataaatacacttacatgctttctcaaacctaagttaaataataattttctttaatccaaacatattaaatatgtagcacatatacattaaaattaaccagatcaagcagtattttaattcagttgcttaattacatcttctggtgaataataggttttaccagaaaaaaaattttagactacttgaaaacgatggtgtaatacatttttcttacagtatctttattaaagatacatcaatgtacacatatatctttgtatttttacaatagcattaactttggttctaccattaacattttataagatcttttcctatgaaaacaaataccaatttactgtaatcatggttacttacagtatctaaatccattattgtaaaaaggcaaacctgtgataggcatcacatggagaataaaaaattaagagactaattgtaatattaaataagcaatcttgttatatagatacatgtaatattgcatgttgcatgtattgttactcagagtagaaagaacatgtgagaagtttcctaacttggataagaacatcacaaatttctattctcacacaaaatgccaatttactagttccctgaagattttacttagataactgttatgcaacaccaaataaacacaagctgaatttctgtttattaacacaatttaagtctaattccaagcttttacaggcttggcccatggtacataagcgtactgagttttatttctggttttacaagaatggaaatttt

SEQ ID NO: 75 - DNA, 750 bp, Mus sp.
  source 1..750: mol_type = genomic DNA
acgtccacttagatgactcaaatcaaatgtgcttggacaggatctctccattttcagcccactcctcatcccagtaaatgataccggcatccactcagtggatcaaacaagaaacccagggatctccatccttccccttccttcctgatgatgcttccgccctgagagagactcgcccgcatcctttccttttctctctcagaccggctttggtcatgctgctgtcagtagttcacttggaaacagaactcatttctgaaatggtttctctgtttcccttccttgacggctctggggaagagggtgccactctgggaggaattggtttcctgctgtgagagacactgccagtggtactaattggattaattgatttgcccaaggggaggtgtgtgtgatgcctttctgctgaacttcctttaaaaacagcacttccattcagaaagccagaccaagtgctattaacgaattaattccgattagtgctgcccaagcactgagaccttgaggcaaccccgttcagtcatcttgggtctggcctgctaataaagtgaaaactatctgggcagcagaggtgattattttgatgcctgcctccattcctggagggaaaaagagcagggtgtgcgttcaccagtctactgagggaattaaccaccttcaaattctaagttattaattccccaacatggcagtcatatccgtgaaaatggatttggggcatatgtctgtgtcctctgtagccttctt

SEQ ID NO: 76 - DNA, 1398 bp, Homo sapiens
  source 1..1398: mol_type = genomic DNA
acgtaacattttacatagagcctcttcccgcatgatttgttgactgtctgagcattttatcttgaatcaggaaatctctcccttgagctgtgatttctactttcatggccaaaactttttcaatgctgagaactgaaattgctagcttcccctgtctgcatttggagtgggaacttcatctccaaacacagtggaatcctgtctcctctagccctgcaaccccggcattaaactggtaacaagtgcttccctcggcagccccatgccctcctccagcatccgcctcaaccccgcaggttgtatttgtgccttcagagtagcactacaagtgaagaagacctgccctccttggcttcatgaccagagctgaagggcaggtaaggcgagacagtacacggattctaagaaataccggagaagcttctgggagatgcctgtgcccagagccctatgtttcagtttggatcagaggcaaatgatgtctgaatcagcattgctgggaaaaatcaccgaggtttcctctgagcagtgggattggggggtgtggggggcaggtgaggtagagggaggaagtttgcagggctattctgccttccgtattgtaaatgtttttagtggtggaattgattttcactttgtatgtaagtgtgtgttaggggtacttttcagataaaaacttaaaatcagggccgggcactatggcttttgcctgtaatcccagcactttgggaatccaaggtgggtggatttgtttgaacacaggagttcgagagcagcctggccaacatggtgaagcctcatctctacaaaaaatacaaaaattagccaggcctggtggcatgcacctgtagtcccagctacttgaggggctgaggcaggaggatcacctgagcccaggagctagaggctgcagcgaacctagatcacgccactgcactccagcctgggtgacaaagtgagaccctgtctcagaaaataaaataaagattttgacatagaagccacctgtctatggccttatcctcctctgtcactgattaagaggaactttctctatggaaatctctgaagaatgtgctgtcatcctcctcctttgcctccctctctcgagcacgcacctccctgcccagaagcaagagacacagcttgctccccatcaaaggcaaaagtgccctgtgcttcaacgtgctagacattcgggcgccttcatgcttttaaaataccactgattaaatcacattttaagttttataagatgtcaagtcaatgtcgttatgattaacatgtaaagaaagtgtattttaaaataaagttagaagcatagctgtgtcttaattttttttgtttttgagacagtcttgctctgtcaccgaggctggagtgcagtggcatgatcat

SEQ ID NO: 77 - DNA, 443 bp, Mus sp.
  source 1..443: mol_type = genomic DNA
gatagttgcctaagcagccacagaacagttggaaagatccttttagagttaggcaccagcttggtggttatcagagaagtctaattgcaattaaaatagaacgttttctggcactgaaggcttttgttttttcaaatacatttgcacaagaatgcacaggtggggacctgtagtttttctttttcataaagcaaagatattcactcaaatgatactataattgtggaacgacttgcttacataaggacatgcatccttatgtaagttagtgtcttagaaatgaatgagttaaaattaactctatgccttgcctaaatgtaaaacccaactgtgacacctccttctgtgatgacagaaaaacaaagtttcagggtatgaatgttttcaacattagaataaaacatatataatgaaggtccccttcgttttaacatttatggtat

SEQ ID NO: 78 - DNA, 444 bp, Homo sapiens
  source 1..444: mol_type = genomic DNA
gatagttgcctaagcctaagcatccacagaacagttggaaagatccttttagatttaggcaccagcttgatggttatcacagaagtctaattgcaattaaaatagtatgttttctggcaataaagatttatctcctaaaatatgtttgtacaagaatgtgcaggtgaggcattatatgcattattttttaaaatacaaatttttggcttaaataaaaatttgaaatttggggatgaccttcctatataaacatttatccatatgtaagttagtgttttagaaataattgagttaaaaaaacatagtctgtcttggctgtaatagccgaattatgacattttcttcgatgatgaaagaaaactaaattttcaggatatgtatgtttctcaatttaggaaaacatatatatataaaataaaggtctcttgttttcaacgtttatat

SEQ ID NO: 79 - DNA, 555 bp, Mus sp.
  source 1..555: mol_type = genomic DNA
gtctctggggaaatggaccttaaaagaatgtctggtcccattgttatgtttagccaaccgaggtatggaccatatggctccaagaaccttgcgagtatcaggcagcctctgtgacctgcaggtcaactgacccctagctactcaagaaatgtctacccaggatgggtccagatgtttttagtgcttcaagagaagtctgcatctgggaaggagctccatggggccaggctactgtcagtgattttggctgaatcagatgtgtttgcccttttgctgctggcacggaaagtcatacacatgttcctaccttttgaagggcaaagatagtcagactgtatactaacaagatgtgtgtataaaaccttgctctctccccctcttctcctctccctctttctctcttttaacctttgggtacacaggacagtgcttacaattcaggaggtctgtaaacttgaattttttttttaaatccttctctctacaacctgctaatggaaagttagcattttgtctcctgtgaaaatgagaagcaaacattttcggcatgctcac

SEQ ID NO: 80 - DNA, 931 bp, Homo sapiens
  source 1..931: mol_type = genomic DNA
ttctcttgaaagctgaaaacatctggacccattttaggtagcttcttcttgcttgagtagccaggggtcagtagacctgcaggtcactgaggttgcctgatctttgcaagttccttagagctatatggtctatatgttggttggctaaacccagcaatgggaccagacattctcctcgaaggacatttccccagagacagcaataagggattacataaggatcttcaaggtatatctttgaaataagccccaaagcagtatccagttcaaggtgatggggcatcaacaactgagaggtatattttagtctttagagagatcattttgagtcttccagagcctgggaagcagggaacccaggctacagagattatcaattgggctggctaaggaatggtgtctcctcagtgctcttcagattttttttccagaaacatctcactatacctgcccccaacaccaaatgcttgactgaaacattctattcctacccatgcctggagatataacattgttaagtctacttttctgcttcatttgtagagaaaaagagtatctcacaacccacagatctttagcacatgtcaaaatgaaacactggaagcttgttttcttggggacctatttggagcatattccaccactagaccccaaatatctcttgatcttcagagcctctgcaaaccagtgcccagaatcatgccctaagcacttttgctcacctacccagacgaatgcagtggcataaacaagtgatttcctagttcttttctagaaatagaacatttggtggccctaccatctgccagagctccttaaatcactggtttgaatgtctgatctcactgccttggaaagaaaaaaaaatggaggatatttcagatcaacagctgggctatttttccattttaatattttaagcaatgctaaa

SEQ ID NO: 81 - protein, 590 aa, synthetic construct
MTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDCVASNASVMNLLVISFDRYFSITRPLTYRAKRTTKRAGVMIGLAWVISFVLWAPAILFWQYFVGKRTVPPGECFIQFLSEPTITFGTAIAGFYMPVTIMTILYWRIYKETEKRTKELAGLQASGTEAETENFVHPTGSSRSCSSYELQQQSMKRSNRRKYGRCHFWFTTKSWKPSSEQMDQDHSSSDSWNNNDAAASLENSASSDEEDIGSETRAIYSIVLKLPGHSTILNSTKLPSSDNLQVPEEELGMVDLERKADKLQAQKSVDDGGSFPKSFSKLPIQLESAVDTAKTSDVNSSVGKSTATLPLSFKEATLAKRFALKTRSQITKRKRMSLVKEKKAAQTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL